(19) United States
(12) Patent Application Publication
(10) Pub. No.: US 2024/0409927 A1
(43) Pub. Date: Dec. 12, 2024

(54) INTEGRIN TARGETING LIGANDS FOR OCULAR DELIVERY OF RNAI COMPOUNDS

(71) Applicant: ALNYLAM PHARMACEUTICALS, INC., CAMBRIDGE, MA (US)

(72) Inventors: JAYAPRAKASH K. NAIR, WAYLAND, MA (US); ELENA CASTELLANOS-RIZALDOS, MELROSE, MA (US); BHAUMIK A. PANDYA, BEDFORD, MA (US); VASANT R. JADHAV, SHARON, MA (US); JUAN SALINAS, CANTON, MA (US); KEVIN DOOLEY, BOSTON, MA (US); SCOTT P. LENTINI, BRAINTREE, MA (US); SHIGEO MATSUDA, WOBURN, MA (US); MARK NEIL TOLENTINO, RANDOLPH, MA (US)

(73) Assignee: ALNYLAM PHARMACEUTICALS, INC., CAMBRIDGE, MA (US)

(21) Appl. No.: 18/576,572

(22) PCT Filed: Jul. 7, 2022
(86) PCT No.: PCT/US2022/073506

§ 371 Date: Jan. 4, 2024

Related U.S. Application Data
(60) Provisional application No. 63/219,182, filed on Jul. 7, 2021.

(51) Int. Cl. C12N 15/113; A61K 47/54; A61P 27/02

(52) U.S. Cl. CPC C12N 15/113; A61K 47/545; A61P 27/02; C12N 2310/11; C12N 2310/351

(57) ABSTRACT

The disclosure relates to RNA agents modified for targeted delivery to the eye. The present invention provides modified double stranded ribonucleic acid (dsRNAi) agents conjugated to an integrin targeting ligand, as well as methods of modulating the expression of a target gene in an ocular cell or tissue and methods of treating subjects having an ocular disease or disorder using such dsRNAi agents.

### CROSS-REFERENCE TO RELATED APPLICATIONS

This application claims the benefit of priority to U.S. Provisional Application No. 63/219,182, filed on Jul. 7, 2021, the entire contents of which are hereby incorporated herein by reference.

### SEQUENCE LISTING

The instant application contains a Sequence Listing which has been submitted electronically in XML format and is hereby incorporated by reference in its entirety. The XML copy, created on Jul. 6, 2022, is named A108868_1230WO_SL.xml and is 56,038 bytes in size.

### FIELD OF THE DISCLOSURE

The disclosure relates to the field of iRNA therapeutic agents for ocular delivery using integrin targeting ligand conjugates.

### BACKGROUND OF THE INVENTION

Oligonucleotide compounds have important therapeutic applications in medicine. siRNA compounds are promising agents for a variety of diagnostic and therapeutic purposes, including for ocular diseases or disorders. Despite the advances in application of oligonucleotides and oligonucleotide analogs as therapeutics, the need exists for oligonucleotides having improved pharmacological properties, e.g. serum stability, delivery to the right organ or cell, and transmembrane delivery.

Efficient delivery of iRNA agents to cells in vivo requires specific targeting. One method of achieving specific targeting is to conjugate a targeting moiety to the iRNA agent. The targeting moiety helps in targeting the iRNA agent to the required target site typically by interacting with specific receptors on cells at the target site.

Ocular delivery of siRNA is challenging because of physiological barriers separating the eye from the other parts of the body. Accordingly, there is a need for targeting therapeutic iRNA agents for ocular delivery such that subjects having an ocular disease or disorder can be effectively treated.

### BRIEF SUMMARY OF THE INVENTION

The present disclosure provides iRNA compositions and methods, which effect the RNA-induced silencing complex (RISC)-mediated cleavage of RNA transcripts of a target gene in the eye. The target gene may be within an ocular cell or tissue, e.g., an ocular cell or tissue within a subject, such as a human. The use of these iRNA agents enables ocular delivery of the iRNA agent and the targeted degradation of mRNAs of the corresponding target gene.

In one aspect, the present invention provides a method of inhibiting the expression of a target gene in an ocular cell or tissue comprising providing to the ocular cell or tissue an iRNA agent comprising a sense strand and an antisense strand, wherein at least one of the strands is conjugated to at least one integrin targeting ligand.

In one embodiment, the integrin targeting ligand is selected from the group consisting of RGD peptide ligands, RGD peptide mimetics, fibronectin, collagen, laminin, vitronectin, fibrinogen, thrombospondin, and glycoproteins (e.g., tenascin C, osteopontin, and nefronectin).

In some embodiments, the integrin targeting ligand is an RGD peptide ligand. In one embodiment, the RGD peptide ligand is a cyclic RGD peptide ligand. In another embodiment, the RGD peptide ligand is a monovalent cyclic RGD peptide. In another embodiment, the RGD peptide ligand is a bivalent cyclic-RGD peptide. In another embodiment, the RGD peptide ligand is a trivalent cyclic-RGD peptide. In yet another embodiment, the RGD peptide ligand is a tetravalent cyclic-RGD peptide.

In another embodiment, the RGD peptide ligand is Cilengitide.

In some embodiments, the integrin targeting ligand is selected from the group consisting of RGD peptide ligands, RGD peptide mimetics, fibronectin, collagen, vitronectin, laminin, fibrinogen, thrombospondin, glycoproteins (e.g., tenascin C, osteopontin, and nefronectin), an RGD peptide ligand, a cyclic RGD peptide ligand, a monovalent cyclic RGD peptide ligand, a bivalent cyclic-RGD peptide, a trivalent cyclic-RGD peptide, a tetravalent cyclic-RGD peptide, Cilengitide, and tetrahydronaphthyridinyl nonanoic acid derivatives. Non-limiting examples of small molecule integrin targeting ligands are described in U.S. Pat. No. 6,410,526, the entire contents of which are hereby incorporated herein by reference.

In one embodiment, the iRNA agent comprises the structure

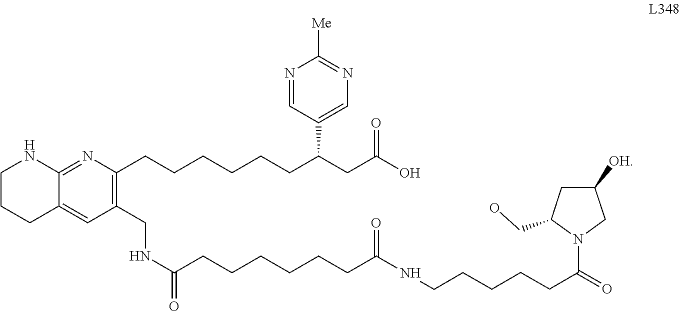

In one embodiment, the iRNA agent comprises the structure

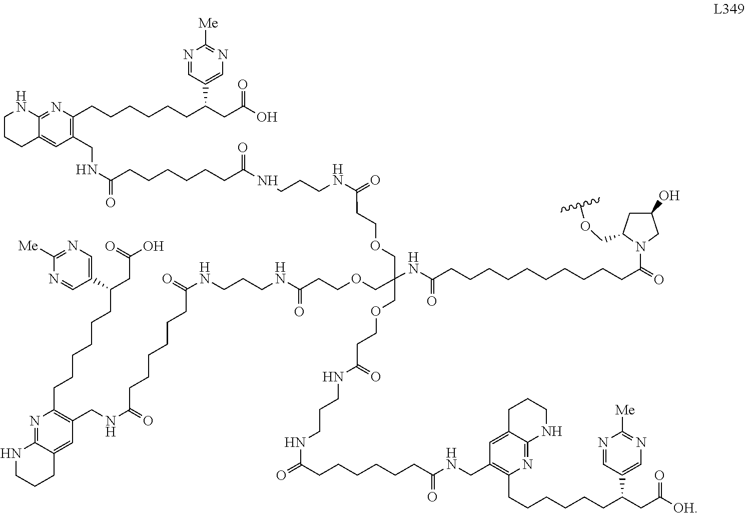 [SMILES: *OC[C@@H]1C[C@@H](O)CN1C(=O)CCCCCCCCCCC(=O)NC(COCCC(=O)NCCCNC(=O)CCCCCCC(=O)NCc1cc2c(nc1CCCCCC[C@@H](CC(=O)O)c1cnc(C)nc1)NCCC2)(COCCC(=O)NCCCNC(=O)CCCCCCC(=O)NCc1cc2c(nc1CCCCCC[C@@H](CC(=O)O)c1cnc(C)nc1)NCCC2)COCCC(=O)NCCCNC(=O)CCCCCCC(=O)NCc1cc2c(nc1CCCCCC[C@@H](CC(=O)O)c1cnc(C)nc1)NCCC2]

In one embodiment, the iRNA agent comprises the structure

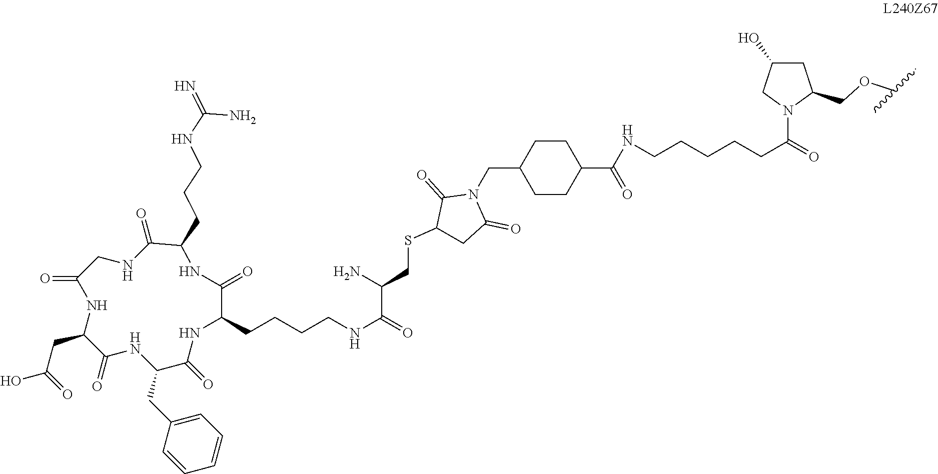

In one embodiment, the iRNA agent comprises the structure

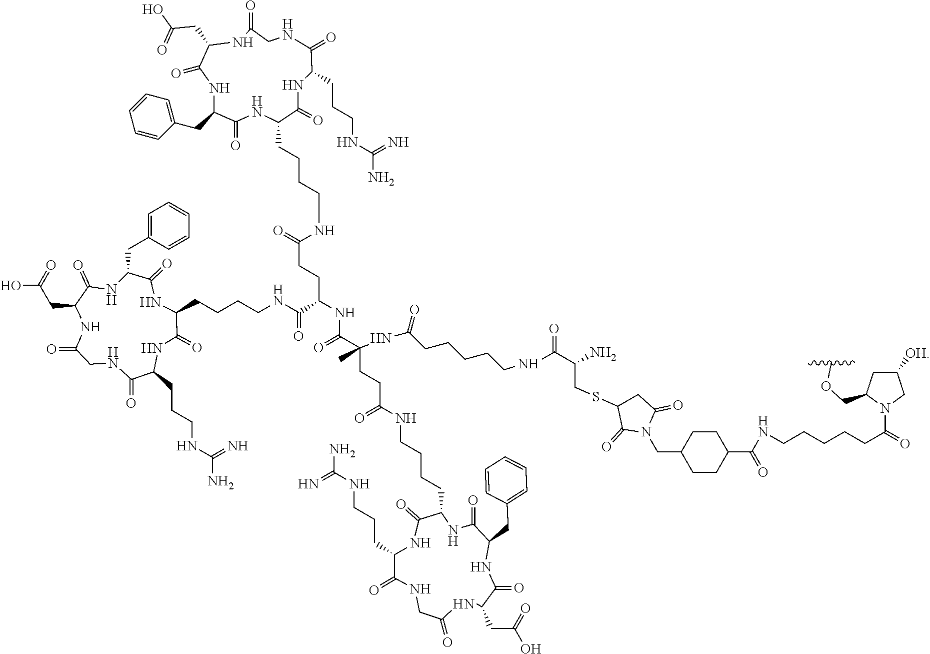 [SMILES: *OC[C@H]1C[C@H](O)CN1C(=O)CCCCCNC(=O)C1CCC(CN2C(=O)CC(SC[C@@H](N)C(=O)NCCCCCC(=O)N[C@](C)(CCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)N[C@@H](CCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C2=O)CC1]

In one embodiment, the iRNA agent comprises the structure

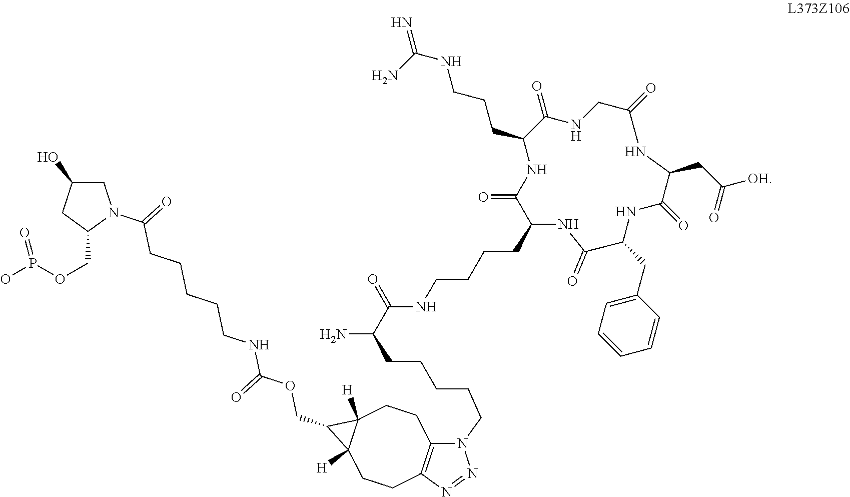

In one embodiment, the iRNA agent comprises the structure

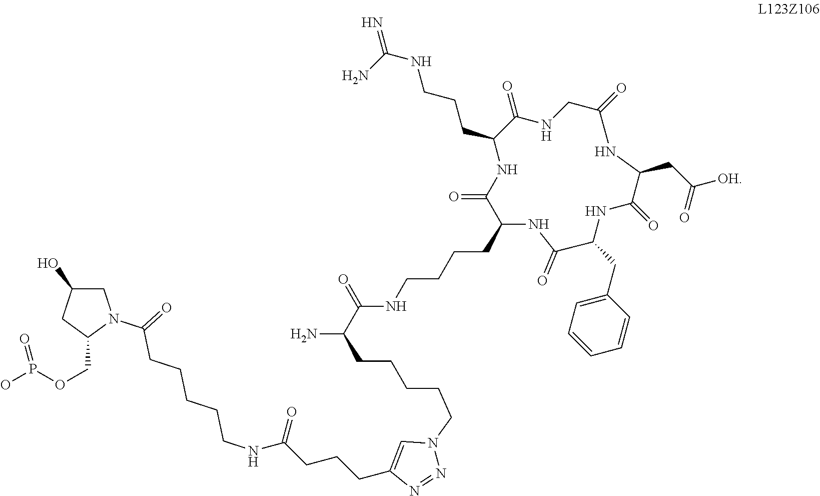

In one embodiment, the iRNA agent comprises the structure

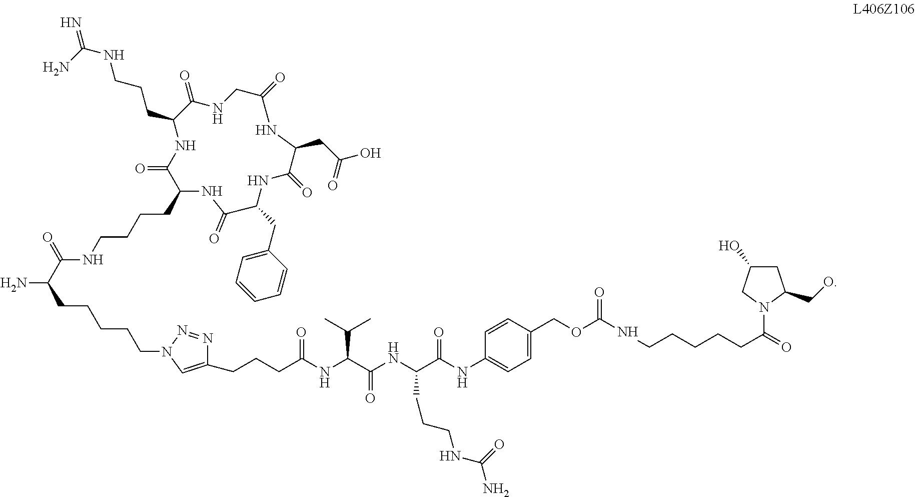

In one embodiment, the iRNA agent comprises the structure

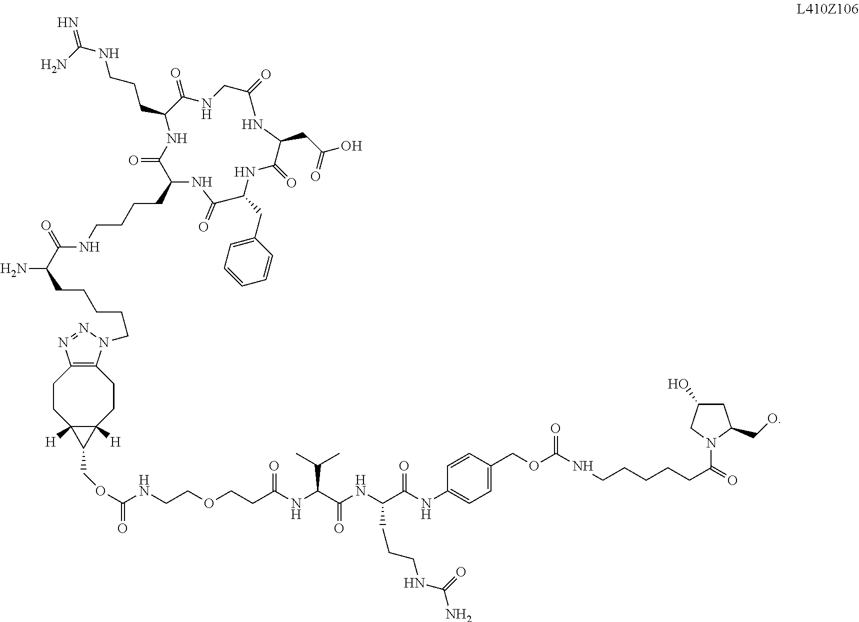

In one embodiment, the iRNA agent comprises the structure

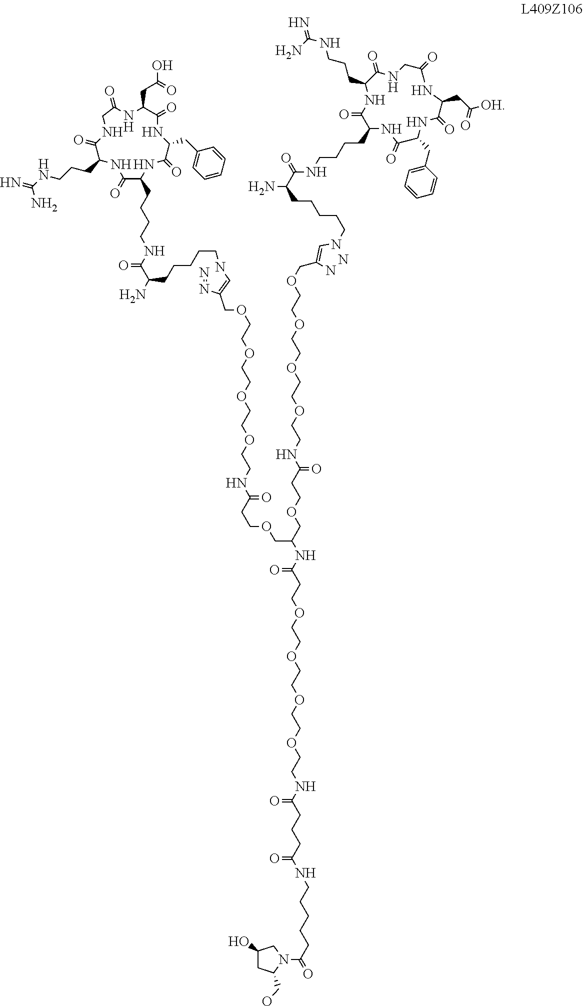 [SMILES: N=C(N)NCCC[C@@H]1NC(=O)[C@H](CCCCNC(=O)[C@H](N)CCCCCn2cc(COCCOCCOCCOCCNC(=O)CCOCC(COCCC(=O)NCCOCCOCCOCCOCc3cn(CCCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)nn3)NC(=O)CCOCCOCCOCCOCCNC(=O)CCCC(=O)NCCCCCC(=O)N3C[C@H](O)C[C@H]3CO)nn2)NC(=O)[C@@H](Cc2ccccc2)NC(=O)[C@H](CC(=O)O)NC(=O)CNC1=O]

In one embodiment, the iRNA agent comprises the structure

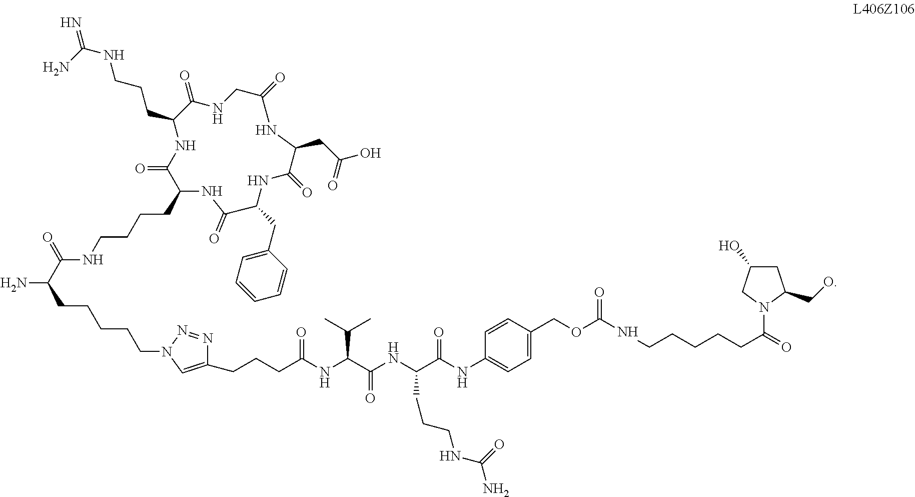

In one embodiment, the iRNA agent comprises the structure

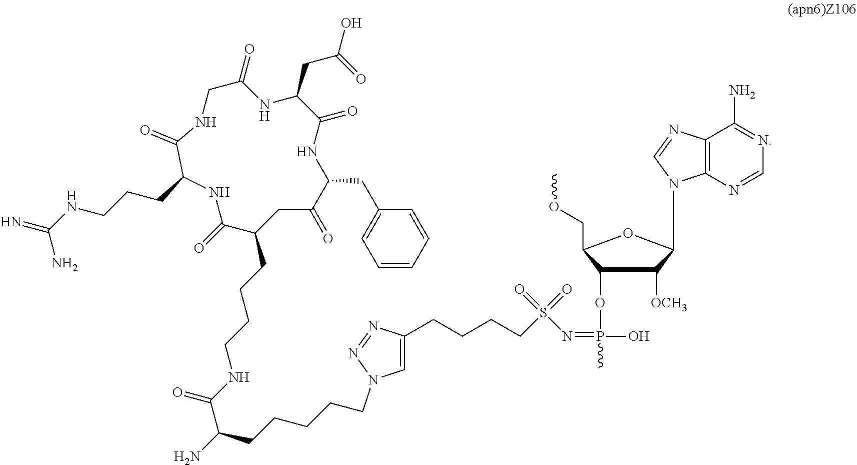

In one embodiment, the iRNA agent comprises the structure

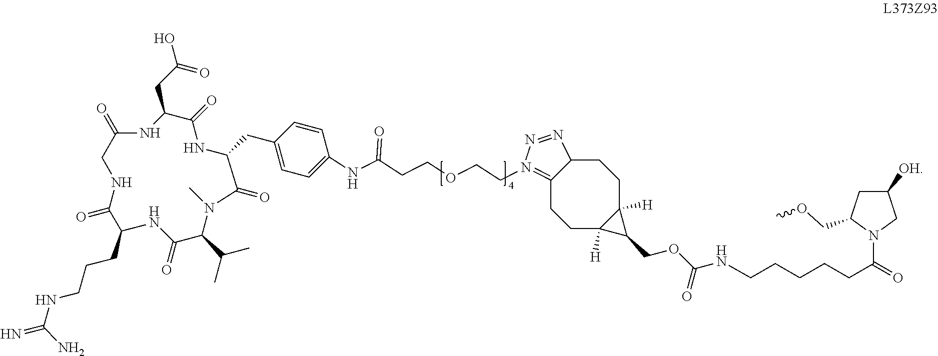

In one embodiment, the iRNA agent comprises the structure

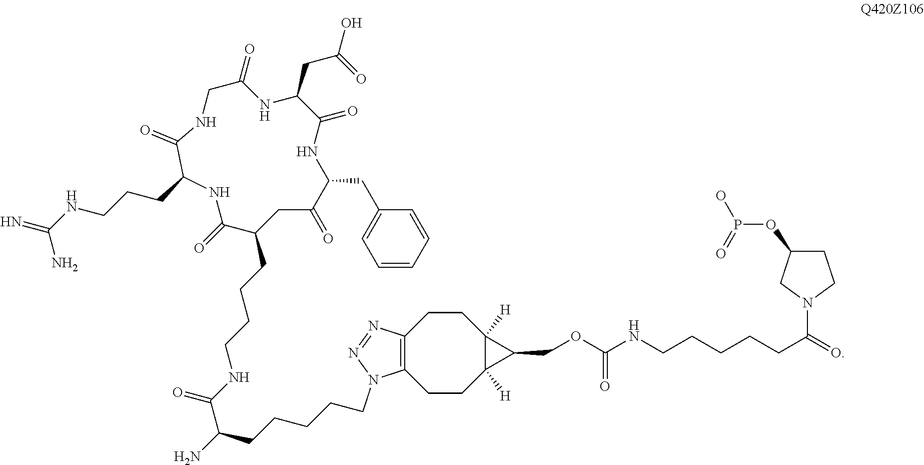

In one embodiment, the iRNA agent is conjugated to the integrin targeting ligand by maleimide-thiol conjugation. In another embodiment, the iRNA agent is conjugated to the integrin targeting ligand by Cu(I)-free click conjugation. In another embodiment, the iRNA agent is conjugated to the integrin targeting ligand by Cu(I)-catalyzed click conjugation. In another embodiment, the iRNA agent is conjugated to the integrin targeting ligand by amide coupling conjugation. In another embodiment, the iRNA agent is conjugated to the integrin targeting ligand by thioether conjugation. In another embodiment, the iRNA agent is conjugated to the integrin targeting ligand by oxime conjugation. In another embodiment, the iRNA agent is conjugated to the integrin targeting ligand by TCO/methyltetrazine (Mtz) peptide conjugation.

In one embodiment, the iRNA agent and integrin targeting ligand conjugate comprises a cleavable cRGD-iRNA conjugate. In another embodiment, the iRNA agent and integrin targeting ligand conjugate comprises a divalent cRGD-iRNA conjugate. In another embodiment, the iRNA agent and integrin targeting ligand conjugate comprises an internal cRGD-iRNA conjugate.

In one embodiment, the integrin targeting ligand is incorporated into the sense strand. In one embodiment, the integrin targeting ligand is conjugated to the 3′ end of the sense strand, to the 5′ end of the sense strand, both 3′ and 5′ positions or to an internal position on the sense strand. In certain embodiments, the integrin targeting ligand is conjugated to the 3′ end of the sense strand. In another embodiment, the integrin targeting ligand is incorporated into the antisense strand. In certain embodiments, the integrin targeting ligand is conjugated to the 3′ end of the antisense strand, to the 5′ end of the antisense strand, both 3′ and 5′ positions or to an internal position on the antisense strand. In certain embodiments, the integrin targeting ligands are conjugated multiple internal positions on sense strand. In certain embodiments, the integrin targeting ligand is conjugated to the 3′ end of the antisense strand.

In one embodiment, the target gene is selected from the group consisting of myocilin (MYOC), Ras homolog family member A (RhoA), vascular endothelial growth factor A (VEGFA), SSB (small RNA binding exonuclease protection factor La), optineurin, Carbonic Anhydrase 2 (CA2), Rho associated coiled-coil containing protein kinase 1 (ROCK1), Rho associated coiled-coil containing protein kinase 2 (ROCK2), Angiopoietin-Like 7 (ANGPTL7), and cytochrome P450 1B1 (CYPIB1), and combinations thereof.

In one embodiment, the ocular cell or tissue is selected from the group consisting of an optic nerve cell, a trabecular meshwork cell, a Schlemm's canal cell, a juxtacanalicular tissue cell, a ciliary muscle cell, a retinal cell, an astrocyte, a pericyte, a Muller cell, a ganglion cell, an endothelial cell, a photoreceptor cell, a retinal blood vessel, episcleral veins or choroid tissue, cornea, pupil, sclera, conjunctiva, optic nerve, iris, lens, aqueous humor, macula, optic disk, retina, ciliary muscle, vitreous humor, vitreous body, choroid, fovea, ciliary body, blood vessels, muscles (lateral rectus muscle, medial rectus muscle, ciliary muscle), ligaments (suspensory ligaments), anterior chamber, posterior chamber, limbal rings, and fovia. In a certain embodiment, the ocular cell or tissue is the trabecular meshwork™.

In another aspect, the present invention provides a method of treating a subject having an ocular disorder comprising administering to the subject a therapeutically effective amount of an iRNA agent comprising a sense strand and an antisense strand. In such methods, at least one of the strands is conjugated to at least one integrin targeting ligand, and the iRNA agent inhibits the expression of a target gene in an ocular cell or tissue.

In one embodiment, the subject is a human.

In one embodiment, the subject has been diagnosed with an ocular disorder selected from the group consisting of glaucoma, primary open angle glaucoma, primary open angle glaucoma, secondary glaucoma, pigmentary glaucoma, pseudoexfoliative glaucoma, traumatic glaucoma, neovascular glaucoma, irido corneal endothelial syndrome, uveitic glaucoma, angle closure glaucoma, normal tension glaucoma, juvenile open angle glaucoma, macular degeneration, cataracts, diabetic retinopathy, dry eyes, blurred vision, red eyes, blindness, night blindness, lazy eye, strabismus (cross eyes), nystagmus, colorblindness, uveitis, ocular inflammation, presbyopia, floaters in the field of vision, retinal disorders, retinal tear or detachment, conjunctivitis (pink eye), corneal diseases, vision changes, bulging eyes (proptosis), retinitis, diabetic macular edema, keratoconus, lazy eye, ocular hypertension, astigmatism, diabetic eye disease, hyperopia, myopia, macular edema, retinoblastoma, stargardt disease, usher syndrome, vitreous detachment, retinal disease, and cancers of the eye.

In a one embodiment, the ocular disorder is glaucoma. In a certain embodiment, the ocular disorder is primary open angle glaucoma.

In one embodiment, administration of the iRNA agent ameliorates of at least one sign or symptom of the disorder.

In one embodiment, the integrin targeting ligand is selected from the group consisting of RGD peptide ligands, RGD peptide mimetics, fibronectin, collagen, laminin, fibrinogen, thrombospondin, vitronectin and glycoproteins (e.g., tenascin C, osteopontin, and nefronectin).

In some embodiments, the integrin targeting ligand is an RGD peptide ligand. In one embodiment, the RGD peptide ligand is a cyclic RGD peptide ligand. In another embodiment, the RGD peptide ligand is a monovalent cyclic RGD peptide. In another embodiment, the RGD peptide ligand is a bivalent cyclic-RGD peptide. In another embodiment, the RGD peptide ligand is a trivalent cyclic-RGD peptide. In yet another embodiment, the RGD peptide ligand is a tetravalent cyclic-RGD peptide.

In one embodiment, the iRNA agent comprises the structure

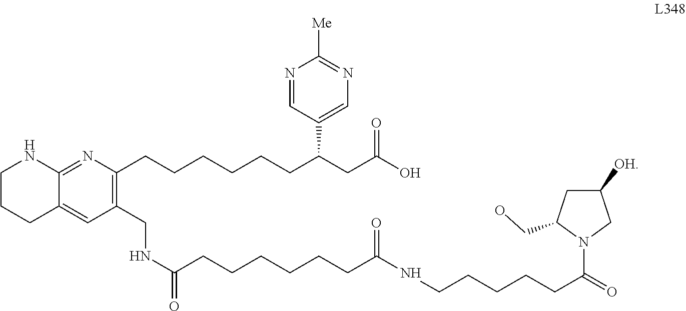 [SMILES: Cc1ncc([C@@H](CCCCCCc2nc3c(cc2CNC(=O)CCCCCCC(=O)NCCCCCC(=O)N2C[C@H](O)C[C@H]2CO)CCCN3)CC(=O)O)cn1]

In one embodiment, the iRNA agent comprises the structure

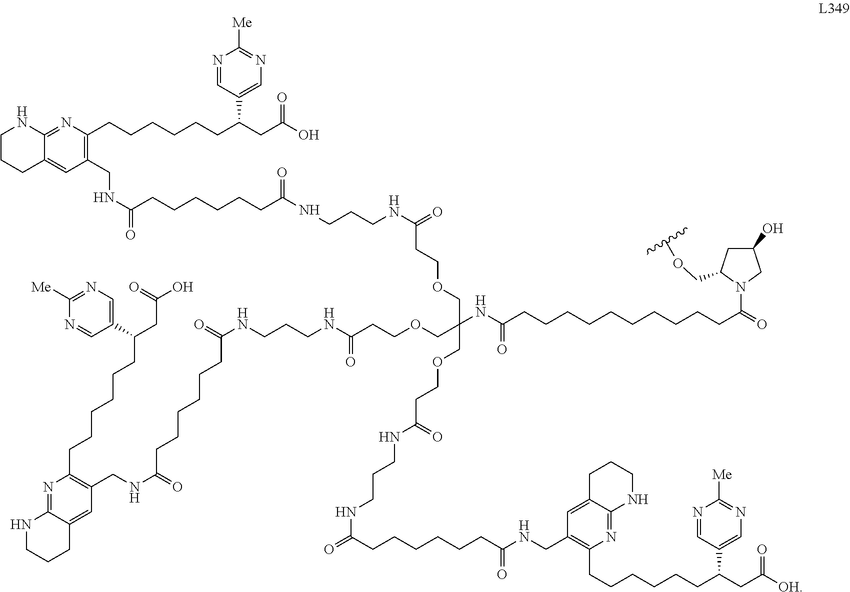 [SMILES: *OC[C@@H]1C[C@@H](O)CN1C(=O)CCCCCCCCCCC(=O)NC(COCCC(=O)NCCCNC(=O)CCCCCCC(=O)NCc1cc2c(nc1CCCCCC[C@@H](CC(=O)O)c1cnc(C)nc1)NCCC2)(COCCC(=O)NCCCNC(=O)CCCCCCC(=O)NCc1cc2c(nc1CCCCCC[C@@H](CC(=O)O)c1cnc(C)nc1)NCCC2)COCCC(=O)NCCCNC(=O)CCCCCCC(=O)NCc1cc2c(nc1CCCCCC[C@@H](CC(=O)O)c1cnc(C)nc1)NCCC2]

In one embodiment, the iRNA agent comprises the structure

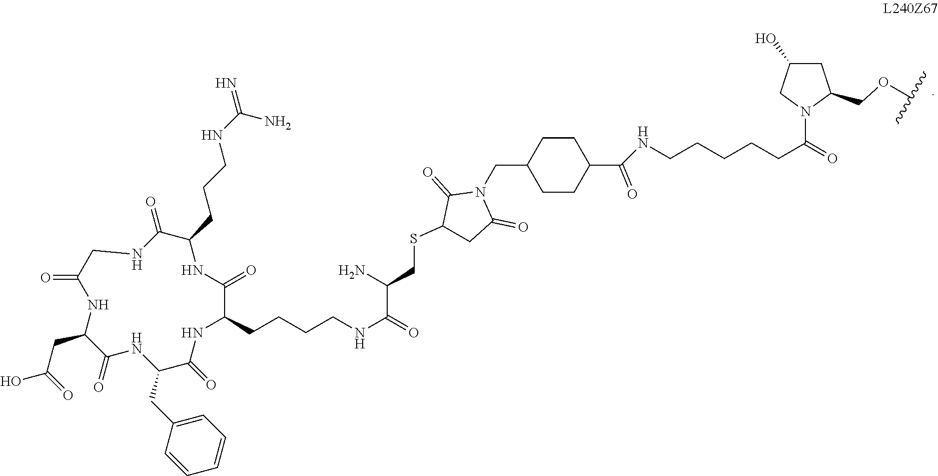 [SMILES: *OC[C@@H]1C[C@@H](O)CN1C(=O)CCCCCNC(=O)C1CCC(CN2C(=O)CC(SC[C@H](N)C(=O)NCCCC[C@H]3NC(=O)[C@H](Cc4ccccc4)NC(=O)[C@@H](CC(=O)O)NC(=O)CNC(=O)[C@@H](CCCNC(=N)N)NC3=O)C2=O)CC1]

In one embodiment, the iRNA agent comprises the structure

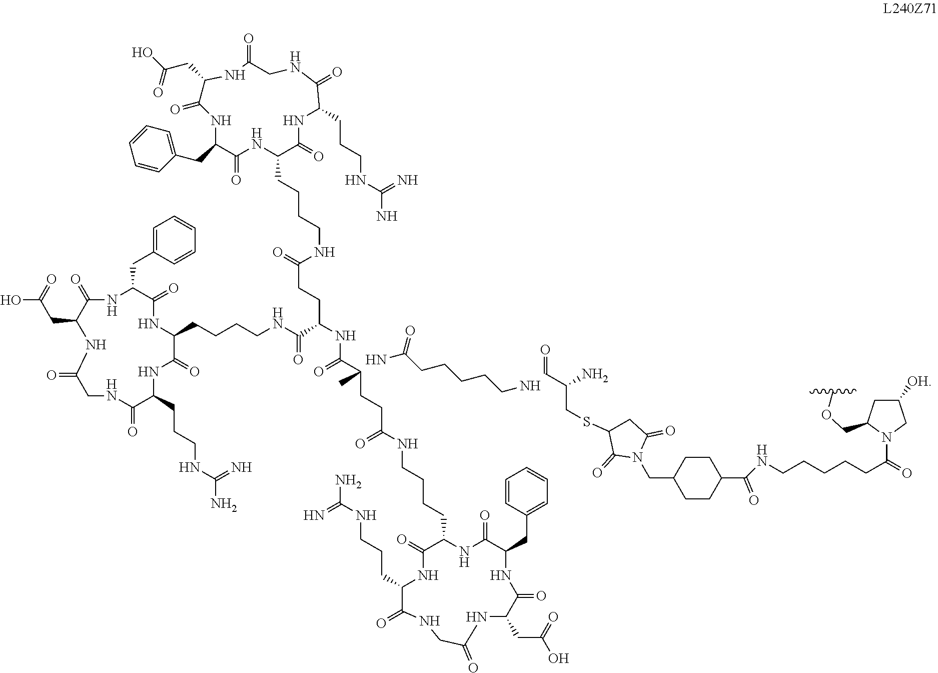

In one embodiment, the iRNA agent comprises the structure

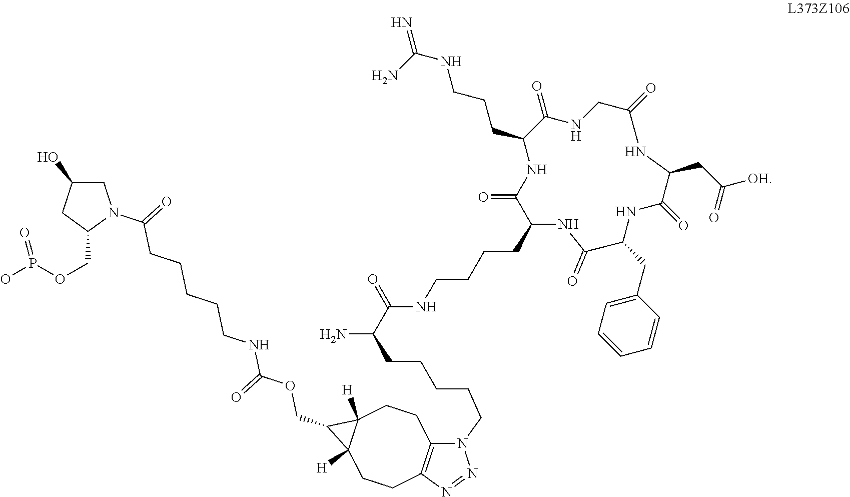

In one embodiment, the iRNA agent comprises the structure

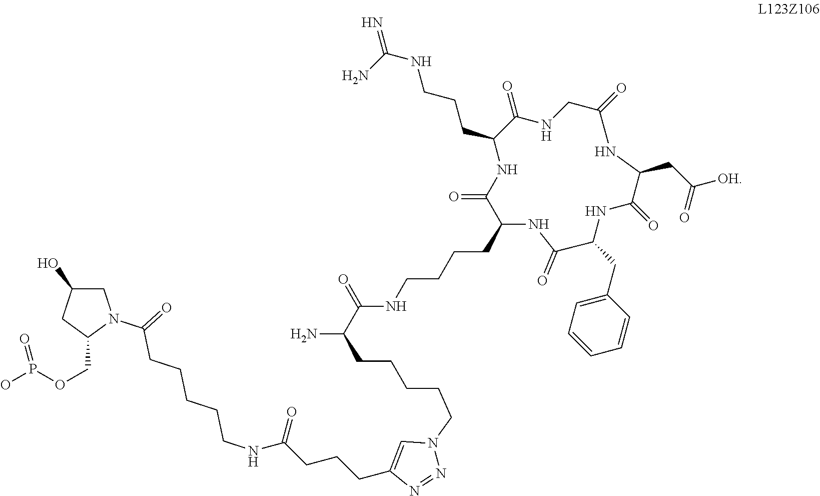

In one embodiment, the iRNA agent comprises the structure

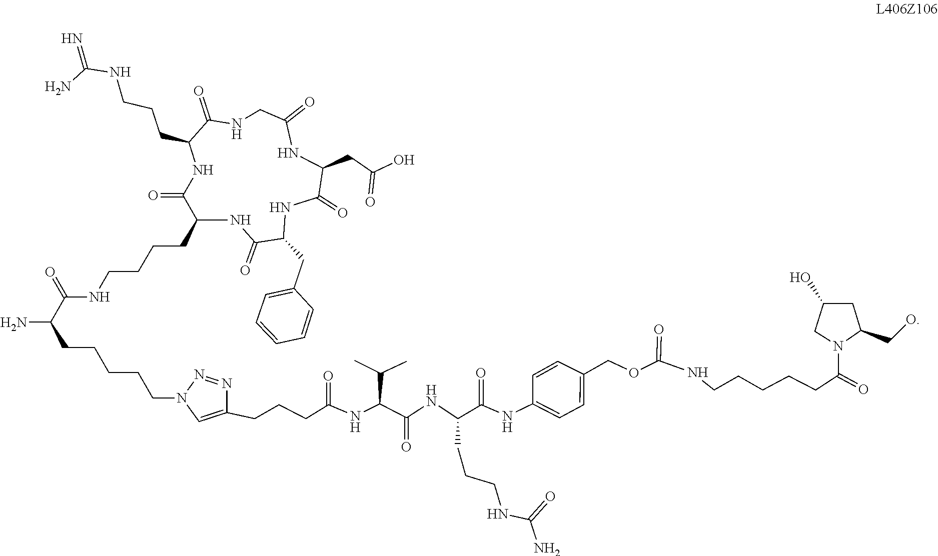

In one embodiment, the iRNA agent comprises the structure

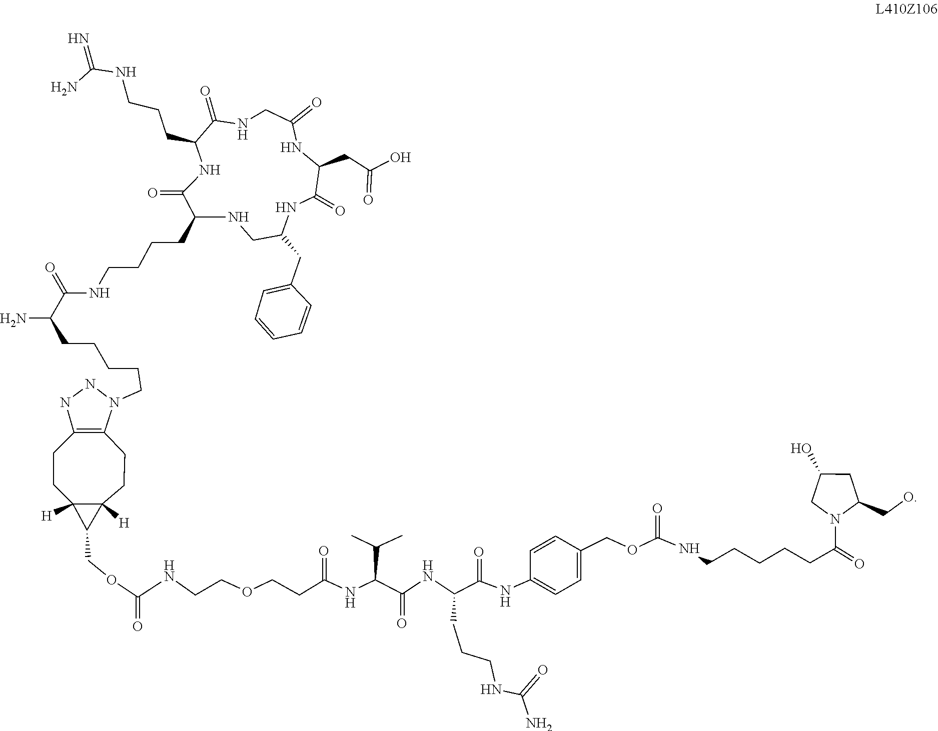

In one embodiment, the iRNA agent comprises the structure

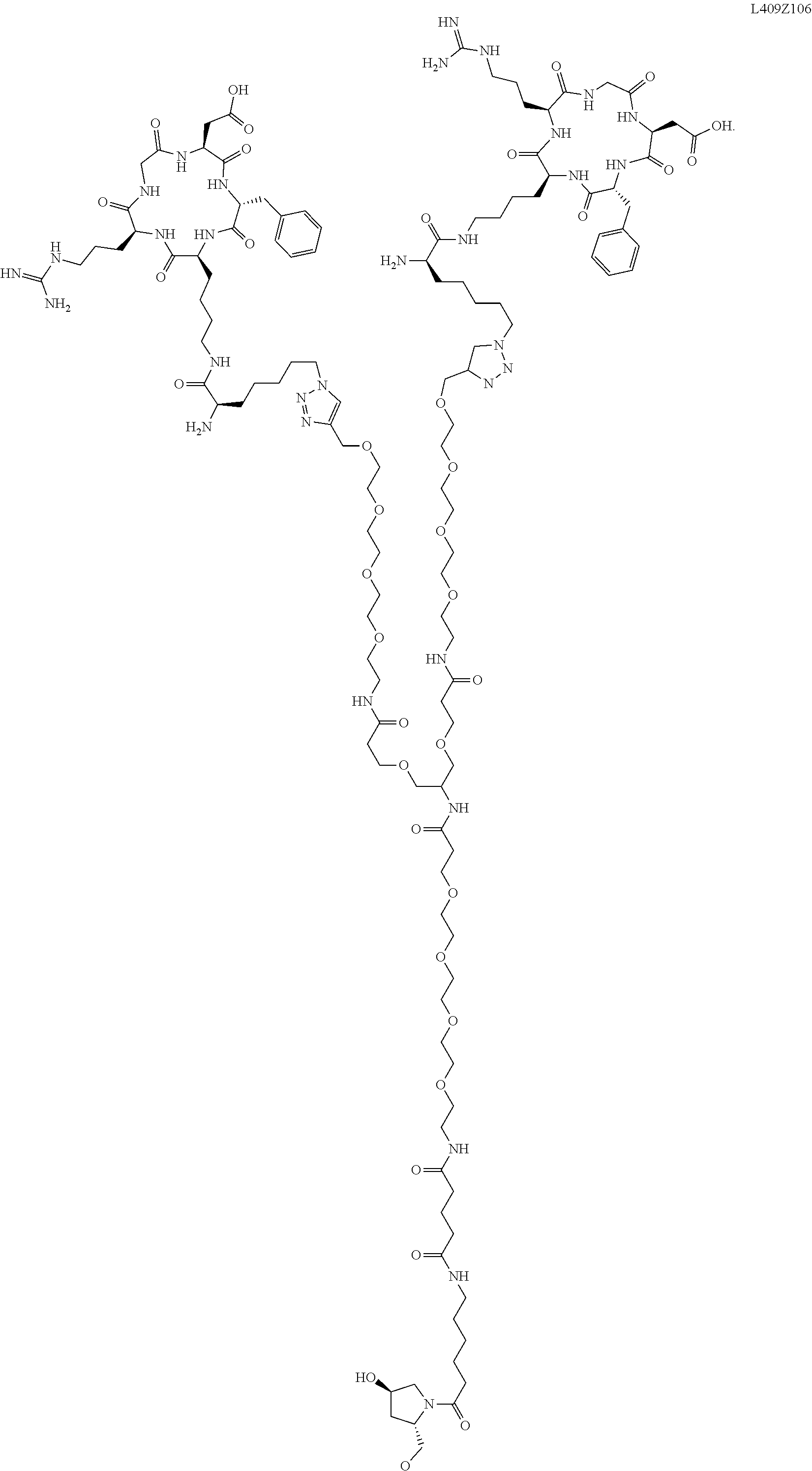 [SMILES: N=C(N)NCCC[C@@H]1NC(=O)[C@H](CCCCNC(=O)[C@H](N)CCCCCN2CC(COCCOCCOCCOCCNC(=O)CCOCC(COCCC(=O)NCCOCCOCCOCCOCc3cn(CCCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)nn3)NC(=O)CCOCCOCCOCCOCCNC(=O)CCCC(=O)NCCCCCC(=O)N3C[C@H](O)C[C@H]3CO)NN2)NC(=O)[C@@H](Cc2ccccc2)NC(=O)[C@H](CC(=O)O)NC(=O)CNC1=O]

In one embodiment, the iRNA agent comprises the structure

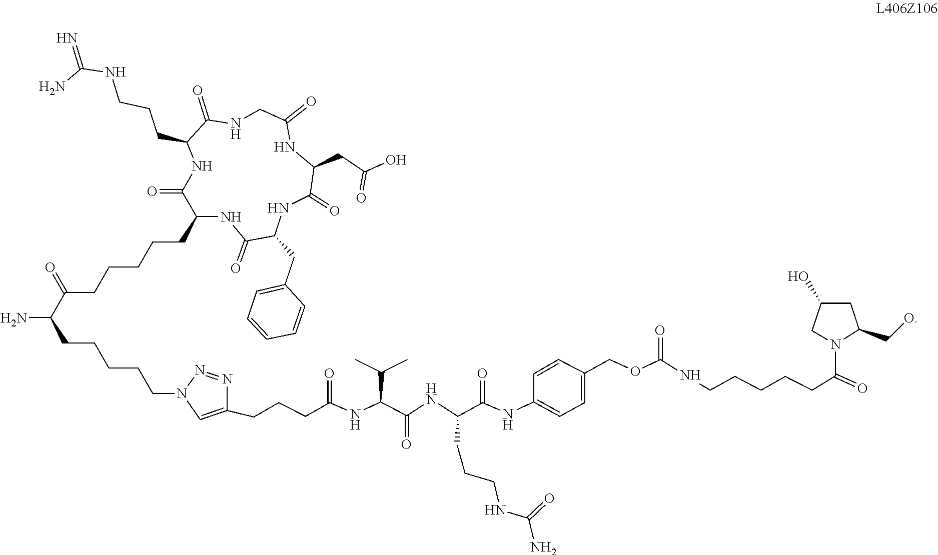

In one embodiment, the iRNA agent comprises the structure

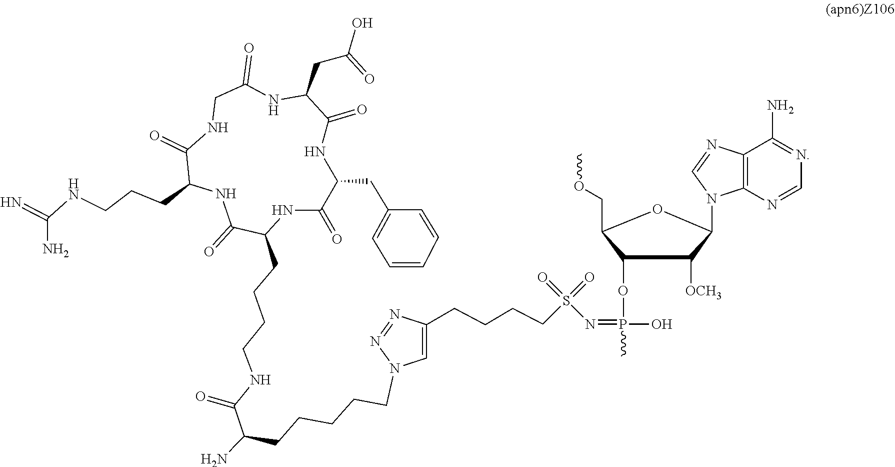

In one embodiment, the iRNA agent comprises the structure

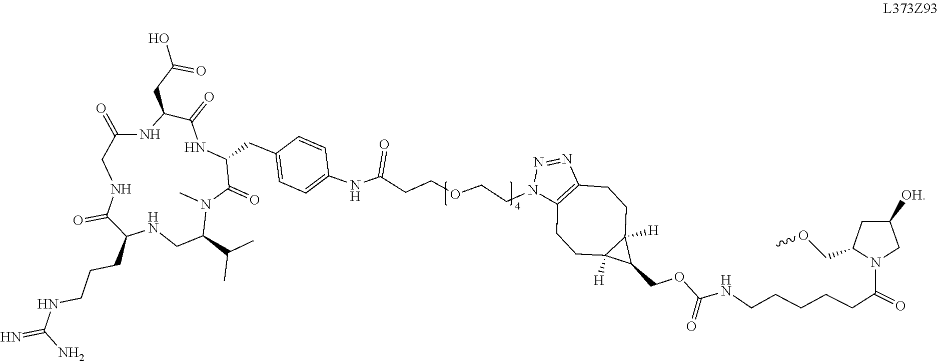

In one embodiment, the iRNA agent comprises the structure

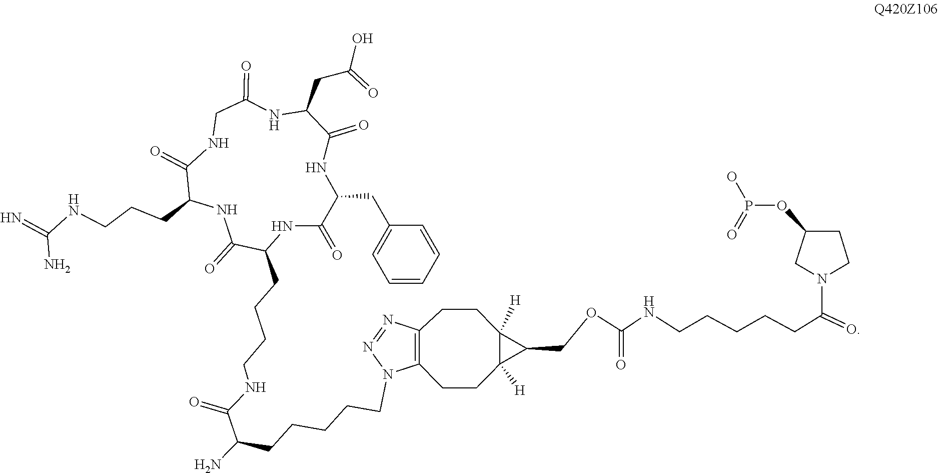

In one embodiment, the integrin targeting ligand is incorporated into the sense strand or anti-sense strand or both strands.

In one embodiment, the integrin targeting ligand is conjugated to the 3′ end of the sense strand, to the 5′ end of the sense strand, 3′ and 5′ end of sense strand, anti-sense strand or both.

In another embodiment, the integrin targeting ligand is conjugated to an internal position of sense strand or anti-sense strand or both.

In certain embodiments, the integrin targeting ligand is conjugated to the 3′ end of the sense strand. In another embodiment, the integrin targeting ligand is incorporated into the antisense strand. In certain embodiments, the integrin targeting ligand is conjugated to the 3′ end of the antisense strand, to the 5′ end of the antisense strand, 3′ and 5′ end of sense strand or to an internal position on the antisense strand. In certain embodiments, the integrin targeting ligand is conjugated to the 3′ end of the antisense strand multiple internal positions of sense or antisense strand or both strands. In certain embodiments, the integrin targeting ligand is conjugated to multiple internal positions of both sense and anti-sense strands.

In one embodiment, the target gene is selected from the group consisting of myocilin (MYOC), Ras homolog family member A (RhoA), vascular endothelial growth factor A (VEGFA), SSB (small RNA binding exonuclease protection factor La), optineurin, Carbonic Anhydrase 2 (CA2), Rho associated coiled-coil containing protein kinase 1 (ROCK1), Rho associated coiled-coil containing protein kinase 2 (ROCK2), Angiopoietin-Like 7 (ANGPTL7), and cytochrome P450 1B1 (CYPIB1), and combinations thereof.

In one embodiment, the ocular cell or tissue is selected from the group consisting of an optic nerve cell, a trabecular meshwork cell, a Schlemm's canal cell (e.g., including an endothelial cell), a juxtacanalicular tissue cell, a ciliary muscle cell, a retinal cell, an astrocyte, a pericyte, a Muller cell, a ganglion cell (e.g., including a retinal ganglion cell), an endothelial cell, a photoreceptor cell, a retinal blood vessel (e.g., including endothelial cells and vascular smooth muscle cells), episcleral veins or choroid tissue, e.g., a choroid vessel, cornea, pupil, sclera, conjunctiva, optic nerve, iris, lens, aqueous humor, macula, optic disk, retina, ciliary muscle, vitreous humor, vitreous body, choroid, fovea, ciliary body, blood vessels, muscles (lateral rectus muscle, medial rectus muscle, ciliary muscle), ligaments (suspensory ligaments), anterior chamber, posterior chamber, limbal rings, and fovia. In a certain embodiment, the ocular cell or tissue is the trabecular meshwork™.

### BRIEF SUMMARY OF THE DRAWINGS

FIGS. 1A and 1B depict schematics showing the conjugation of a αvβ₃ ligand or cyclic RGD peptide to a MYOC siRNA agent. FIG. 1A depicts the amide coupling of a monovalent αvβ₃ ligand (L348) or a trivalent αvβ₃ ligand (L349) to the 3′-end of an siRNA agent. FIG. 1B depicts the thio-malemide coupling of monovalent cyclic RGD (L240Z67) or trivalent cyclic RGD (L240Z71) to the 3′-end of the siRNA agent.

FIG. 1C depicts the copper-free click mediated conjugation of monovalent cyclic RGD to the 3′-end of an siRNA agent. FIG. 1D depicts the copper-free click mediated conjugation of cilengitide to the 3′-end of an siRNA agent.

FIG. 2 graphically depicts the percentage of MYOC mRNA remaining (relative to PBS) in the limbal ring in rats administered via single intravitreal injection of PBS, 50 μg MYOC targeting siRNA including internal C16 (AD-579842), or 50 μg of different MYOC siRNA conjugates.

FIG. 3 Graphically shows the inhibition of ocular MYOC mRNA expression by qPCR in rat eye (limbal ring-trabecular meshwork, ciliary body and iris) after a single IVT administration of AD-1488236 in a dose responsive manner (AD-579820-50 μg, AD-1488236-10, 50 and 100 μg).

FIG. 4 Graphically represents the inhibition of ocular MYOC mRNA expression by qPCR in rat eye (limbal ring-trabecular meshwork, iris and ciliary body) after a single IVT administration of AD-1488236 in a dose responsive manner (AD-579820-50 μg, AD-1488236-0.1, 1 and 10 μg).

FIG. 5 Graphically sumarizes the inhibition of ocular MYOC mRNA expression by qPCR in rat eye (limbal ring-trabecular meshwork, iris and ciliary body) after a single IVT administration of parent duplex AD-579820 at 50 μg/eye and AD-1488236 at 10 μg/eye overtime up to day 56.

FIG. 6A Graphically depicts the inhibition of ocular RhoA mRNA expression by qPCR in rat eye (limbal ring-trabecular meshwork, iris and ciliary body) after a single IVT administration of parent duplex AD-1096125 (C16) at 50 μg/eye versus AD-1565882 (cRGD) at 50 μg/eye. Administration of MYOC cRGD siRNA did not lower RhoA mRNA levels. FIG. 6B. RhoA targeting siRNAs (C16 or cRGD) did not lower MYOC mRNA. MYOC targeting siRNA lowered MYOC mRNA in a dose response manner (eyes were dosed at 0.1, 1 and 10 μg/eye).

FIG. 7 Graphically depicts the inhibition of ocular VEGFA protein expression by ELISA in rat eye (posterior cup-retina and RPE/choroid) after a single IVT administration of either parent duplex AD-901113 (C16) at 10 or 1 μg/eye, AD-1711739 (3′ mono cRGD) or AD-1711740 (Internal Sense strand position 6 cRGD) at 1 and 10 μg/eye.

FIG. 8A Summary of inhibition of ocular MYOC mRNA expression by qPCR in rat eye (limbal ring-trabecular meshwork, iris and ciliary body) after a single IVT administration of either parent duplex AD-579820 at 50 μg/eye, AD-1488236 at 10 μg/eye, cRGD SAR (3′-5′, 5′, 5′ Tri-valent, 3′ 1+1, Internal Sense strand 6, Internal SS6-Tri-valent) at 50 μg/eye or scrambled controls (dosed at 10 and 50 μg/eye). FIG. 8B. MYOC mRNA expression by qPCR in rat eye (limbal ring-trabecular meshwork, iris and ciliary body) after a single IVT administration of either cRGD 3′ mono AD-1488236 at 10 μg/eye and cRGD SAR (3′-5′, 5′, 5′ Tri-valent, 3′ 1+1, Internal Sense strand 6, Internal SS6-Tri-valent) at 10 μg/eye. FIG. 8C. MYOC mRNA expression by qPCR in limbal ring of rat after a single IVT administration of cRGD SAR at 1 and 10 μg/eye doses.

FIG. 9. Graphically depicts the inhibition of ocular MYOC mRNA expression by qPCR in rat eye (limbal ring-trabecular meshwork, iris and ciliary body) after a single IVT administration of either parent duplex AD-1751755 (Cilengitide 3′ mono) at 10 μg/eye, Cilengitide SAR AD-1751756 (5′-3′) or AD-1751757 (3′ 1+1) at 10 μg/eye.

FIG. 10. Reduction of ocular MYOC (trabecular meshwork) expression by qPCR in rat eye (limbal ring-trabecular meshwork, ciliary body and iris) after a single IVT administration of either parent duplex AD-488236 (cRGD 3′ mono maleimide linker) or AD-1751755 (3′ mono cRGD BCN azide) at 1 and 10 μg/eye.

FIG. 11A. Inhibition of ocular MYOC protein and FIG. 11B. mRNA expression in NHP trabecular meshwork 64 days after a single IVT administration of either parent duplex AD-822899 (C16) or AD-1646735 (3′ mono cRGD maleimide) at 10 and 100 μg/eye.

FIG. 12 Graphically depicts higher lysosomal uptake of either Alexa-646 labeled cRGD, Cilengitide 3′ mono, αvβ3 targeting small molecule or parent MYOC siRNA duplex (C16) at day 3.

All publications, patent applications, patents, and other references mentioned herein are incorporated by reference in their entirety.

The details of various embodiments of the disclosure are set forth in the description below. Other features, objects, and advantages of the disclosure will be apparent from the description and the drawings, and from the claims.

### DETAILED DESCRIPTION

iRNA directs the sequence-specific degradation of mRNA through a process known as RNA interference (RNAi). The present disclosure provides iRNA compositions and methods for targeting iRNA agents to the eye. The iRNA agents provided herein are designed to comprise a targeting ligand for ocular delivery. Further provided herein are methods of inhibiting expression of a target gene in an ocular cell or tissue by providing to the ocular cell or tissue an iRNA agent comprising a sense strand and an antisense strand, wherein at least one of the strands is conjugated to at least one integrin targeting ligand. The present disclosure also provides methods of treating a subject having an ocular disorder or disease comprising administering to the subject a therapeutically effective amount of an iRNA agent comprising a sense strand and an antisense strand, wherein at least one of the strands is conjugated to at least one integrin targeting ligand, thereby inhibiting the expression of a target gene in an ocular cell or tissue.

Integrins are cell surface receptors that, upon ligand binding, activate signal transduction pathways including signaling pathways involved in cytoskeleton organization and cell cycle regulation. Integrins are also involved in cell attachment to the extracellular matrix and the integrin targeting ligands comprise common extracellular matrix components, including fibronectin, collagen, laminin, vitronectin, fibrinogen, thrombospondin, and glycoproteins (e.g., tenascin C, osteopontin, and nefronectin). In humans there are at least twenty-four known integrin heterodimers composed of an alpha and a beta subunit. It is the combination of the alpha and beta subunits which determines the ligand specificity and function of the integrin. Nearly all cells express at least one integrin and the expression and/or activity of integrins can be influenced by other signal inducing molecules, such as cytokines or steroids.

Integrin targeting ligands comprise binding sequences that are recognized and bound by integrins. One such binding sequence is an arginine-glycine-aspartic acid (RGD) sequence that is present in integrin targeting ligands, including fibronectin and fibrinogen. Various integrins bind to the RGD sequence including αvβ3, αvβ5, αvβ6, αvβ8, α5β1, and αIIbβ3 integrins. The RGD sequence has been used to design various RGD ligands including RGD peptide mimetics and cyclic RGD peptides.

A small molecule antagonist of the αvβ3 integrin, compound 5f, was studied in tumor targeted siRNA delivery. However, while siRNA targeting and uptake was achieved, no RNAi activity was observed in tumor, kidney or liver (see, Coleman, et al., 2004, J. Med. Chem., 47(20):4829-4837). Another study performed in tumor cells in vitro utilized multivalent cRGD (bi-, tri, and tetra-valent) to target siRNA to tumor cells expressing αvβ3 integrin and found that increasing the valency of the cRGD peptide ligand had little effect on overall siRNA uptake, but enhanced pharmacological effectiveness was seen with an increase in valency (See, Alam, R. W., et al., Bioconjug. Chem., 2011, 22(8): 1673-1681, hereby incorporated by reference in its entirety).

Integrins are expressed in various tissues and cells of the eye, (e.g., trabecular meshwork, Schlemm's canal, and limbal ring) and dysregulated integrin expression and/or signaling activity may be involved in establishment of ocular disorders or diseases. For example, activation of the αvβ3 integrin has been linked to the pathogenesis of glaucoma (see, for example, Filla, et. al., 2017, Exp. Eye Res., 158:124-136). Accordingly, integrins expressed in the eye may be utilized as a target to direct therapeutic iRNA to ocular tissues and cells.

The following description discloses how to make and use compositions containing iRNAs to inhibit the expression of a target gene in the eye, as well as compositions and methods for treating ocular disorders or diseases related to expression of a target gene in the eye.

### I. Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a chemical structure and a chemical name, the chemical structure takes precedence.

As used herein, “target sequence” refers to a contiguous portion of the nucleotide sequence of an mRNA molecule formed during the transcription of a target gene, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of a target gene.

The target sequence is about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature. “G,” “C,” “A,” “T”, and “U” each generally stand for a nucleotide that contains guanine, cytosine, adenine, thymidine, and uracil as a base, respectively in the context of a modified or unmodified nucleotide. However, it will be understood that the term “ribonucleotide” or “nucleotide” can also refer to a modified nucleotide, as further detailed below, or a surrogate replacement moiety (see, e.g., Table 2). The skilled person is well aware that guanine, cytosine, adenine, thymidine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of a target gene in a cell, e.g., a cell within a subject, such as a mammalian subject.

In one embodiment, an RNAi agent of the disclosure includes a single stranded RNAi that interacts with a target RNA sequence, e.g., any target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes this dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (ssRNA) (the antisense strand of a siRNA duplex) generated within a cell and which promotes the formation of a RISC complex to effect silencing of the target gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, a “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In general, a dsRNA molecule can include ribonucleotides, but as described in detail herein, each or both strands can also include one or more non-ribonucleotides, e.g., a deoxyribonucleotide, a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims. In certain embodiments of the instant disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 15-36 base pairs in length, for example, about 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure may be different portions of one larger RNA molecule, or they may be separate RNA molecules. Where the two strands are part of one larger molecule, and they may be connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not, but can be covalently connected. In certain embodiments where the two strands are connected covalently by means other than an uninterrupted chain of nucleotides between the 3′-end of one strand and the 5′-end of the respective other strand forming the duplex structure, the connecting structure is referred to as a “linker” (though it is noted that certain other structures defined elsewhere herein can also be referred to as a “linker”). The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide.

In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, an RNAi agent of the disclosure is a dsRNA, each strand of which independently comprises 19-23 nucleotides, that interacts with a target RNA sequence to direct the cleavage of the target RNA.

In some embodiments, an iRNA of the invention is a dsRNA of 24-30 nucleotides that interacts with a target RNA sequence to direct the cleavage of the target RNA.

As used herein, the term “nucleotide overhang” refers to at least one unpaired nucleotide that protrudes from the duplex structure of an RNAi agent, e.g., a dsRNA. For example, when a 3′-end of one strand of a dsRNA extends beyond the 5-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-15 nucleotide, e.g., 0-3, 1-3, 2-4, 2-5, 4-10, 5-10, 6-12 or e.g., a 1, 2, 3, 4, 5, 6, 7, 8, 9, 10, 11, 12, 13, 14, or 15 nucleotide, overhang at the 3′-end. In another embodiment, one or more of the nucleotides in the overhang is replaced with a nucleoside thiophosphate.

In certain embodiments, the overhang on the sense strand or the antisense strand, can include extended lengths longer than 10 or 15 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an RNAi agent, e.g., a dsRNA, which includes a region that is substantially complementary to a target sequence.

As used herein, the term “region of complementarity” refers to the region on the antisense strand that is substantially complementary to a sequence, for example a target sequence. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent. In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, when the antisense strand of the RNAi agent contains mismatches to the target sequence, then the mismatch can optionally be restricted to be within the last 5 nucleotides from either the 5′- or 3′-end of the region of complementarity. For example, in such embodiments, for a 23 nucleotide RNAi agent, the strand which is complementary to a region of a target gene, generally does not contain any mismatch within the central 13 nucleotides. The methods described herein or methods known in the art can be used to determine whether an RNAi agent containing a mismatch to a target sequence is effective in inhibiting the expression of a target gene. For example, Jackson et al. (Nat. Biotechnol. 2003;21: 635-637) described an expression profile study where the expression of a small set of genes with sequence identity to the MAPK14 siRNA only at 12-18 nt of the sense strand, was down-regulated with similar kinetics to MAPK14. Similarly, Lin et al., (Nucleic Acids Res. 2005; 33(14): 4527-4535) using qPCR and reporter assays, showed that a 7 nt complementation between a siRNA and a target is sufficient to cause mRNA degradation of the target. Consideration of the efficacy of RNAi agents with mismatches in inhibiting expression of a target gene is important, especially if the particular region of complementarity in a target gene is known to have polymorphic sequence variation within the population.

As used herein, “substantially all of the nucleotides are modified” are largely but not wholly modified and can include not more than 5, 4, 3, 2, or 1 unmodified nucleotides.

The term “sense strand” or “passenger strand” as used herein, refers to the strand of an RNAi agent that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can be, for example, “stringent conditions”, including but not limited to, 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). As used herein, “stringent conditions” or “stringent hybridization conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs. In some embodiments, the “substantially complementary” sequences disclosed herein comprise a contiguous nucleotide sequence which is at least about 80% complementary over its entire length to the equivalent region of the target sequence, such as about 85%, about 90%, about 91%, about 92%, about 93%, about 94%, about 95%, about 96%, about 97%, about 98%, or about 99% complementary. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between two oligonucleotides or polynucleotides, such as the sense strand and the antisense strand of a dsRNA, or between the antisense strand of an RNAi agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a target gene). For example, a polynucleotide is complementary to at least a part of a target mRNA if the sequence is substantially complementary to a non-interrupted portion of an mRNA encoding the target.

Accordingly, in some embodiments, the antisense polynucleotides disclosed herein are fully complementary to the target sequence. In other embodiments, the antisense polynucleotides disclosed herein are substantially complementary to the target sequence and comprise a contiguous nucleotide sequence which is at least 80% complementary over its entire length to the equivalent region of the nucleotide sequence of the target sequence, such as about 85%, about 90%, about 91%, about 92%, about 93%, about 94%, about 95%, about 96%, about 97%, about 98%, or about 99% complementary.

In one embodiment, at least partial suppression of the expression of a target gene, is assessed by a reduction of the amount of the target mRNA, e.g., sense mRNA, antisense mRNA, total mRNA, which can be isolated from or detected in a first cell or group of cells in which a target gene is transcribed and which has or have been treated such that the expression of a target gene is inhibited, as compared to a second cell or group of cells substantially identical to the first cell or group of cells but which has or have not been so treated (control cells). The degree of inhibition may be expressed in terms of:

$$\frac{( mRNA in control cells ) - ( mRNA in treated cells )}{( mRNA in control cells )} \cdot 100 \%$$

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, optionally via intraocular injection, intrathecal, intravitreal or other injection, to the bloodstream (i.e., intravenous) or the subcutaneous space, or administered topically (e.g., by an eye drop solution) such that the agent will subsequently reach the tissue where the cell to be contacted is located. For example, the RNAi agent may contain or be coupled to a ligand, e.g., a lipophilic moiety or moieties as described below and further detailed, e.g., in PCT/US2019/031 170, which is incorporated herein by reference, that directs or otherwise stabilizes the RNAi agent at a site of interest, e.g., the eye. Combinations of in vitro and in vivo methods of contacting are also possible. For example, a cell may also be contacted in vitro with an RNAi agent and subsequently transplanted into a subject.

In one embodiment, contacting a cell with an RNAi agent includes “introducing” or “delivering the RNAi agent into the cell” by facilitating or effecting uptake or absorption into the cell. Absorption or uptake of an RNAi agent can occur through unaided diffusive or active cellular processes, or by auxiliary agents or devices. Introducing an RNAi agent into a cell may be in vitro or in vivo. For example, for in vivo introduction, an RNAi agent can be injected into a tissue site or administered systemically. In vitro introduction into a cell includes methods known in the art such as electroporation and lipofection. Further approaches are described herein below or are known in the art.

The term “lipophile” or “lipophilic moiety” broadly refers to any compound or chemical moiety having an affinity for lipids. One way to characterize the lipophilicity of the lipophilic moiety is by the octanol-water partition coefficient, logK_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko, et. al., J. Chem. Inf Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its logK_{ow} exceeds 0. Typically, the lipophilic moiety possesses a logK_{ow} exceeding 1, exceeding 1.5, exceeding 2, exceeding 3, exceeding 4, exceeding 5, or exceeding 10. For instance, the logK_{ow} of 6-amino hexanol, for instance, is predicted to be approximately 0.7. Using the same method, the logK_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., logK_{ow}) value of the lipophilic moiety.

Alternatively, the hydrophobicity of the double-stranded RNAi agent, conjugated to one or more lipophilic moieties, can be measured by its protein binding characteristics. For instance, in certain embodiments, the unbound fraction in the plasma protein binding assay of the double-stranded RNAi agent could be determined to positively correlate to the relative hydrophobicity of the double-stranded RNAi agent, which could then positively correlate to the silencing activity of the double-stranded RNAi agent.

In one embodiment, the plasma protein binding assay determined is an electrophoretic mobility shift assay (EMSA) using human serum albumin protein. An exemplary protocol of this binding assay is illustrated in detail in, e.g., PCT/US2019/031170. The hydrophobicity of the double-stranded RNAi agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded RNAi agent provides optimal hydrophobicity for the enhanced in vivo delivery of siRNA. The term “lipid nanoparticle” or “LNP” is a vesicle comprising a lipid layer encapsulating a pharmaceutically active molecule, such as a nucleic acid molecule, e.g., a RNAi agent or a plasmid from which an RNAi agent is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a rat, a mouse, or a rabbit). In one embodiment, the subject is a human, such as a human being treated or assessed for an ocular disease, disorder, or condition that would benefit from reduction in target gene expression; a human at risk for an ocular disease, disorder, or condition that would benefit from reduction in target gene expression; a human having an ocular disease, disorder, or condition that would benefit from reduction in target gene expression as described herein. In some embodiments, the subject is a female human. In other embodiments, the subject is a male human. In one embodiment, the subject is an adult subject. In one embodiment, the subject is a pediatric subject. In another embodiment, the subject is a juvenile subject, i.e., a subject below 20 years of age.

As used herein, the terms “treating” or “treatment” refer to a beneficial or desired result including, but not limited to, alleviation or amelioration of one or more signs or symptoms associated with target gene expression or target protein production, e.g., an ocular disorder or disease. “Treatment” can also mean prolonging survival as compared to expected survival in the absence of treatment.

The term “lower” or “decrease” in the context of the level of a target gene in a subject or a disease marker or symptom refers to a statistically significant decrease in such level. The decrease can be, for example, at least 10%, 15%, 20%, 25%, 30%, 35%, 40%, 45%, 50%, 55%, 60%, 65%, 70%, 75%, 80%, 85%, 90%, 95%, or more. In certain embodiments, a decrease is at least about 20%. In certain embodiments, the decrease is at least about 30% in a disease marker, e.g., a decrease of 30%, 35%, 40%, 45%, 50%, 55%, 60%, 65%, 70%, 75%, 80%, 85%, 90%, 95%, 99%, or more. In certain embodiments, the decrease is at least about 50% in a disease marker. “Lower” in the context of the level of the target gene in a subject is preferably down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in a sign or symptom of an ocular disorder or disease as compared to the accepted normal level. In another embodiment “lower” is the decrease in the difference between the level of a marker (or alternative read outs i.e. intraocular pressure) compared to its own individual baseline prior to treatment.

As used herein, “prevention” or “preventing,” when used in reference to a disease, disorder, or condition thereof, that would benefit from a reduction in expression of a target gene or production of a target protein, refers to a reduction in the likelihood that a subject will develop a symptom or a sign associated with such a disease, disorder, or condition, e.g., a symptom or a sign of an ocular disorder or disease, such as glaucoma. The failure to develop a disease, disorder, or condition, or the reduction in the development of a symptom associated with such a disease, disorder, or condition (e.g., by at least about 10% on a clinically accepted scale for that disease or disorder), or the exhibition of delayed symptoms delayed (e.g., by days, weeks, months or years) is considered effective prevention.

As used herein, the term “ocular disorder or disease,” includes any ocular disease, condition, or disorder that would benefit from reduction in the expression and/or activity of a target gene. Exemplary ocular disorders or diseases include glaucoma, primary open angle glaucoma, secondary glaucoma, pigmentary glaucoma, pseudoexfoliative glaucoma, traumatic glaucoma, neovascular glaucoma, irido corneal endothelial syndrome, uveitic glaucoma, angle closure glaucoma, normal tension glaucoma, juvenile open angle glaucoma, macular degeneration, cataracts, diabetic retinopathy, dry eyes, blurred vision, red eyes, blindness, night blindness, lazy eye, strabismus (cross eyes), nystagmus, colorblindness, uveitis, ocular inflammation, presbyopia, floaters in the field of vision, retinal diseases or disorders, retinal tear or detachment, conjunctivitis (pink eye), corneal diseases, vision changes, bulging eyes (proptosis), retinitis, diabetic macular edema, keratoconus, lazy eye, ocular hypertension, astigmatism, diabetic eye disease, hyperopia, myopia, macular edema, retinoblastoma, stargardt disease, usher syndrome, vitreous detachment, and cancers of the eye.

“Therapeutically effective amount,” as used herein, is intended to include the amount of an RNAi agent that, when administered to a subject having an ocular disorder or disease, is sufficient to effect treatment of the disorder or disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an RNAi agent that, when administered to a subject having an ocular disorder or disease, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. An RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the eye (e.g., ocular fluids or cells). In some embodiments, a “sample derived from a subject” refers to blood drawn from the subject or plasma or serum derived therefrom. In further embodiments, a “sample derived from a subject” refers to eye tissue or fluid (or subcomponents thereof) or retinal tissue (or subcomponents thereof) derived from the subject.

The term “substituted” refers to the replacement of one or more hydrogen radicals in a given structure with the radical of a specified substituent including, but not limited to: alkyl, alkenyl, alkynyl, aryl, heterocyclyl, halo, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent can be further substituted.

The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, “(C1-C6)alkyl” means a radical having from 1 to 6 carbon atoms in a linear or branched arrangement. “(C1-C6)alkyl” includes, for example, methyl, ethyl, propyl, iso-propyl, n-butyl, tert-butyl, pentyl and hexyl. In certain embodiments, a lipophilic moiety of the instant disclosure can include a C6-C18 alkyl hydrocarbon chain.

The term “alkylene” refers to an optionally substituted saturated aliphatic branched or straight chain divalent hydrocarbon radical having the specified number of carbon atoms. For example, “(C1-C6)alkylene” means a divalent saturated aliphatic radical having from 1-6 carbon atoms in a linear arrangement, e.g., [(CH₂)ₙ], where n is an integer from 1 to 6. “(C1-C6)alkylene” includes methylene, ethylene, propylene, butylene, pentylene and hexylene. Alternatively, “(C1-C6)alkylene” means a divalent saturated radical having from 1-6 carbon atoms in a branched arrangement, for example: [(CH₂CH₂CH₂CH₂CH(CH₃)], [(CH₂CH₂CH₂CH₂C(CH₃)₂], [(CH₂C(CH₃)₂CH(CH₃))], and the like. The term “alkylenedioxo” refers to a divalent species of the structure —O—R—O—, in which R represents an alkylene.

The term “mercapto” refers to an —SH radical. The term “thioalkoxy” refers to an —S— alkyl radical.

The term “halo” refers to any radical of fluorine, chlorine, bromine or iodine. “Halogen” and “halo” are used interchangeably herein.

As used herein, the term “cycloalkyl” means a saturated or unsaturated nonaromatic hydrocarbon ring group having from 3 to 14 carbon atoms, unless otherwise specified. For example, “(C3-C10) cycloalkyl” means a hydrocarbon radical of a (3-10)-membered saturated aliphatic cyclic hydrocarbon ring. Examples of cycloalkyl groups include, but are not limited to, cyclopropyl, methyl-cyclopropyl, 2,2-dimethyl-cyclobutyl, 2-ethyl-cyclopentyl, cyclohexyl, etc. Cycloalkyls may include multiple spiro- or fused rings. Cycloalkyl groups are optionally mono-, di-, tri-, tetra-, or penta-substituted on any position as permitted by normal valency.

As used herein, the term “alkenyl” refers to a non-aromatic hydrocarbon radical, straight or branched, containing at least one carbon-carbon double bond, and having from 2 to 10 carbon atoms unless otherwise specified. Up to five carbon-carbon double bonds may be present in such groups. For example, “C2-C6” alkenyl is defined as an alkenyl radical having from 2 to 6 carbon atoms. Examples of alkenyl groups include, but are not limited to, ethenyl, propenyl, butenyl, and cyclohexenyl. The straight, branched, or cyclic portion of the alkenyl group may contain double bonds and is optionally mono-, di-, tri-, tetra-, or penta-substituted on any position as permitted by normal valency. The term “cycloalkenyl” means a monocyclic hydrocarbon group having the specified number of carbon atoms and at least one carbon-carbon double bond.

As used herein, the term “alkynyl” refers to a hydrocarbon radical, straight or branched, containing from 2 to 10 carbon atoms, unless otherwise specified, and containing at least one carbon-carbon triple bond. Up to 5 carbon-carbon triple bonds may be present. Thus, “C2-C6 alkynyl” means an alkynyl radical having from 2 to 6 carbon atoms. Examples of alkynyl groups include, but are not limited to, ethynyl, 2-propynyl, and 2-butynyl. The straight or branched portion of the alkynyl group may contain triple bonds as permitted by normal valency, and may be optionally mono-, di-, tri-, tetra-, or penta-substituted on any position as permitted by normal valency.

As used herein, “alkoxyl” or “alkoxy” refers to an alkyl group as defined above with the indicated number of carbon atoms attached through an oxygen bridge. For example, “(C1-C3)alkoxy” includes methoxy, ethoxy, and propoxy. For example, “(C1-C6)alkoxy”, is intended to include C1, C2, C3, C4, C5, and C6 alkoxy groups. For example, “(C1-C8)alkoxy”, is intended to include C1, C2, C3, C4, C5, C6, C7, and C8 alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy. “Alkylthio” means an alkyl radical attached through a sulfur linking atom. The terms “alkylamino” or “aminoalkyl”, means an alkyl radical attached through an NH linkage. “Dialkylamino” means two alkyl radical attached through a nitrogen linking atom. The amino groups may be unsubstituted, monosubstituted, or di-substituted. In some embodiments, the two alkyl radicals are the same (e.g., N,N-dimethylamino). In some embodiments, the two alkyl radicals are different (e.g., N-ethyl-N-methylamino).

As used herein, “aryl” or “aromatic” means any stable monocyclic or polycyclic carbon ring of up to 7 atoms in each ring, wherein at least one ring is aromatic. Examples of aryl groups include, but are not limited to, phenyl, naphthyl, anthracenyl, tetrahydronaphthyl, indanyl, and biphenyl. In cases where the aryl substituent is bicyclic and one ring is non-aromatic, it is understood that attachment is via the aromatic ring. Aryl groups are optionally mono-, di-, tri-, tetra-, or penta-substituted on any position as permitted by normal valency. The term “arylalkyl” or the term “aralkyl” refers to alkyl substituted with an aryl. The term “arylalkoxy” refers to an alkoxy substituted with aryl.

“Hetero” refers to the replacement of at least one carbon atom in a ring system with at least one heteroatom selected from N, S and O. “Hetero” also refers to the replacement of at least one carbon atom in an acyclic system. A hetero ring system or a hetero acyclic system may have, for example, 1, 2 or 3 carbon atoms replaced by a heteroatom.

As used herein, the term “heteroaryl” represents a stable monocyclic or polycyclic ring of up to 7 atoms in each ring, wherein at least one ring is aromatic and contains from 1 to 4 heteroatoms selected from the group consisting of O, N and S. Examples of heteroaryl groups include, but are not limited to, acridinyl, carbazolyl, cinnolinyl, quinoxalinyl, pyrrazolyl, indolyl, benzotriazolyl, furanyl, thienyl, benzothienyl, benzofuranyl, benzimidazolonyl, benzoxazolonyl, quinolinyl, isoquinolinyl, dihydroisoindolonyl, imidazopyridinyl, isoindolonyl, indazolyl, oxazolyl, oxadiazolyl, isoxazolyl, indolyl, pyrazinyl, pyridazinyl, pyridinyl, pyrimidinyl, pyrrolyl, tetrahydroquinoline. “Heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring. Heteroaryl groups are optionally mono-, di-, tri-, tetra-, or penta-substituted on any position as permitted by normal valency.

As used herein, the term “heterocycle,” “heterocyclic,” or “heterocyclyl” means a 3- to 14-membered aromatic or nonaromatic heterocycle containing from 1 to 4 heteroatoms selected from the group consisting of O, N and S, including polycyclic groups. As used herein, the term “heterocyclic” is also considered to be synonymous with the terms “heterocycle” and “heterocyclyl” and is understood as also having the same definitions set forth herein. “Heterocyclyl” includes the above mentioned heteroaryls, as well as dihydro and tetrahydro analogs thereof. Examples of heterocyclyl groups include, but are not limited to, azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyridinonyl, pyrimidyl, pyrimidinonyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom. Heterocyclyl groups are optionally mono-, di-, tri-, tetra-, or penta-substituted on any position as permitted by normal valency.

“Heterocycloalkyl” refers to a cycloalkyl residue in which one to four of the carbons is replaced by a heteroatom such as oxygen, nitrogen or sulfur. Examples of heterocycles whose radicals are heterocyclyl groups include tetrahydropyran, morpholine, pyrrolidine, piperidine, thiazolidine, oxazole, oxazoline, isoxazole, dioxane, tetrahydrofuran and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

As used herein, “keto” refers to any alkyl, alkenyl, alkynyl, cycloalkyl, cycloalkenyl, heterocyclyl, heteroaryl, or aryl group as defined herein attached through a carbonyl bridge.

Examples of keto groups include, but are not limited to, alkanoyl (e.g., acetyl, propionyl, butanoyl, pentanoyl, hexanoyl), alkenoyl (e.g., acryloyl)alkynoyl (e.g., ethynoyl, propynoyl, butynoyl, pentynoyl, hexynoyl), aryloyl (e.g., benzoyl), heteroaryloyl (e.g., pyrroloyl, imidazoloyl, quinolinoyl, pyridinoyl).

As used herein, “alkoxycarbonyl” refers to any alkoxy group as defined above attached through a carbonyl bridge (i.e., —C(O)O-alkyl). Examples of alkoxycarbonyl groups include, but are not limited to, methoxycarbonyl, ethoxycarbonyl, iso-propoxycarbonyl, n-propoxycarbonyl, t-butoxycarbonyl, benzyloxycarbonyl or n-pentoxycarbonyl.

As used herein, “aryloxycarbonyl” refers to any aryl group as defined herein attached through an oxycarbonyl bridge (i.e., —C(O)O-aryl). Examples of aryloxycarbonyl groups include, but are not limited to, phenoxycarbonyl and naphthyloxycarbonyl.

As used herein, “heteroaryloxycarbonyl” refers to any heteroaryl group as defined herein attached through an oxycarbonyl bridge (i.e., —C(O)O-heteroaryl). Examples of heteroaryloxycarbonyl groups include, but are not limited to, 2-pyridyloxycarbonyl, 2-oxazolyloxycarbonyl, 4-thiazolyloxycarbonyl, or pyrimidinyloxycarbonyl.

The term “oxo” refers to an oxygen atom, which forms a carbonyl when attached to carbon, an N-oxide when attached to nitrogen, and a sulfoxide or sulfone when attached to sulfur.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the pH of the environment, as would be readily understood by the person of ordinary skill in the art.

II. iRNA Agents of the Disclosure

Described herein are iRNA agents that inhibit the expression of a target gene in an ocular cell or tissue. In one embodiment, the iRNA agent includes double stranded ribonucleic acid (dsRNA) molecules for inhibiting the expression of a target gene in an ocular cell or tissue, such as a cell or tissue within a subject, e.g., a mammal, such as a human having an ocular disorder or disease. Any target gene can be inhibited by the iRNA agents provided herein. In one embodiment, the target gene is any gene involved in an ocular disorder or disease. In a certain embodiment, the target gene is any gene involved in glaucoma.

Non-limiting examples of ocular target genes include any genes involved in an ocular disorder or disease, such as, for example, myocilin (MYOC), Ras homolog family member A (RhoA), vascular endothelial growth factor A (VEGFA), SSB (small RNA binding exonuclease protection factor La), optineurin, Carbonic Anhydrase 2 (CA2), Rho associated coiled-coil containing protein kinase 1 (ROCK1), Rho associated coiled-coil containing protein kinase 2 (ROCK2), Angiopoietin-Like 7 (ANGPTL7), and cytochrome P450 1B1 (CYPIB1), and combinations thereof.

The iRNA agents provided herein comprise a sense strand and an antisense strand and at least one of the strands is modified for targeting delivery to the eye. In one embodiment, the iRNA agents are modified by conjugation to an integrin targeting ligand. By “integrin targeting ligand” is meant any ligand that binds to an integrin. The iRNA agents may be conjugated to any integrin targeting ligand including, but not limited to, RGD peptide ligands, cyclic RGD peptide ligands, RGD peptide mimetics, vitronectin, cilingetide, small molecule RGD mimic, fibronectin, collagen, laminin, fibrinogen, thrombospondin, and glycoproteins (e.g., tenascin C, osteopontin, and nefronectin). The integrin targeting ligands can be conjugated to any iRNA agent targeting any ocular target gene.

In one embodiment, the iRNA agents provided herein are conjugated to an RGD peptide ligand. An RGD peptide ligand for use in the compositions and methods of the invention may be linear or cyclic, and may be modified, e.g., glycosylated or methylated. RGD-containing peptides and peptidiomimetics may include D-amino acids, as well as synthetic RGD mimetics.

In some embodiments, the RGD peptide ligand is multivalent. The valency may be mono-, bi-tri-, tetra- or higher valency. In certain embodiments, the RGD peptide ligand is a monovalent cRGD peptide ligand, a bivalent cRGD peptide ligand, a trivalent cRGD peptide ligand, or a tetravalent cRGD peptide ligand.

The amino acid residues of the mono-, bi-, tri- and tetra-valent cRGD peptide ligands respectively are: mono sequence H-Cys-Aca-Glu-[cyclo(Arg-Gly-Asp-D-Phe-Lys)], H-Cys-Aca-Glu-[cyclo(Arg-Gly-Asp-D-Phe-Lys)]₂, H-Cys-Aca-Glu-[cyclo(Arg-Gly-Asp-D-Phe-Lys)]-Glu [cyclo(Arg-Gly-Asp-D-Phe-Lys)]₂ and H-Cys-Aca-Glu-{Glu-[cyclo(Arg-Gly-Asp-D-Phe-Lys)]₂}2. A thiol group is introduced to the cRGDs by attaching a cysteine residue via the spacer -6-aminocaproic acid, to the α-amino group of glutamic acid to enable conjugation of the peptide to a maleimide containing oligonucleotide under Michael addition conditions. Methods of synthesizing the various cRGD peptides and conjugating the RGD peptides to siRNA are known in the art and include the methods provided in the examples elsewhere herein and those methods provided in Alam, R. W., et al., Bioconjug. Chem., 2011, 22(8): 1673-1681, hereby incorporated by reference in its entirety.

In one embodiment, the iRNA agent and the integrin tarteting ligand can be attached via a linker. In non-limiting examples, the linkers can comprise a cleavable linker, a peptide, DNA, RNA, disulfide, amide, functionalized monosaccharides or oligosaccharides of galactosamine, glucosamine, glucose, galactose, mannose, an ether, a thioether, a urea, a carbonate, an amine, an amide, a maleimide-thioether, a disulfide, a phosphodiester, a sulfonamide linkage, a product of a click reaction (e.g., a triazole from the azide-alkyne cycloaddition), or a carbamate. In some embodiments, the linker can be a bivalent or trivalent branched linker. In some embodiments, a cleaviable linker comprises a redox cleavable linking group, a phosphate-based cleavable linking group, an acid cleavable linking group, an ester-based cleavable linking group, or a peptide-based cleavable linking group. Non-limiting examples of cleavable linkers useful for preparing a conjugate and/or ligand of the present invention include those described in WO2009/073809 and WO 2018/136620, which are hereby incorporated by reference in their entirety.

In one embodiment, the integrin targeting ligand is a monovalent cRGD peptide ligand. In a certain embodiment, the integrin targeting ligand is a monovalent cRGD peptide ligand having the structure:

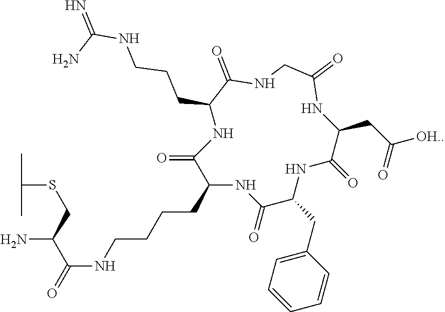 [SMILES: CC(C)SC[C@H](N)C(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O]

In one embodiment, the integrin targeting ligand is a trivalent cRGD peptide ligand. In some embodiments, the integrin targeting ligand is a trivalent cRGD peptide ligand having the structure:

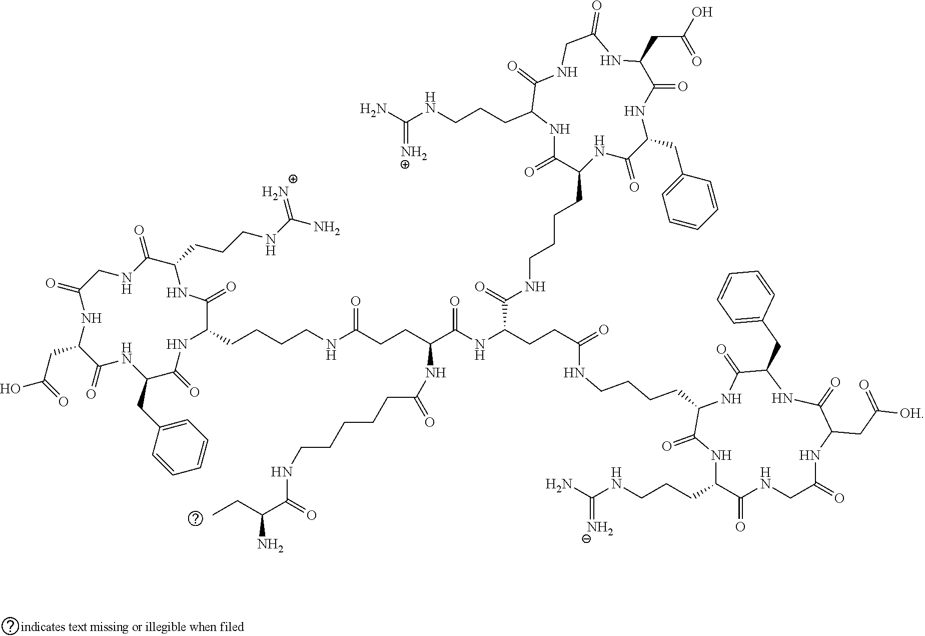 [SMILES: CC[C@H](N)C(=O)NCCCCCC(=O)N[C@@H](CCC(=O)NCCCC[C@@H]1NC(=O)[C@@H](Cc2ccccc2)NC(=O)[C@H](CC(=O)O)NC(=O)CNC(=O)[C@H](CCCNC(N)=[NH2+])NC1=O)C(=O)N[C@@H](CCC(=O)NCCCC[C@@H]1NC(=O)[C@@H](Cc2ccccc2)NC(=O)C(CC(=O)O)NC(=O)CNC(=O)[C@H](CCCNC(=[N-])N)NC1=O)C(=O)NCCCC[C@@H]1NC(=O)[C@@H](Cc2ccccc2)NC(=O)[C@H](CC(=O)O)NC(=O)CNC(=O)C(CCCNC(N)=[NH2+])NC1=O]

In one embodiment, the integrin targeting ligand is a bivalent cRGD peptide ligand. In another embodiment, the integrin targeting ligand is a tetravalent cRGD peptide ligand.

### Non-Limiting Examples of Integrin Targeting Ligands Include:

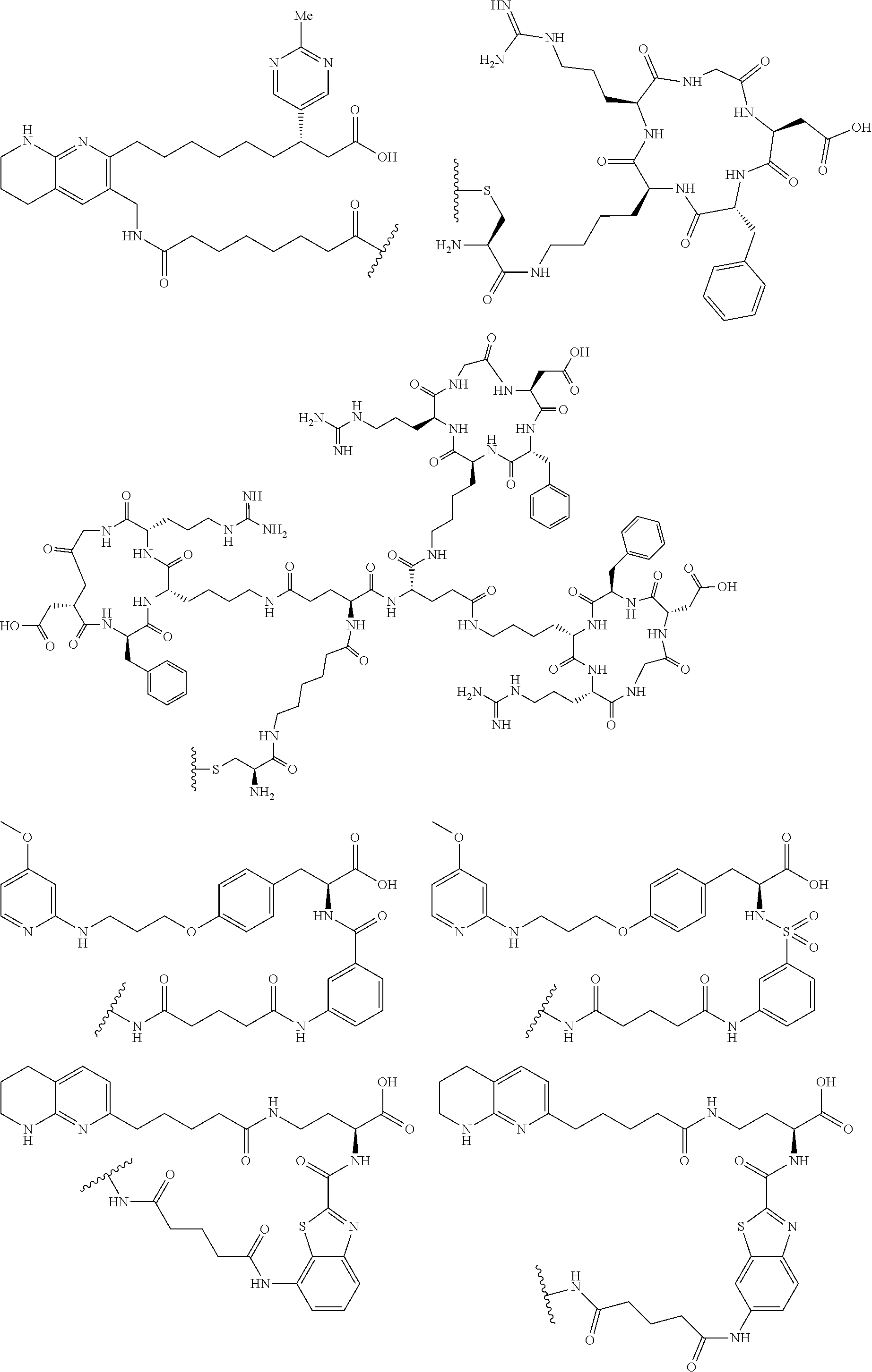

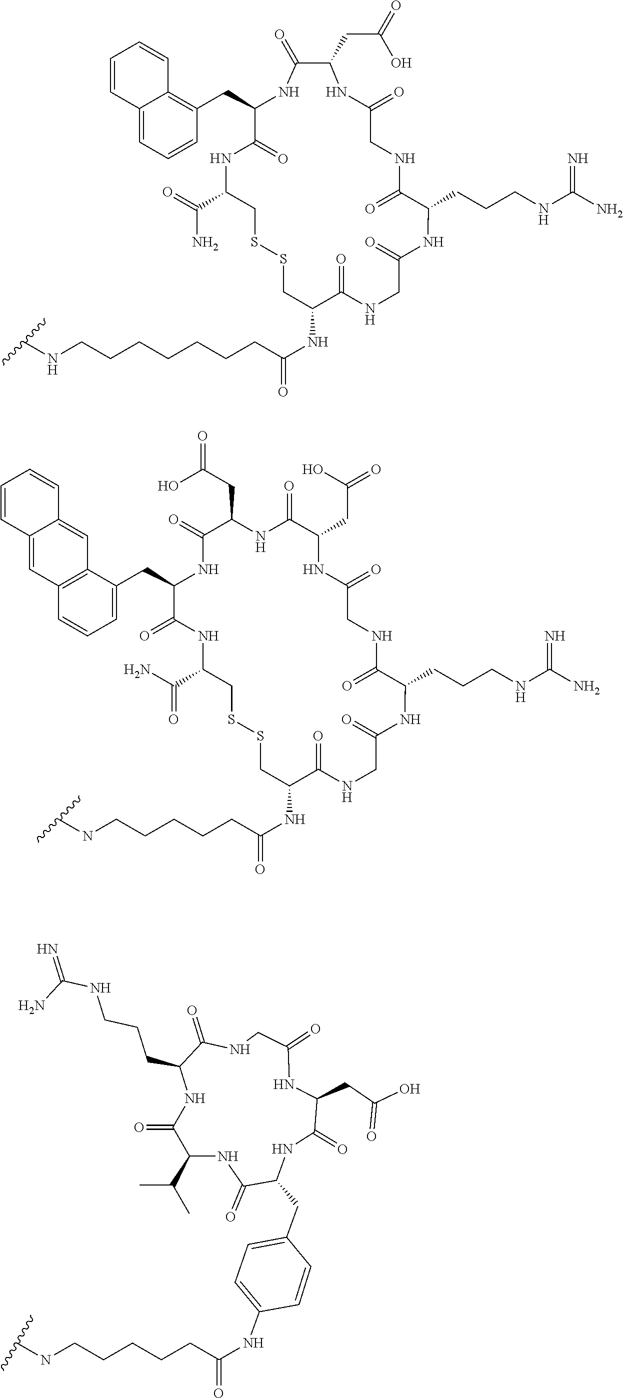 [SMILES: *NCCCCCCCC(=O)N[C@@H]1CSSC[C@H](C(N)=O)NC(=O)[C@@H](Cc2cccc3ccccc23)NC(=O)[C@H](CC(=O)O)NC(=O)CNC(=O)[C@H](CCCNC(=N)N)NC(=O)CNC1=O ; *NCCCCCC(=O)N[C@@H]1CSSC[C@H](C(N)=O)NC(=O)[C@@H](Cc2cccc3cc4ccccc4cc23)NC(=O)[C@@H](CC(=O)O)NC(=O)[C@H](CC(=O)O)NC(=O)CNC(=O)[C@H](CCCNC(=N)N)NC(=O)CNC1=O ; *NCCCCCC(=O)Nc1ccc(C[C@H]2NC(=O)[C@H](CC(=O)O)NC(=O)CNC(=O)[C@H](CCCNC(=N)N)NC(=O)[C@H](C(C)C)NC2=O)cc1]

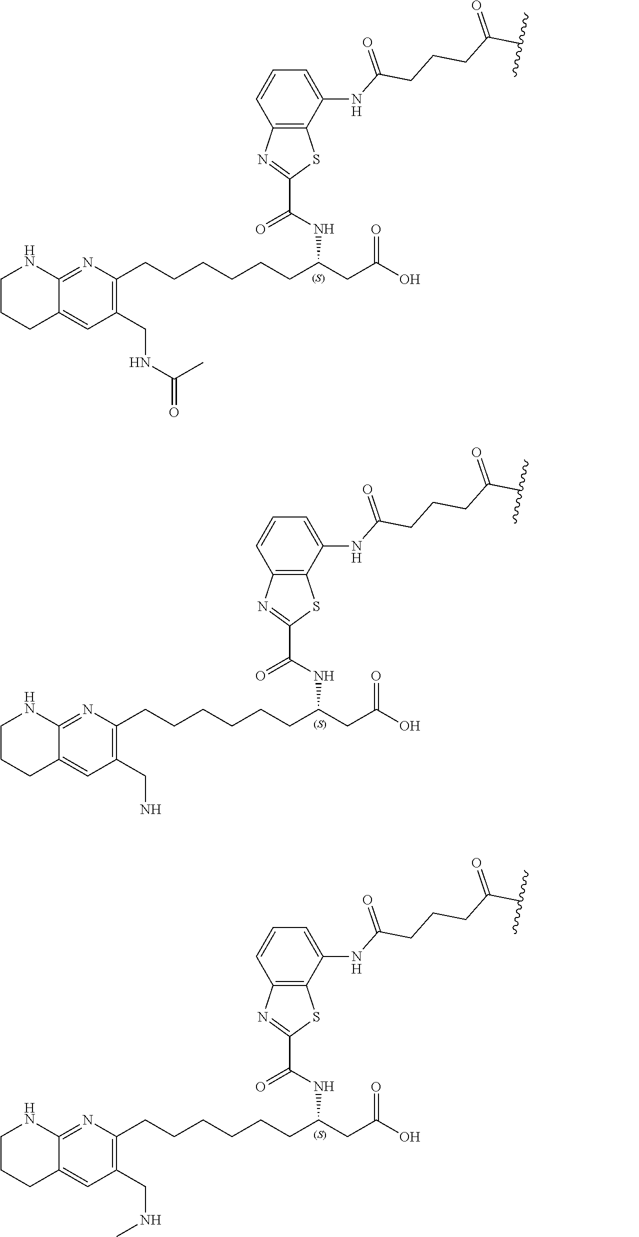

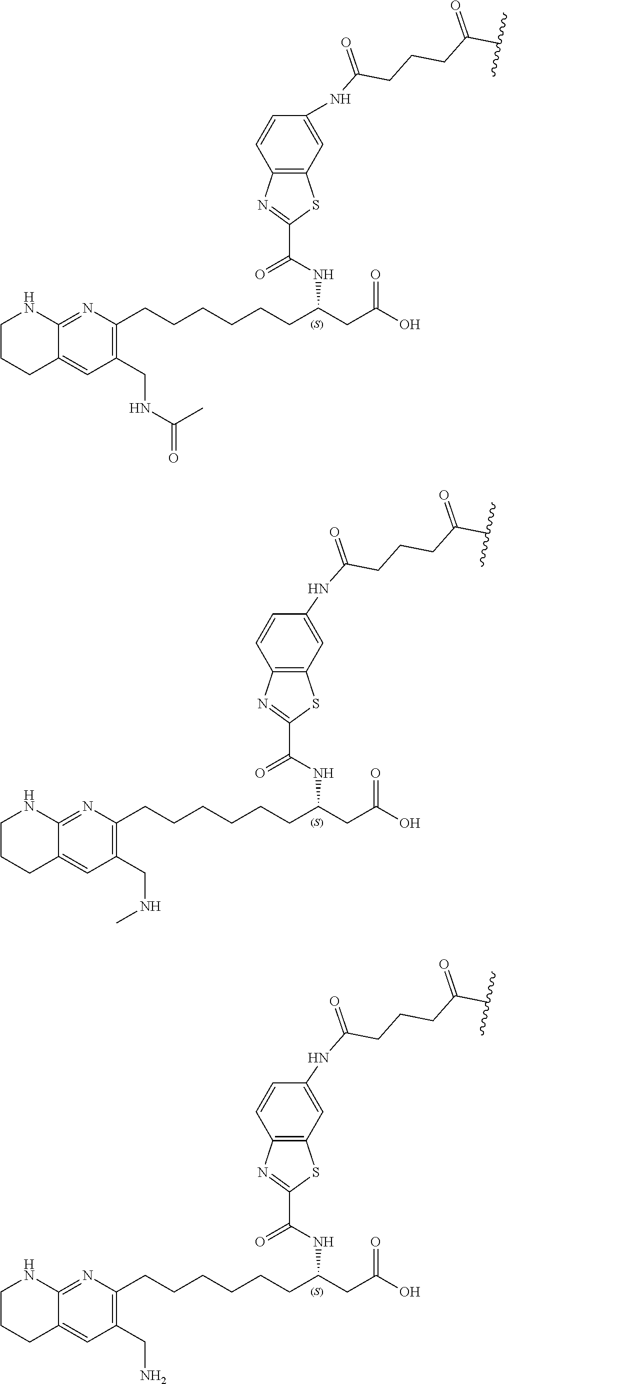

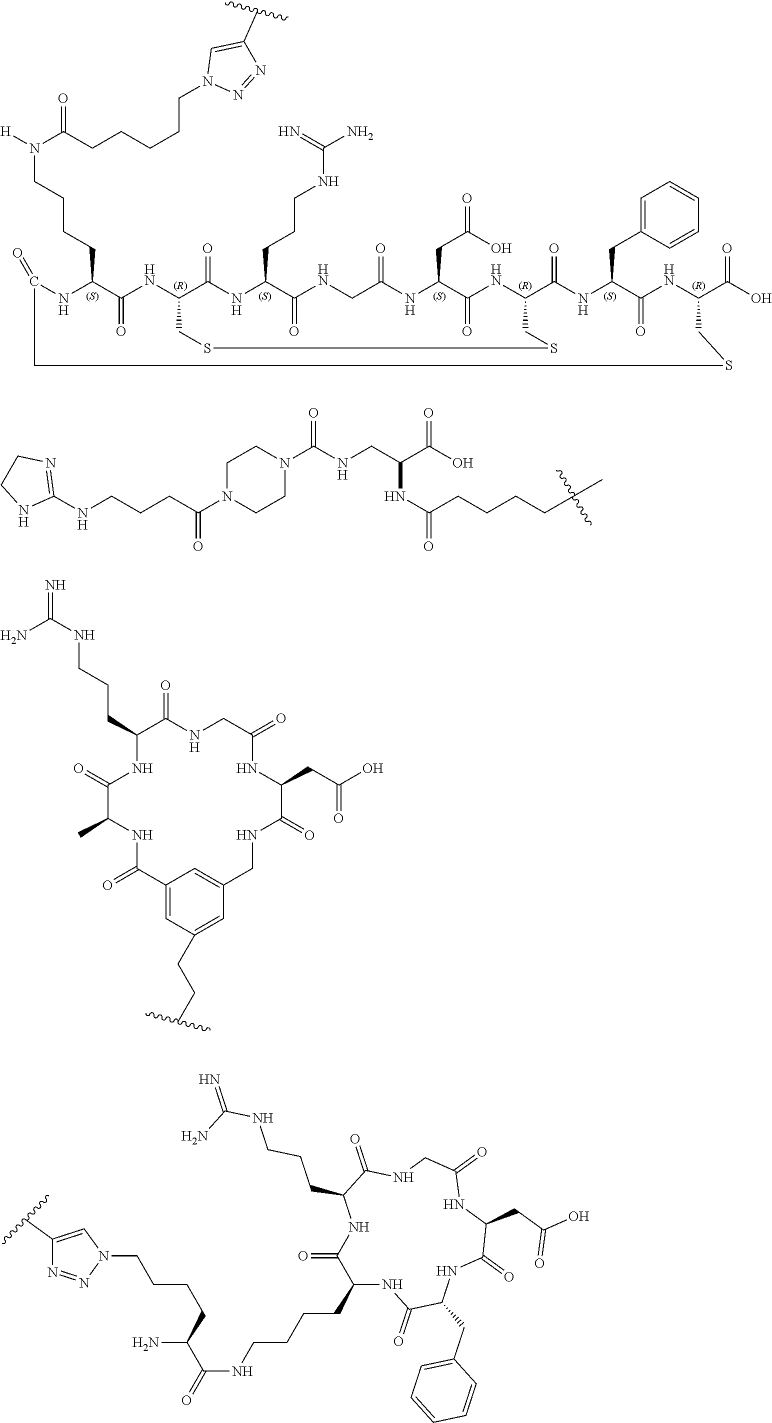

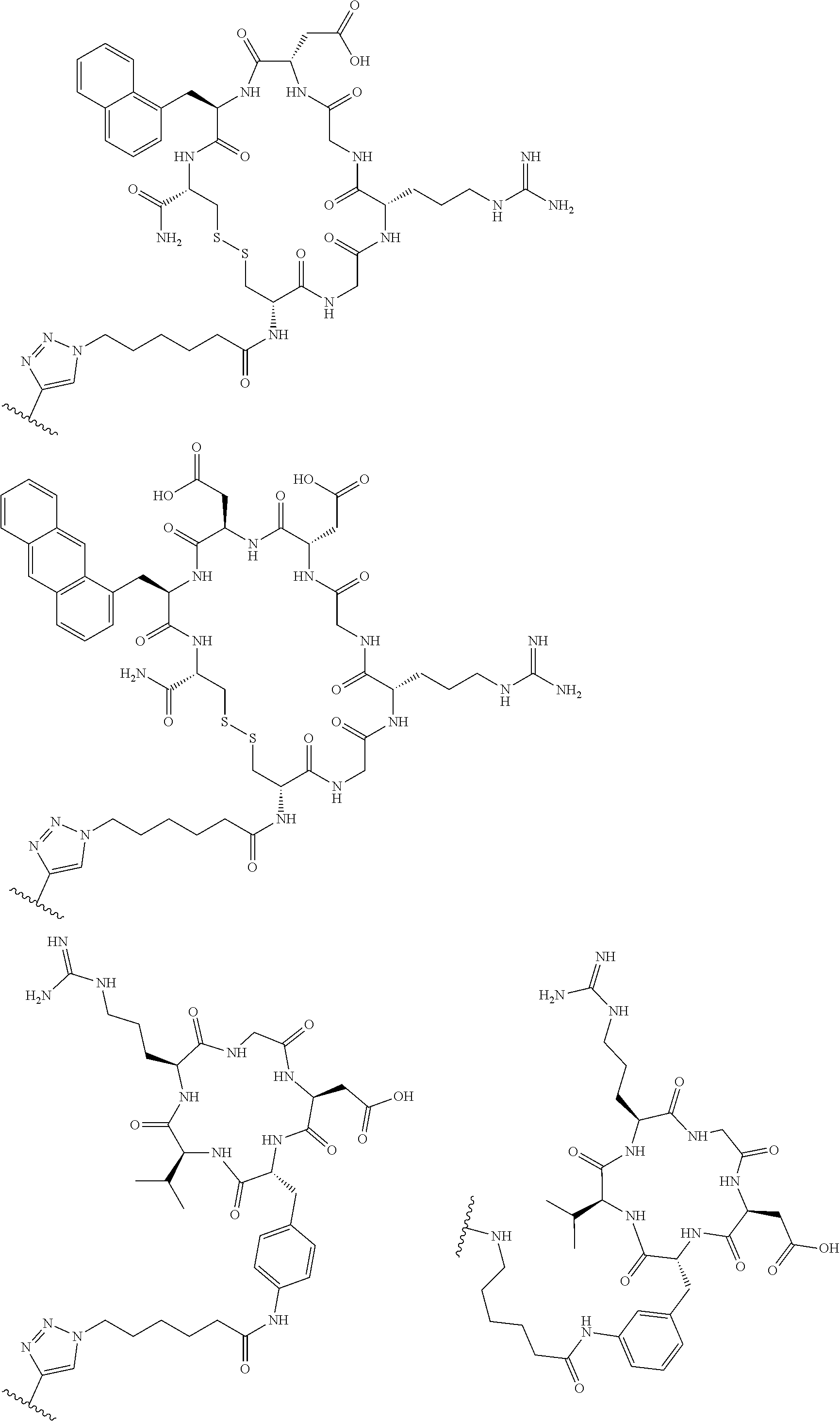

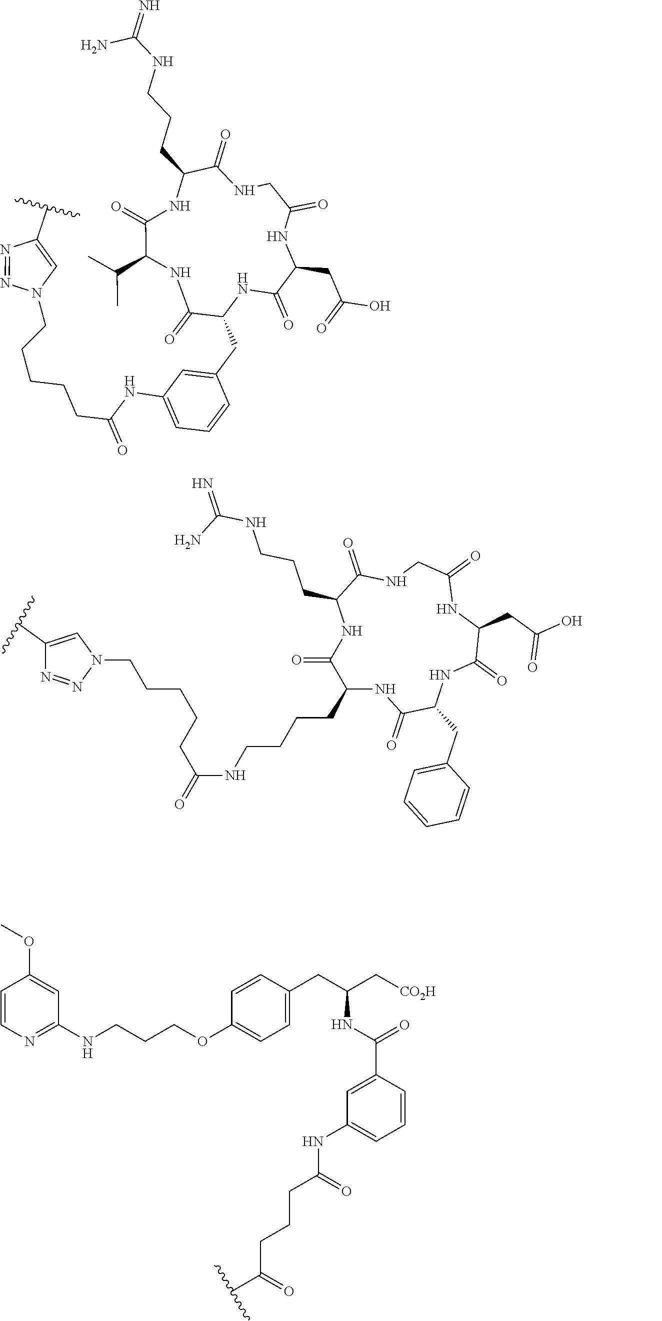

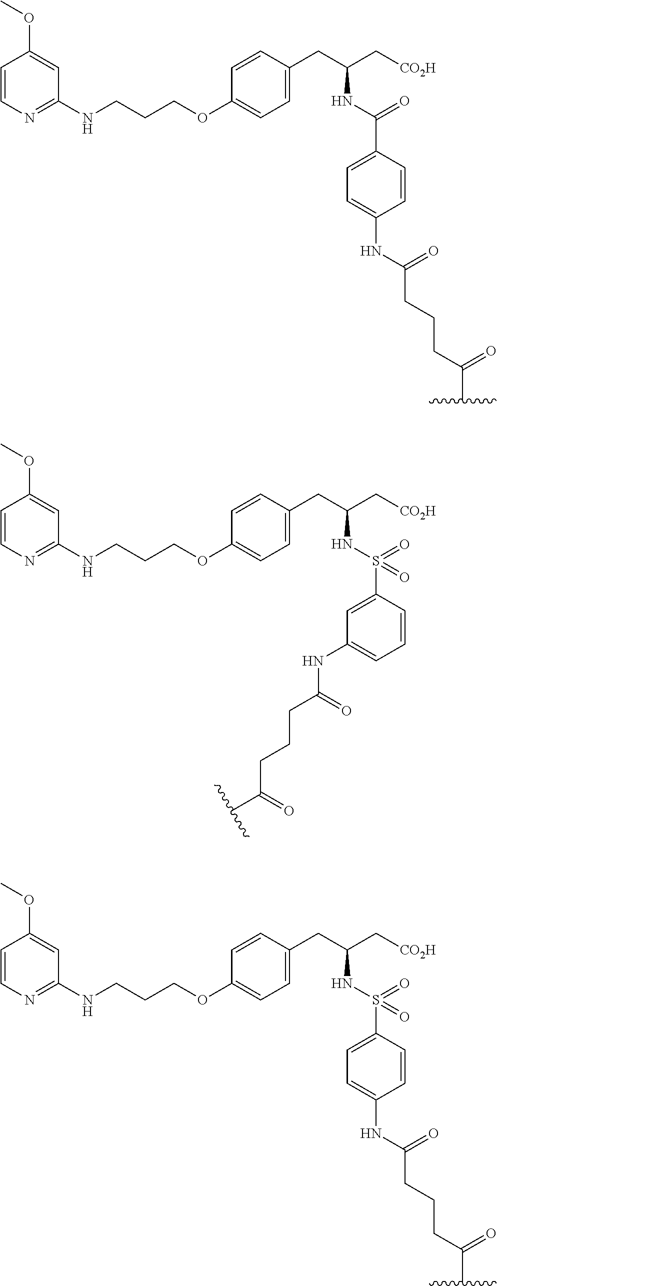 [SMILES: *C(=O)CCCC(=O)Nc1ccc(C(=O)N[C@H](CC(=O)O)Cc2ccc(OCCCNc3cc(OC)ccn3)cc2)cc1 ; *C(=O)CCCC(=O)Nc1cccc(S(=O)(=O)N[C@H](CC(=O)O)Cc2ccc(OCCCNc3cc(OC)ccn3)cc2)c1 ; *C(=O)CCCC(=O)Nc1ccc(S(=O)(=O)N[C@H](CC(=O)O)Cc2ccc(OCCCNc3cc(OC)ccn3)cc2)cc1]

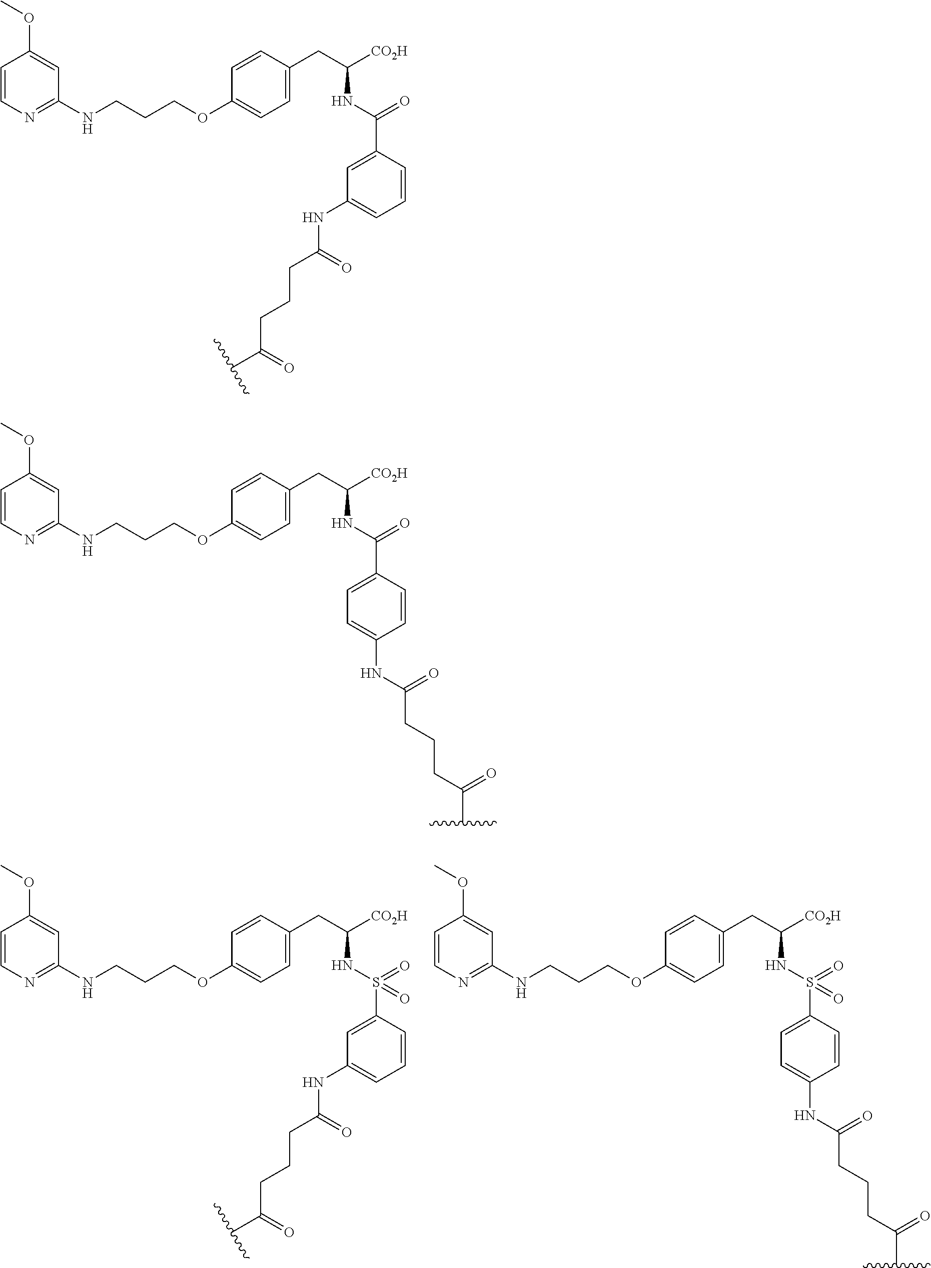

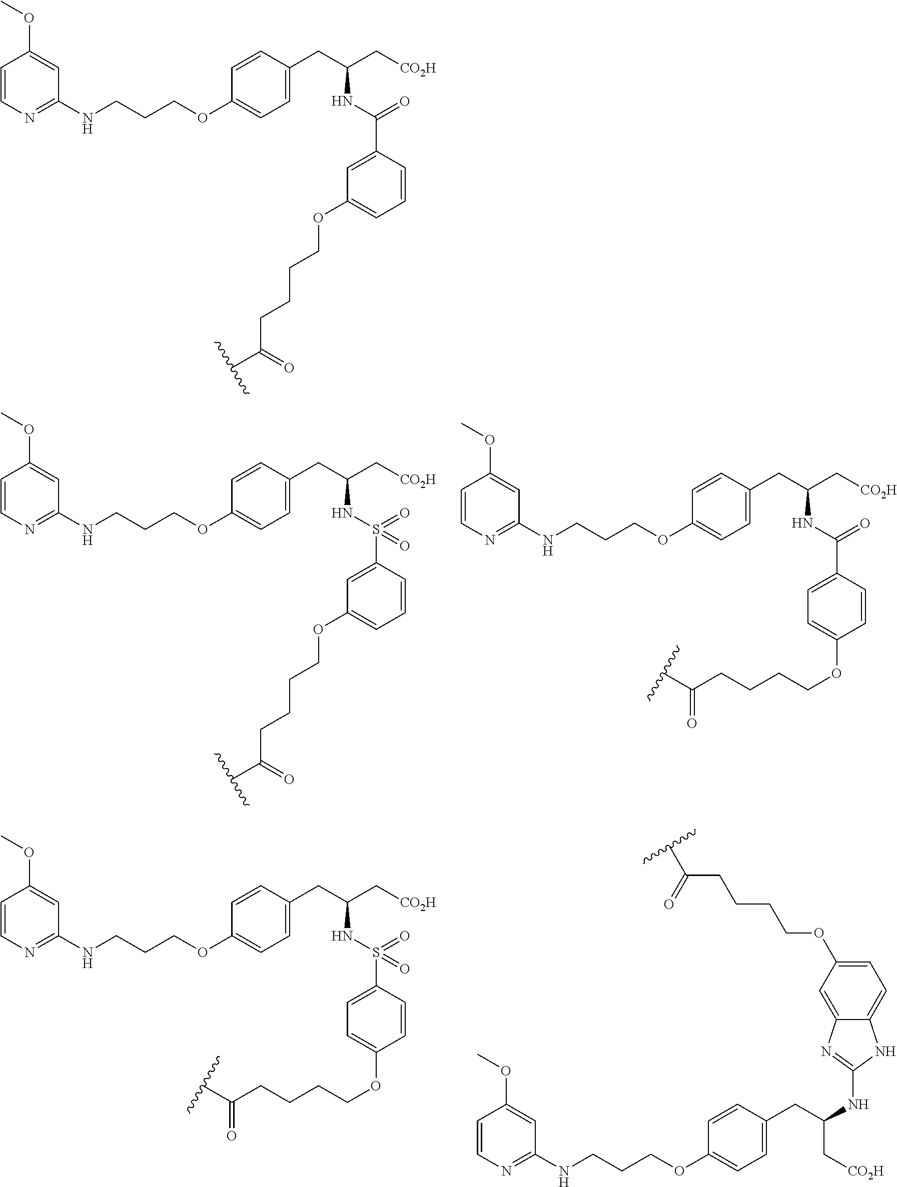 [SMILES: *C(=O)CCCCOc1cccc(C(=O)N[C@H](CC(=O)O)Cc2ccc(OCCCNc3cc(OC)ccn3)cc2)c1 ; *C(=O)CCCCOc1cccc(S(=O)(=O)N[C@H](CC(=O)O)Cc2ccc(OCCCNc3cc(OC)ccn3)cc2)c1 ; *C(=O)CCCCOc1ccc(C(=O)N[C@H](CC(=O)O)Cc2ccc(OCCCNc3cc(OC)ccn3)cc2)cc1 ; *C(=O)CCCCOc1ccc(S(=O)(=O)N[C@H](CC(=O)O)Cc2ccc(OCCCNc3cc(OC)ccn3)cc2)cc1 ; *C(=O)CCCCOc1ccc2[nH]c(N[C@@H](CC(=O)O)Cc3ccc(OCCCNc4cc(OC)ccn4)cc3)nc2c1]

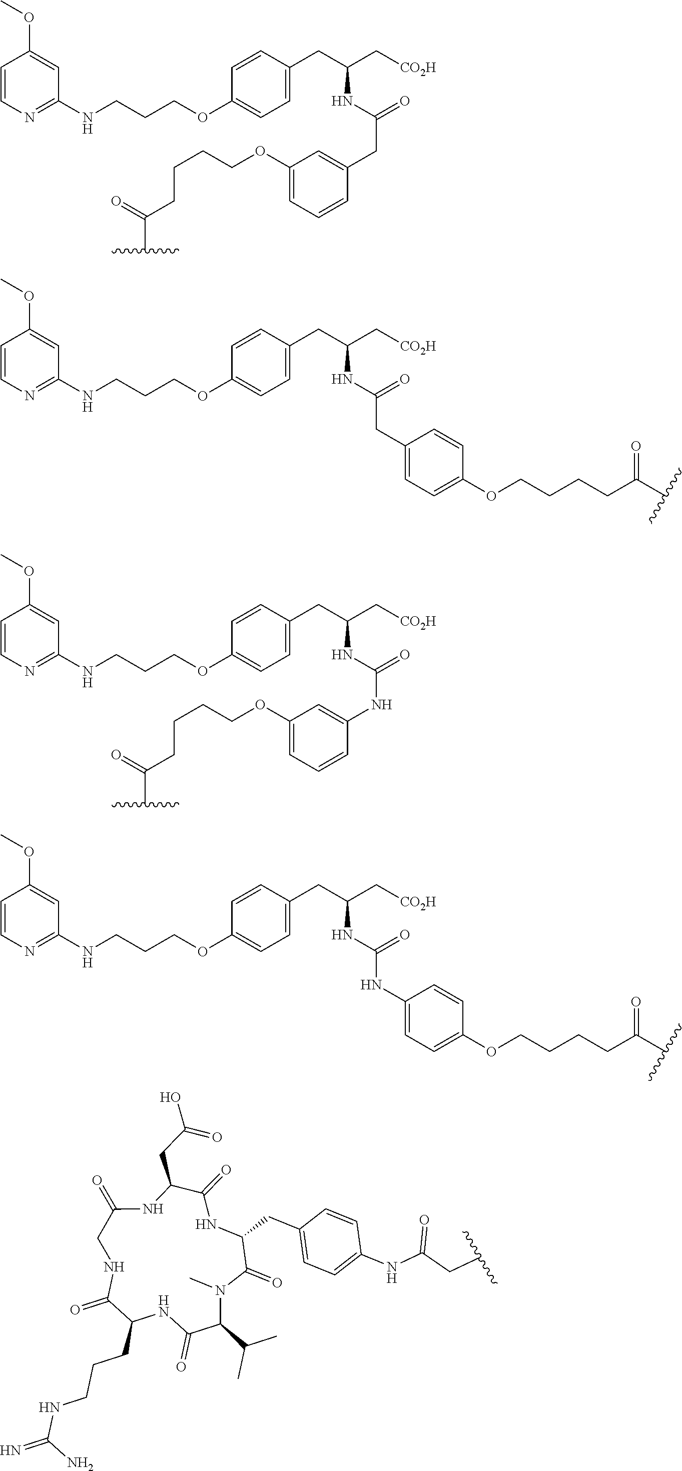 [SMILES: *C(=O)CCCCOc1cccc(CC(=O)N[C@H](CC(=O)O)Cc2ccc(OCCCNc3cc(OC)ccn3)cc2)c1 ; *C(=O)CCCCOc1ccc(CC(=O)N[C@H](CC(=O)O)Cc2ccc(OCCCNc3cc(OC)ccn3)cc2)cc1 ; *C(=O)CCCCOc1cccc(NC(=O)N[C@H](CC(=O)O)Cc2ccc(OCCCNc3cc(OC)ccn3)cc2)c1 ; *C(=O)CCCCOc1ccc(NC(=O)N[C@H](CC(=O)O)Cc2ccc(OCCCNc3cc(OC)ccn3)cc2)cc1 ; *CC(=O)Nc1ccc(C[C@H]2NC(=O)[C@H](CC(=O)O)NC(=O)CNC(=O)[C@H](CCCNC(=N)N)NC(=O)[C@H](C(C)C)N(C)C2=O)cc1]

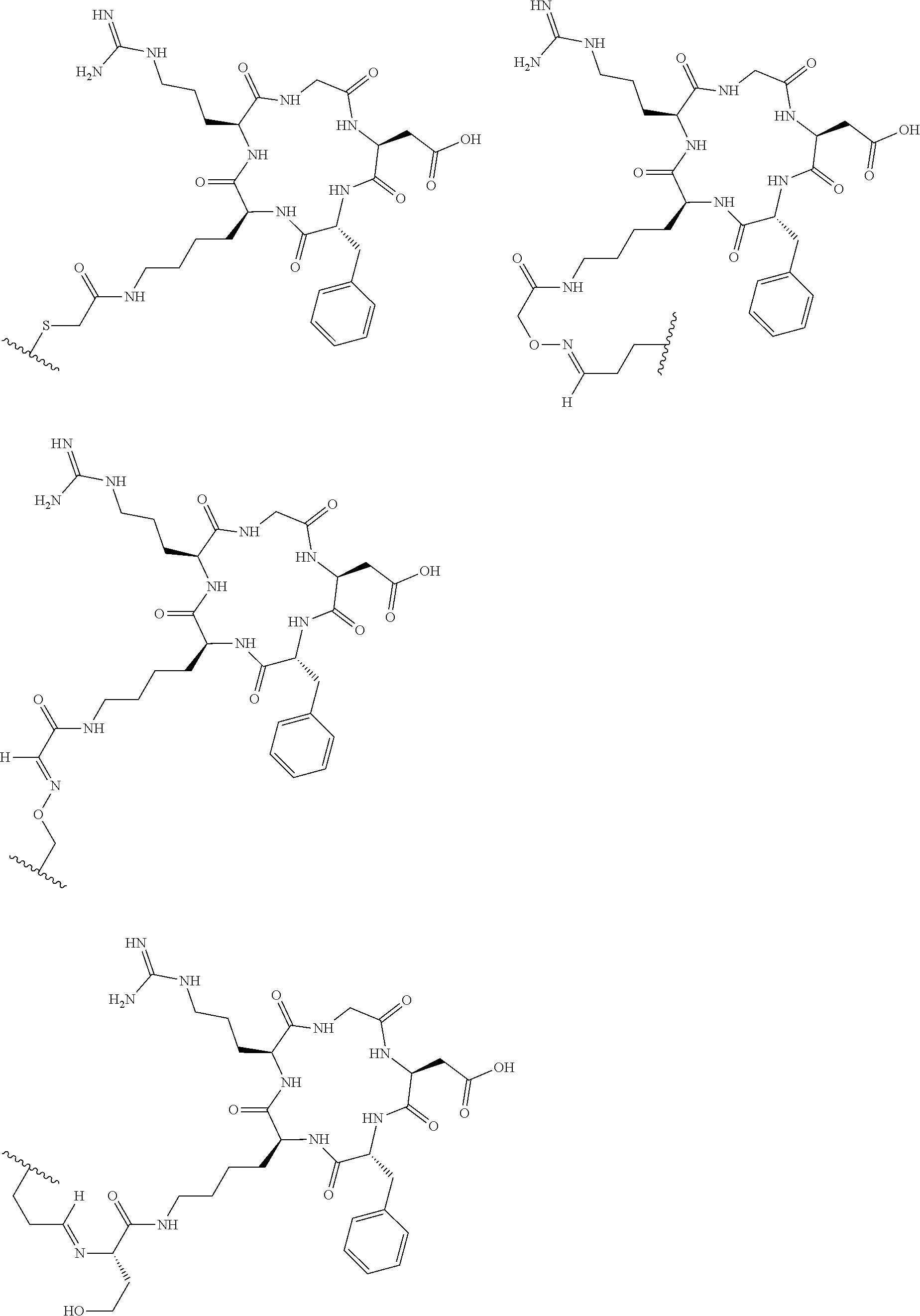 [SMILES: *SCC(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O ; *CC/C=N/OCC(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O ; *CO/N=C/C(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O ; *CC/C=N/[C@@H](CCO)C(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O]

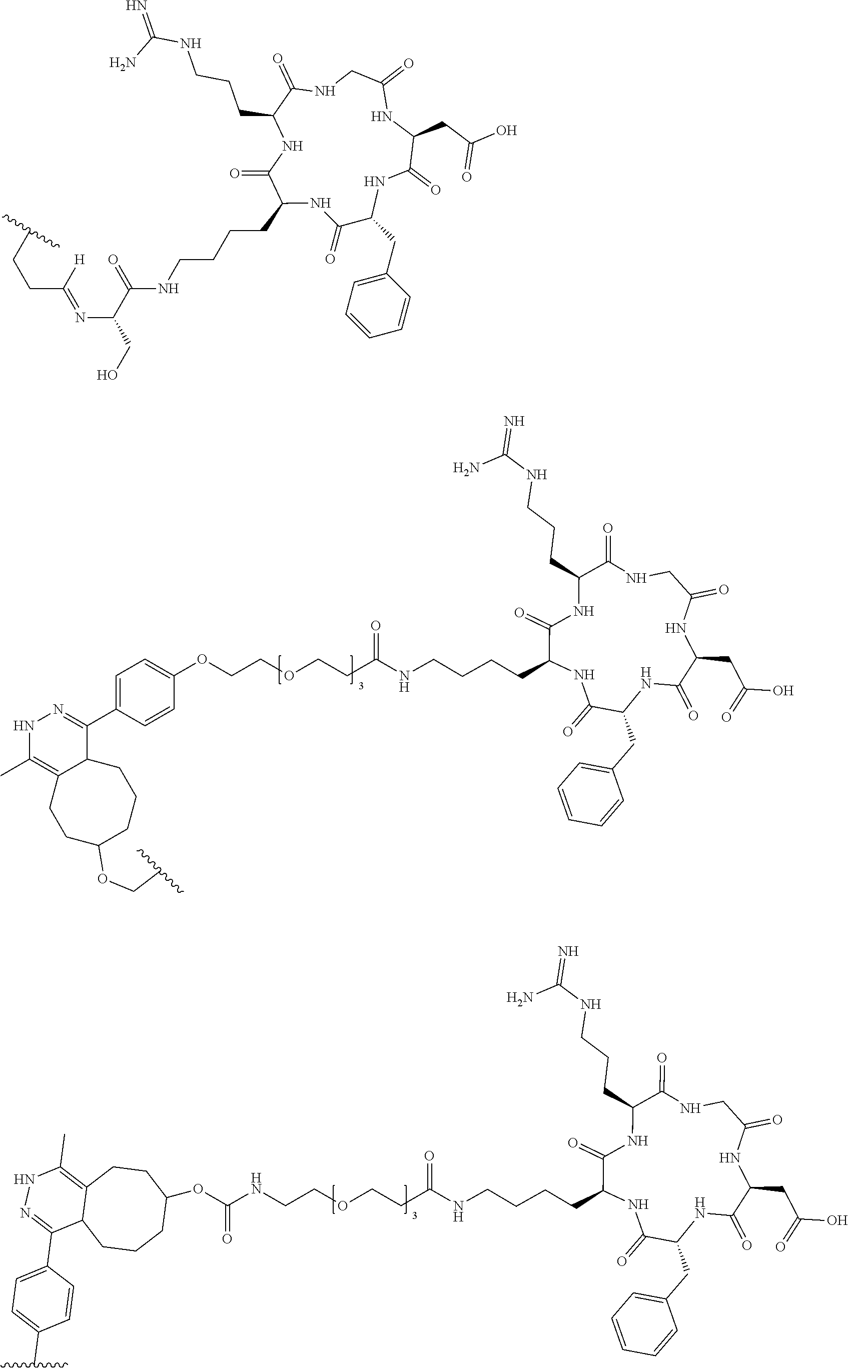

In some embodiments, the ligand is conjugated to the sense strand. In some embodiments, the ligand is conjugated to the 3′ end of the sense strand, to the 5′ end of the sense strand, or to an internal position on the sense strand. In some embodiments, the ligand is conjugated to the 3′ end of the sense strand. In other embodiments, the ligand is conjugated to the 5′ end of the sense strand.

In some embodiments. the ligand is conjugated to the antisense strand. In some embodiments. the ligand is conjugated to the 3′ end of the antisense strand, to the 5′ end of the antisense strand, or to an internal position on the antisense strand. In some embodiments, the ligand is conjugated to the 3′ end of the antisense strand. In some embodiments, the ligand is conjugated to the 5′ end of the antisense strand.

In one embodiment, at least one of the strands of the iRNA agent is conjugated to at least one integrin targeting ligand. The iRNA agent may be conjugated to one, two, three, four, or more integrin targeting ligands.

In some embodiments, the integrin targeting ligand is selected from the group consisting of RGD peptide ligands, RGD peptide mimetics, fibronectin, collagen, vitronectin, laminin, fibrinogen, thrombospondin, glycoproteins (e.g., tenascin C, osteopontin, and nefronectin), an RGD peptide ligand, a cyclic RGD peptide ligand, a monovalent cyclic RGD peptide ligand, a bivalent cyclic-RGD peptide, a trivalent cyclic-RGD peptide, a tetravalent cyclic-RGD peptide, Cilengitide, and tetrahydronaphthyridinyl nonanoic acid derivatives. Non-limiting examples of small molecule integrin targeting ligands are described in U.S. Pat. No. 6,410,526, the entire contents of which are hereby incorporated herein by reference.

In one embodiment, the iRNA agent comprises the structure:

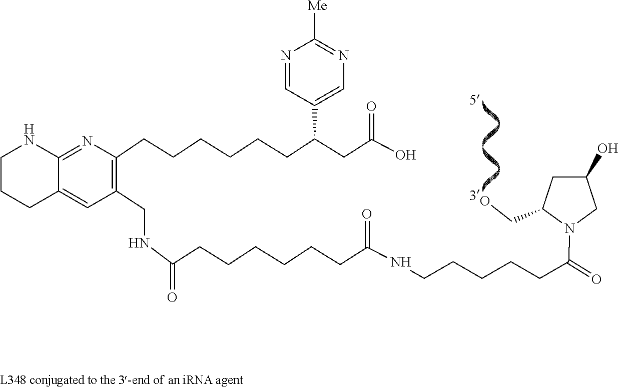 [SMILES: Cc1ncc([C@@H](CCCCCCc2nc3c(cc2CNC(=O)CCCCCCC(=O)NCCCCCC(=O)N2C[C@H](O)C[C@H]2CO)CCCN3)CC(=O)O)cn1]

In one embodiment, the iRNA agent comprises the structure:

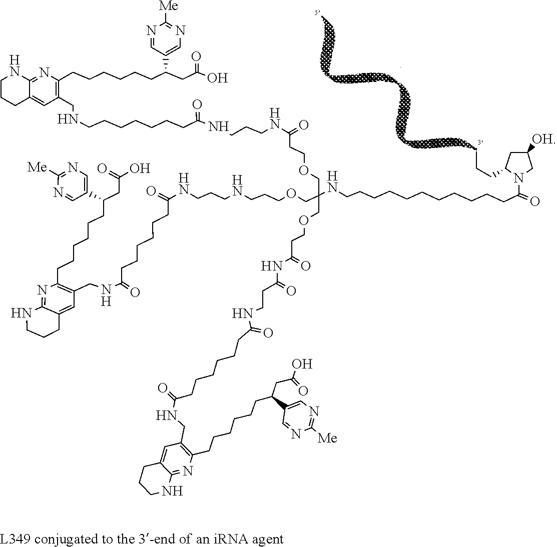 [SMILES: CCC[C@@H]1C[C@@H](O)CN1C(=O)CCCCCCCCCCCNC(COCCCNCCCNC(=O)CCCCCCC(=O)NCc1cc2c(nc1CCCCCC[C@@H](CC(=O)O)c1cnc(C)nc1)NCCC2)(COCCC(=O)NCCCNC(=O)CCCCCCCNCc1cc2c(nc1CCCCCC[C@@H](CC(=O)O)c1cnc(C)nc1)NCCC2)COCCC(=O)NC(=O)CCNC(=O)CCCCCCC(=O)NCc1cc2c(nc1CCCCCC[C@@H](CC(=O)O)c1cnc(C)nc1)NCCC2]

In one embodiment, the iRNA agent comprises the structure:

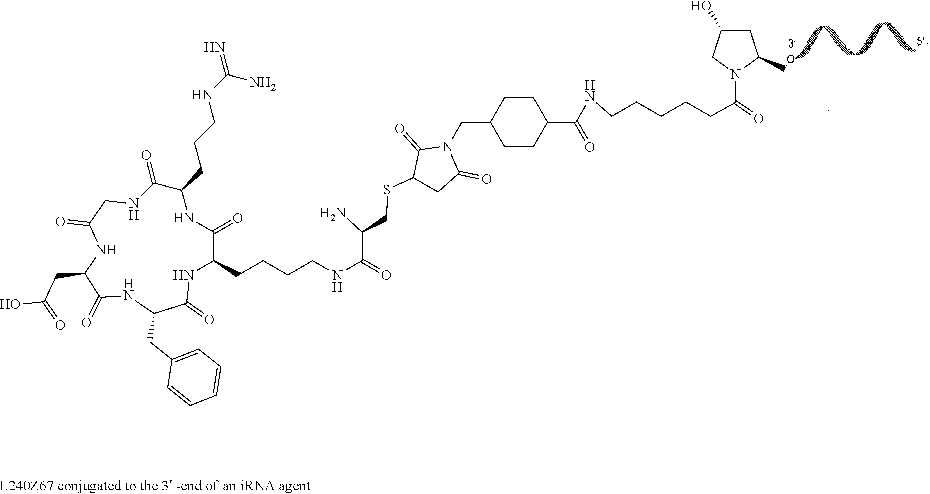 [SMILES: C[C@@H]1C[C@@H](O)CN1C(=O)CCCCCNC(=O)C1CCC(CN2C(=O)CC(SC[C@H](N)C(=O)NCCCC[C@H]3NC(=O)[C@H](Cc4ccccc4)NC(=O)[C@@H](CC(=O)O)NC(=O)CNC(=O)[C@@H](CCCNC(=N)N)NC3=O)C2=O)CC1]

In one embodiment, the iRNA agent comprises the structure:

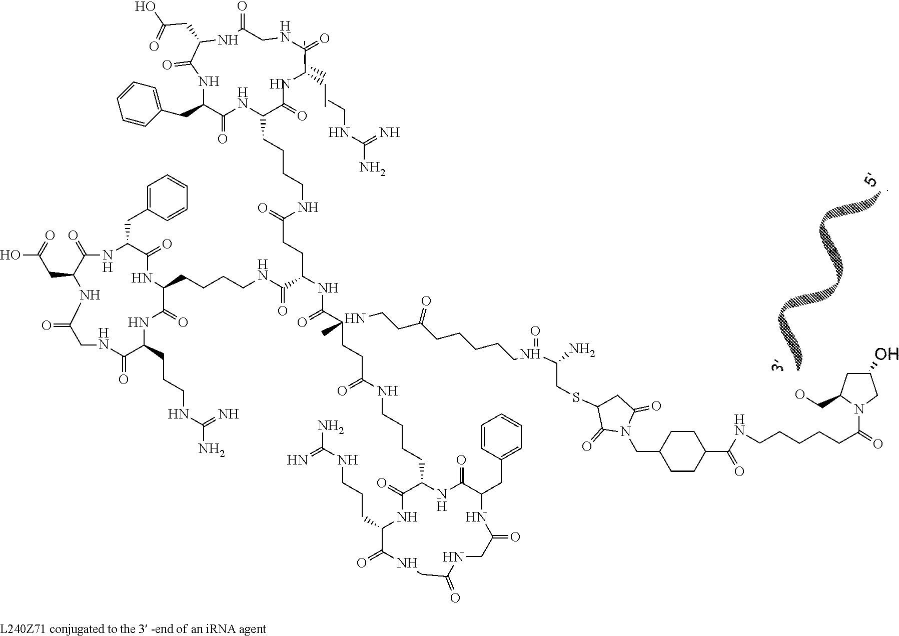

In one embodiment, the iRNA agent comprises the structure:

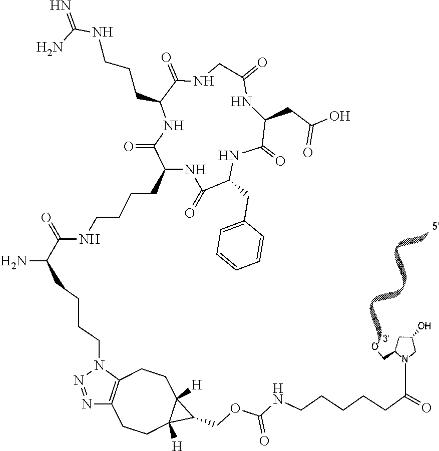 [SMILES: CC(=O)CCCCCNC(=O)OC[C@@H]1[C@@H]2CCc3nnn(CCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)c3CC[C@@H]21]

In one embodiment, the iRNA agent comprises the structure:

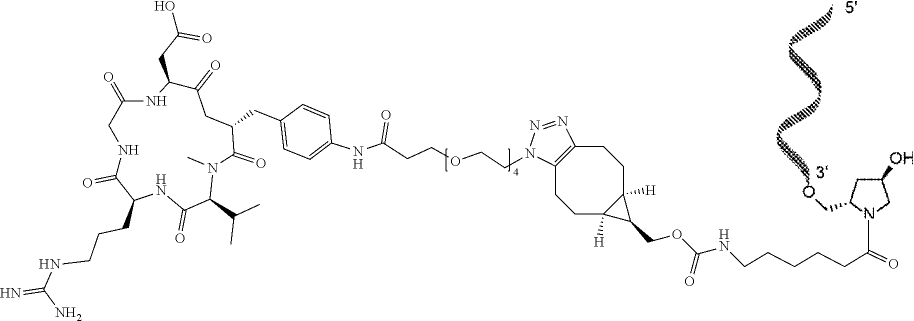

The dsRNA includes an antisense strand having a region of complementarity which is complementary to at least a part of an mRNA formed in the expression of a target gene. The region of complementarity is about 15-30 nucleotides or less in length. Upon contact with a cell expressing the target gene, the RNAi agent inhibits the expression of the target gene (e.g., a human gene, a primate gene, a non-primate gene) by at least 30% as compared to a similar cell not contacted with the RNAi agent or an RNAi agent not complementary to the target gene. Expression of the gene may be assayed by, for example, a PCR or branched DNA (bDNA)-based method, In situ hybridization (RNA) and immuno histochemistry (protein) and ELISA and all its derivatives (includes chemiluminescent method-Meso Scale Detection System) or by a protein-based method, such as by immunofluorescence analysis, using, for example, western blotting or flowcytometric techniques. In one embodiment, the level of knockdown is assayed in a rat eye model using an assay method provided in Example 2 below.

A dsRNA includes two RNA strands that are complementary and hybridize to form a duplex structure under conditions in which the dsRNA will be used. One strand of a dsRNA (the antisense strand) includes a region of complementarity that is substantially complementary, or fully complementary, to a target sequence. The target sequence can be derived from the sequence of an mRNA formed during the expression of a target gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 base pairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is 15 to 23 nucleotides in length, 19 to 23 nucleotides in length, or 25 to 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an RNAi agent useful to target expression or a target gene is not generated in the target cell by cleavage of a larger dsRNA.

A dsRNA as described herein can further include one or more single-stranded nucleotide overhangs e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the dsRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In one aspect, a dsRNA of the disclosure includes at least two nucleotide sequences, a sense sequence and an antisense sequence. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand (passenger strand), and the second oligonucleotide is described as the corresponding antisense strand (guide strand). Exemplary dsRNA agents of the invention are provided in Tables 2 and 3.

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences in Tables 2 and 3 are described as modified or conjugated sequences, the RNA of the RNAi agent of the disclosure e.g., a dsRNA of the disclosure, may comprise any one of the sequences set forth in Tables 2 and 3 that is un-modified, un-conjugated, or modified or conjugated differently than described therein. For example, although the sense strands of the agents of the invention may be conjugated to an integrin targeting ligand, these agents may also be conjugated to another moiety, as described herein. A lipophilic ligand can be included in any of the positions provided in the instant application.

The skilled person is well aware that dsRNAs having a duplex structure of about 20 to 23 base pairs, e.g., 21, base pairs have been hailed as particularly effective in inducing RNA interference (Elbashir et al., (2001) EMIBO J, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of a target gene by not more than 10, 15, 20, 25, 30, 35, 40, 45 or 50% inhibition from a dsRNA comprising the full sequence using the in vitro assay with, e.g., ocular cells and a 10 nM concentration of the RNA agent and the PCR assay as provided in the examples herein, are contemplated to be within the scope of the present disclosure.

In addition, the RNA agents described herein identify a site(s) in a target gene mRNA transcript that is susceptible to RISC-mediated cleavage. As such, the present disclosure further features RNAi agents that target within this site(s). As used herein, an RNAi agent is said to “target within” a particular site of an mRNA transcript if the RNAi agent promotes cleavage of the mRNA transcript anywhere within that particular site. Such an RNAi agent will generally include at least about 15 contiguous nucleotides, preferably at least 19 nucleotides, from one of the sequences provided herein coupled to additional nucleotide sequences taken from the region contiguous to the selected sequence in a target gene.

### III. Modifications of the RNAi Agents of the Disclosure

In one embodiment, the RNA of an RNAi agent of the disclosure, e.g., a dsRNA, is chemically modified to enhance stability or other beneficial characteristics. In certain embodiments of the disclosure, substantially all of the nucleotides of an RNAi agent of the disclosure are modified. In other embodiments of the disclosure, all of the nucleotides of an RNAi agent of the disclosure are modified. RNAi agents of the disclosure in which “substantially all of the nucleotides are modified” are largely but not wholly modified and can include not more than 5, 4, 3, 2, or unmodified nucleotides. In still other embodiments of the disclosure, RNAi agents of the disclosure can include not more than 5, 4, 3, 2 or 1 modified nucleotides.

The nucleic acids featured in the disclosure can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothioate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion.

In some embodiments, when the dsRNA agents of the invention are in the sodium salt form, sodium ions are present in the agent as counterions for all of the phosphodiester and/or phosphorothioate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in RNAi agents, in which both the sugar and the internucleoside linkage, i.e., the backbone, of the nucleotide units are replaced with alternate groups. The nucleobase units are maintained for hybridization with an appropriate nucleic acid target compound. One such oligomeric compound, a RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the RNAi agents of the disclosure are described in, for example, in Nielsen et al., Science, 1991, 254, 1497-1500.

Some embodiments featured in the disclosure include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—and —N(CH₃)—CH₂—CH₂—of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as —O—P(O){OH)—OCH₂—. Modified RNAs can also contain one or more substituted sugar moieties. The RNAi agents, e.g., dsRNAs, featured herein can include one of the following at the 2′-position: OH; F; O—, S—, or N-alkyl; O—, S—, or N-alkenyl; O—, S— or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₘOCH₃, O(CH₂)ₙNH₂, O(CH₂) ˜CH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ alkyl, substituted alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent, or a group for improving the pharmacodynamic properties of an RNAi agent, and other substituents having similar properties. In some embodiments, the modification includes a 2′-methoxyethoxy (2′-O—CH₂CH₂OCH₃, also known as 2′-O—(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)2ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)2. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions on the RNA of an RNAi agent, particularly the 3′ position of the sugar on the 3′ terminal nucleotide or in 2′-5′ linked dsRNAs and the 5′ position of 5′ terminal nucleotide. RNAi agents can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An RNAi agent of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further modified nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these modified nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

An RNAi agent of the disclosure can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′—CH2-O—2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH₂)—O—2′ (LNA); 4′-(CH₂)—S-2′; 4′—(CH₂)2-O—2′ (ENA); 4′—CH(CH₃)—O—2′ (also referred to as “constrained ethyl” or “cEt”) and 4′—CH(CH₂OCH₃)—O—2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′—CH₂—N(OCH₃)—2′ (and analogs thereof; see e.g., U.S. Pat. No. 8,278,425); 4′—CH₂—O—N(CH₃)—2′ (see, e.g., U.S. Patent Publication No. 2004/0171570); 4′—CH₂—N(R)—O—2′, wherein R is H, C₁-C₁₂ alkyl, or a protecting group (see, e.g., U.S. Pat. No. 7,427,672); 4′—CH₂—C(H)(CH₃)—2′ (see, e.g., Chattopadhyaya et al., J. Org. Chem., 2009, 74, 118-134); and 4′—CH₂—C(═CH₂)-2′ (and analogs thereof; see, e.g., U.S. Pat. No. 8,278,426). The entire contents of each of the foregoing are hereby incorporated herein by reference.

Additional representative US Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133;7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An RNAi agent of the disclosure can also be modified to include one or more constrained ethyl nucleotides. As used herein, a “constrained ethyl nucleotide” or “cEt” is a locked nucleic acid comprising a bicyclic sugar moiety comprising a 4′—CH(CH3)—O—2′ bridge. In one embodiment, a constrained ethyl nucleotide is in the S conformation referred to herein as “S-cEt.”

An RNAi agent of the disclosure may also include one or more “conformationally restricted nucleotides” (“CRN”). CRN are nucleotide analogs with a linker connecting the C2′ and C4′ carbons of ribose or the C3 and -C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US 2013/0190383; and WO 2013/036868, the entire contents of each of which are hereby incorporated herein by reference.

In some embodiments, an RNAi agent of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C₁′-C4′ have been removed (i.e. the covalent carbon-oxygen-carbon bond between the C₁′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

An RNAi agent of the disclosure may also include one or more “cyclohexene nucleic acids” or (“CeNA”). CeNA are nucleotide analogs with a replacement of the furanose moiety of DNA by a cyclohexene ring. Incorporation of cylcohexenyl nucleosides in a DNA chain increases the stability of a DNA/RNA hybrid. CeNA is stable against degradation in serum and a CeNA/RNA hybrid is able to activate E. Coli RNase H, resulting in cleavage of the RNA strand. (see Wang et al., Am. Chem. Soc. 2000, 122, 36, 8595-8602, hereby incorporated by reference).

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3”-phosphate, inverted base dT(idT) and others. Disclosure of this modification can be found in WO 2011/005861.

Other modifications of an RNAi agent of the disclosure include a 5′ phosphate or 5′ phosphate mimic, e.g., a 5′-terminal phosphate or phosphate mimic on the antisense strand of an RNAi agent. Suitable phosphate mimics are disclosed in, for example US 2012/0157511, the entire contents of which are incorporated herein by reference.

A. Modified RNAi agents Comprising Motifs of the Disclosure

In certain aspects of the disclosure, the double-stranded RNAi agents of the disclosure include agents with chemical modifications as disclosed, for example, in WO 2013/075035, the entire contents of which are incorporated herein by reference. As shown herein and in WO 2013/075035, a superior result may be obtained by introducing one or more motifs of three identical modifications on three consecutive nucleotides into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally conjugated with a lipophilic ligand, e.g., a C16 ligand, for instance on the sense strand. The RNAi agent may be optionally modified with a (S)-glycol nucleic acid (GNA) modification, for instance on one or more residues of the antisense strand. The resulting RNAi agents present superior gene silencing activity.

Accordingly, the disclosure provides double stranded RNAi agents capable of inhibiting the expression of a target gene in vivo. The RNAi agent comprises a sense strand and an antisense strand. Each strand of the RNAi agent may be 15-30 nucleotides in length. For example, each strand may be 16-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. In certain embodiments, each strand is 19-23 nucleotides in length.

The sense strand and antisense strand typically form a duplex double stranded RNA (“dsRNA”), also referred to herein as an “RNAi agent.” The duplex region of an RNAi agent may be 15-30 nucleotide pairs in length. For example, the duplex region can be 16-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length. In another embodiment, the duplex region is 19-21 nucleotide pairs in length.

In one embodiment, the RNAi agent may contain one or more overhang regions or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In another embodiment, the nucleotide overhang region is 2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-O-methyl, thymidine (T), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands.

In one embodiment, this 3′-overhang is present in the antisense strand. In one embodiment, this 3′-overhang is present in the sense strand.

The dsRNAi agent may contain only a single overhang, which can strengthen the interference activity of the RNAi, without affecting its overall stability. For example, the single-stranded overhang may be located at the 3-terminal end of the sense strand or, alternatively, at the 3-terminal end of the antisense strand. The RNAi may also have a blunt end, located at the 5′-end of the antisense strand (i.e., the 3′-end of the sense strand) or viceversa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double blunt-ended of 19 nucleotides in length, wherein the sense strand contains at least one motif of three 2′-F modifications on three consecutive nucleotides at positions 7, 8, and 9 from the 5′end. The antisense strand contains at least one motif of three 2′-O-methyl modifications on three consecutive nucleotides at positions 11, 12, and 13 from the 5′end.

In another embodiment, the RNAi agent is a double blunt-ended of 20 nucleotides in length, wherein the sense strand contains at least one motif of three 2′-F modifications on three consecutive nucleotides at positions 8, 9, and 10 from the 5′end. The antisense strand contains at least one motif of three 2′-O-methyl modifications on three consecutive nucleotides at positions 11, 12, and 13 from the 5′end.

In yet another embodiment, the RNAi agent is a double blunt-ended of 21 nucleotides in length, wherein the sense strand contains at least one motif of three 2′-F modifications on three consecutive nucleotides at positions 9, 10, and 11 from the 5′end. The antisense strand contains at least one motif of three 2′-O-methyl modifications on three consecutive nucleotides at positions 11, 12, and 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, and 11 from the 5′end; the antisense strand contains at least one motif of three 2′-O-methyl modifications on three consecutive nucleotides at positions 11, 12, and 13 from the 5′end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three 3′-nucleotides of the antisense strand, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 2′-fluoro, e.g., in an alternating motif. Optionally, the RNAi agent further comprises a ligand (e.g., a lipophilic ligand, optionally a C16 ligand).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, and 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, and 11 positions; 10, 11, and 12 positions; 11, 12, and 13 positions; 12, 13, and 14 positions; or 13, 14, and 15 positions of the antisense strand, the count starting from the 1P nucleotide from the 5′-end of the antisense strand, or, the count starting from the 1P paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other, then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two, or three nucleotides in the duplex region.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxythimidine (dT). In another embodiment, the nucleotide at the 3′-end of the antisense strand is deoxythimidine (dT). In one embodiment, there is a short sequence of deoxythimidine nucleotides, for example, two dT nucleotides on the 3′-end of the sense or antisense strand.

In one embodiment, the sense strand sequence may be represented by formula (I):

(I)
5′nₚ-Nₐ-(X X X)ᵢ-N_{b}-Y Y Y -N_{b}-(Z Z Z)ⱼ-Nₐ-n_{q}3′

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each np and nq independently represent an overhang nucleotide;
wherein Nb and Y do not have the same modification; and
XXX, YYY and ZZZ each independently represent one motif of three identical modifications on three consecutive nucleotides. Preferably YYY is all 2′-F modified nucleotides.

In one embodiment, the Nₐ or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11,12 or 11, 12, 13) of -the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

(Ib)
5′nₚ-Nₐ-YYY-N_{b}-ZZZ-Nₐ-n_{q}3′;
(Ic)
5′nₚ-Nₐ-XXX-N_{b}-YYY-Nₐ-n_{q}3′;
or
(Id)
5′nₚ-Nₐ-XXX-N_{b}-YYY-N_{b}-ZZZ-Nₐ-n_{q}3′.

When the sense strand is represented by formula (Ib), N_{b} represents an oligonucleotide sequence comprising 0-10, 0-7, 0-5, 0-4, 0-2 or 0 modified nucleotides.

Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)
5′nₚ-Nₐ-YYY- Nₐ-n_{q}3′.

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

(II)
5′n_{q}′-Nₐ′-(Z′Z′Z′)ₖ-N_{b}′-Y′Y′Y′-N_{b}′-(X′X′X′)ₗ-N′ₐ-
nₚ′3′

wherein:
k and 1 are each independently 0 or 1;
p′ and q′ are each independently 0-6;
each Nₐ′ independently represents an oligonucleotide sequence comprising 0-25 modified nucleotides, each sequence comprising at least two differently modified nucleotides; each N_{b}′ independently represents an oligonucleotide sequence comprising 0-10 modified nucleotides; each np′ and nq' independently represent an overhang nucleotide; wherein N_{b}′ and Y′ do not have the same modification;
and X′X′X′, Y′Y′Y' and Z′Z′Z′ each independently represent one motif of three identical modifications on three consecutive nucleotides.

In one embodiment, the Nₐ′ or N_{b}′ comprise modifications of alternating pattern.

The Y′Y′Y' motif occurs at or near the cleavage site of the antisense strand. For example, when the RNAi agent has a duplex region of 17-23nucleotidein length, the Y′Y′Y' motif can occur at positions 9, 10, 11;10, 11, 12; 11, 12, 13; 12, 13, 14; or 13, 14, 15 of the antisense strand, with the count starting from the 1St nucleotide, from the 5′-end; or optionally, the count starting at the 1^{st} paired nucleotide within the duplex region, from the 5′-end. Preferably, the Y′Y′Y' motif occurs at positions 11, 12, 13.

In one embodiment, Y′Y′Y' motif is all 2′-OMe modified nucleotides.

In one embodiment, k is 1 and 1 is 0, or k is 0 and 1 is 1, or both k and 1 are 1.

The antisense strand can therefore be represented by the following formulas:

(IIb)
5′n_{q}′-Nₐ′-Z′Z′Z′-N_{b}′-Y′Y′Y′-Nₐ′-nₚ′3′;
(IIc)
5′n_{q}′-Nₐ′-Y′Y′Y′-N_{b}′-X′X′X′-nₚ′3′;
or
(IId)
5′n_{q}′-Nₐ′- Z′Z′Z′-N_{b}′-Y′Y′Y′-N_{b}′-X′X′X′-Nₐ′-nₚ′3′.

When the antisense strand is represented by formula (IIb), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and 1 is 0 and the antisense strand may be represented by the formula:

(Ia)
5′n_{p′}-N_{a′}-Y′Y′Y′- N_{a′}-n_{q′}3′.

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z', in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y' motif occurring at positions 11, 12, 13 of the strand, the count starting from the 1^{st} nucleotide from the 5′-end, or optionally, the count starting at the 1^{st} paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with an antisense strand being represented by any one of formulas (IIa), (IIb), (IIc), and (IId), respectively.

Accordingly, the RNAi agents for use in the methods of the disclosure may comprise a sense strand and an antisense strand, each strand having 14 to 30 nucleotides, the RNAi duplex represented by formula (III):

(III)
sense:
5′nₚ-Nₐ-(X X X)ᵢ-N_{b}- Y Y Y -N_{b}-(ZZZ)ⱼ-Nₐ-n_{q}3′
antisense:
3′nₚ′-Nₐ′-(X′X′X′)ₖ-N_{b}′-Y′Y′Y′-N_{b}′-(Z′Z′Z′)ₗ-Nₐ′-
n_{q}′5′

wherein:
i, j, k, and 1 are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein
each np′, np, nq′, and nq, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and 1 are 0; or both k and 1 are 1.

Exemplary combinations of the sense strand and antisense strand forming an RNAi duplex include the formulas below:

(IIIa)
5′nₚ-Nₐ-Y Y Y -Nₐ-n_{q}3′
3′nₚ′-Nₐ′-Y′Y′Y′ -Nₐ′n_{q}′5′
(IIIb)
5′nₚ-Nₐ-Y Y Y -N_{b} -ZZZ-Nₐ-n_{q}3′
3′nₚ′-Nₐ′-Y′Y′Y′-N_{b}′-Z′Z′Z′-Nₐ′n_{q}′5′
(IIIc)
5′nₚ-Nₐ- X X X -N_{b} -Y Y Y -Nₐ-n_{q}3′
3′nₚ′-Nₐ′-X′X′X′-N_{b}′-Y′Y′Y′-Nₐ′-n_{q}′5′
(IIId)
5′nₚ -Nₐ-X X X-N_{b}-Y Y Y -N_{b}-ZZZ -Nₐ-n_{q}3′
3′nₚ′-Nₐ′-X′X′X′-N_{b}′-Y′Y′Y′-N_{b}′-Z′Z′Z′-Nₐ-n_{q}′5′

When the RNAi agent is represented by formula (IIIa), each Nₐ independently represents an oligonucleotide sequence comprising 2-20, 2-15, or 2-10 modified nucleotides.

When the RNAi agent is represented by formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as formula (IIc), each N_{b}, N_{b}′ independently represents an oligonucleotide sequence comprising 0-10, 0-7, 0-10, 0-7, 0-5, 0-4, 0-2 or 0 modified nucleotides. Each Nₐindependently represents an oligonucleotide sequence comprising 2-20, 2-15, or 2-10 modified nucleotides.

When the RNAi agent is represented as formula (IIId), each N_{b}, N_{b}′ independently represents an oligonucleotide sequence comprising 0-10, 0-7, 0-10, 0-7, 0-5, 0-4, 0-2 or 0 modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern.

In one embodiment, when the RNAi agent is represented by formula (IIId), the Nₐ modifications are 2′-O-methyl or 2′-fluoro modifications. In another embodiment, when the RNAi agent is represented by formula (IIId), the Nₐ modifications are 2′-O-methyl or 2′-fluoro modifications and np′>0 and at least one np′ is linked to a neighboring nucleotide a via phosphorothioate linkage. In yet another embodiment, when the RNAi agent is represented by formula (IIId), the Nₐ modifications are 2′-O-methyl or 2′-fluoro modifications, np′>0 and at least one np′ is linked to a neighboring nucleotide via phosphorothioate linkage, and the sense strand is conjugated to one or more C16 (or related) moieties attached through a bivalent or trivalent branched linker (described below). In another embodiment, when the RNAi agent is represented by formula (IIId), the Nₐ modifications are 2′-O-methyl or 2′-fluoro modifications, np′>0 and at least one np′ is linked to a neighboring nucleotide via phosphorothioate linkage, the sense strand comprises at least one phosphorothioate linkage, and the sense strand is conjugated to one or more lipophilic, e.g., C16 (or related) moieties, optionally attached through a bivalent or trivalent branched linker.

In one embodiment, when the RNAi agent is represented by formula (IIIa), the Nₐ modifications are 2′-O-methyl or 2′-fluoro modifications, np′>0 and at least one np′ is linked to a neighboring nucleotide via phosphorothioate linkage, the sense strand comprises at least one phosphorothioate linkage, and the sense strand is conjugated to one or more lipophilic, e.g., C16 (or related) moieties attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to to a ligand. Each of the agents can target the same gene or two different genes; or each of the agents can target same gene at two different target sites.

Various publications describe multimeric RNAi agents that can be used in the methods of the disclosure. Such publications include WO2007/091269, WO2010/141511, WO2007/117686, WO2009/014887, and WO2011/031520; and U.S. Pat. No. 7,858,769, the entire contents of each of which are hereby incorporated herein by reference.

In certain embodiments, the compositions and methods of the disclosure include a vinyl phosphonate (VP) modification of an RNAi agent as described herein. In exemplary embodiments, a vinyl phosphonate of the disclosure has the following structure:

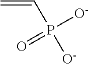

For example, when the phosphate mimic is a 5′-vinyl phosphonate (VP), the 5′-terminal nucleotide can have the following structure,

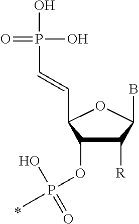

wherein * indicates the location of the bond to 5′-position of the adjacent nucleotide:
R is hydrogen, hydroxy, methoxy, fluoro (e.g., hydroxy or methoxy), or another modification described herein; and
B is a nucleobase or a modified nucleobase, optionally where B is adenine, guanine, cytosine, thymine or uracil.

In one embodiment, the double stranded RNAi agent of the invention further comprises a 5′-phosphate or a 5′-phosphate mimic at the 5′ nucleotide of the antisense strand. In another embodiment, the double stranded RNAi agent further comprises a 5′-phosphate mimic at the 5′ nucleotide of the antisense strand. In a specific embodiment, the 5′-phosphate mimic is a 5′-vinyl phosphonate (5′-VP). In one embodiment, the phosphate mimic is a 5′-cyclopropyl phosphonate (VP). In some embodiments, the 5′-end of the antisense strand of the double-stranded iRNA agent does not contain a 5′-vinyl phosphonate (VP).

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphate modifications are also contemplated for the compositions and methods of the instant disclosure. An exemplary vinyl phosphate structure is:

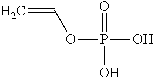

For example, when the phosphate mimic is a 5′-vinyl phosphate, the 5′-terminal nucleotide can have the immediately structure, where the phosphonate group is replaced by a phosphate.
i. Thermally Destabilizing Modifications

In certain embodiments, a dsRNA molecule can be optimized for RNA interference by incorporating thermally destabilizing modifications in the seed region of the antisense strand. As used herein “seed region” means at positions 2-9 of the 5′-end of the referenced strand. For example, thermally destabilizing modifications can be incorporated in the seed region of the antisense strand to reduce or inhibit off-target gene silencing.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1-4° C., such as one, two, three or four degrees Celsius. And, the term “thermally destabilizing nucleotide” refers to a nucleotide containing one or more thermally destabilizing modifications.

It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, or preferably positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. In some embodiments, the thermally destabilizing modification of the duplex is located at position 2, 3, 4, 5 or 9 from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). Exemplified abasic modifications include, but are not limited to the following.

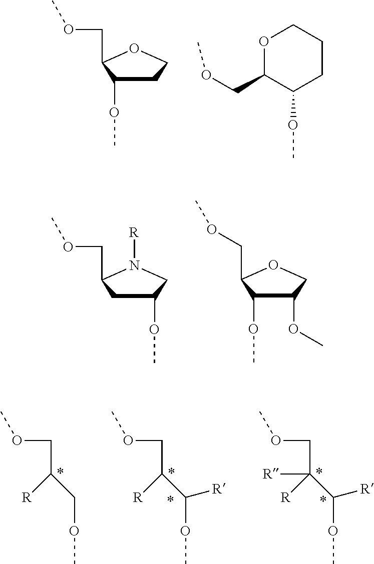

### Wherein R═H, Me, Et or OMe; R′=H, Me, Et or OMe; R″=H, Me, Et or OMe

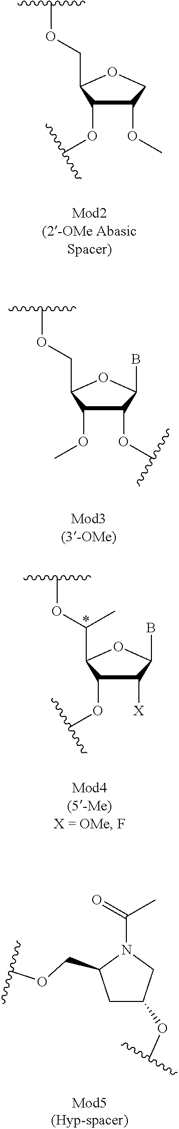

wherein B is a modified or unmodified nucleobase.

Exemplified sugar modifications include, but are not limited to the following:

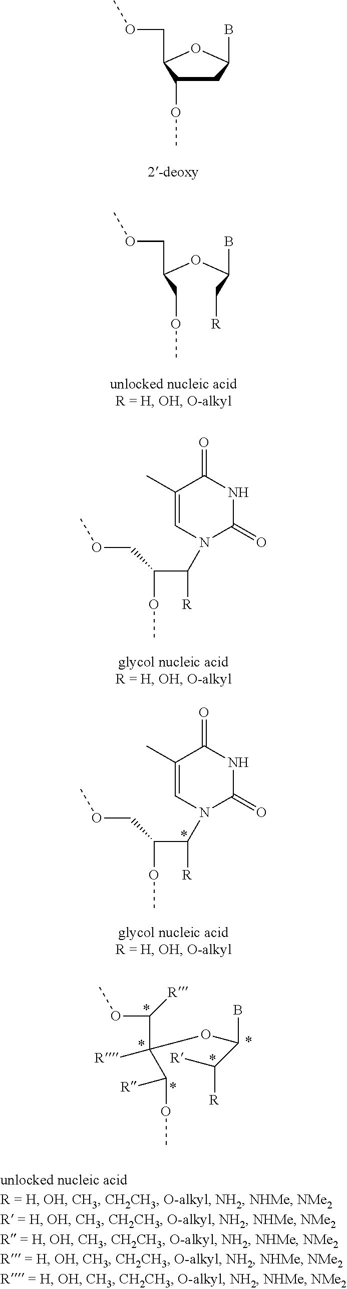

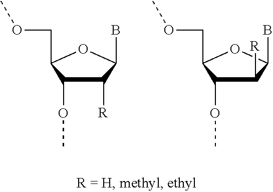

wherein B is a modified or unmodified nucleobase.

In some embodiments the thermally destabilizing modification of the duplex is selected from the group consisting of:

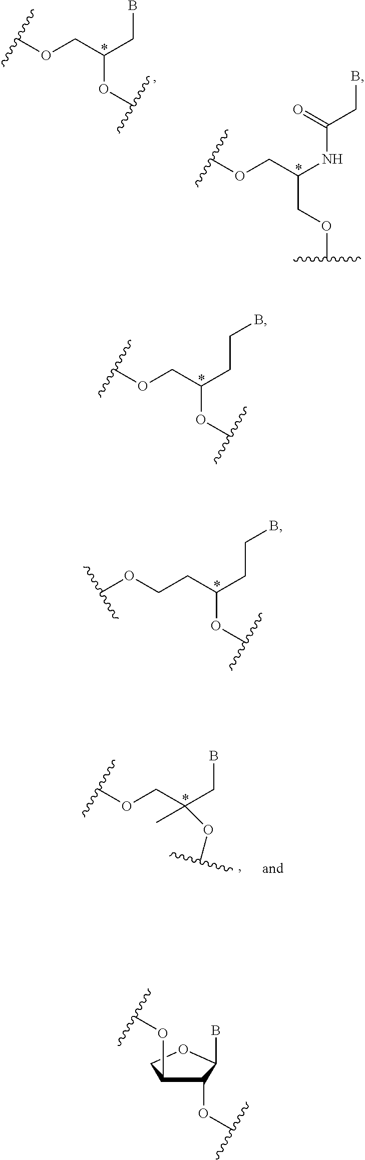

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2′, C2′-C3′, C3′-C4′, C4′-04′, or C₁′-04′) is absent or at least one of ribose carbons or oxygen (e.g., C1′, C2′, C3′, C4′ or 04′) are independently or in combination absent from the nucleotide. In some embodiments, acyclic nucleotide is

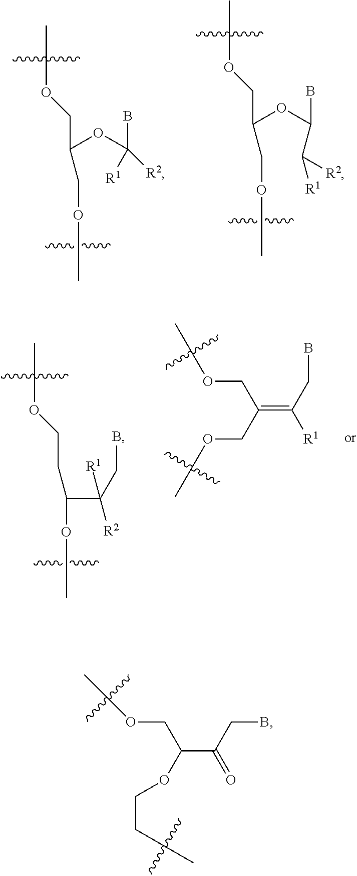

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C₁′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

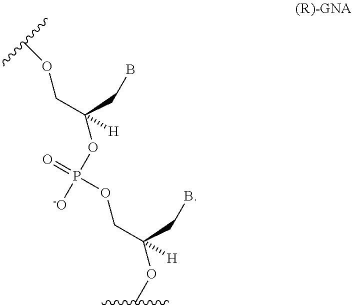

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W-C H-bonding to complementary base on the target mRNA such as:

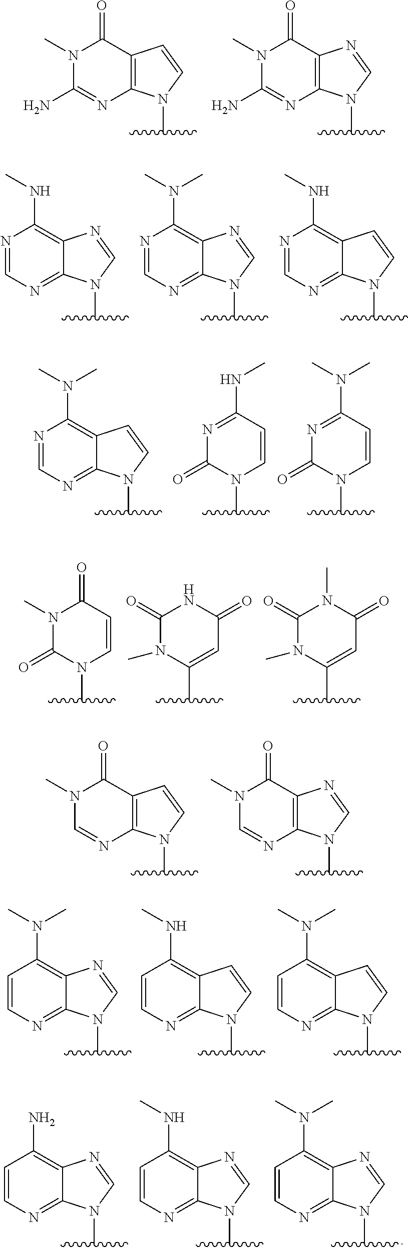

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

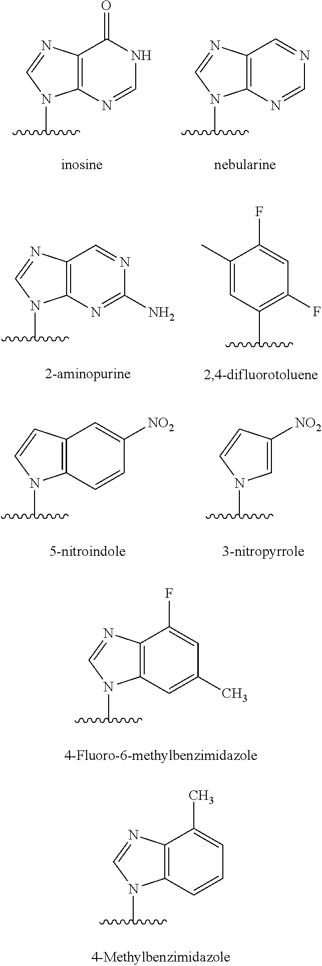

In some embodiments the thermally destabilizing modification of the duplex in the seed region of the antisense strand includes one or more [symbol] nucleotide complementary to the base on the target mRNA, such as:

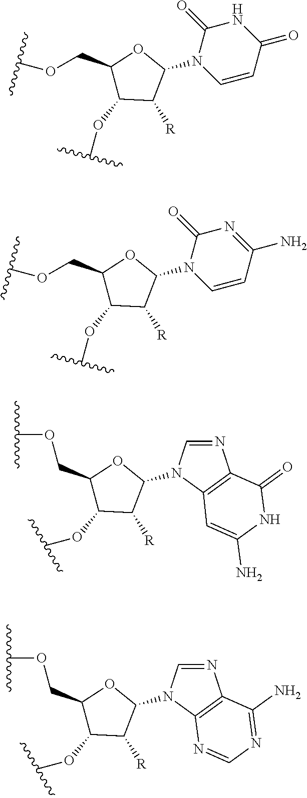

wherein R is H, OH, OCH₃, F, NH₂, NHMe, NMe₂ or O-alkyl.

Exemplary phosphate modifications known to decrease the thermal stability of dsRNA duplexes compared to natural phosphodiester linkages are:

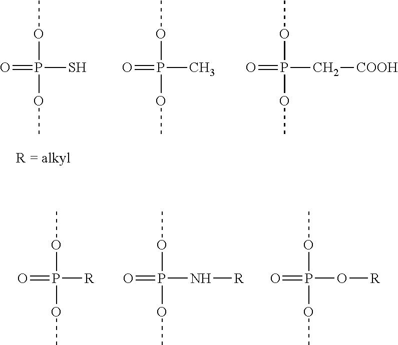

The alkyl for the R group can be a C₁-C₆alkyl. Specific alkyls for the R group include, but are not limited to methyl, ethyl, propyl, isopropyl, butyl, pentyl and hexyl.

As the skilled artisan will recognize, in view of the functional role of nucleobases is defining specificity of an RNAi agent of the disclosure, while nucleobase modifications can be performed in the various manners as described herein, e.g., to introduce destabilizing modifications into an RNAi agent of the disclosure, e.g., for purpose of enhancing on-target effect relative to off-target effect, the range of modifications available and, in general, present upon RNAi agents of the disclosure tends to be much greater for non-nucleobase modifications, e.g., modifications to sugar groups or phosphate backbones of polyribonucleotides. Such modifications are described in greater detail in other sections of the instant disclosure and are expressly contemplated for RNAi agents of the disclosure, either possessing native nucleobases or modified nucleobases as described above or elsewhere herein.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14, and 16 from the 5′-end. In some other embodiments, the antisense comprises stabilizing modifications at positions 2, 6, 14, and 16 from the 5′-end. In still some other embodiments, the antisense comprises stabilizing modifications at positions 2, 14, and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10, and 11 from the 5′-end. In some other embodiments, the sense strand comprises stabilizing modifications at positions 7, 9, 10, and 11 from the 5′-end. In some embodiments, the sense strand comprises stabilizing modifications at positions opposite or complementary to positions 11, 12, and 15 of the antisense strand, counting from the 5′-end of the antisense strand. In some other embodiments, the sense strand comprises stabilizing modifications at positions opposite or complementary to positions 11, 12, 13, and 15 of the antisense strand, counting from the 5′-end of the antisense strand. In some embodiments, the sense strand comprises a block of two, three, or four stabilizing modifications.

In some embodiments, the sense strand does not comprise a stabilizing modification in position opposite or complementary to the thermally destabilizing modification of the duplex in the antisense strand. Exemplary thermally stabilizing modifications include, but are not limited to, 2′-fluoro modifications. Other thermally stabilizing modifications include, but are not limited to, LNA.

In some embodiments, the dsRNA of the disclosure comprises at least four (e.g., four, five, six, seven, eight, nine, ten, or more) 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten, or more) 2′-fluoro nucleotides. Without limitations, a 2′-fluoro modification in the antisense strand can be present at any positions. In some embodiments, the antisense comprises 2′-fluoro nucleotides at positions 2, 6, 8, 9, 14, and 16 from the 5′-end. In some other embodiments, the antisense comprises 2′-fluoro nucleotides at positions 2, 6, 14, and 16 from the 5′-end. In still some other embodiments, the antisense comprises 2′-fluoro nucleotides at positions 2, 14, and 16 from the 5′-end.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) 2′-fluoro nucleotides. Without limitations, a 2′-fluoro modification in the sense strand can be present at any positions. In some embodiments, the antisense comprises 2′-fluoro nucleotides at positions 7, 10, and 11 from the 5′-end. In some other embodiments, the sense strand comprises 2′-fluoro nucleotides at positions 7, 9, 10, and 11 from the 5′-end. In some embodiments, the sense strand comprises 2′-fluoro nucleotides at positions opposite or complementary to positions 11, 12, and 15 of the antisense strand, counting from the 5′-end of the antisense strand. In some other embodiments, the sense strand comprises 2′-fluoro nucleotides at positions opposite or complementary to positions 11, 12, 13, and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complementary to the thermally destabilizing modification of the duplex in the antisense strand.

In some embodiments, the dsRNA molecule of the disclosure comprises a 21 nucleotides (nt) sense strand and a 23 nucleotides (nt) antisense, wherein the antisense strand contains at least one thermally destabilizing nucleotide, where the at least one thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; and (vii) the dsRNA comprises a blunt end at 5′-end of the antisense strand. Preferably, the 2 nt overhang is at the 3′-end of the antisense.

In some embodiments, the dsRNA molecule of the disclosure comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand). For example, the thermally destabilizing nucleotide occurs between positions opposite or complementary to positions 14-17 of the 5′-end of the sense strand, and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA molecule of the disclosure comprises a sense and antisense strands, wherein said dsRNA molecule comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′end, wherein the 3′ end of said sense strand and the 5′ end of said antisense strand form a blunt end and said antisense strand is 1-4 nucleotides longer at its 3′ end than the sense strand, wherein the duplex region which is at least 25 nucleotides in length, and said antisense strand is sufficiently complementary to a target mRNA along at least 19 nt of said antisense strand length to reduce target gene expression when said dsRNA molecule is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA has a duplex region of 12-29 nucleotide pairs in length.

In some embodiments, every nucleotide in the sense strand and antisense strand of the dsRNA molecule may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxy-2′-fluoro nucleotide, 2′-O-N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the dsRNA molecule of the disclosure comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule of the disclosure comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

The dsRNA molecule of the disclosure may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13, or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11, or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9, or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7, or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5, or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3, or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the disclosure further comprises one or more phosphorothioate or methylphosphonate internucleotide linkage modification within 1-10 of the termini position(s) of the sense or antisense strand. For example, at least 2, 3, 4, 5, 6, 7, 8, 9, or 10 nucleotides may be linked through phosphorothioate or methylphosphonate internucleotide linkage at one end or both ends of the sense or antisense strand.

In some embodiments, the dsRNA molecule of the disclosure further comprises one or more phosphorothioate or methylphosphonate internucleotide linkage modification within 1-10 of the internal region of the duplex of each of the sense or antisense strand. For example, at least 2, 3, 4, 5, 6, 7, 8, 9, or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the disclosure further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA molecule of the disclosure further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, compound of the disclosure comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the disclosure comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the disclosure comprises a 5′-block is an Sp block wherein each sugar moiety comprises a 2′-F modification. In some embodiments, a 5′-block is an Sp block wherein each of internucleotidic linkage is a modified internucleotidic linkage and each sugar moiety comprises a 2′-F modification. In some embodiments, a 5′-block is an Sp block wherein each of internucleotidic linkage is a phosphorothioate linkage and each sugar moiety comprises a 2′-F modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-F modification. In some embodiments, a 3′-block is an Sp block wherein each of internucleotidic linkage is a modified internucleotidic linkage and each sugar moiety comprises a 2′-F modification. In some embodiments, a 3′-block is an Sp block wherein each of internucleotidic linkage is a phosphorothioate linkage and each sugar moiety comprises a 2′-F modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the disclosure comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2, 3, 4 or 5 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3, 4 or 5 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In some embodiments, the dsRNA molecule of the disclosure comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA molecule of the disclosure comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

It was found that introducing 4′-modified or 5′-modified nucleotide to the 3′-end of a phosphodiester (PO), phosphorothioate (PS), or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of single stranded or double stranded siRNA. For instance, a 5′-alkylated nucleoside may be introduced at the 3′-end of a dinucleotide at any position of single stranded or double stranded siRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, 4′-modified nucleoside is introduced at the 3′-end of a dinucleotide at any position of single stranded or double stranded siRNA. For instance, a 4′-alkylated nucleoside may be introduced at the 3′-end of a dinucleotide at any position of single stranded or double stranded siRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, 5′-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or S isomer.

In some embodiments, 4′-alkylated nucleoside is introduced at any position on the sense strand or antisense strand of a dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, 4′-O-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or S isomer.

In some embodiments, the dsRNA molecule of the disclosure can comprise 2′-5′ linkages (with 2′-H, 2′-OH and 2′-OMe and with P=0 or P=S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA molecule of the disclosure can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC.

Various publications describe multimeric siRNA which can all be used with the dsRNA of the disclosure. Such publications include WO2007/091269, U.S. Pat. No. 7,858,769, WO2010/141511, WO2007/117686, WO2009/014887, and WO2011/031520 which are hereby incorporated by their entirely.

As described in more detail below, the RNAi agent that contains conjugations of one or more carbohydrate moieties to an RNAi agent may improve one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group. The cyclic group can be selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalinyl. The acyclic group can be a serinol backbone or diethanolamine backbone.

IV. iRNAs Conjugated to Additional Ligands

Another modification of the RNA of an iRNA of the invention involves chemically linking to the iRNA one or more additional ligands, moieties or conjugates that enhance the activity, cellular distribution or cellular uptake of the iRNA, e.g., into a cell. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBSLett., 1990, 259:327-330; Svinarchuk et al., Biochimie, 1993, 75:49-54), aphospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting or lifetime of an iRNA agent into which it is incorporated. In some embodiments, a ligand provides an enhanced affinity for a selected target, e.g., molecule, cell or cell type, compartment, e.g., a cellular or organ compartment, tissue, organ or region of the body, as, e.g., compared to a species absent such a ligand. Typical ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an a helical peptide.

The iRNA of the invention can include ligands, including additional targeting groups, e.g., a cell or tissue targeting agent, e.g., a lectin, glycoprotein, lipid or protein, e.g., an antibody, that binds to a specified cell type such as an ocular cell. The additional targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, or biotin. In certain embodiments, the additional ligand is a multivalent galactose, e.g., an N-acetyl-galactosamine.

In one embodiment, the ligand is an integrin targeting ligand. In another embodiment, the integrin targeting ligand is selected from the group consisting of RGD peptide ligands, RGD peptide mimetics including small molecule RGD mimics, vitronectin, cilingetide, fibronectin, collagen, laminin, fibrinogen, thrombospondin, and glycoproteins (e.g., tenascin C, osteopontin, and nefronectin).

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), mPEG, [mPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or ocular cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, polyethylene glycol (PEG), vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs of the invention may be synthesized by the use of an oligonucleotide that bears a pendant reactive functionality, such as that derived from the attachment of a linking molecule onto the oligonucleotide (described below). This reactive oligonucleotide may be reacted directly with commercially available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the additional ligand or conjugate is a lipid or lipid-based molecule. Such a lipid or lipid-based molecule can typically bind a serum protein, such as human serum albumin (HSA). An HSA binding ligand allows for distribution of the conjugate to a target tissue of the body. For example, the target tissue can be the eye, including cells of the eye. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

In certain embodiments, additional molecules that binds with in vitreous humor like hyaluronic acid, glucose, anions, cations, ions, collagen.

A lipid-based ligand can be used to modulate, e.g., control (e.g., inhibit) the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid-based ligand binds HSA. For example, the ligand can bind HSA with a sufficient affinity such that distribution of the conjugate to a non-kidney tissue is enhanced. However, the affinity is typically not so strong that the HSA-ligand binding cannot be reversed.

In certain embodiments, the lipid-based ligand binds HSA weakly or not at all, such that distribution of the conjugate to the kidney is enhanced. Other moieties that target to kidney cells can also be used in place of or in addition to the lipid-based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

### B. Cell Permeation Agents

In another aspect, the additional ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is typically an α-helical agent and can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 1). An RFGF analogue (e.g., amino acid sequence AALLPVLLAAP (SEQ ID NO: 2)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 3)) and the Drosophila Antennapedia protein (RQIKIWFQNRRMKWKK (SEQ ID NO: 4)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). In some embodiments, the peptide or peptidomimetic is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., a-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, an iRNA further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di—and tri-saccharides include sugars having two or three monosaccharide units (e.g., C5, C6, C7, or C8).

In certain embodiments, a carbohydrate conjugate comprises a monosaccharide. In certain embodiments, the monosaccharide is an N-acetylgalactosamine (GalNAc). GalNAc conjugates, which comprise one or more N-acetylgalactosamine (GalNAc) derivatives, are described, for example, in U.S. Pat. No. 8,106,022, the entire content of which is hereby incorporated herein by reference.

Additional carbohydrate conjugates and linkers suitable for use in the present invention include those described in WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by 0, S, S(0), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In certain embodiments, the linker is between about 1-24 atoms, 2-24, 3-24, 4-24, 5-24, 6-24, 6-18, 7-18, 8-18 atoms, 7-17, 8-17, 6-16, 7-16, or 8-16 atoms.

Non-limiting examples of linkers useful for preparing a conjugate and/or ligand of the present invention include those described in WO 2019/217459, which is hereby incorporated by reference in its entirety.

Additional non-limiting examples of linkers are shown in Table 1.

The linkers are shown with the protecting group DMTr. When conjugated, the DMTr group is removed and the adjacent oxygen atom is the site of attachment of the Linker to the Cleavable Linkage and oligonucleotide. The sqiggly line is the point of attachment for the Ligand. X is hydrogen. When the Linker group is incorporated into an intermediate compound useful for preparing a conjugate of the present invention, X can be a reactive phosphoramidite

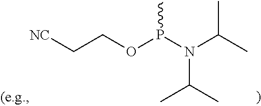

compatible with solid phase oligonucleotide synthesis and deprotection or attached to a solid support

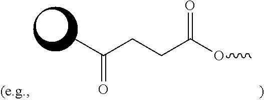

that enable solid phase oligonucleotide synthesis.

TABLE 1

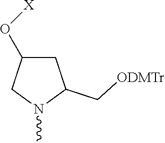

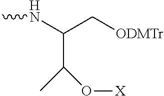

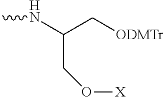

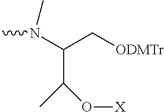

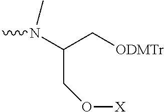

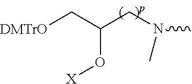

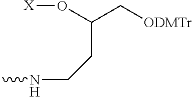

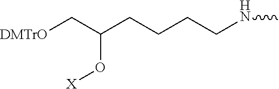

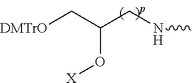

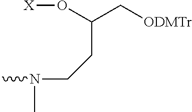

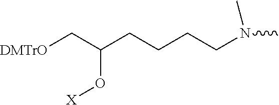

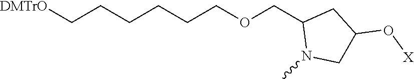

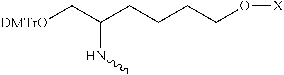

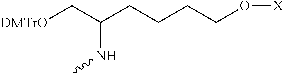

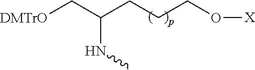

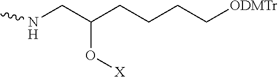

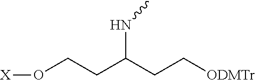

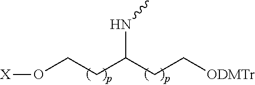

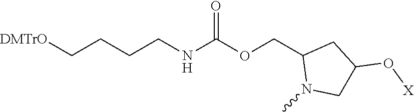

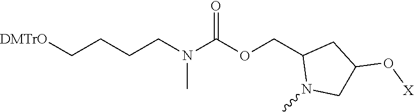

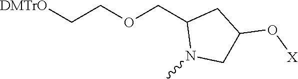

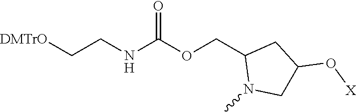

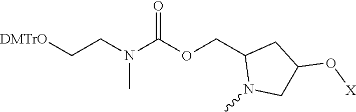

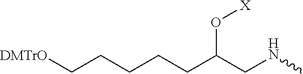

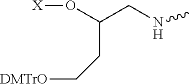

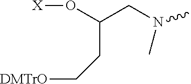

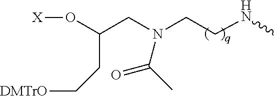

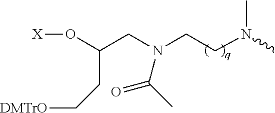

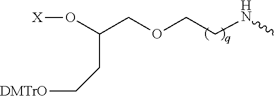

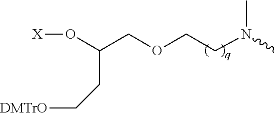

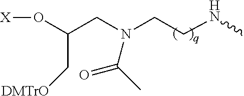

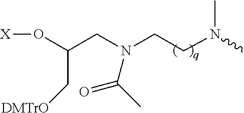

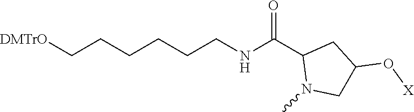

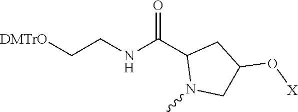

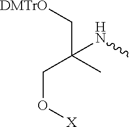

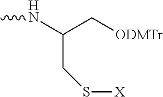

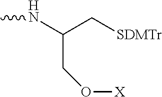

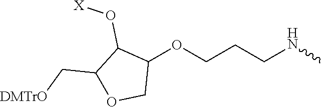

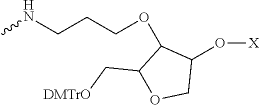

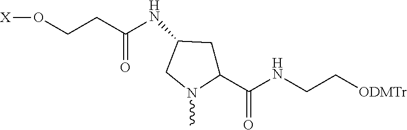

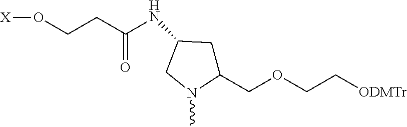

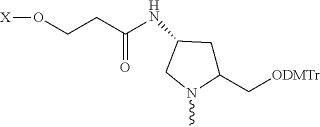

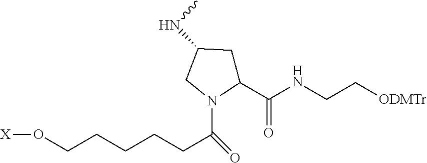

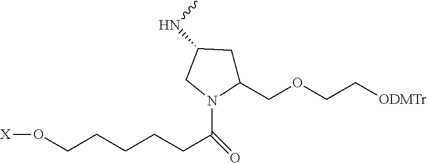

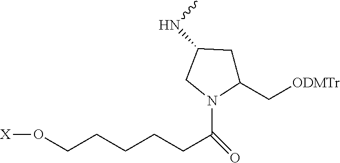

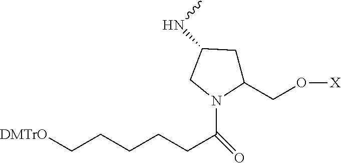

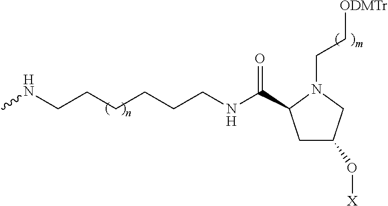

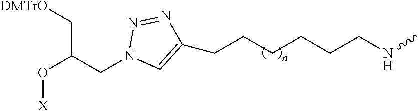

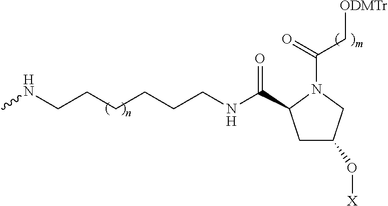

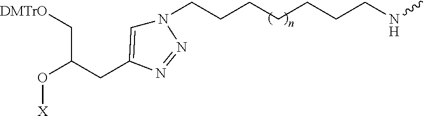

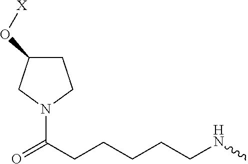

When conjuagated, the DMTr group is removed and the adjacent oxygen is the site of attachment of the Linker to the oligonucleotide via the Linkage (e.g., phosphate or phosphorothioate)
^{a} [symbol] indicates the site of attachment of the Ligand.
^{b}Each structure represents chirally pure or racemic isomers when one or more asymmetric centers are present.

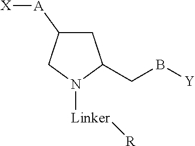

R is LG or has the structure shown below

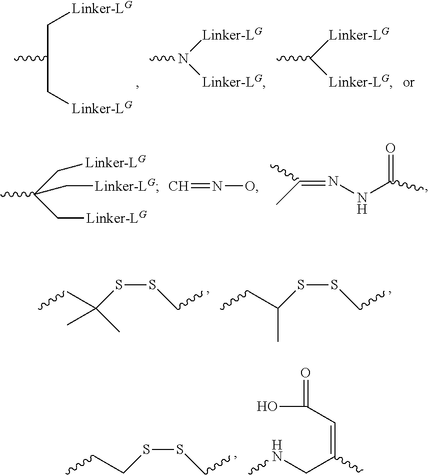

or heterocyclyl.

A cleavable linking group is one which is sufficiently stable outside the cell, but which upon entry into a target cell is cleaved to release the two parts the linker is holding together. In another embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a selected pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

Non-limiting examples of cleavable linkers useful for preparing a conjugate and/or ligand of the present invention include those described in WO2009/073809 and WO 2018/136620, which are hereby incorporated by reference in their entirety.

Exemplary bio-cleavable linkers include:

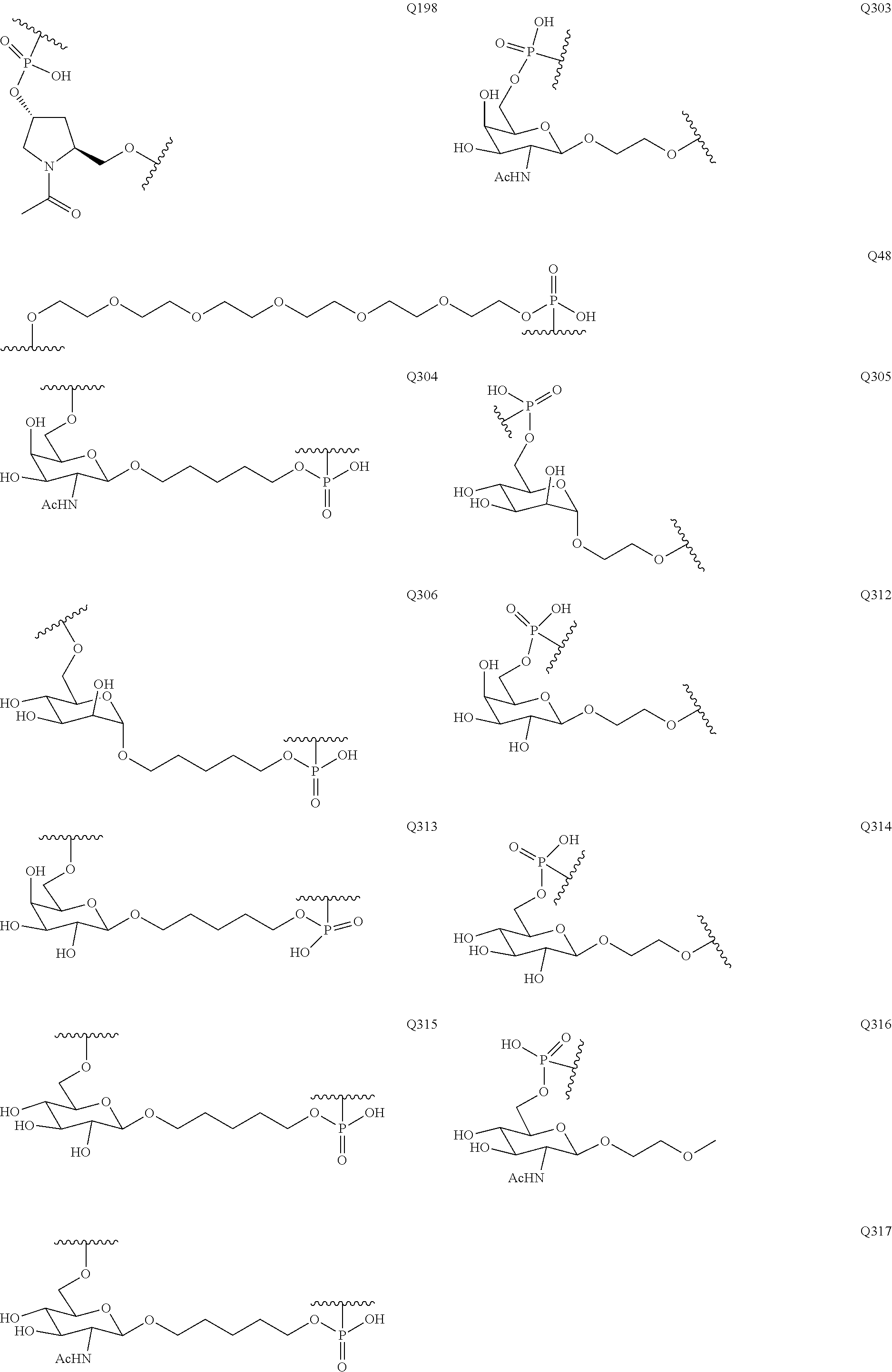

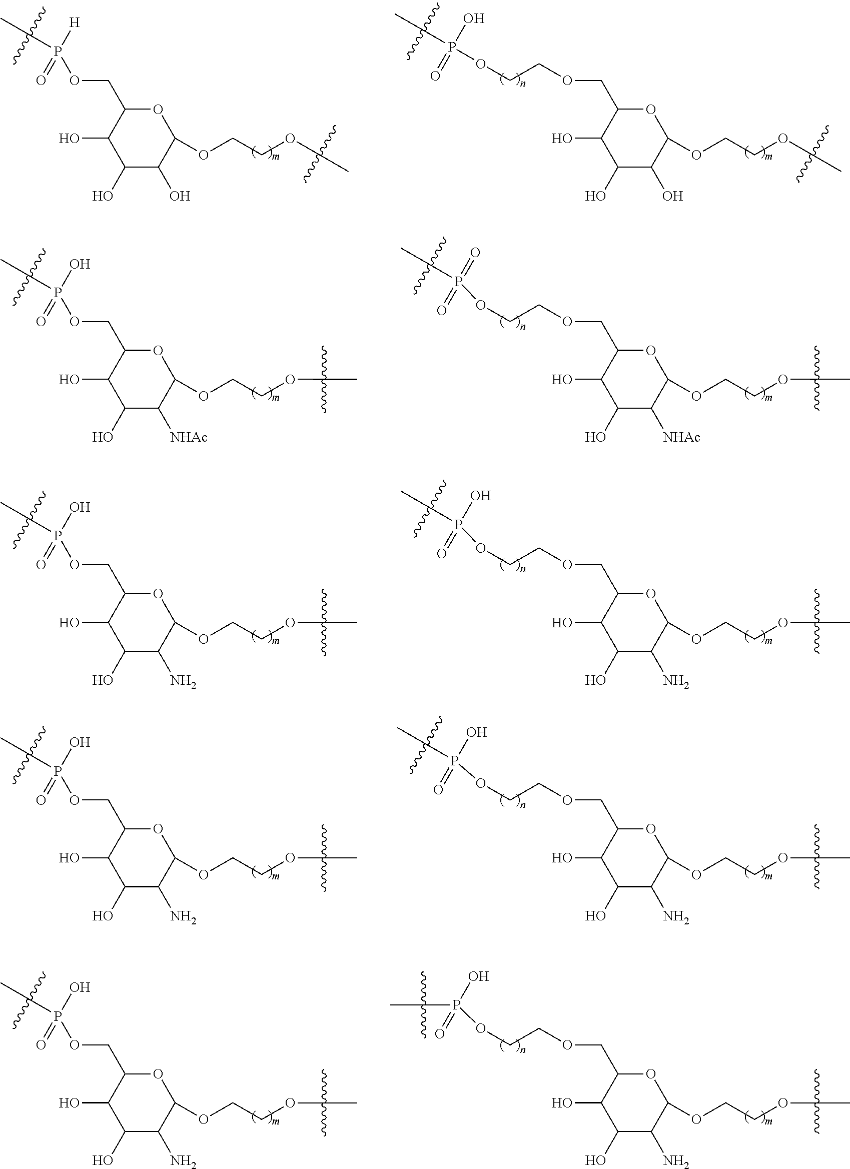

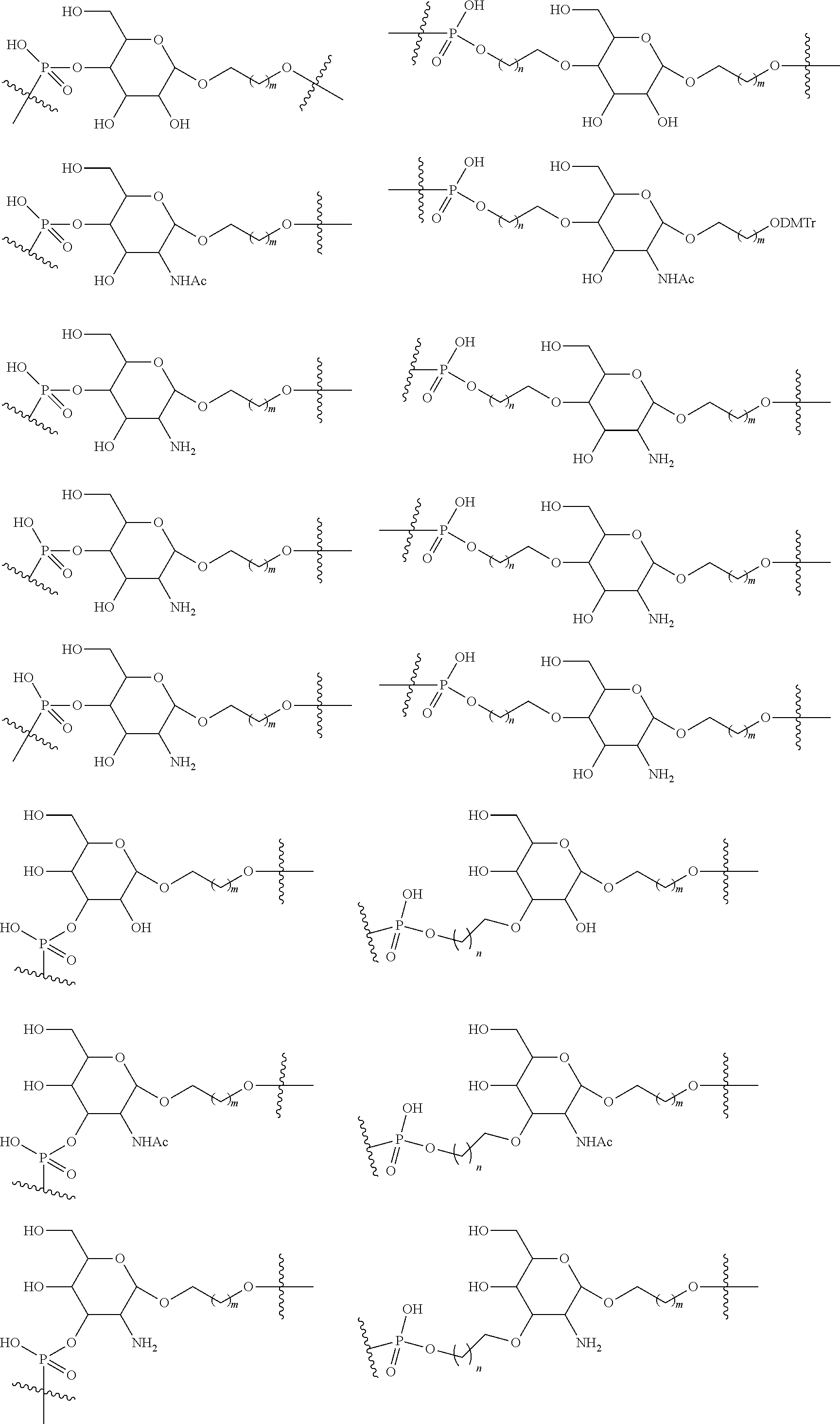

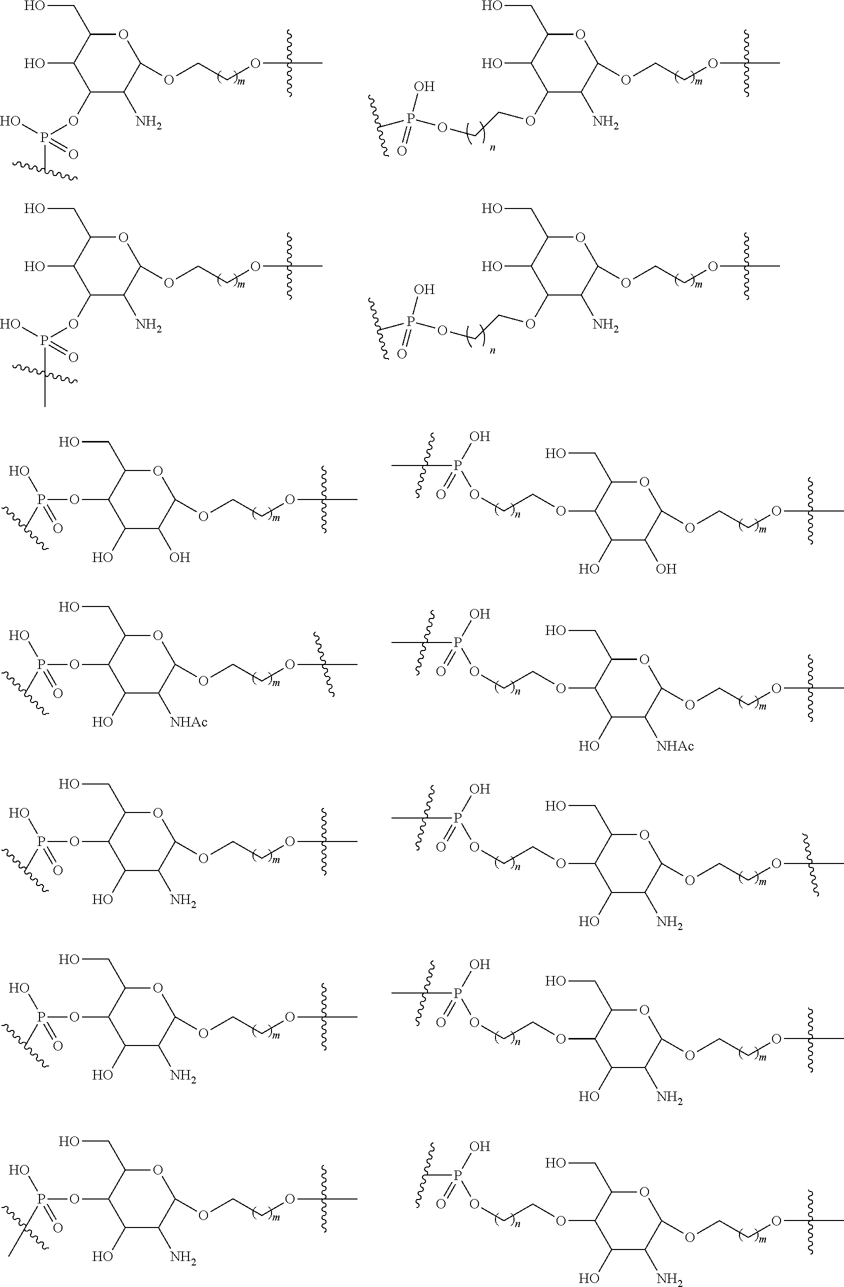

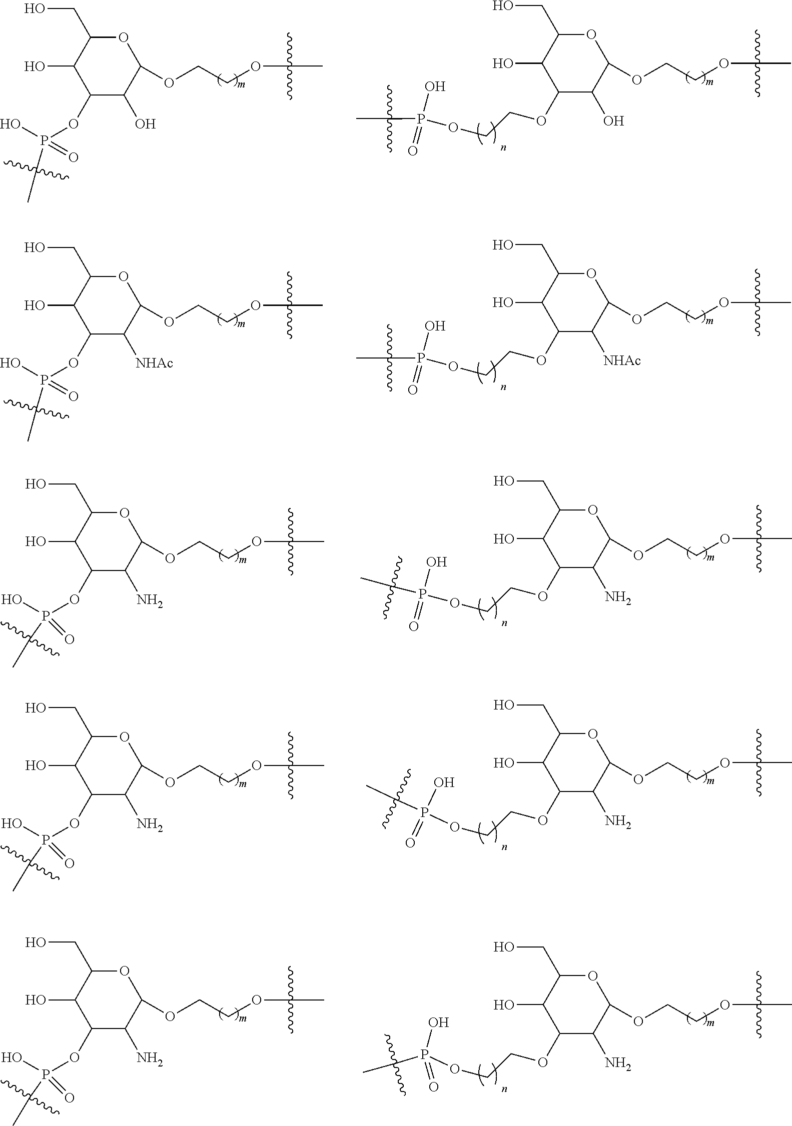

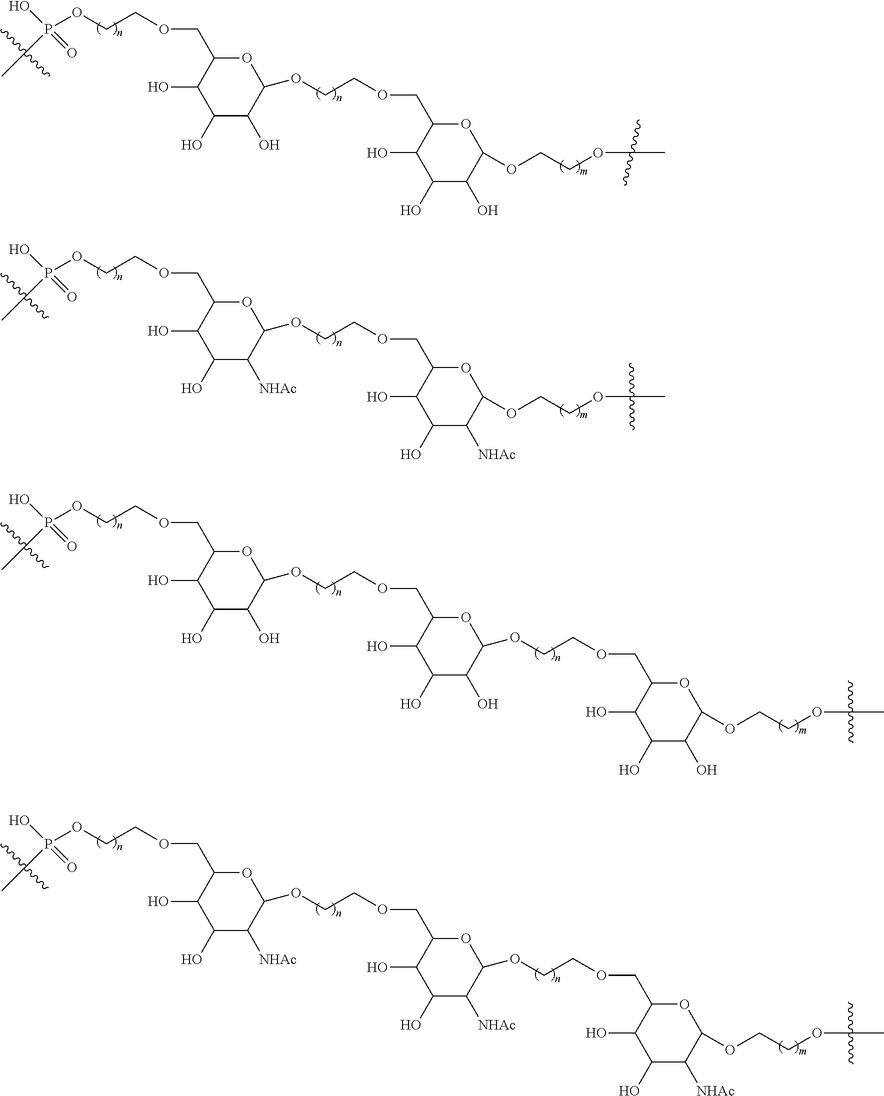

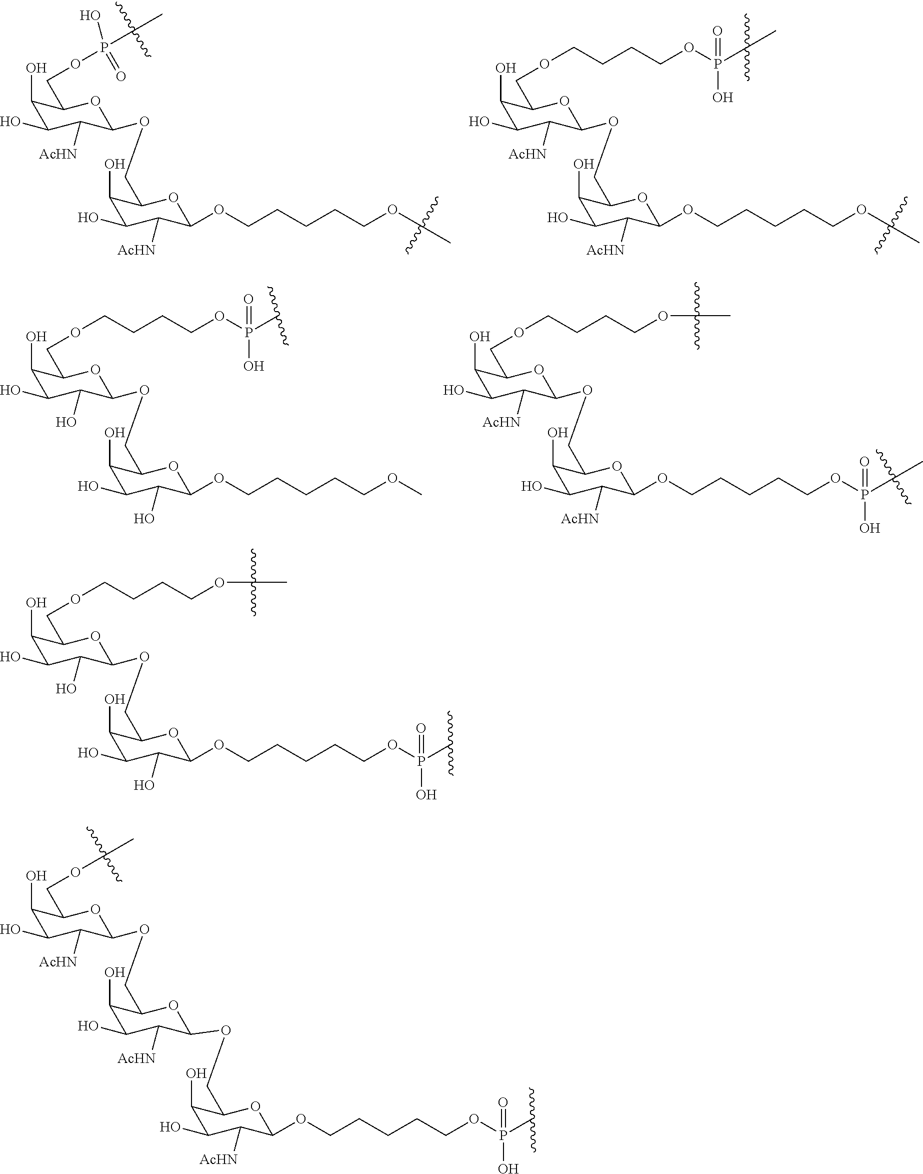 [SMILES: *OCCCCCOC1OC(COC2OC(COP(*)(=O)O)C(O)C(O)C2NC(C)=O)C(O)C(O)C1NC(C)=O ; *OCCCCCOC1OC(COC2OC(COCCCCOP(*)(=O)O)C(O)C(O)C2NC(C)=O)C(O)C(O)C1NC(C)=O ; *P(=O)(O)OCCCCOCC1OC(OCC2OC(OCCCCCOC)C(O)C(O)C2O)C(O)C(O)C1O ; *OCCCCOCC1OC(OCC2OC(OCCCCCOP(*)(=O)O)C(NC(C)=O)C(O)C2O)C(NC(C)=O)C(O)C1O ; *OCCCCOCC1OC(OCC2OC(OCCCCCOP(*)(=O)O)C(O)C(O)C2O)C(O)C(O)C1O ; *OCC1OC(OCC2OC(OCC3OC(OCCCCCOP(*)(=O)O)C(NC(C)=O)C(O)C3O)C(NC(C)=O)C(O)C2O)C(NC(C)=O)C(O)C1O]

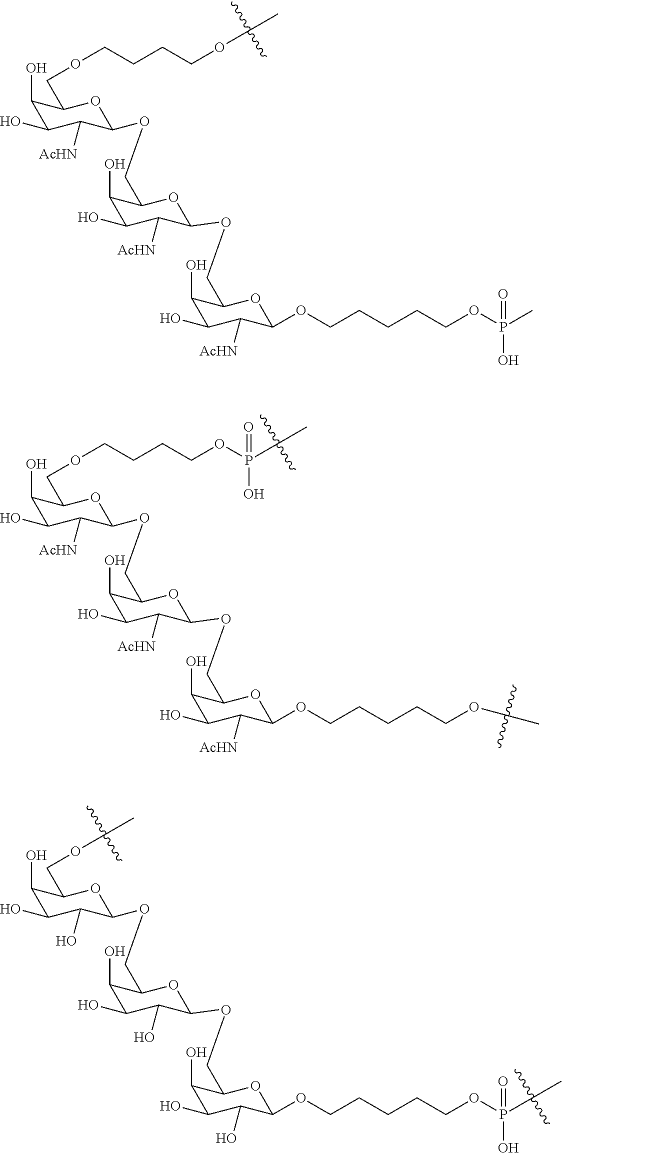 [SMILES: *OCCCCOCC1OC(OCC2OC(OCC3OC(OCCCCCOP(C)(=O)O)C(NC(C)=O)C(O)C3O)C(NC(C)=O)C(O)C2O)C(NC(C)=O)C(O)C1O ; *OCCCCCOC1OC(COC2OC(COC3OC(COCCCCOP(*)(=O)O)C(O)C(O)C3NC(C)=O)C(O)C(O)C2NC(C)=O)C(O)C(O)C1NC(C)=O ; *OCC1OC(OCC2OC(OCC3OC(OCCCCCOP(*)(=O)O)C(O)C(O)C3O)C(O)C(O)C2O)C(O)C(O)C1O]

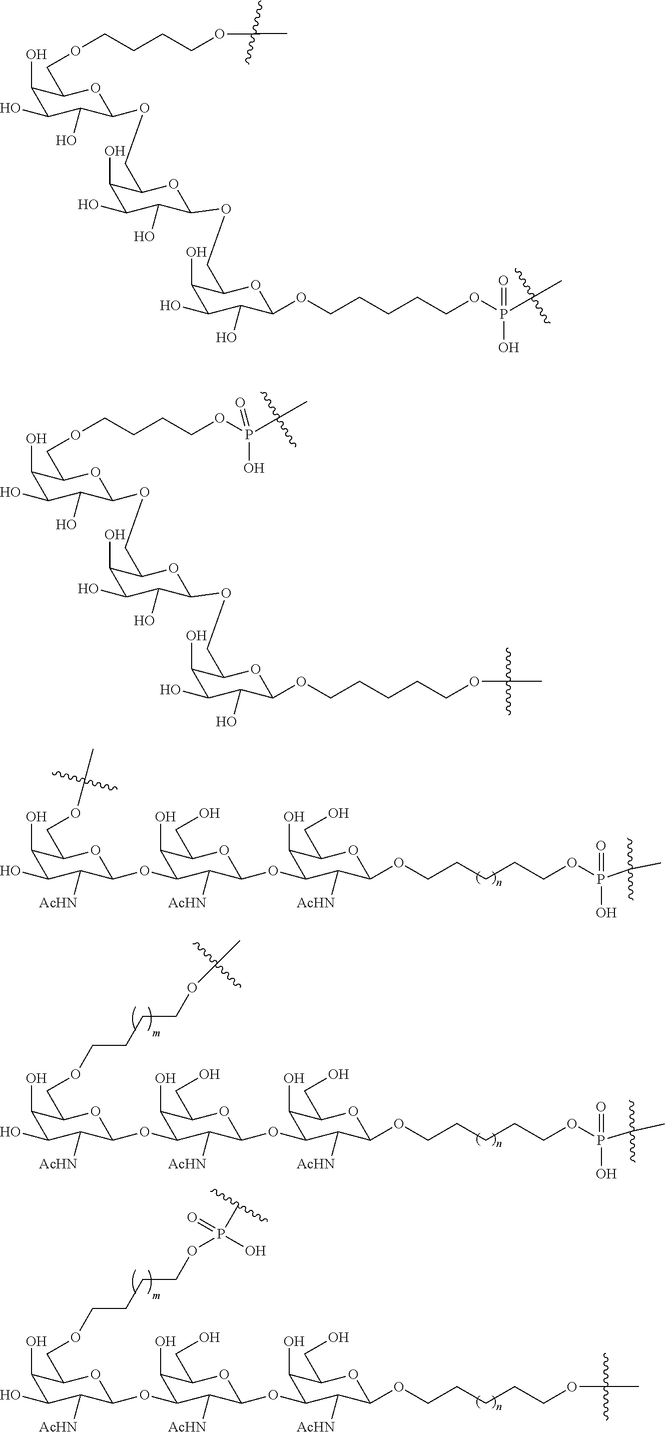

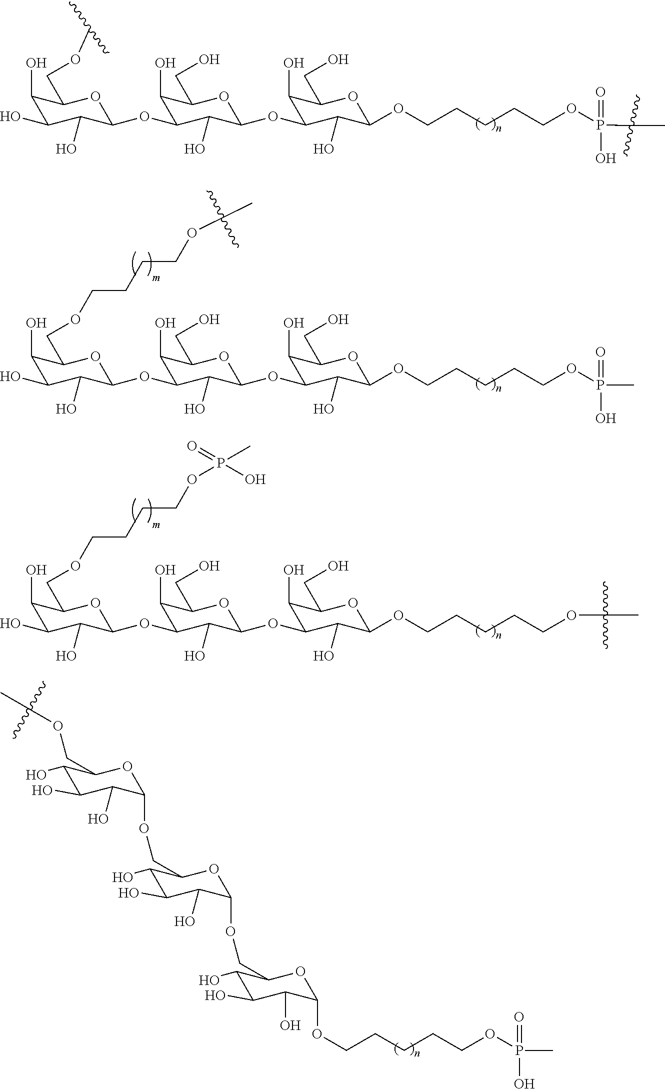

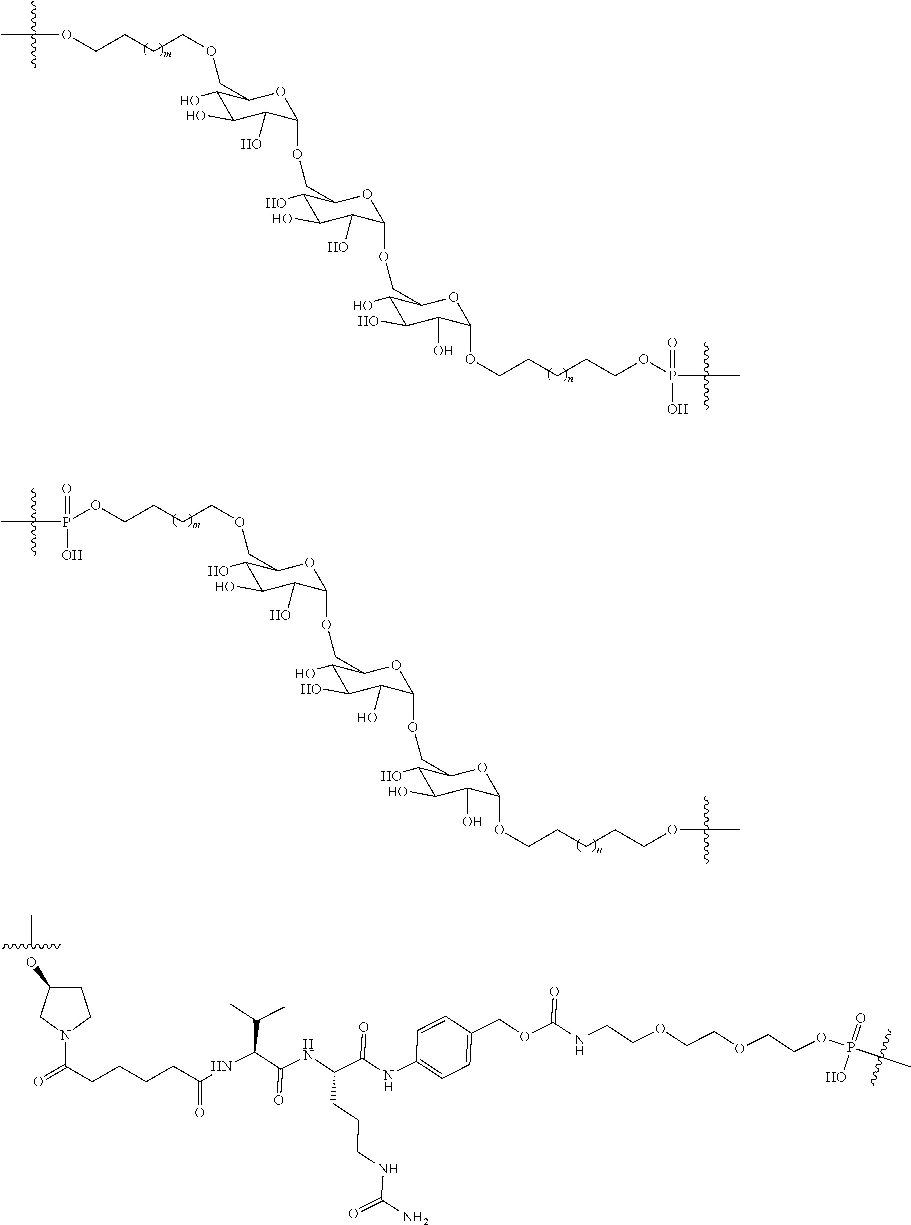

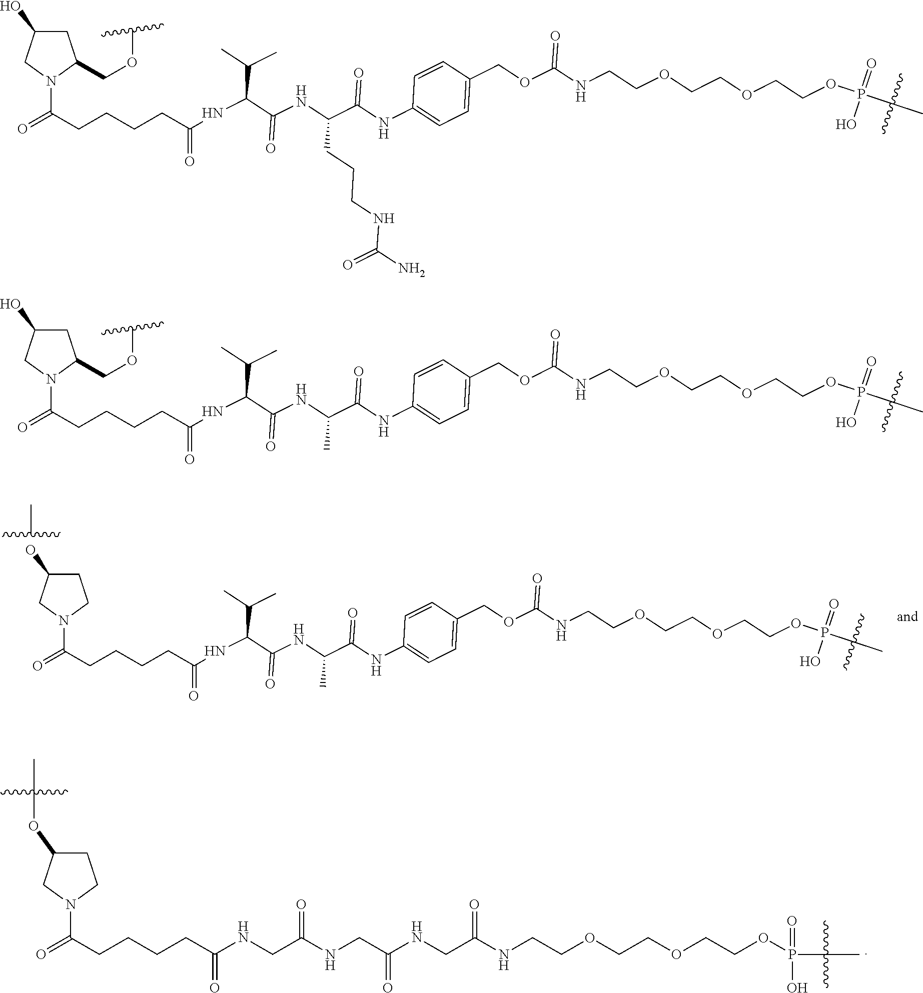

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In certain embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)—O—, —O—P(S)(ORk)—O—, —O—P(S)(SRk)—O—, —S—P(O)(ORk)—O—, —O—P(O)(ORk)-S—, —S—P(O)(ORk)-S—, —O—P(S)(ORk)-S—, —S—P(S)(ORk)—O—, —O—P(O)(Rk)—O—, —O—P(S)(Rk)—O—, —S—P(O)(Rk)—O—, —S—P(S)(Rk)—O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Additional embodiments include —O—P(O){OH)—O—, —O—P(S){OH)—O—, —O—P(S)(SH)—O—, —S—P(O){OH)—O—, —O—P(O){OH)-S—, —S—P(O){OH)-S—, —O—P(S){OH)-S—, —S—P(S){OH)—O—, —O—P(O)(H)—O—, —O—P(S)(H)—O—, —S—P(O)(H)-O, —S—P(S)(H)—O—, —S—P(O)(H)-S—, —O—P(S)(H)-S—, wherein Rk at each occurrence can be, independently, C₁-C20 alkyl, C₁-C20 haloalkyl, C6-C10 aryl, or C7-C12 aralkyl. In certain embodiments, a phosphate-based linking group is —O—P(O){OH)—O—. These candidates can be evaluated using methods analogous to those described above.

iii. Acid Cleavable Linking Groups

In certain embodiments, a cleavable linker comprises an acid cleavable linking group. An acid cleavable linking group is a linking group that is cleaved under acidic conditions. In one embodiment, acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). Another embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Cleavable Linking Groups

In certain embodiments, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)-. These candidates can be evaluated using methods analogous to those described above.

v. Peptide-Based Cleavable Linking Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH-). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula -NHCHR^{A}C(O)NHCHR^{B}C(O)—, where R^{A} and R^{B} are the R groups of the two adjacent amino acids. These candidates can be evaluated using methods analogous to those described above. Representative U.S. Patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928;5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNA agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S— tritylthiol (Manoharan et al., Ann. N. Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBSLett., 1990, 259:327; Svinarchuk et al., Biochimie, 1993, 75:49), aphospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### V. Delivery of an RNAi Agent of the Disclosure

The delivery of an RNAi agent of the disclosure to a cell or tissue e.g., an ocular cell or tissue within a subject, such as a human subject (e.g., a subject in need thereof, such as a subject having an ocular disorder or disease, e.g., glaucoma, can be achieved in a number of different ways. For example, delivery may be performed by contacting an ocular cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with an RNAi agent of the disclosure (see e.g., Akhtar S. and Julian RL., (1992) Trends Cell. Biol. 2(5):139-144 and WO94/02595, which are incorporated herein by reference in their entireties). For in vivo delivery, factors to consider in order to deliver an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, intraocular delivery of a VEGF dsRNA by intravitreal injection in cynomolgus monkeys (Tolentino, M J. et al., (2004) Retina 24:132-138) and subretinal injections in mice (Reich, S J. et al. (2003) Mol. Vis. 9:210-216) were both shown to prevent neovascularization in an experimental model of age-related macular degeneration. In addition, direct intratumoral injection of a dsRNA in mice reduces tumor volume (Pille, J. et al. (2005) Mol. Ther. 11:267-274) and can prolong survival of tumor-bearing mice (Kim, W J. et al., (2006) Mol. Ther. 14:343-350; Li, S. et al., (2007) Mol. Ther. 15:515-523). RNA interference has also shown success with local delivery to the CNS by direct injection (Dorn, G. et al., (2004) Nucleic Acids 32:e49; Tan, P H. et al. (2005) Gene Ther. 12:59-66; Makimura, H. et al. (2002) BMC Neurosci. 3:18; Shishkina, G T., et al. (2004) Neuroscience 129:521-528; Thakker, E R., et al. (2004) Proc. Natl. Acad. Sci. U.S.A. 101:17270-17275; Akaneya, Y., et al. (2005) J. Neurophysiol. 93:594-602) and to the lungs by intranasal administration (Howard, K A. et al., (2006) Mol. Ther. 14:476-484; Zhang, X. et al., (2004) J Biol. Chem. 279:10677-10684; Bitko, V. et al., (2005) Nat. Med. 11:50-55). For administering an RNAi agent systemically for the treatment of a disease, the RNA can be modified or alternatively delivered using a drug delivery system; both methods act to prevent the rapid degradation of the dsRNA by endo- and exo-nucleases in vivo.

Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). RNAi agents can be modified by chemical conjugation to lipophilic groups such as cholesterol to enhance cellular uptake and prevent degradation. For example, an RNAi agent directed against ApoB conjugated to a lipophilic cholesterol moiety was injected systemically into mice and resulted in knockdown of ApoB mRNA in both the liver and jejunum (Soutschek, J. et al., (2004) Nature 432:173-178). Conjugation of an RNAi agent to an aptamer has been shown to inhibit tumor growth and mediate tumor regression in a mouse model of prostate cancer (McNamara, J O. et al., (2006) Nat. Biotechnol. 24:1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent or induced to form a vesicle or micelle (see e.g., Kim SH. et al., (2008) Journal of Controlled Release 129(2):107-116) that encases an RNAi agent. The formation of vesicles or micelles further prevents degradation of the RNAi agent when administered systemically. Methods for making and administering cationic—RNAi agent complexes are well within the abilities of one skilled in the art (see e.g., Sorensen, D R., et al. (2003) J Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of RNAi agents include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet ME. et al., (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an RNAi agent forms a complex with cyclodextrin for systemic administration. Methods for administration and pharmaceutical compositions of RNAi agents and cyclodextrins can be found in U.S. Patent No. 7, 427, 605, which is herein incorporated by reference in its entirety.

The iRNA compositions of the present disclosure may be administered in a number of ways depending upon whether local or systemic treatment is desired and upon the area to be treated. Administration may be topical (including ophthalmic, vaginal, rectal, intranasal, transdermal), intrathecal, oral, or parenteral. Parenteral administration includes intravenous drip, subcutaneous, intraperitoneal or intramuscular injection, or intrathecal or intraventricular administration. The route and site of administration may be chosen to enhance targeting. For example, to target the eye, intraocular injection or ocular topical administration would be a logical choice.

In one embodiment, the administration of the siRNA compound, e.g., a double-stranded siRNA compound, or siRNA compound, composition is intraocular, parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, or urethral.

In another aspect, iRNA targeting a target gene can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the ocular target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

An iRNA expression vector is typically a DNA plasmid or viral vector. An expression vector compatible with eukaryotic cells, e.g., with vertebrate cells, can be used to produce recombinant constructs for the expression of an iRNA as described herein. Eukaryotic cell expression vectors are well known in the art and are available from a number of commercial sources.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an RNAi agent will generally require regulatory elements, e.g., promoters, enhancers, etc., to ensure the expression of the RNAi agent in target cells. Other aspects to consider for vectors and constructs are known in the art.

### VI. Pharmaceutical Compositions of the Invention

The present disclosure also includes pharmaceutical compositions and formulations which include the RNAi agents of the disclosure. In one embodiment, provided herein are pharmaceutical compositions containing an RNAi agent, as described herein, and a pharmaceutically acceptable carrier. The pharmaceutical compositions containing the RNAi agent are useful for treating an ocular disorder, disease, or condition treatable by reduction or inhibition of the expression or activity of a target gene, e.g., an ocular disorder or disease, such as glaucoma.

In some embodiments, the pharmaceutical compositions of the invention are sterile. In another embodiment, the pharmaceutical compositions of the invention are pyrogen free.

Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery. Another example is compositions that are formulated for direct delivery into the eye, e.g., by intraocular delivery (e.g., intravitreal administration, e.g., intravitreal injection; transscleral administration, e.g., transscleral injection; subconjunctival administration, e.g., subconjunctival injection; retrobulbar administration, e.g., retrobulbar injection; intracameral administration, e.g., intracameral injection; or subretinal administration, e.g., subretinal injection suprachoroidal, peribulbar, sub-tenon, intravitreal implant, retrobulbar, posterior juxtascleral). In other embodiments, compositions can be formulated for topical delivery.

The pharmaceutical compositions of the disclosure may be administered in dosages sufficient to inhibit expression of a target gene. In general, a suitable dose of an RNAi agent of the disclosure will be in the range of about 0.01 μg/eye to about 10 mg/eye per day. The dose can be about 0.01 μg/eye to about 0.1 g/eye, about 0.01 μg/eye to about 1 μg/eye, about 0.1 μg/eye to about 1 μg/eye, about 0.1 μg/eye to about g/eye, about 1 μg/eye to about 10 μg/eye, about 1 μg/eye to about 100 μg/eye, about 10 μg/eye to about 100 μg/eye, about 10 μg/eye to about 1 mg/eye, about 100 μg/eye to about 1 mg/eye, about 100 μg/eye to about 10 mg/eye, about 1 mg/eye to about 10 mg/eye, or about 5 mg/eye to about 10 mg/eye per day. In some cases, a suitable dose of an RNAi agent of the disclosure will be in the range of about 0.001 to about 200.0 milligrams per kilogram body weight of the recipient per day, generally in the range of about 1 to 50 mg per kilogram body weight per day.

A repeat-dose regimen may include administration of a therapeutic amount of an RNAi agent on a regular basis, such as monthly to once every six months. In certain embodiments, the RNAi agent is administered about once per quarter (i.e., about once every three months) to about twice per year.

After an initial treatment regimen (e.g., loading dose), the treatments can be administered on a less frequent basis.

In other embodiments, a single dose of the pharmaceutical compositions can be long lasting, such that subsequent doses are administered at not more than 1, 2, 3, or 4 or more month intervals. In some embodiments of the disclosure, a single dose of the pharmaceutical compositions of the disclosure is administered once per month. In other embodiments of the disclosure, a single dose of the pharmaceutical compositions of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a composition can include a single treatment or a series of treatments.

Estimates of effective dosages and in vivo half-lives for the iRNAs encompassed by the disclosure can be made using conventional methodologies or on the basis of in vivo testing using a suitable animal model. A suitable animal model, e.g., a mouse or a rat, e.g., an animal containing a transgene expressing an ocular target gene, can be used to determine the therapeutically effective dose and/or an effective dosage regimen for administration of an iRNA agent of the invention.

The pharmaceutical compositions of the present disclosure may be administered in a number of ways depending upon whether local or systemic treatment is desired and upon the area to be treated. Administration may be local (e.g., by intraocular injection), topical (e.g., by an eye drop solution), or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal, or intraventricular administration.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the RNAi agents featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). RNAi agents featured in the disclosure can be encapsulated within liposomes or can form complexes thereto, in particular to cationic liposomes. Alternatively, RNAi agents can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

### A. RNAi Agent Formulations Comprising Membranous Molecular Assemblies

An RNAi agent for use in the compositions and methods of the disclosure can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the RNAi agent composition. The lipophilic material isolates the aqueous interior from an aqueous exterior, which typically does not include the RNAi agent composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the RNAi agent are delivered into the cell where the RNAi agent can specifically bind to a target RNA and can mediate RNAi. In some cases, the liposomes are also specifically targeted, e.g., to direct the RNAi agent to particular cell types.

A liposome containing an RNAi agent can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The RNAi agent preparation is then added to the micelles that include the lipid component. The cationic groups on the lipid interact with the RNAi agent and condense around the RNAi agent to form a liposome. After condensation, the detergent is removed, e.g., by dialysis, to yield a liposomal preparation of RNAi agent.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Methods for producing stable polynucleotide delivery vehicles, which incorporate a polynucleotide/cationic lipid complex as structural components of the delivery vehicle, are further described in, e.g., WO 96/37194, the entire contents of which are incorporated herein by reference. Liposome formation can also include one or more aspects of exemplary methods described in Felgner, P. L. et al., (1987) Proc. Natl. Acad. Sci. USA 8:7413-7417; U.S. Pat. Nos. 4,897,355; 5,171,678; Bangham et al., (1965) M Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging RNAi agent preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid or phosphatidylcholine or cholesterol.

Examples of other methods to introduce liposomes into cells in vitro and in vivo include U.S. Pat. Nos. 5,283,185; 5,171,678; WO 94/00569; WO 93/24640; WO 91/16024; Felgner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GMi, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglioside GMi, galactocerebroside sulfate and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver RNAi agents to macrophages.

Further advantages of liposomes include: liposomes obtained from natural phospholipids are biocompatible and biodegradable; liposomes can incorporate a wide range of water and lipid soluble drugs; liposomes can protect encapsulated RNAi agents in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of RNAi agent (see, e.g., Felgner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™ Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration; liposomes present several advantages over other formulations. Such advantages include reduced side effects related to high systemic absorption of the administered drug, increased accumulation of the administered drug at the desired target, and the ability to administer RNAi agent into the skin. In some implementations, liposomes are used for delivering RNAi agent to epidermal cells and also to enhance the penetration of RNAi agent into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with RNAi agent are useful for treating a dermatological disorder.

Liposomes that include RNAi agents can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include RNAi agent can be delivered, for example, subcutaneously by infection in order to deliver RNAi agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present disclosure are described in PCT application number PCT/US2008/088588, filed Dec. 13, 2008; PCT/US2008/088587, filed Dec. 13, 2008; PCT/US2009/041442, filed Apr. 22, 2009; and PCT/US2007/080331, filed Oct. 3, 2007.

Transfersomes, yet another type of liposomes, are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as those described herein, particularly in emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in Pharmaceutical Dosage Forms, Marcel Dekker, Inc., New York, N.Y., 1988, p. 285).

The RNAi agent for use in the methods of the disclosure can also be provided as micellar formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol or m-cresol may be added to the mixed micellar composition to stabilize the formulation and protect against bacterial growth. Alternatively, phenol or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the disclosure are administered in conjunction with one or more penetration enhancer surfactants and chelators. Suitable surfactants include fatty acids or esters or salts thereof, bile acids or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcamitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the disclosure can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, U.S. 2003/0027780, and U.S. Pat. No. 6,747,014, each of which is incorporated herein by reference.

Compositions and formulations for intraocular, parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions can be generated from a variety of components that include, but are not limited to, preformed liquids, self-emulsifying solids and self-emulsifying semisolids. Exemplary formulations include those that target the eye when treating or preventing ocular diseases or disorders.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, eye drops, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

### B. Additional Formulations

i. Emulsions

The compositions of the present disclosure can be prepared and formulated as emulsions. Emulsions are typically heterogeneous systems of one liquid dispersed in another in the form of droplets usually exceeding 0.1 m in diameter (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present disclosure, the compositions of RNAi agents and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used, and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (P0310), hexaglycerol pentaoleate (P0500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or RNAi agents. Microemulsions have also been effective in the transdermal delivery of active components in both cosmetic and pharmaceutical applications. It is expected that the microemulsion compositions and formulations of the present disclosure will facilitate the increased systemic absorption of RNAi agents and nucleic acids from the gastrointestinal tract, as well as improve the local cellular uptake of RNAi agents and nucleic acids.

Microemulsions of the present disclosure can also contain additional components and additives such as sorbitan monostearate (Grill 3), Labrasol, and penetration enhancers to improve the properties of the formulation and to enhance the absorption of the RNAi agents and nucleic acids of the present disclosure. Penetration enhancers used in the microemulsions of the present disclosure can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

An RNAi agent of the disclosure may be incorporated into a particle, e.g., a microparticle. Microparticles can be produced by spray-drying, but may also be produced by other methods including lyophilization, evaporation, fluid bed drying, vacuum drying, or a combination of these techniques.

iv. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

v. Other Components

The compositions of the present disclosure can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings or aromatic substances and the like which do not deleteriously interact with the nucleic acid(s) of the formulation.

Aqueous suspensions can contain substances which increase the viscosity of the suspension including, for example, sodium carboxymethylcellulose, sorbitol or dextran. The suspension can also contain stabilizers.

In some embodiments, pharmaceutical compositions featured in the disclosure include (a) one or more RNAi agents and (b) one or more agents which function by a non-RNAi mechanism and which are useful in treating an ocular disorder or disease, e.g., glaucoma. Examples of such agents include, but are not limited to, orlistat (Alli, Xenical), phentermine and topiramate (Qsymia), bupropion and naltrexone (Contrave), liraglutide (Saxenda, Victoza), and agents that decrease or otherwise affect target gene activity.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agents featured in the disclosure can be administered in combination with other known agents effective in treatment of pathological processes mediated by nucleotide repeat expression. In any event, the administering physician can adjust the amount and timing of RNAi agent administration on the basis of results observed using standard measures of efficacy known in the art or described herein.

### VII. Kits

In certain aspects, the instant disclosure provides kits that include a suitable container containing a pharmaceutical formulation of a siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof).

Such kits include one or more dsRNA agent(s) and instructions for use, e.g., instructions for administering a prophylactically or therapeutically effective amount of a dsRNA agent(s) provided herein. The dsRNA agent may be in a vial or a pre-filled syringe. The kits may optionally further comprise means for administering the dsRNA agent (e.g., an injection device, such as a pre-filled syringe or an intrathecal pump), or means for measuring the inhibition of the target gene (e.g., means for measuring the inhibition of the target gene mRNA, target gene protein, and/or target gene activity). Such means for measuring the inhibition of the target gene may comprise a means for obtaining a sample from a subject, such as, e.g., a plasma sample and/or or ocular fluid sample. The kits of the invention may optionally further comprise means for determining the therapeutically effective or prophylactically effective amount.

In certain embodiments the individual components of the pharmaceutical formulation may be provided in one container. Alternatively, it may be desirable to provide the components of the pharmaceutical formulation separately in two or more containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device. In one embodiment, the kit comprises a delivery device suitable for ocular delivery.

### VIII. Methods for Inhibiting Ocular Target Gene Expression

In some aspects, the disclosure provides a method for inhibiting the expression of a target gene in the eye, e.g., in an ocular cell or tissue. In some embodiments, the cell or tissue is ex vivo, in vitro, or in vivo.

In some embodiments, the ocular cell or tissue includes an optic nerve cell, a trabecular meshwork cell, a limbal ring cell, a Schlemm's canal cell (e.g., including an endothelial cell), a juxtacanalicular tissue cell, a ciliary muscle cell, a retinal cell, an astrocyte, a pericyte, a Muller cell, a ganglion cell (e.g., including a retinal ganglion cell), an endothelial cell, a photoreceptor cell, a retinal blood vessel (e.g., including endothelial cells and vascular smooth muscle cells), episcleral veins or choroid tissue, e.g., a choroid vessel), cornea, pupil, sclera, conjunctiva, optic nerve, iris, lens, aqueous humor, macula, optic disk, retina, ciliary muscle, vitreous humor, vitreous body, choroid, fovea, ciliary body, blood vessels, muscles (lateral rectus muscle, medial rectus muscle, ciliary muscle), ligaments (suspensory ligaments), anterior chamber, posterior chamber, limbal rings, or fovia.

In some embodiments, the cell or tissue is in a subject (e.g., a mammal, such as, for example, a human). In some embodiments, the subject (e.g., the human) is at risk, or is diagnosed with an ocular disorder or disease related to expression of a target gene, as described herein.

In some embodiments, the method includes contacting the ocular cell or tissue with an iRNA as described herein, in an amount effective to decrease the expression of the target gene in the ocular cell or tissue. In some embodiments, contacting a cell with an RNAi agent includes contacting a cell in vitro with the RNAi agent or contacting a cell in vivo with the RNAi agent. In some embodiments, the RNAi agent is put into physical contact with the cell by the individual performing the method, or the RNAi agent may be put into a situation that will permit or cause it to subsequently come into contact with the cell. Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., ocular tissue. For example, the RNAi agent may contain or be coupled to a ligand, e.g., an integrin targeting ligand, such as an RGD peptide ligand. Combinations of in vitro and in vivo methods of contacting are also possible. For example, a cell may also be contacted in vitro with an RNAi agent and subsequently transplanted into a subject.

The expression of the target gene may be assessed based on the level of expression of target gene mRNA, target gene protein, or the level of another parameter functionally linked to the level of expression of the target gene, e.g., activity of the target gene. In some embodiments, the expression of the target gene is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, the iRNA has an IC₅₀ in the range of about 0.0001-100 nM, about 0.0001-0.001 nM, about 0.001-0.01 nM, about 0.001-0.10 nM, about 0.001-1.0 nM, about 0.001-10 nM, about 0.01-0.05 nM, about 0.01-0.50 nM, about 0.02-0.60 nM, about 0.01-1.0 nM, about 0.01-1.5 nM, about 0.01-10 nM, about 0.01-100 nM, about 0.1-10 nM, about 0.1-100 nM, or about 1-100 nM. The IC₅₀ value may be normalized relative to an appropriate control value, e.g., the IC₅₀ of a non-targeting iRNA.

In some embodiments, the method includes introducing into the ocular cell or tissue an iRNA as described herein and maintaining the cell or tissue for a time sufficient to obtain degradation of the mRNA transcript of the target gene, thereby inhibiting the expression of the target gene in the ocular cell or tissue.

In some embodiments, the method includes administering a composition described herein, e.g., a composition comprising an iRNA conjugated to an integrin targeting ligand, to the mammal such that expression of the target gene is decreased, such as for an extended duration, e.g., at least two, three, four days or more, e.g., one week, two weeks, three weeks, or four weeks or longer. In some embodiments, the decrease in expression of the target gene is detectable within 1 hour, 2 hours, 4 hours, 8 hours, 12 hours, or 24 hours of the first administration.

The iRNAs useful for the methods and compositions featured in the disclosure specifically target RNAs (primary or processed) of the target gene in the eye. Compositions and methods for inhibiting the expression of the target gene using iRNAs can be prepared and performed as described elsewhere herein. In some embodiments, the method includes administering a composition containing an iRNA, where the iRNA includes a nucleotide sequence that is complementary to at least a part of an RNA transcript of the target gene of the subject, e.g., the mammal, e.g., the human, to be treated. The composition may be administered by any appropriate means known in the art including, but not limited to ocular (e.g., intraocular), topical, and intravenous administration.

In certain embodiments, the composition is administered intraocularly (e.g., by intravitreal administration, e.g., intravitreal injection; transscleral administration, e.g., transscleral injection; subconjunctival administration, e.g., subconjunctival injection; retrobulbar administration, e.g., retrobulbar injection; intracameral administration, e.g., intracameral injection; or subretinal administration, e.g., subretinal injection, suprachoroidal, peribulbar, sub-tenon, intravitreal implant, retrobulbar, posterior juxtascleral. In other embodiments, the composition is administered topically. In other embodiments, the composition is administered by intravenous infusion or injection.

In some embodiments, the composition comprises a lipid formulated siRNA (e.g., an LNP formulation) for intravenous infusion.

### IX. Methods of Treating or Preventing Ocular Disorders or Diseases

The present disclosure relates to the use of an iRNA targeting a target gene in an ocular cell or tissue to inhibit target gene expression and/or to treat an ocular disease, disorder, or pathological process that is related to target gene expression (e.g., glaucoma or other ocular disorders).

In some aspects, a method of treatment of a disorder related to expression of an ocular target gene is provided, the method comprising administering an iRNA (e.g., a dsRNA) disclosed herein to a subject in need thereof. In some embodiments, the iRNA inhibits (decreases) target gene expression in the eye.

In some embodiments, the subject is an animal that serves as a model for a disorder related to the target gene expression in the eye, e.g., glaucoma.

Non-limiting examples of ocular disorders or diseases that are treatable using the methods described herein include glaucoma, primary open angle glaucoma, secondary glaucoma, pigmentary glaucoma, pseudoexfoliative glaucoma, traumatic glaucoma, neovascular glaucoma, irido corneal endothelial syndrome, uveitic glaucoma, angle closure glaucoma, normal tension glaucoma, juvenile open angle glaucoma, macular degeneration, cataracts, diabetic retinopathy, dry eyes, blurred vision, red eyes, blindness, night blindness, lazy eye, strabismus (cross eyes), nystagmus, colorblindness, uveitis, ocular inflammation, presbyopia, floaters in the field of vision, retinal disorders, retinal tear or detachment, conjunctivitis (pink eye), corneal diseases, vision changes, bulging eyes (proptosis), retinitis, diabetic macular edema, keratoconus, lazy eye, ocular hypertension, astigmatism, diabetic eye disease, hyperopia, myopia, macular edema, retinoblastoma, stargardt disease, usher syndrome, vitreous detachment, retinal disease, and cancers of the eye.

In some embodiments, the disorder related to the ocular target gene expression is glaucoma. Clinical and pathological features of glaucoma include, but are not limited to, intraocular pressure, vision loss, a reduction in visual acuity (e.g., characterized by floating spots, blurriness around the edges or center of field of vision (e.g., scotoma), ocular inflammation, and/or optic nerve damage.

In some embodiments, the subject with an ocular disorder or disease is than 18 years old. In some embodiments, the subject with an ocular disorder or disease is an adult. In some embodiments, the subject has, or is identified as having, elevated levels of a target gene mRNA or protein relative to a reference level (e.g., a level of a target gene that is greater than a reference level).

In some embodiments, the ocular disorder or disease is diagnosed using analysis of a sample from the subject (e.g., an eye tissue or fluid sample). In some embodiments, the sample is analyzed using a method selected from one or more of: fluorescent in situ hybridization (FISH), immunohistochemistry, immunoassay, electron microscopy, laser microdissection, and mass spectrometry. In some embodiments, an ocular disorder or disease, e.g., glaucoma, is diagnosed using any suitable diagnostic test or technique, e.g., tonometry, pachymetry, evaluation of the retina, gonioscopy, angiography (e.g., fluorescein angiography or indocyanine green angiography), electroretinography, ultrasonography, optical coherence tomography (OCT), computed tomography (CT), magnetic resonance imaging (MRI), color vision testing, visual field testing, slit-lamp examination, ophthalmoscopy, and physical examination (e.g., to assess visual acuity (e.g., by fundoscopy or optical coherence tomography (OCT)).

### A. Combination Therapies

In some embodiments, an iRNA (e.g., a dsRNA) disclosed herein is administered in combination with a second therapy (e.g., one or more additional therapies) known to be effective in treating an ocular disorder or disease (e.g., glaucoma) or a symptom of such a disorder. The iRNA may be administered before, after, or concurrent with the second therapy. In some embodiments, the iRNA is administered before the second therapy. In some embodiments, the iRNA is administered after the second therapy. In some embodiments, the iRNA is administered concurrent with the second therapy.

The second therapy may be an additional therapeutic agent. The iRNA and the additional therapeutic agent can be administered in combination in the same composition or the additional therapeutic agent can be administered as part of a separate composition.

In some embodiments, the second therapy is a non-iRNA therapeutic agent that is effective to treat the ocular disorder or symptoms of the disorder.

In some embodiments, the iRNA is administered in conjunction with a therapy.

Exemplary combination therapies include, but are not limited to, medication to reduce intraocular pressure, eye drops, laser treatment, surgery, or trabeculectomy. In some embodiments, the additional therapeutic agent comprises an antibiotic, antiviral medication, a prostaglandin analog, a beta blocker, an alpha-adrenergic agonist, a carbonic anhydrase inhibitor, a small molecule inhibitor of the target gene, or a monoclonal antibody therapy.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the iRNAs and methods featured in the disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The invention now being generally described, it will be more readily understood by reference to the following examples which are included merely for purposes of illustration of certain aspects and embodiments of the present invention and are not intended to limit the invention.

### Example 1. Synthesis of Integrin Targeting Ligands

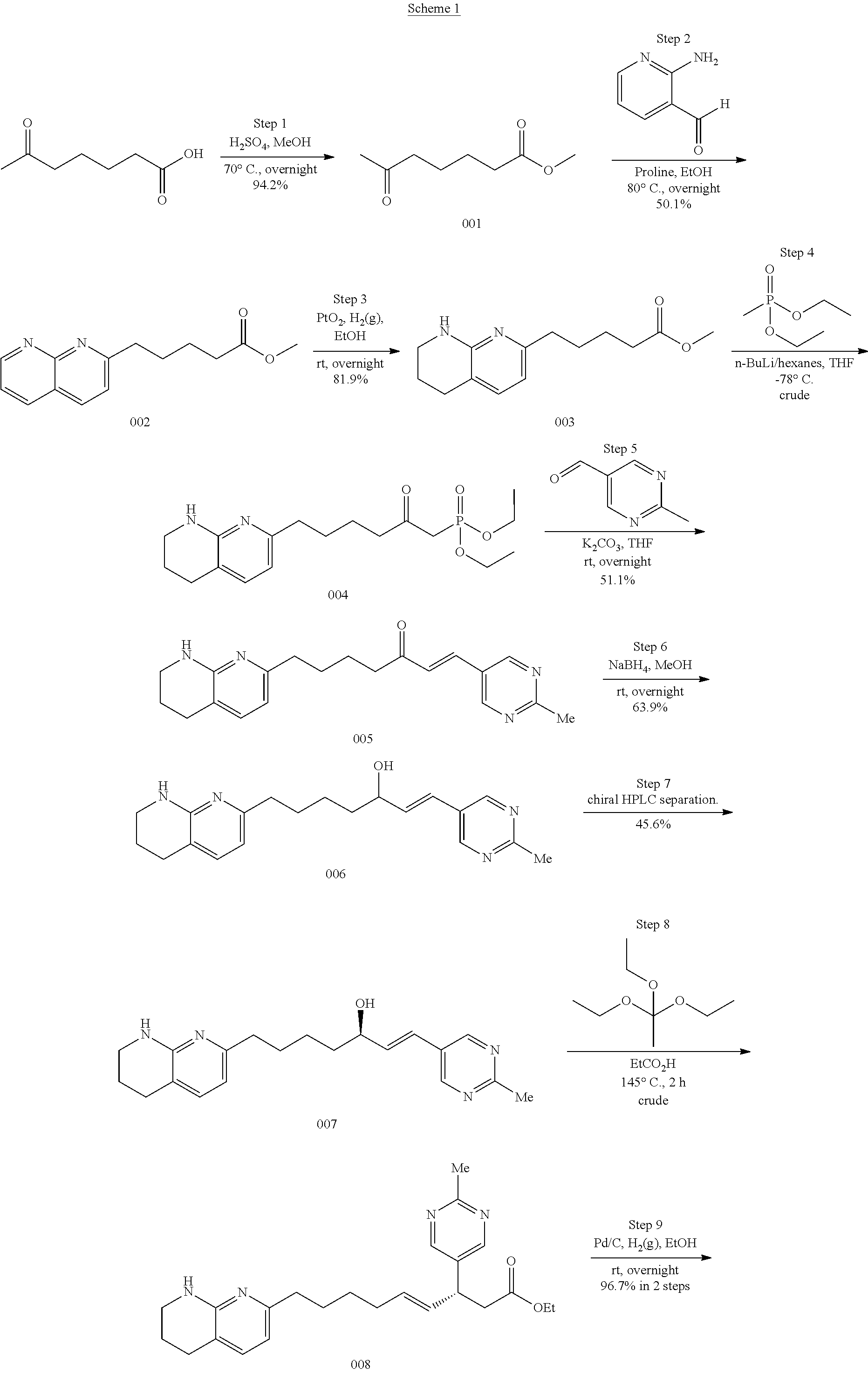

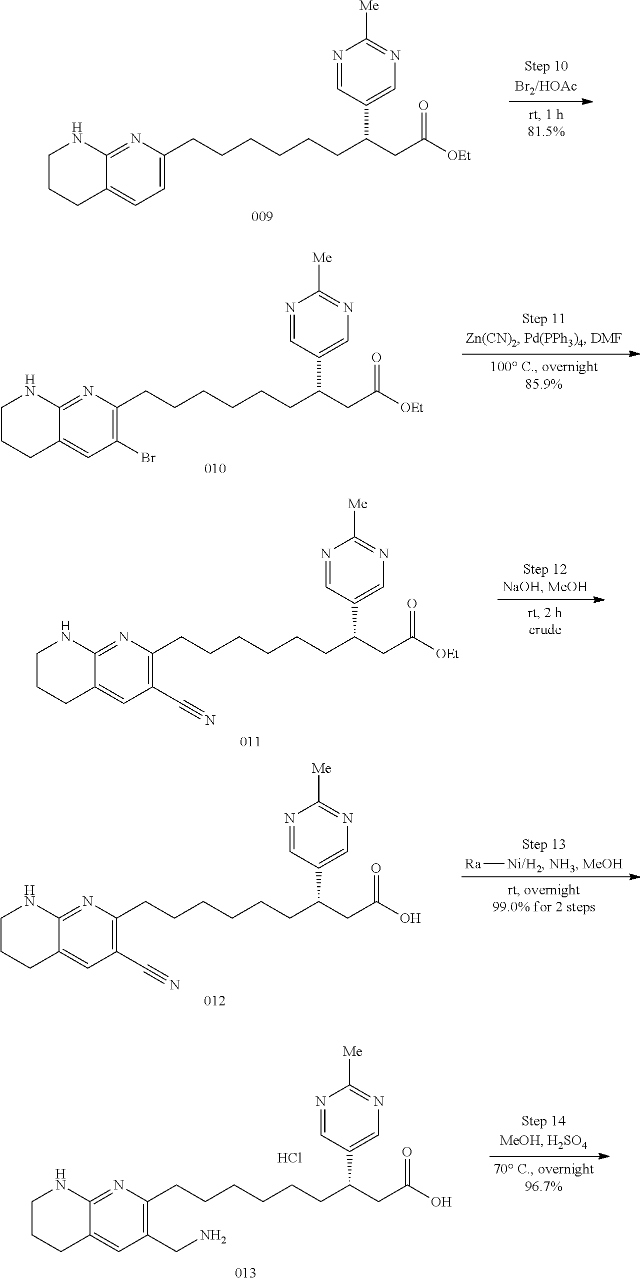

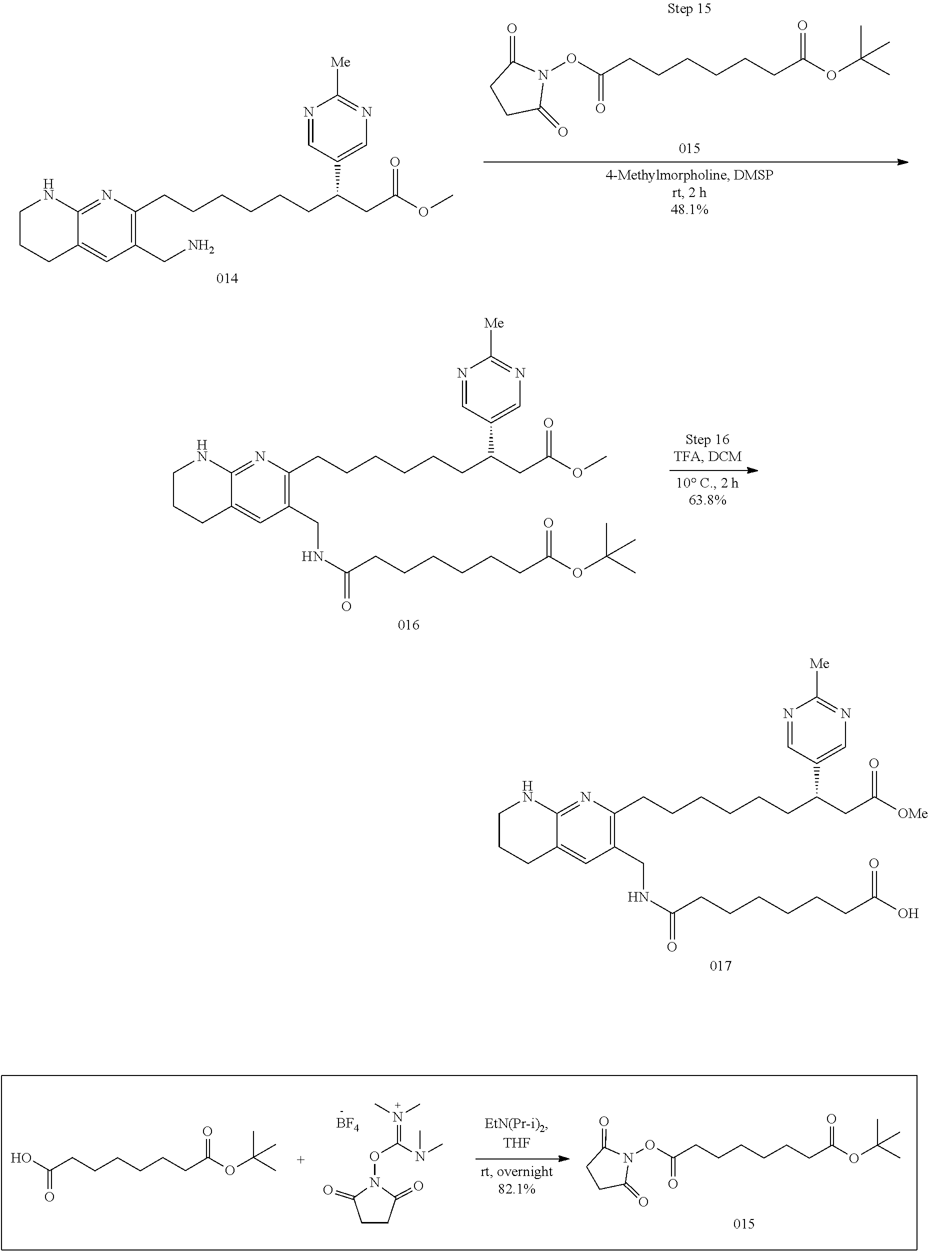

The synthetic procedures of Compound 014 have been reported in Journal of Medicinal Chemistry 2004, 47, 4829-4837 and WO 2009/114776 A2. The details are described below.

Compound 001: To a stirred solution of 5-aceto valeric acid (300.00 g, 2080.877 mmol, 1.00 equiv) in MeOH (3000 mL) were added conc. H₂SO₄ (10.00 mL) dropwise portions at room temperature under nitrogen atmosphere. The resulting mixture was stirred for overnight at 70 degrees C. under nitrogen atmosphere. The mixture was allowed to cool down to room temperature. The resulting mixture was concentrated under vacuum. This resulted in 6-keto methylheptanoate 1 (310 g, 94.2%) as a yellow oil.

Compound 002: To a stirred mixture of 6-keto methylheptanoate 1 (310.00 g, 1959.582 mmol, 1.00 equiv), L-proline (56.40 g, 489.896 mmol, 0.25 equiv) and 2-aminopyridine-3-carbaldehyde (215.39 g, 1763.624 mmol, 0.90 equiv) in EtOH (3000 mL) at room temperature under nitrogen atmosphere. The resulting mixture was stirred for overnight at 80 degrees C. under nitrogen atmosphere. The mixture was allowed to cool down to room temperature. The resulting mixture was extracted with EtOAc (3 ×2 L). The combined organic layers were washed with water (2×1 L) and brine (1×1000 mL), dried over anhydrous Na₂SO₄. The combined organic layers were concentrated under vacuum. The residue was purified by silica gel column chromatography, eluted with CH₂C₁₂/EtOH (10:1) to afford methyl 5-(1,8-naphthyridin-2-yl)pentanoate 2 (240 g, 50.1%) as a yellow oil.

Compound 003: To a solution of methyl 5-(1,8-naphthyridin-2-yl) pentanoate 2 (240.00 g, 982.423 mmol, 1.00 equiv) in EtOH (2 L) was added PtO₂ (24.00 g, 105.688 mmol, 0.11 equiv) under nitrogen atmosphere in a 5L 3-necked round-bottom flask. The mixture was hydrogenated at room temperature for overnight under hydrogen atmosphere using a hydrogen balloon, filtered through a Celite pad and concentrated under reduced pressure. This resulted in methyl 5-(5,6,7,8-tetrahydro-1,8-naphthyridin-2-yl) pentanoate 3 (200 g, 81.9%) as a white solid.

Compound 004: In a solution of diethyl methylphosphonate (490.10 g, 3221.572 mmol, 4.00 equiv) in THF (2500 mL) was added dropwise n-BuLi in hexanes (1288.00 mL, 3221.572 mmol, 4.00 equiv) at −78 degrees C. under N₂ atmosphere. The reaction mixture was stirred at −78 degrees C. for 30 min. Then a solution of methyl 5-(5,6,7,8-tetrahydro-1,8-naphthyridin-2-yl) pentanoate 3 (200.00 g, 805.393 mmol, 1.00 equiv) in THF (2500 mL) was added dropwise and the mixture was stirred for another 60 min. The reaction was quenched with sat. NH₄Cl (1000 mL), and then the mixture was extracted with ether/EtOAc (2×2000 mL). The combined organic layers were washed with brine (1000 mL), dried over anhydrous Na₂SO₄, and concentrated under vacuum. This crude product in diethyl 2-oxo-6-(5,6,7,8-tetrahydro-1,8-naphthyridin-2-yl) hexylphosphonate 4 (300 g, crude) as a yellow oil. The crude product was used in the next step directly without further purification.

Compound 005: To a stirred solution of diethyl 2-oxo-6-(5,6,7,8-tetrahydro-1,8-naphthyridin-2-yl) hexylphosphonate 4 (300.00 g, 814.301 mmol, 1.00 equiv), K₂CO₃ (281.35 g, 2035.753 mmol, 2.50 equiv) and 2-methylpyrimidine-5-carbaldehyde (99.45 g, 814.301 mmol, 1.00 equiv) in THF (3000.00 mL) at room temperature under nitrogen atmosphere. The resulting mixture was stirred for overnight at room temperature under nitrogen atmosphere. The resulting mixture was diluted with EtOAc (1000 mL). The combined organic layers were washed with water (2×1000 mL) and brine (1×1000 mL), dried over anhydrous Na₂SO₄. After filtration, the filtrate was concentrated under reduced pressure. The residue was purified by silica gel column chromatography, eluted with CH₂C₁₂/MeOH (15:1) to afford (1E)-1-(2-methylpyrimidin-5-yl)-7-(5,6,7,8-tetrahydro-1,8-naphthyridin-2-yl) hept-1-en-3-one 5 (140 g, 51.1%) as a yellow oil.

Compound 006: To a stirred solution of (1E)-1-(2-methylpyrimidin-5-yl)-7-(5,6,7,8-tetrahydro-1,8-naphthyridin-2-yl) hept-1-en-3-one 5 (140.00 g, 416.123 mmol, 1.00 equiv) in MeOH (2000.00 mL) was added NaBH₄ (18.89 g, 0.000 mmol, 1.20 equiv) in portions at 0 degrees C. under nitrogen atmosphere. The resulting mixture was stirred for overnight at room temperature under nitrogen atmosphere. The mixture was acidified to pH 3 with conc. HCl. The mixture was basified to pH 9 with saturated NaHCO₃ (aq.). The resulting mixture was extracted with CH₂C₁₂ (2×1000 mL). The combined organic layers were washed with water (2×500 mL), dried over anhydrous Na₂SO₄. After filtration, the filtrate was concentrated under reduced pressure. The residue was purified by silica gel column chromatography, eluted with CH₂C₁₂/MeOH (20:1) to afford (1E)-1-(2-methylpyrimidin-5-yl)-7-(5,6,7,8-tetrahydro-1,8-naphthyridin-2-yl) hept-1-en-3-ol 6 (90 g, 63.9%) as a yellow oil.

Compound 007: (1E)-1-(2-methylpyrimidin-5-yl)-7-(5,6,7,8-tetrahydro-1,8-naphthyridin-2-yl)hept-1-en-3-ol 6 (90.00 g, 0.266 mmol, 1.00 equiv) was purified by Prep-HPLC with the following conditions (Column: XA-CHIRALPAK AD-3, 4.6*50 mm, 3 um; Mobile Phase A: Hex (0.1% DEA)/EtOH=50:50; Flow rate:1 mL/min) to afford (1E,3R)-1-(2-methylpyrimidin-5-yl)-7-(5,6,7,8-tetrahydro-1,8-naphthyridin-2-yl) hept-1-en-3-ol 7 (41 g, 45.6%) as a yellow solid.

Compound 008: To a stirred mixture of (1E,3R)-1-(2-methylpyrimidin-5-yl)-7-(5,6,7,8-tetrahydro-1,8-naphthyridin-2-yl) hept-1-en-3-ol (41.00 g, 121.139 mmol, 1.00 equiv) and propionic acid (5 mL) in triethyl orthoacetate (400.00 mL) at room temperature under nitrogen atmosphere. The resulting mixture was stirred for 2 h at 145 degrees C. under nitrogen atmosphere. The resulting mixture was concentrated under reduced pressure. The crude product was used in the next step directly without further purification.

Compound 009: To a solution of ethyl (3R,4E)-3-(2-methylpyrimidin-5-yl)-9-(5,6,7,8-tetrahydro-1,8-naphthyridin-2-yl) non-4-enoate 8 (36.00 g, 88.117 mmol, 1.00 equiv) in EtOH (400.00 mL) was added Pd/C (10%, 3.6 g) under nitrogen atmosphere in a 1000 mL 3-necked round-bottom flask. The mixture was hydrogenated at room temperature for overnight under hydrogen atmosphere using a hydrogen balloon, filtered through a Celite pad and concentrated under reduced pressure. This resulted in ethyl (3S)-3-(2-methylpyrimidin-5-yl)-9-(5,6,7,8-tetrahydro-1,8-naphthyridin-2-yl) nonanoate 9 (35 g, 96.7%) as a white solid.

Compound 010: To a stirred solution of ethyl (3S)-3-(2-methylpyrimidin-5-yl)-9-(5,6,7,8-tetrahydro-1,8-naphthyridin-2-yl) nonanoate 9 (35.00 g, 85.249 mmol, 1.00 equiv) in AcOH (400.00 mL) was added Br₂ (14.99 g, 93.774 mmol, 1.10 equiv) dropwise portions at 10 degrees C. under nitrogen atmosphere. The resulting mixture was stirred for 1 h at room temperature under nitrogen atmosphere. The reaction was quenched with sat. aq. Na₂SO₃ at 10 degrees C. The mixture was basified to pH 11 with saturated aq. Na₂CO₃. The resulting mixture was extracted with EtOAc (2×500 mL). The combined organic layers were washed with water (3×200 mL) and brine (1×1000 mL), dried over anhydrous Na₂SO₄. After filtration, the filtrate was concentrated under reduced pressure. The residue was purified by silica gel column chromatography, eluted with CH₂C₁₂/MeOH (50:1) to afford ethyl (3S)-9-(3-bromo-5,6,7,8-tetrahydro-1,8-naphthyridin-2-yl)-3-(2-methylpyrimidin-5-yl) nonanoate (34 g, 81.5%) as a yellow oil.

Compound 011: To a stirred solution of ethyl (3S)-9-(3-bromo-5,6,7,8-tetrahydro-1,8-naphthyridin-2-yl)-3-(2-methylpyrimidin-5-yl)nonanoate 10 (34.00 g, 69.465 mmol, 1.00 equiv), Zn(CN)₂ (24.47 g, 208.395 mmol, 3.00 equiv) and Pd(PPh₃)₄(24.08 g, 20.840 mmol, 0.30 equiv) in DMF (400.00 mL) at room temperature under nitrogen atmosphere. The resulting mixture was stirred for overnight at 100 degrees C. under nitrogen atmosphere. The resulting mixture was extracted with EtOAc (2×400 mL). The combined organic layers were washed with water (2×200 mL) and brine (1×100 mL), dried over anhydrous Na₂SO₄. After filtration, the filtrate was concentrated under reduced pressure. The residue was purified by silica gel column chromatography, eluted with CH₂C₁₂/MeOH (10:1) to afford ethyl (3S)-9-(3-cyano-5,6,7,8-tetrahydro-1,8-naphthyridin-2-yl)-3-(2-methylpyrimidin-5-yl) nonanoate 11 (26 g, 85.9%) as a yellow oil.

Compound 012: To a stirred solution/mixture of ethyl (3S)-9-(3-cyano-5,6,7,8-tetrahydro-1,8-naphthyridin-2-yl)-3-(2-methylpyrimidin-5-yl) nonanoate 11 (26.00 g, 59.692 mmol, 1.00 equiv) in MeOH (200.00 mL) was added NaOH (1M, 20.00 mL) dropwise portions at room temperature under nitrogen atmosphere. The resulting mixture was stirred for 2h at room temperature under nitrogen atmosphere. The resulting mixture was concentrated under reduced pressure. The mixture was acidified to pH 3 with HCl in MeOH. The crude product was used in the next step directly without further purification.

Compound 013: To a solution of (3S)-9-(3-cyano-5,6,7,8-tetrahydro-1,8-naphthyridin-2-yl)-3-(2-methylpyrimidin-5-yl) nonanoic acid 12 (25.00 g, 61.347 mmol, 1.00 equiv) in NH₃ (g) in MeOH (300.00 mL) was added Raney-Ni (2.50 g, 29.180 mmol, 0.48 equiv) under nitrogen atmosphere in a 500 mL 3-necked round-bottom flask. The mixture was hydrogenated at room temperature for overnight under hydrogen atmosphere using a hydrogen balloon, filtered through a Celite pad and concentrated under reduced pressure. This resulted in (3S)-9-[3-(aminomethyl)-5,6,7,8-tetrahydro-1,8-naphthyridin-2-yl]-3-(2-methylpyrimidin-5-yl) nonanoic acid 13 (25 g, 99.0%) as a yellow oil.

Compound 014: To a stirred solution of (3S)-9-[3-(aminomethyl)-5,6,7,8-tetrahydro-1,8-naphthyridin-2-yl]-3-(2-methylpyrimidin-5-yl) nonanoic acid 13 (25.00 g, 60.746 mmol, 1.00 equiv) and H₂SO₄ (5.00 mL) in MeOH (300.00 mL) at room temperature under nitrogen atmosphere. The resulting mixture was stirred for overnight at 70 degrees C. under nitrogen atmosphere. The resulting mixture was concentrated under reduced pressure. This resulted in methyl (3S)-9-[3-(aminomethyl)-5,6,7,8-tetrahydro-1,8-naphthyridin-2-yl]-3-(2-methylpyrimidin-5-yl) nonanoate 14 (25 g, 96.7%) as a yellow oil.

Compound 015: To a stirred solution of 8-(tert-butoxy)-8-oxooctanoic acid (30.00 g, 130.263 mmol, 1.00 equiv) and TSTU (47.06 g, 156.320 mmol, 1.20 equiv) in THF (300.00 mL) were added DIEA (30.00 mL, 172.233 mmol, 1.32 equiv) in portions at room temperature under nitrogen atmosphere. The resulting mixture was stirred for overnight at room temperature under nitrogen atmosphere. The resulting mixture was extracted with EtOAc (2×100 mL). The combined organic layers were washed with conc. HCl (1×200 mL) and brine (1×100 mL), dried over anhydrous Na₂SO₄. After filtration, the filtrate was concentrated under reduced pressure. This resulted in 1-tert-butyl 2,5-dioxopyrrolidin-1-yl octanedioate 15 (35 g, 82.1%) as a white solid.

Compound 016: To a stirred solution of methyl (3S)-9-[3-(aminomethyl)-5,6,7,8-tetrahydro-1,8-naphthyridin-2-yl]-3-(2-methylpyrimidin-5-yl)nonanoate 14 (25.00 g, 58.744 mmol, 1.00 equiv),1-tert-butyl 2,5-dioxopyrrolidin-1-yl octanedioate (19.23 g, 58.744 mmol, 1.00 equiv) and 4-methyl morpholine (23.77 g, 234.976 mmol, 4.00 equiv) in DMSO (300.00 mL) at 10 degrees C. under nitrogen atmosphere. The resulting mixture was stirred for 2h at room temperature under nitrogen atmosphere. The resulting mixture was extracted with CH₂C₁₂ (2×500 mL). The combined organic layers were washed with water (2×200 mL) and brine(1×100 mL), dried over anhydrous Na₂SO₄. After filtration, the filtrate was concentrated under reduced pressure. The residue was purified by silica gel column chromatography, eluted with CH₂C₁₂/MeOH (15:1) to afford methyl (3S)-9-(3-[[8-(tert-butoxy)-8-oxooctanamido]methyl]-5,6,7,8-tetrahydro-1,8-naphthyridin-2-yl)-3-(2-methylpyrimidin-5-yl)nonanoate 16 (18 g, 48.1%) as a yellow solid. ¹H NMR (400 MHz, CDCl₃, ppm) δ8.47 (s, 2H), 7.03 (s, 1H), 5.51 (brs, 1H), 5.30 (s, 3H), 4.24 (d, J=5.2 Hz, 2H), 3.72 (t, J=4.8 Hz, 1H) 3.59 (s, 3H), 3.41-3.39 (m, 2H), 3.06 (brs, 1H), 2.72-2.41 (m, 12H), 2.29-2.14 (m, 4H), 1.89 (t, J=5.6 Hz, 2H), 1.67-1.53 (m, 8H), 1.43 (s, 9H), 1.33-1.16 (m, 12H). LCMS-016: (ES, m/z): 638.5 [M+H].

Compound 017: To a stirred solution of methyl (3S)-9-(3-[[8-(tert-butoxy)-8-oxooctanamido]methyl]-5,6,7,8-tetrahydro-1,8-naphthyridin-2-yl)-3-(2-methylpyrimidin-5-yl)nonanoate 16 (18.00 g, 28.219 mmol, 1.00 equiv) in DCM (200 mL) were added TFA (40.00 mL) dropwise portions at 10 degrees C. under nitrogen atmosphere. The resulting mixture was stirred for 2 h at 10 degrees C. The resulting mixture was concentrated under vacuum. The residue was purified by reverse flash chromatography with the following conditions: column, C18; mobile phase, ACN in water, 10% to 50% gradient in 20 min; detector, UV 254 nm. This resulted in 7-[([2-[(7S)-9-methoxy-7-(2-methylpyrimidin-5-yl)-9-oxononyl]-5,6,7,8-tetrahydro-1,8-naphthyridin-3-yl]methyl)carbamoyl]heptanoic acid 17 (10.4742 g, 63.8%) as a white solid. ¹H NMR-017: (400 MHz, DMSO-d₆, ppm) δ8.58 (s, 2H), 7.92 (t, J=5.4 Hz, 1H), 6.93 (s, 1H), 6.21 (s, 1H), 4.03 (d, J=5.3 Hz, 2H), 3.49 (s, 3H), 3.22 (d, J=6.0 Hz, 2H), 2.98 (hept, J=5.8 Hz, 1H), 2.77 (dd, J=16.0, 6.1 Hz, 1H), 2.66 (dd, J=16.0, 9.0 Hz, 1H), 2.57 (d, J=4.7 Hz, 5H), 2.40 (t, J=7.8 Hz, 2H), 2.16 (t, J=7.4 Hz, 2H), 2.04 (t, J=7.4 Hz, 2H), 1.74 (q, J=5.8 Hz, 2H), 1.68-1.53 (m, 2H), 1.46 (h, J=6.6, 6.1 Hz, 6H), 1.27-1.18 (m, 8H), 1.06 (d, J=43.2 Hz, 2H). LCMS-017: (ES, m/z): 582 [M+1].

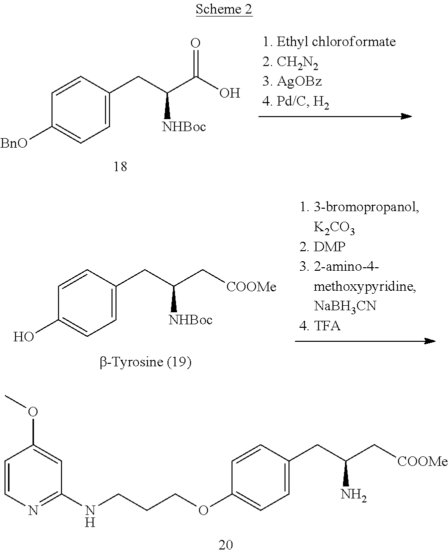 [SMILES: CC(C)(C)OC(=O)N[C@@H](Cc1ccc(OCc2ccccc2)cc1)C(=O)O ; COC(=O)C[C@H](Cc1ccc(O)cc1)NC(=O)OC(C)(C)C ; COC(=O)C[C@@H](N)Cc1ccc(OCCCNc2cc(OC)ccn2)cc1]

Beta tyrosine 19 was prepared as described in Chem Biochem 2008, 9:1397; and Tetrahedron 1995, 45:12337. Elaboration of beta tyrosine to advanced intermediate 20 was completed in 4 steps as described in J. MED 2014, 57:3410.

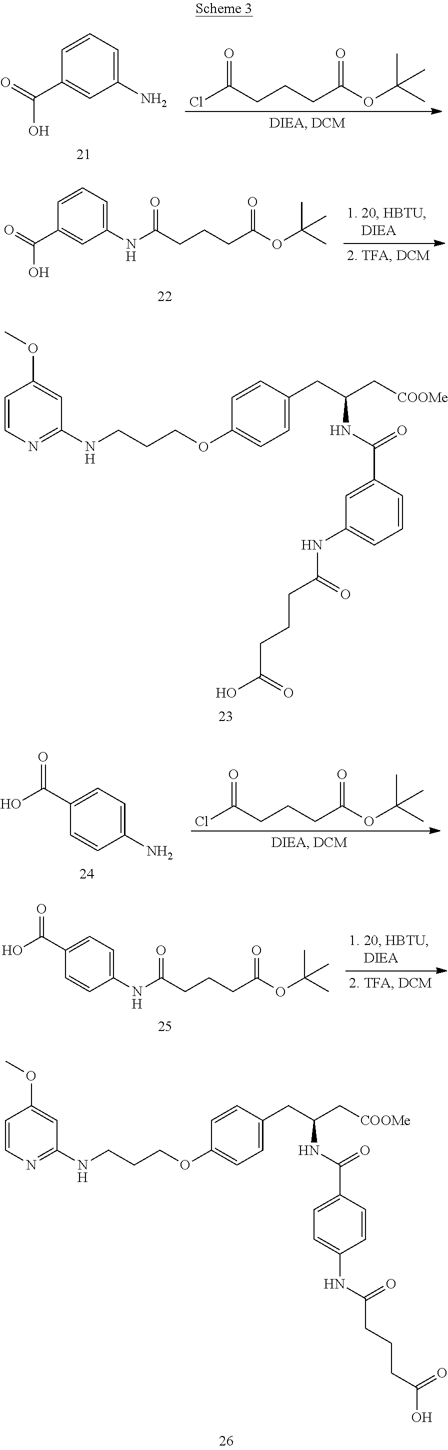

Compound 23 and 24 (Scheme 3) were synthesized from ¾-aminobenzoic acid (21 or 24) in 3 steps via sequential amidation reactions followed by tert-butyl ester deprotection with trifluoro acetic acid. A similar sequence beginning with 4-aminobenzoic acid provides compound 24.

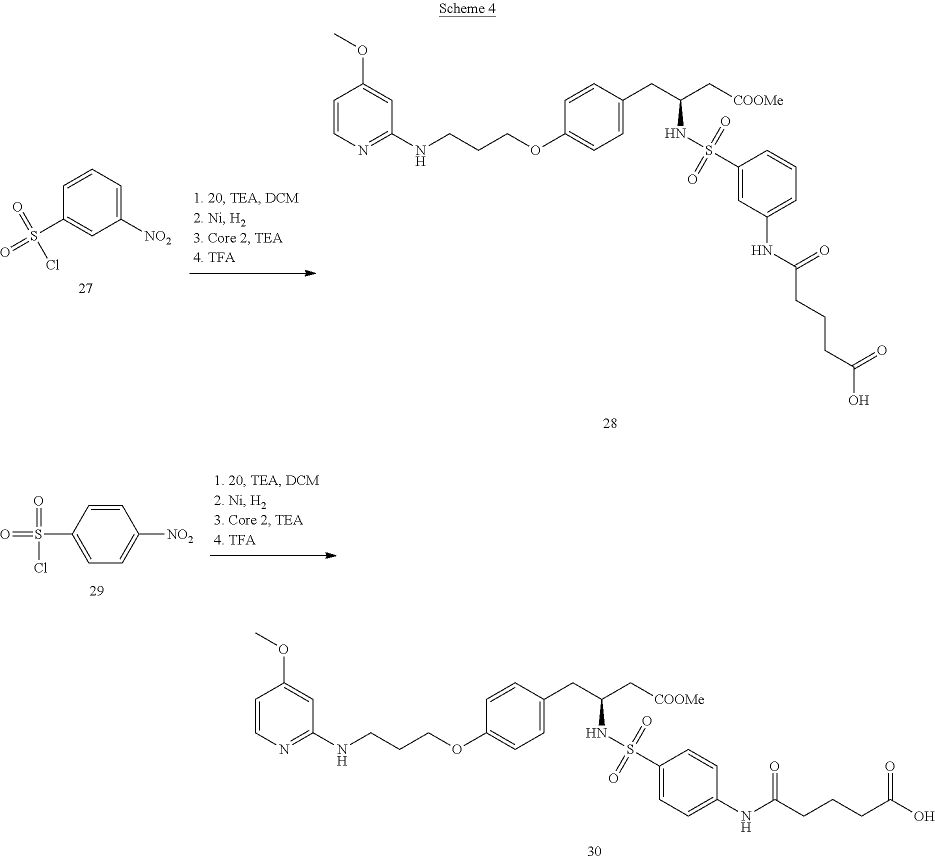 [SMILES: O=[N+]([O-])c1cccc(S(=O)(=O)Cl)c1 ; COC(=O)C[C@H](Cc1ccc(OCCCNc2cc(OC)ccn2)cc1)NS(=O)(=O)c1cccc(NC(=O)CCCC(=O)O)c1 ; O=[N+]([O-])c1ccc(S(=O)(=O)Cl)cc1 ; COC(=O)C[C@H](Cc1ccc(OCCCNc2cc(OC)ccn2)cc1)NS(=O)(=O)c1ccc(NC(=O)CCCC(=O)O)cc1]

Sulfonamide containing compounds 28 and 30 were synthesized (Scheme 4) from 3-nitrobenzenesulfonyl chloride or 4-nitrobenzenesulfonyl chloride respectively. Sulfonamide formation with 20 is followed by nitro reduction and subsequent amidation with tert-butyl 5-chloro-5-oxopentanoate provided a tert-butyl ester which was saponified under acidic conditions to provide acid functional group. Coupling of compounds 28 and 30 to sense strands of oligonucleotide sequences was performed with standard peptide coupling agents such as EDCI or T3P.

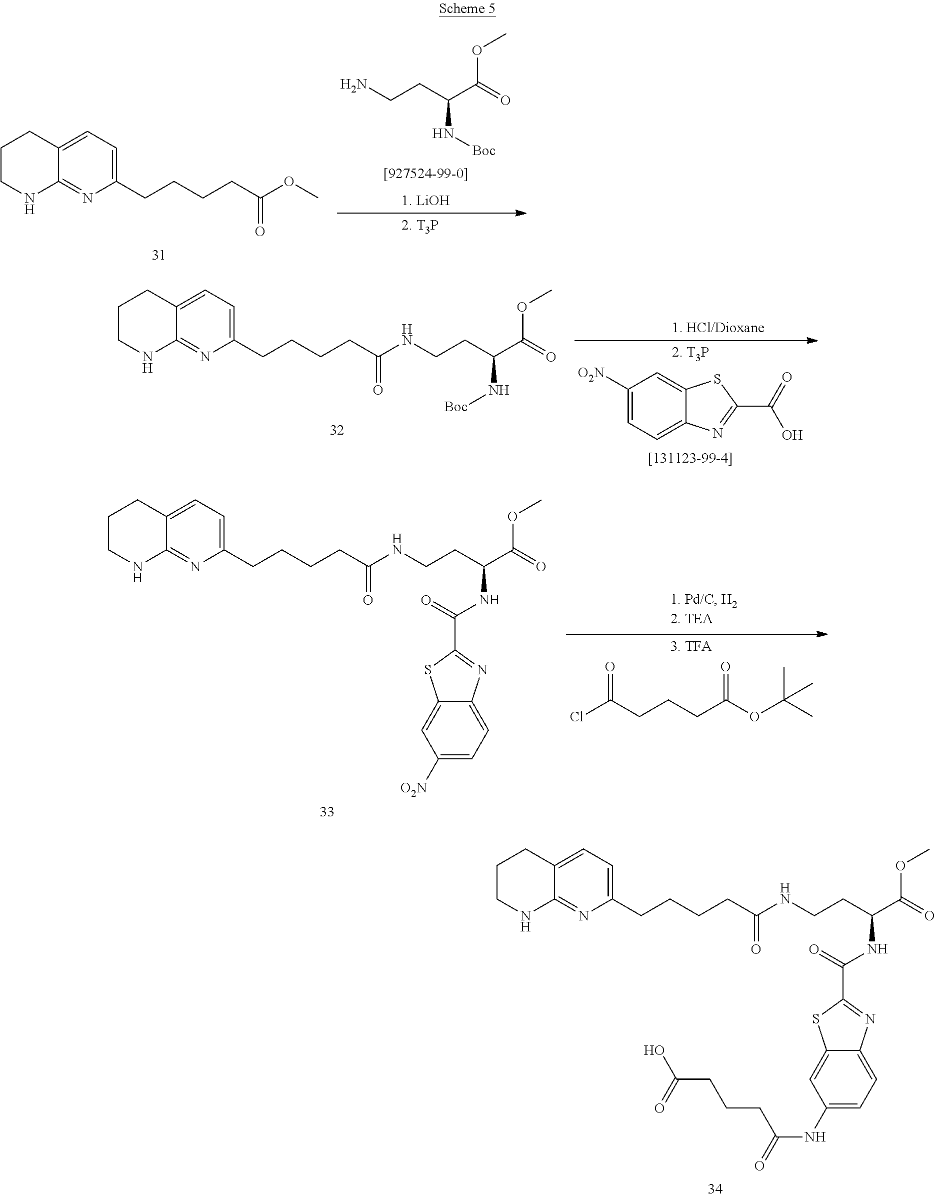

Preparation of compound 34 (Scheme 5) commenced from Intermediate 31. The methyl ester was saponified under basic conditions and then amidated methyl (S)-4-amino-2-((tert-butoxycarbonyl)amino) butanoate. Deprotection of the tert-butyl carbamate was followed by amidation with 6-nitrobenzo[d]thiazole-2-carboxylic acid. Reduction of the nitro group provided an aniline ready for amidation with tert-butyl 5-chloro-5-oxopentanoate and subsequent tert-butylester deprotection with trifluoroacetic acid. Coupling of compound 34 to sense strands of oligonucleotide sequences was performed with standard peptide coupling agents such as EDCI or T3P.

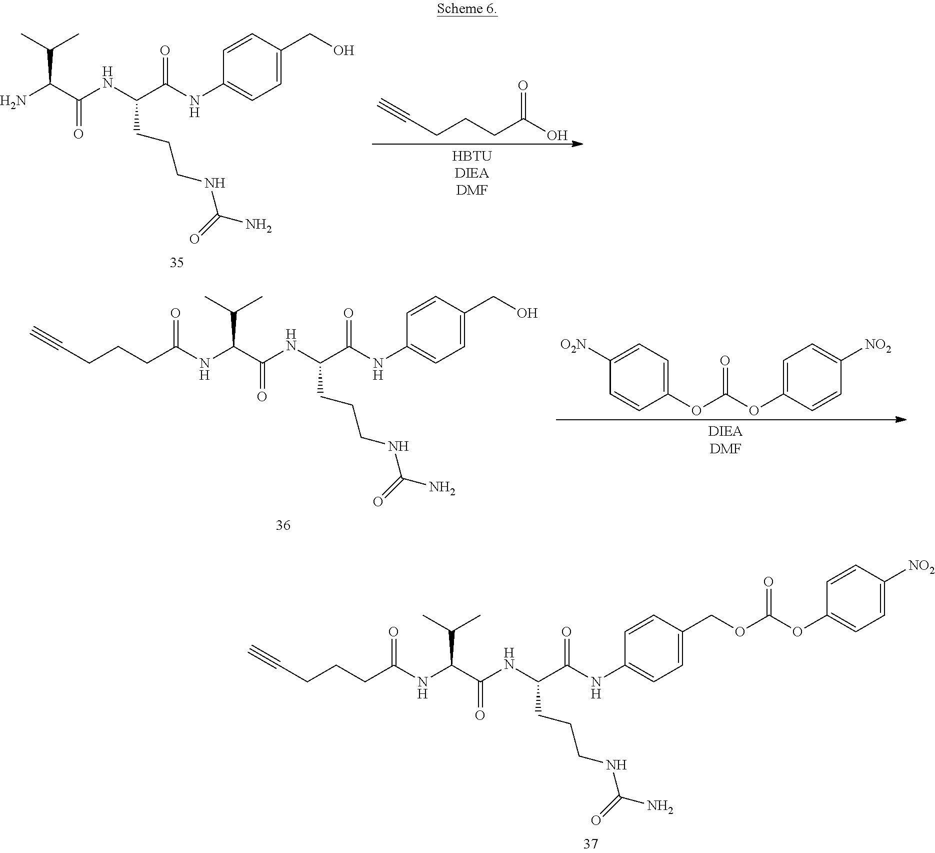

Compound 36: To a solution of hexynoic acid (434 ul, 3.95 mmol) in DMF (25 mL) under argon was added diisopropylethylamine (1.84 ml, 10.54 mmol) via syringe and stirred the reaction mixture for 5 minutes. A solution of a solution of compound 35 (lg, 2.64 mmol) in DMF was added via syringe. The reaction was stirred at room temperature overnight. After checking the TLC (10% MeOH/DCM) for completeness, the reaction was concentrated on rotavapor. The crude was dissolved in minimal MeOH/DCM and loaded onto silica gel. This slurry was concentrated on the rotavapor and dried on high vacuum. This was purified by flash chromatography on silica gel (0% to 10% MeOH/DCM) and the product fractions combined and concentrated on reduced pressure to yield (0.608g 49%) of 36. ¹H NMR (600 MHz, DMSO) δ 9.92 (s, 1H), 8.16-8.09 (m, 1H), 7.91 (d, J=8.6 Hz, 1H), 7.75 (d, J=8.2 Hz, 1H), 7.58-7.52 (m, 2H), 7.47 (dd, J=8.0, 1.3 Hz, 1H), 7.26-7.17 (m, 3H), 5.99 (t, J=5.7 Hz, 1H), 5.43 (s, 2H), 5.12 (s, 1H), 4.43 (s, 2H), 4.38 (m, 1H), 4.19 (m, 1H), 3.02 (m, 2H), 2.94 (m, 2H), 2.81 (m, 1H), 2.33-2.27 (m, 1H), 2.27-2.21 (m, 1H), 2.21-2.12 (m, 3H), 1.98 (m, 1H), 1.74-1.54 (m, 5H), 1.44 (m, 1H), 1.40-1.29 (m, 2H), 1.19 (d, J=6.5 Hz, 2H), 1.13 (s, 2H), 0.92-0.81 (m, 7H). Mass calc. for C₂₄H₃₅N₅O₅: 473.57, found: 474.2 (M+1), 472.2 (M-1)

Compound 37: Compound (36) (0.385g, 812.98umol) and Bis(4-nitrophenyl) carbonate (741.96 mg, 2.44 mmol) was dissolved in DMF (15 mL) in a round bottom flask under argon. Diisopropylethylamine (158 ul, 894 umol) was added via syringe into reaction flask. Let the reaction stir at room temp for 5 hours. Reaction checked by MS and concentrated on rotavapor, ethyl acetate was added to precipitate product. The product was filtered off, washed with ethyl acetate and dried on high vacuum to yield (0.288g 55%) of 37 ¹H NMR (600 MHz, DMSO) δ 10.07 (s, 1H), 8.34-8.29 (m, 1H), 8.12 (dd, J=8.4, 2.3 Hz, 1H), 7.88 (d, J=8.6 Hz, 1H), 7.70-7.62 (m, 2H), 7.62-7.54 (m, 2H), 7.44-7.38 (m, 2H), 5.98 (t, J=5.7 Hz, 1H), 5.42 (s, 2H), 5.25 (s, 1H), 4.39 (m, 1H), 4.20 (m, 1H), 3.04 (m, 1H), 2.95 (m, 1H), 2.80 (m, 1H), 2.34-2.21 (m, 2H), 2.21-2.12 (m, 2H), 2.02-1.94 (m, 2H), 1.75-1.64 (m, 3H), 1.61 (m, 1H), 1.48-1.41 (m, 1H), 1.41-1.33 (m, 1H), 1.18 (t, J=7.1 Hz, 1H), 0.89-0.82 (m, 6H). Mass calc. for C₃₁H3sN₆09: 638.68, found: 639.2 (M+1), 661.2 (M+Na)

### Synthesis of cleavable linker 42

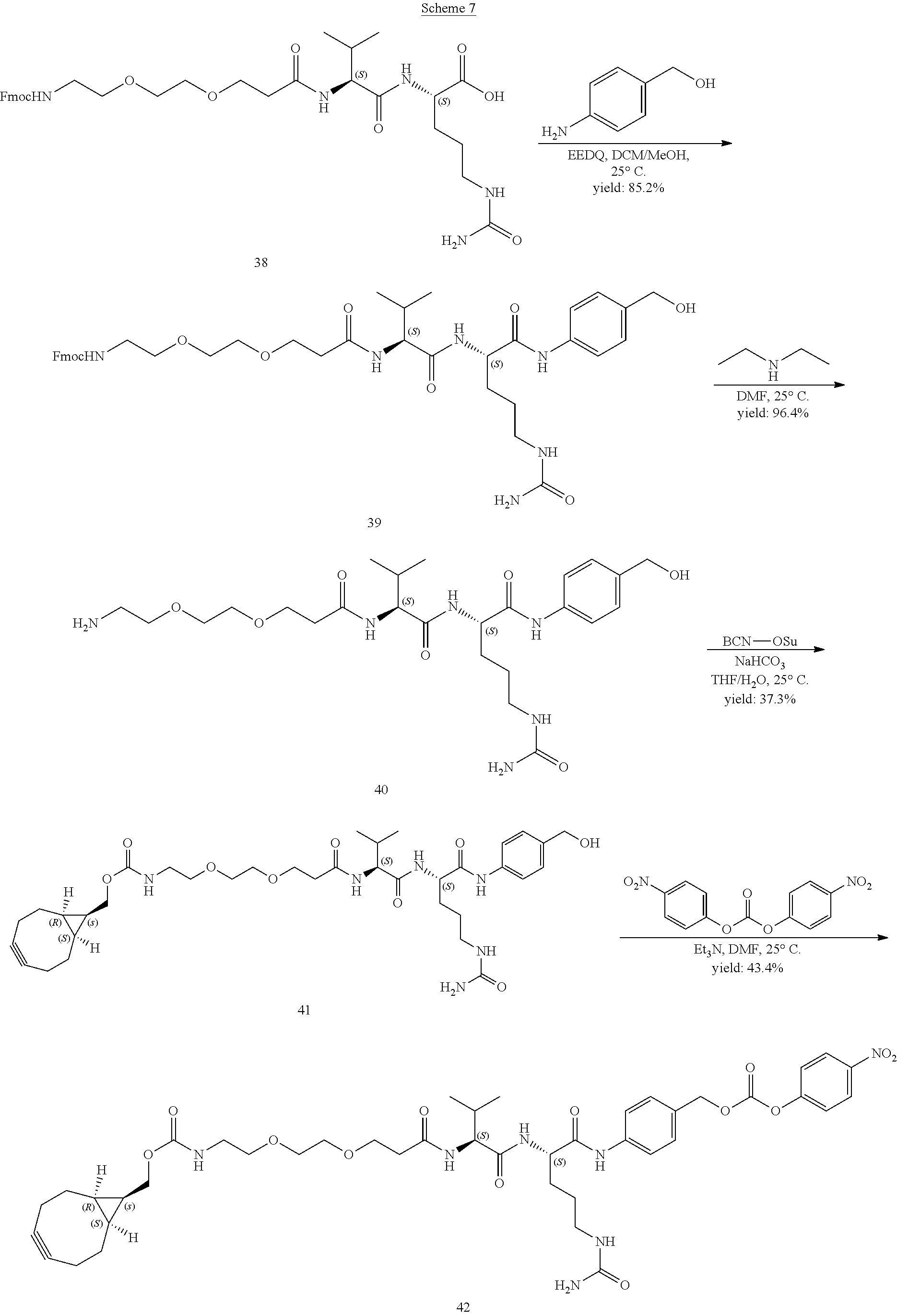

Compound 39: To a solution of compound 38 (15.0 g, 21.2 mmol, obtained by standard Fmoc peptide synthesis) in DCM (75.0 mL) and MeOH (75.0 mL) was added 4-aminobenzyl alcohol (5.23 g, 42.5 mmol) and EEDQ (10.5 g, 42.5 mmol). The mixture was stirred at 25° C. for 16.0 h and exclusion of light. LCMS showed the raw material was consumed and one peak with desired mass was detected. The reaction solution was added dropwise to a solution of stirring isopropyl ether (1.50 L) to precipitate a white solid and filtered, dried to give the residue. The residue was triturated with MTBE (500 mL) to yield (30.0 g, 85.2%) of 39 ¹H NMR (400 MHz, MeOD) 5: 7.80-7.78 (d, J=7.6 Hz, 2H), 7.65-7.63 (d, J=7.6 Hz, 2H), 7.57-7.55 (d, J=8.4 Hz, 2H), 7.40-7.36 (t, J=8.4 Hz, 2H), 7.32-7.28 (t, J=8.4 Hz, 4H), 4.54 (s, 2H), 4.52-4.49 (m, 1H), 4.36-4.34 (d, J=8.4 Hz, 2H), 4.24-4.18 (m, 2H), 3.75-3.72 (m, 2H), 3.60-3.58 (m, 4H), 3.29-3.26 (m, 2H), 3.12-3.06 (m, 2H), 2.55-2.49 (m, 2H), 2.14-2.05 (m, 1H), 1.97-1.85 (m, 1H), 1.77-1.69 (m, 1H), 1.62-1.53 (m, 1H), 1.01-0.95 (m, 6H). Mass calc. for C₄₀H₅₂N₆O₉: 760.4, found: 761.3 (M+H)

Compound 40: To a solution of compound 39 (15.0 g, 19.7 mmol) in DMF (150 mL) was added N-ethylethanamine (7.20 g, 98.6 mmol). The mixture was stirred at 25° C. for 4 h. LCMS showed the starting material was consumed and one peak with desired mass was detected. Two batch of reaction solution was combined and diluted with 200 mL DCM. The mixture was dropped into isopropyl ether (2.00 L) to precipitate a white solid and filtered. The filter cake was washed with MTBE (200 mL) and concentrated under reduced pressure to yield (23.0 g, 96.4%) of 40 ¹H NMR (400 MHz, D₂O) 5:7.45-7.40 (m, 4H), 4.62 (s, 2H), 4.45-4.42 (m, 1H), 4.17-4.14 (m, 1H), 3.80-3.77 (m, 2H), 3.64-3.62 (m, 4H), 3.18-3.13 (t, J=6.8 Hz, 2H), 3.06-3.04 (m, 2H), 3.02-3.01 (m, 2H), 2.68-2.55 (m, 2H), 2.13-2.04 (m, 1H), 1.96-1.90 (m, 1H), 1.87-1.79 (m, 1H), 1.68-1.53 (m, 2H), 0.97-0.95 (m, 6H). Mass calc. for C₂₅H₄₂N₆O₇: 538.3, found: 539.3 (M+H) Compound 41: To a solution of compound 40 (10.0 g, 18.6 mmol) in DI-water (18.0 mL) and THE (20.0 mL) was added NaHCO₃ (1.56 g, 18.6 mmol) and BCN-OSu (5.95 g, 20.4 mmol). The mixture was stirred at 25° C. for 12.0 h. LCMS showed the starting material was consumed and one major peak with desired mass was detected. Two batches of the reaction solution were combined and adjusted to pH around-5-6, concentrated under reduced pressure and the aqueous layer was purified by prep-HPLC ([water (0.1% FA)-ACN]; B %: 30%-50%, 100 min) to yield (5.10 g, 93.0% yield) of 41 ¹H NMR (400 MHz, D₂O) δ:7.44-7.36 (m, 4H), 4.59 (s, 2H), 4.41-4.38 (m, 1H), 4.18-4.07 (m, 3H), 3.76-3.74 (m, 2H), 3.65-3.56 (m, 4H), 3.54-3.53 (m, 2H), 3.31-3.26 (m, 2H), 3.13-3.10 (t, J=7.2 Hz, 2H), 2.59-2.56 (t, J=5.6 Hz, 2H), 2.22-2.12 (m, 6H), 2.13-2.03 (m, 1H), 1.88-1.75 (m, 2H), 1.59-1.44 (m, 4H), 1.33-1.28 (m, 1H), 0.93-0.87 (m, 8H). Mass calc. for C₃₆H₅₄N₆O₉: 714.4, found: 715.4 (M+H)

Compound 42: To a solution of compound 41 (4.60 g, 6.43 mmol) in DMF (20.0 mL) was added Et₃N (985ul, 7.08 mmol) and bis(4-nitrophenyl) carbonate (2.15 g, 7.08 mmol). The reaction mixture was stirred at 25° C. for 12.0 h. LCMS showed the starting material was consumed and one peak with desired mass was detected. The reaction solution was diluted with 100 mL DCM and then washed with DI-water (500 ml 2x). Most of the organic phase was concentrated and then diluted with 50 ml acetonitrile, followed by addition of 300 mL isopropyl ether to form a white precipitate. The crude product was collected by filtration after 0.5 h trituration, two batches of crude product (about 7.00 g) were combined for further purification with prep-HPLC ([Water-ACN];B %: 30.0%-60.0%, 20.0 min) to yield (5.10 g, 43.4%) of 42 ¹H NMR (400 MHz, DMSO) δ:10.05 (s, 1H), 8.33-8.30 (m, 2H), 8.13-8.11 (d, J=7.6 Hz, 1H), 7.87-7.85 (d, J=8.4 Hz, 1H), 7.66-7.64 (d, J=8.4 Hz, 2H), 7.59-7.55 (m, 2H), 7.42-7.40 (d, J=8.4 Hz, 2H), 7.09-7.06 (m, 1H), 5.99-5.96 (m, 1H), 5.41 (s, 2H), 5.24 (s, 2H), 4.42-4.36 (m, 1H), 4.25-4.19 (m, 1H), 4.04-4.02 (d, J=8.0 Hz, 2H), 3.64-3.57 (m, 2H), 3.51-3.44 (m, 4H), 3.40-3.37 (m, 2H), 3.29 (s, 2H), 3.13-3.08 (m, 2H), 3.06-2.91 (m, 2H), 2.41-2.32 (m, 2H), 2.26-2.12 (m, 6H), 2.03-1.93 (m, 1H), 1.74-1.68 (m, 1H), 1.63-1.34 (m, 5H), 1.30-1.20 (m, 1H), 0.87-0.83 (m, 8H). Mass calc. for C₄₃H₅₇N₇O₁₃: 879.4, found: 880.6 (M+H)

### Example 2. Design and synthesis of siRNA conjugates

This Example describes methods for the design, synthesis, and conjugation of siRNA agents. Nucleic acid sequences provided herein are represented using standard nomenclature. See the abbreviations of Table 2.

TABLE 2
Abbreviations of nucleotide monomers
used in nucleic acid sequence representation.
It will be understood that these monomers, when present in an
oligonucleotide, are mutually linked by 5′-3′-phosphodiester bonds.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| P | Phosphate |
| VP | Vinyl-phosphate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| Y34 | 2-hydroxymethyl-tetrahydrofurane-4-methoxy-3-phosphate |
| | (abasic 2′-OMe furanose) |
| Y44 | inverted abasic DNA (2-hydroxymethyl-tetrahydrofurane-5- |
| | phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| (Aam) | 2′-O-(N-methylacetamide)adenosine-3′-phosphate |
| (Aams) | 2′-O-(N-methylacetamide)adenosine-3′-phosphorothioate |
| (Gam) | 2′-O-(N-methylacetamide)guanosine-3′-phosphate |
| (Gams) | 2′-O-(N-methylacetamide)guanosine-3′-phosphorothioate |
| (Tam) | 2′-O-(N-methylacetamide)thymidine-3′-phosphate |
| (Tams) | 2′-O-(N-methylacetamide)thymidine-3′-phosphorothioate |
| (Aeo) | 2′-O-methoxyethyladenosine-3′-phosphate |
| (Aeos) | 2′-O-methoxyethyladenosine-3′-phosphorothioate |
| (Geo) | 2′-O-methoxyethylguanosine-3′-phosphate |
| (Geos) | 2′-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2′-O-methoxyethyl-5-methylcytidine-3′-phosphorothioate |
| (A3m) | 3′-O-methyladenosine-2′-phosphate |
| (A3mx) | 3′-O-methyl-xylofuranosyladenosine-2′-phosphate |
| (G3m) | 3′-O-methylguanosine-2′-phosphate |
| (G3mx) | 3′-O-methyl-xylofuranosylguanosine-2′-phosphate |
| (C3m) | 3′-O-methylcytidine-2′-phosphate |
| (C3mx) | 3′-O-methyl-xylofuranosylcytidine-2′-phosphate |
| (U3m) | 3′-O-methyluridine-2′-phosphate |
| U3mx) | 3′-O-methyl-xylofuranosyluridine-2′-phosphate |
| (m5Cam) | 2′-O-(N-methylacetamide)-5-methylcytidine-3′-phosphate |
| (m5Cams) | 2′-O-(N-methylacetamide)-5-methylcytidine-3′- |
| | phosphorothioate |
| (Chd) | 2′-O-hexadecyl-cytidine-3′-phosphate |
| (Chds) | 2′-O-hexadecyl-cytidine-3′-phosphorothioate |
| (Uhd) | 2′-O-hexadecyl-uridine-3′-phosphate |
| (Uhds) | 2′-O-hexadecyl-uridine-3′-phosphorothioate |
| (pshe) | Hydroxyethylphosphorothioate |

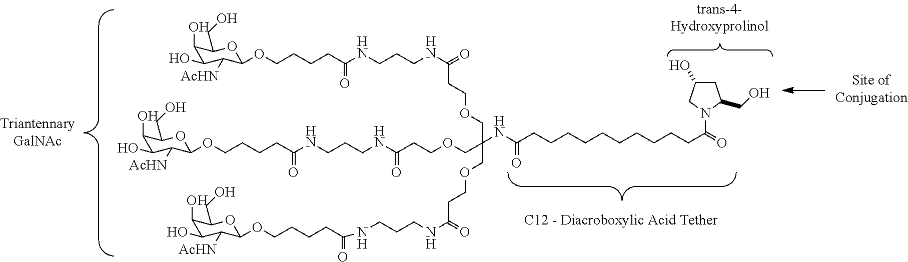

### General Conditions of Oligonucleotide Synthesis:

Oligonucleotides were synthesized by solid-phase synthesis using an RNA synthesizer. Sterling solvents/reagents from Glen Research, 500-A controlled pore glass (CPG) solid supports from Prime Synthesis, and 2′-OMe, 2′-F nucleoside 3′-phosphoramidites from Hongene were all used as received. Low-water content acetonitrile was purchased from EMD Chemicals. RNA oligonucleotides were synthesized using modified synthesis cycles, based on those provided with the instrument. A solution of 0.6 M 5-(S-ethylthio)-1H-tetrazole in acetonitrile was used as the activator. Terminal 3′-amino groups of the sense strands were generated using the mono- or tris-(aminoalkyl)-4-hydroxyprolinol scaffold attached via succinate linker on in-house prepared CPG supports. The phosphoramidite solutions were 0.15 M in anhydrous acetonitrile with 15% DMF as a co-solvent for 2′-OMe uridine and cytidine. The 2′-OMe-uridine-5′-bis-POM-(E) vinylphosphonate (VP) 3′-phosphoramidite was synthesized according to previously published procedures, dissolved to 0.15 M in 85% acetonitrile 15% dimethylformamide (DMF) and coupled using standard conditions on the synthesizer. The oxidizing reagent was 0.02 M I₂ in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM). After completion of the solid-phase syntheses, the CPG solid support was washed with 5% (v/v) piperidine in anhydrous acetonitrile three times with 5-minute holds after each flow. The support was then washed with anhydrous acetonitrile and dried with argon. The oligonucleotides were then incubated with 28-30% (w/v) NH₄0H, at 35° C. for 20 h; or with a mixture of ammonia and methylamine (AMA) -50:50 at room temperature for 3 h (for 3′-amino oligonucleotides). For VP-containing oligonucleotides, after completion of the solid phase synthesis, the CPG solid support was incubated with 28-30% (w/v) NH4OH, where 5% (v/v) of diethylamine was added, at 35° C. for 20 h. In all cases, the solvents were collected by filtration and the support was rinsed with water. Crude oligonucleotides were purified using strong anion exchange HPLC with phosphate buffers (pH=8.5 or 11) containing 1 M sodium bromide, the appropriate fractions were pooled, and finally desalted. The identities and purities of all oligonucleotides were confirmed using ESI-LC/MS and IEX HPLC. Oligonucleotide solutions of ˜1 OD₂₆₀ Units/mL were used for analysis of the crudes. LC/ESI-MS was performed on an Agilent 6130 single quadrupole LC/MS system using an XBridge C8 column (2.1×50 mm, 2.5 μm). Buffer A consisted of 200 mM 1,1,1,3,3,3-hexafluoro-2-propanol (HFIP) and 16.3 mM triethylamine (TEA) in water, and buffer B was 100% methanol. A gradient from 0% to 40% of buffer B over 10 min followed by washing and recalibration at a flow rate of 0.70 mL/min. The column temperature was 75° C. IEX-HPLC was performed on an Agilent 1200 HPLC system using an DNA Pac column (9×250 mm, 13 μm). Buffer A consisted of 20 mM sodium phosphate in water, pH 11, and buffer B was buffer A plus 1 M sodium bromide. A gradient from 0% to 65% of buffer B over 20 min followed by washing and recalibration at a flow rate of 1 mL/min was applied and the column temperature was 35° C.

If a backbone modification like cyclic guanidinium and/or sulfonyl phosphoramidite are required to be part of the oligonucleotide, the procedure described above is modified as follows: immediately after the coupling steps, the oligonucleotide is oxidized 5 times for the cyclic guanidinium with a 0.5 M solution of the corresponding guanidinium azide in ACN per incorporation. For the sulfonyl phosphoramidite modification, the oligonucleotide is oxidized 5 times at a concentration of 0.200-0.5 M. Each oxidation cycle is 10 minutes. The solid support is then washed with CAN followed by a capping step.

### Post-Synthetic Conjugation for siRNA conjugates having small molecule ligands

A stock solution of 100 mg/mL (˜30 mM) of either the 3′-mono or 3′-tris aminoalkyl oligonucleotide precursor in 50/50 (v/v) water and n-methylpyrrolidone (NMP) was prepared. Separately, a solution with 3 equivalents of 17, 3 equivalents of PyAOP, and 12 equivalents of diisopropylethylamine in NMP (12.5 μL per mg of 17) was prepared. Equivalents were calculated based on each amine on the oligonucleotide precursor. First, half of the activated solution of 17 is added to the oligonucleotide solution, the solution is vortexed at room temperature, and after a wait for 3 minutes, the remaining activated solution is added to the reaction mixture, the vial is again vortexed and followed by a 3-minute wait. After confirming the product formation by LC/MS, the methyl ester of the ligand was hydrolyzed by adding 1 M aq. piperidine (125 μL per mg of starting oligo precursor) and shaking at room temperature for 24 hrs. The crude material was then purified using reverse phase chromatography (Buffer A: 20 mM sodium acetate with 3% acetonitrile. Buffer B: 20 mM sodium acetate with 80% acetonitrile). The purified material was then desalted to the final conjugated oligonucleotide product, lyophilized from water.

### Post-Synthetic Conjugation for siRNA conjugates having peptide ligands

A. siRNA's with Maleimide

The purified, desalted sense strand, containing the 3′-mono(aminoalkyl) linker was activated via conjugation of the maleimide SMCC (succinimidyl 4-(N-maleimidomethyl)cyclohexane-1-carboxylate, Thermo Scientific, Catalog #22360), added to the 3′-C6 amino group: 60 mg of lyophilized and powdered sense strand were dissolved in 500 μL of pH 8.0 100 mM sodium phosphate buffer; separately, 25 mg of powdered SMCC maleimide were dissolved in 2 mL of ACN and added to the reaction mixture. The resulting solution was well mixed in a vortex and shaken for 15 minutes at room temperature. The reaction progress was analyzed by LC/MS to confirm the reaction completion, the mixture was then neutralized to pH 7.0, and desalted by SEC to remove the excess SMCC. The final oligonucleotide was then lyophilized from water. All the starting 3′-mono (aminoalkyl) oligonucleotide should be consumed, with some side product of hydrolyzed maleimide present (typically less than 5%). The resulting lyophilized activated oligonucleotide was then used in the next step without further purification.

### B. RGD Peptide-Maleimide Conjugation

The maleimide activated oligonucleotide was reconstituted in 1 mL of water. Approximately 1.2 equivalents of RGD peptides (peptides are generated based on the reported procedure Bioconjugate Chem. 2011, 22, 1673-1681) was dissolved in 2 mL of 0.1M HEPES buffer. The two solutions were mixed well and shaken for 1-2 hours at room temperature. After confirming completion of the product formation by LC/MS reverse phase HPLC purification was performed. For purification, Buffer A consisted of 50 mM TEAA (Triethylammonium acetate) and 2% ACN, buffer B consisted of 50 mM TEAA and 80% ACN. Waters C-18 Reverse phase resin was used with a gradient ranging from 0-30 to 0-50% B in 90 to 120 minutes. Fractions were analyzed by LC/MS and any with greater than 70% purity were pooled, concentrated, subjected to a large excess of NaCl for sodium ion exchange on the phosphate backbone, then desalted again. Final product was pooled and evaporated to dryness, filtered through 0.2 μm polyethersulfone filters, and quantified by UV spectrophotometer. The final oligonucleotide conjugates were lyophilized from water.

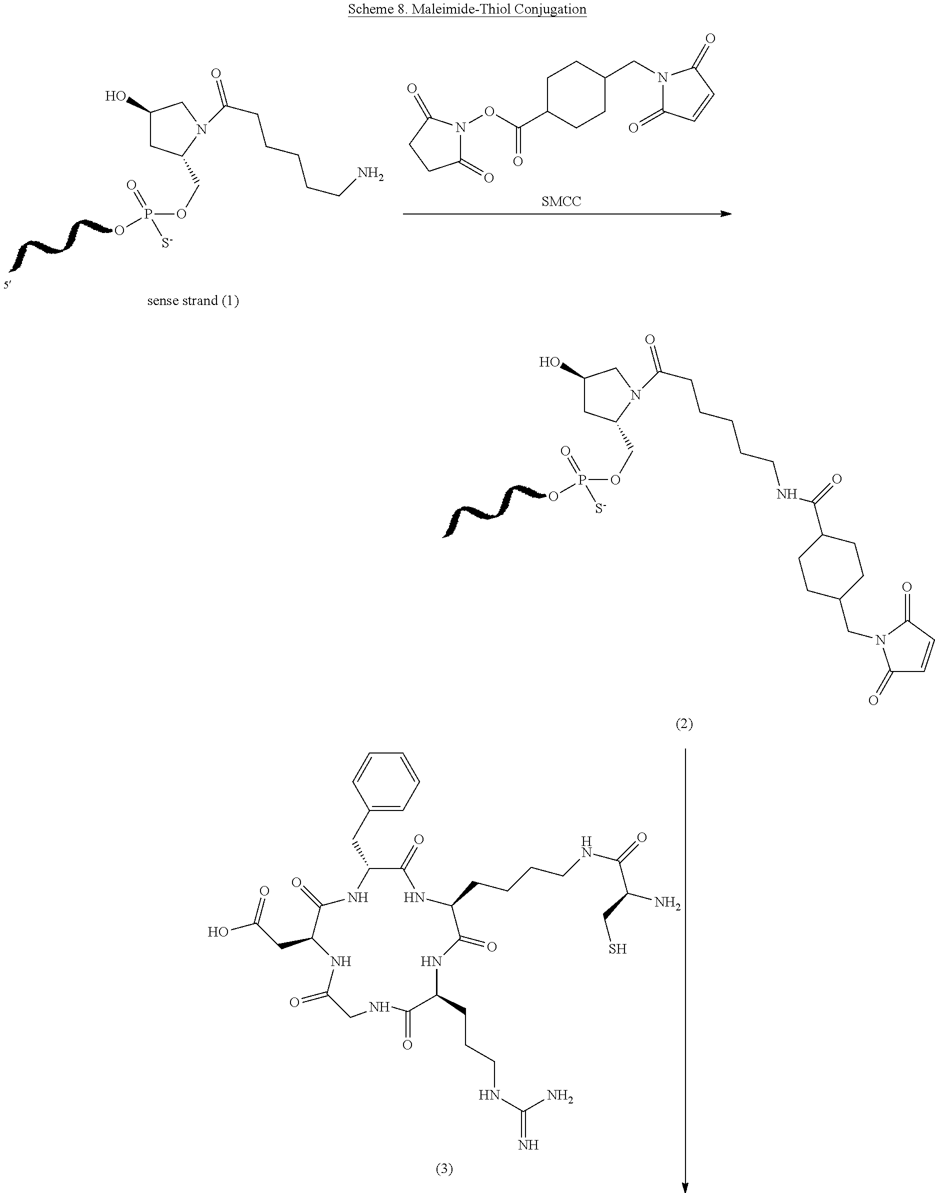 [SMILES: NCCCCCC(=O)N1C[C@H](O)C[C@H]1COP(=O)(O)[S-] ; O=C(ON1C(=O)CCC1=O)C1CCC(CN2C(=O)C=CC2=O)CC1 ; O=C(NCCCCCC(=O)N1C[C@H](O)C[C@H]1COP(=O)(O)[S-])C1CCC(CN2C(=O)C=CC2=O)CC1 ; N=C(N)NCCC[C@@H]1NC(=O)[C@H](CCCCNC(=O)[C@@H](N)CS)NC(=O)[C@@H](Cc2ccccc2)NC(=O)[C@H](CC(=O)O)NC(=O)CNC1=O]

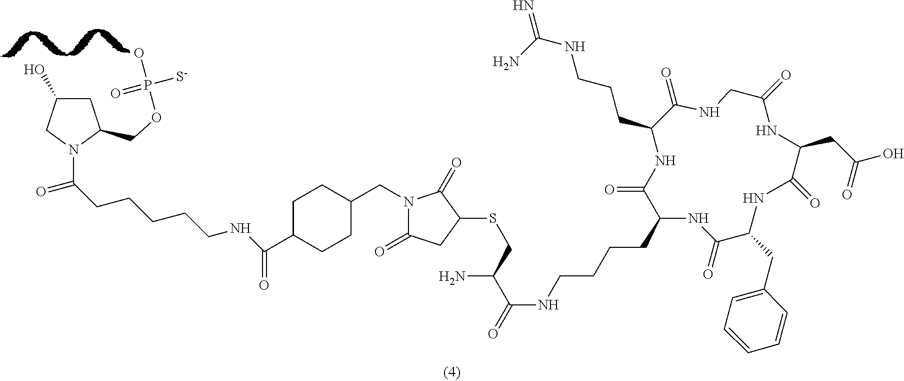 [SMILES: N=C(N)NCCC[C@@H]1NC(=O)[C@H](CCCCNC(=O)[C@@H](N)CSC2CC(=O)N(CC3CCC(C(=O)NCCCCCC(=O)N4C[C@H](O)C[C@H]4COP(=O)(O)[S-])CC3)C2=O)NC(=O)[C@@H](Cc2ccccc2)NC(=O)[C@H](CC(=O)O)NC(=O)CNC1=O]

In a 40 mg scale synthesis, 1 equivalent of sense strand (1) containing 3′-amine was diluted to 1 mL with pH 7.4 1X phosphate buffer saline (PBS). To this solution, 3-5 equivalents of sulfosuccinimidyl 4-(N-maleimidomethly)cyclohexane-1-carboxylate (SMCC), dissolved in 0.5 mL of anhydrous acetonitrile (ACN), was added. The reaction mixture was incubated at room temperature overnight prior to desalting via size exclusion chromatography (SEC). Afterwards, the maleimide-modified sense strand (2) was resuspended with 1.5 mL of 0.5 M pH 7.5 HEPES buffer, followed by the addition of 1.5 equivalents of thiol-modified cRGD (3) in 1:1 NMP: 1M pH 7.5 HEPES buffer. The resulting mixture was incubated at 37° C. for 2 hrs. Purification was performed using reverse phase chromatography (C18 column) followed by desalting using SEC. [M] calculated for (4) 8219.045 Da, found 8217.53 Da (LC-MS). The purified sense strand conjugate (21 mg, 52% yield, 94% purity via LCMS) was then lyophilized before annealing with the antisense strand.

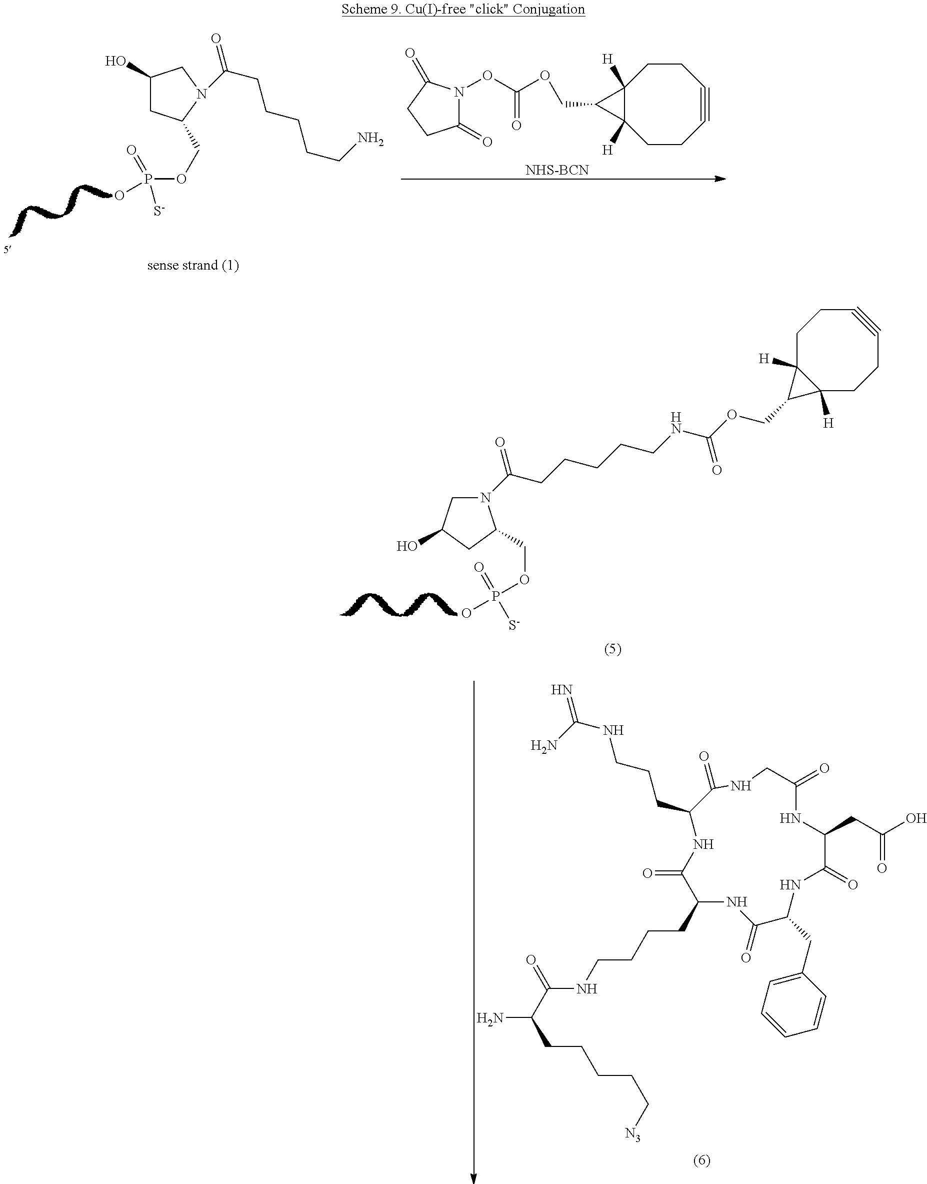

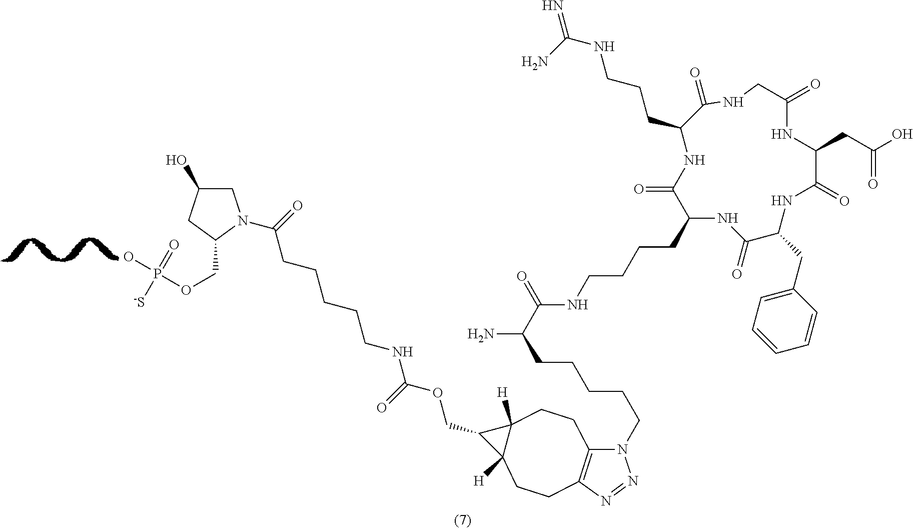

In a 40 mg scale synthesis, 1 equivalent of sense strand (1) containing 3′-amine was diluted to 1 mL with 1M pH 8.5 NaHCO₃ buffer. To this solution, 2 equivalents of N-[((1R,8S,9s)-Bicyclo [6.1.0]non-4-yn-9-yl)methyloxycarbonyloxy]succinimide (NHS-BCN), dissolved in 0.5 mL of anhydrous acetonitrile (ACN), was added. The reaction mixture was incubated at room temperature for 2 hours prior to desalting via size exclusion chromatography (SEC). Afterwards, the BCN-modified sense strand (5) was resuspended with 1.5 mL of 0.5 M pH 7.5 HEPES buffer, followed by the addition of 1.5 equivalents of azide-modified cRGD (6) in 1:1 NMP: 1M pH 7.5 HEPES buffer. The resulting mixture was incubated at 37° C. for 2 hrs. Purification was performed using reverse phase chromatography (C18 column) followed by desalting using SEC. [M] calculated for (7) 8222.678 Da, found 8222.05 Da (LC-MS). The purified sense strand conjugate (28 mg, 70% yield, 97% purity via LCMS) was then lyophilized before annealing with the antisense strand.

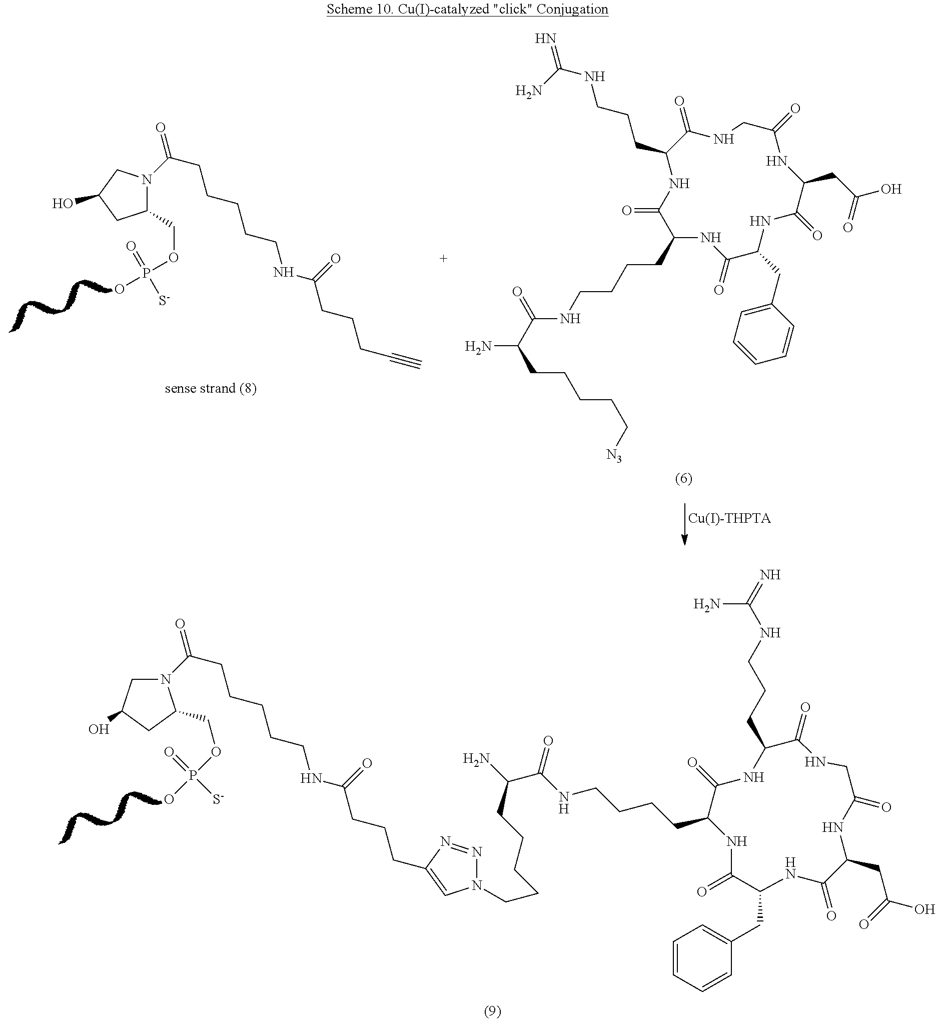 [SMILES: C#CCCCC(=O)NCCCCCC(=O)N1C[C@H](O)C[C@H]1COP(=O)(O)[S-] ; [N-]=[N+]=NCCCCC[C@@H](N)C(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O ; N=C(N)NCCC[C@@H]1NC(=O)[C@H](CCCCNC(=O)[C@H](N)CCCCCn2cc(CCCC(=O)NCCCCCC(=O)N3C[C@H](O)C[C@H]3COP(=O)(O)[S-])nn2)NC(=O)[C@@H](Cc2ccccc2)NC(=O)[C@H](CC(=O)O)NC(=O)CNC1=O ; "Cu(I)-THPTA"]

In a 40 mg scale synthesis, 1 equivalent of sense strand (8) containing a hexynyl group was diluted to 1.0 mL 1M pH 7.5 HEPES buffer. A 1 mL solution of azide-modified cRGD (6) (1.3 equivalents) was prepared in 1:1 NMP: 1M pH 7.5 HEPES buffer and then added to the initial solution. Next, Cu(I) complex was formulated by combining 2.6 equivalents of Tetrakis(acetonitrile)copper(I) hexafluorophosphate and 10.4 equivalents of tris-hydroxypropyitriazolylmethylamine (THPTA) in anhydrous NMP. The Cu-THPTA solution was then added to the original mixture, followed by incubation at 37° C. for 4 hrs. After the reaction was complete, Cu( ) was removed by using copper chelator (QuadraPure IDA9) Purification was performed using reverse phase chromatography (C18 column) followed by desalting using SEC. [M] calculated for (9) 8144.946 Da, found 8142.99 Da (LC-MS). The purified sense strand conjugate (17 mg, 42% yield, 99% purity via LCMS) was then lyophilized before annealing with the antisense strand.

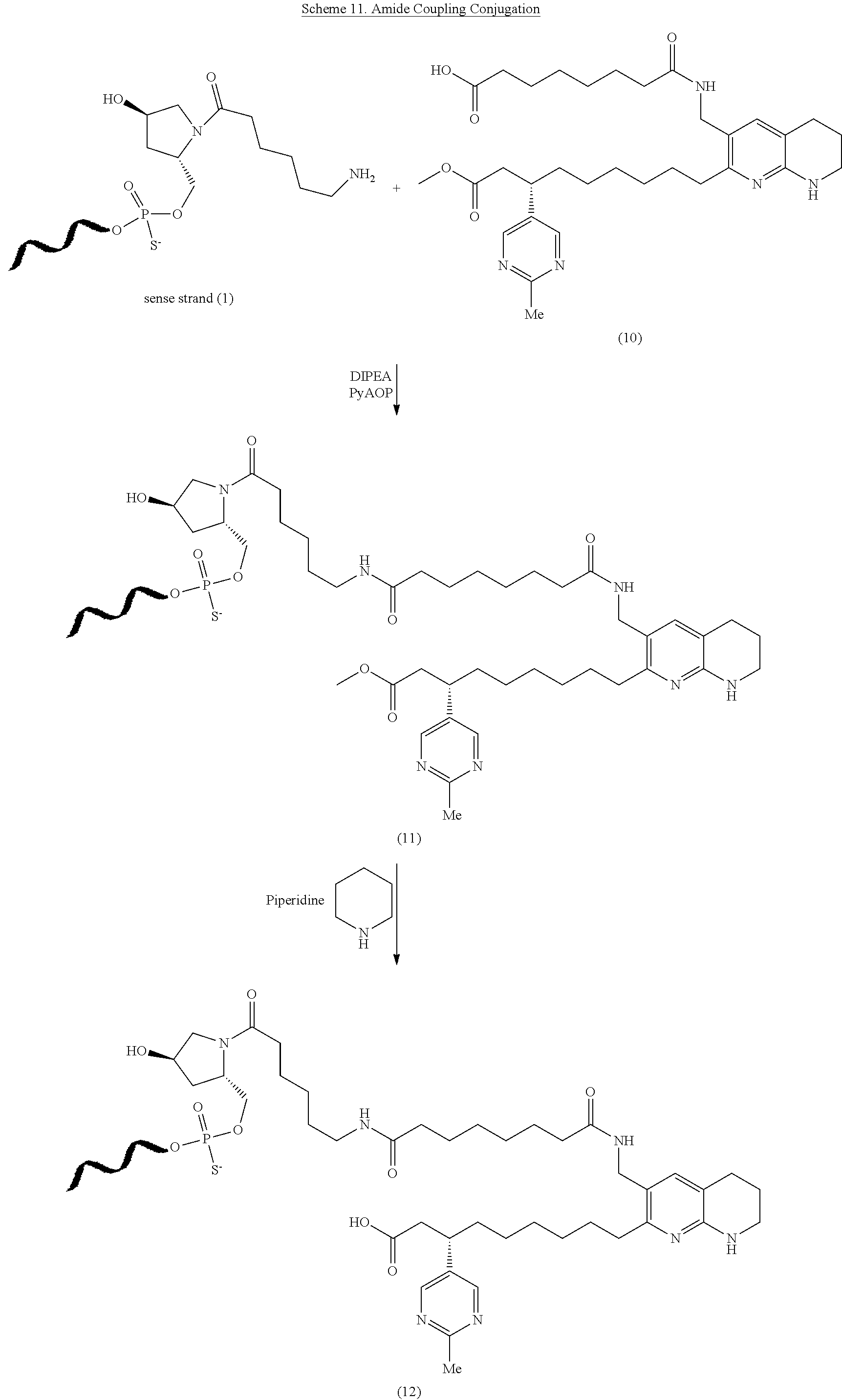 [SMILES: NCCCCCC(=O)N1C[C@H](O)C[C@H]1COP(=O)(O)[S-] ; COC(=O)C[C@H](CCCCCCc1nc2c(cc1CNC(=O)CCCCCCC(=O)O)CCCN2)c1cnc(C)nc1 ; COC(=O)C[C@H](CCCCCCc1nc2c(cc1CNC(=O)CCCCCCC(=O)NCCCCCC(=O)N1C[C@H](O)C[C@H]1COP(=O)(O)[S-])CCCN2)c1cnc(C)nc1 ; C1CCNCC1 ; Cc1ncc([C@@H](CCCCCCc2nc3c(cc2CNC(=O)CCCCCCC(=O)NCCCCCC(=O)N2C[C@H](O)C[C@H]2COP(=O)(O)[S-])CCCN3)CC(=O)O)cn1]

In a 60 mg scale synthesis, sense strand (1) was diluted to 1.25 mL with anhydrous NMP. The integrin targeting ligand (10) solution was prepared by combining 3 equivalents of (10), 3 equivalents of 7-Azabenzotriazol-1-yloxy)tripyrrolidinophosphonium hexafluorophosphate (PyAOP) and 9 equivalents of N-Ethyldiisopropylamine (DIPEA) in anhydrous NMP. The two solutions were then mixed and incubated at room temperature for 5 minutes to afford (11). After the reaction reached completion, methyl ester deprotection was performed by adding 5 mL of 10% (v/v) aqueous piperidine to the crude mixture, followed by incubation at 40° C. for 16 hours. Purification was performed using reverse phase chromatography (C18 column) followed by desalting using SEC. [M] calculated for (12) 7810.65 Da, found 7810.06 Da (LC-MS). The purified sense strand conjugate (26 mg, 43% yield, 98% purity via LCMS) was then lyophilized before annealing with the antisense strand.

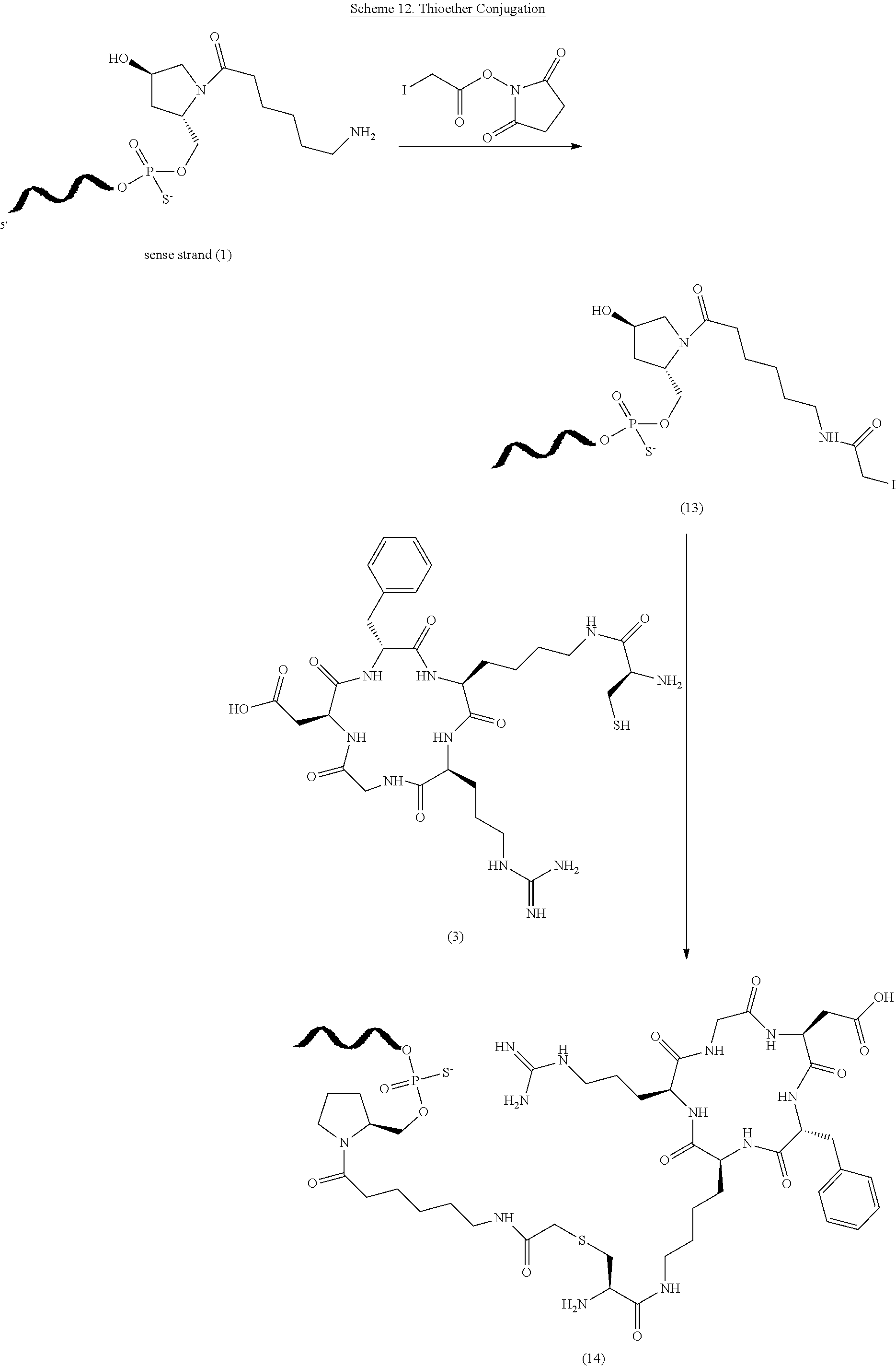 [SMILES: NCCCCCC(=O)N1C[C@H](O)C[C@H]1COP(=O)(O)[S-] ; O=C(CI)ON1C(=O)CCC1=O ; O=C(CI)NCCCCCC(=O)N1C[C@H](O)C[C@H]1COP(=O)(O)[S-] ; N=C(N)NCCC[C@@H]1NC(=O)[C@H](CCCCNC(=O)[C@@H](N)CS)NC(=O)[C@@H](Cc2ccccc2)NC(=O)[C@H](CC(=O)O)NC(=O)CNC1=O ; N=C(N)NCCC[C@@H]1NC(=O)[C@H](CCCCNC(=O)[C@@H](N)CSCC(=O)NCCCCCC(=O)N2CCC[C@H]2COP(=O)(O)[S-])NC(=O)[C@@H](Cc2ccccc2)NC(=O)[C@H](CC(=O)O)NC(=O)CNC1=O]

In a typical synthesis, 1 equivalent of sense strand (1) containing 3′-amine was diluted with pH 10 sodium carbonate buffer. To this solution, 2 equivalents of iodoacetic acid N-hydroxysuccinimide ester, dissolved in N-methyl-2-pyrrolidone (NMP) was added. The reaction mixture was incubated at room temperature for 1 hour, followed by the addition of 2 equivalents of thiol-modified cRGD (3) in 1:1 NMP: water. The resulting mixture was incubated at 37° C. for 2 hrs. Purification was performed using reverse phase chromatography (C18 column) followed by desalting using SEC.

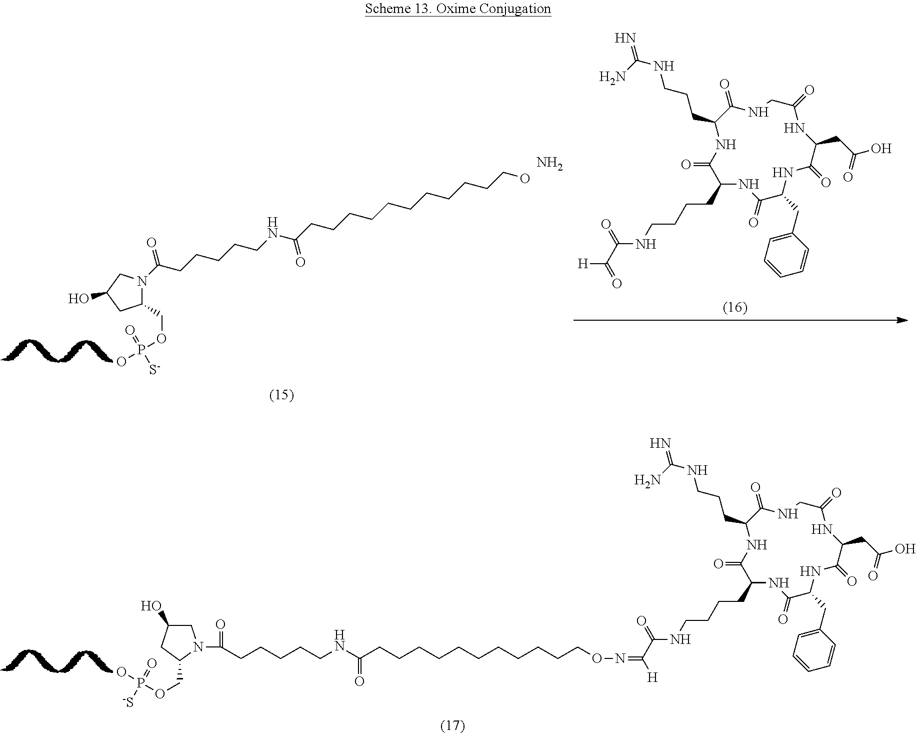

In a typical synthesis, 1 equivalent of sense strand (15) containing 3′-aminooxy would be diluted to with 1M pH 8.5 NaHCO₃ buffer. To this solution, 1.5 equivalents of aldehyde-modified cRGD (16) in 1:1 NMP: water would be added. The resulting mixture would be reacted for 2 hrs. Purification would be performed using reverse phase chromatography (C18 column) followed by desalting using SEC. The purified sense strand would then lyophilize before annealing with the antisense strand.

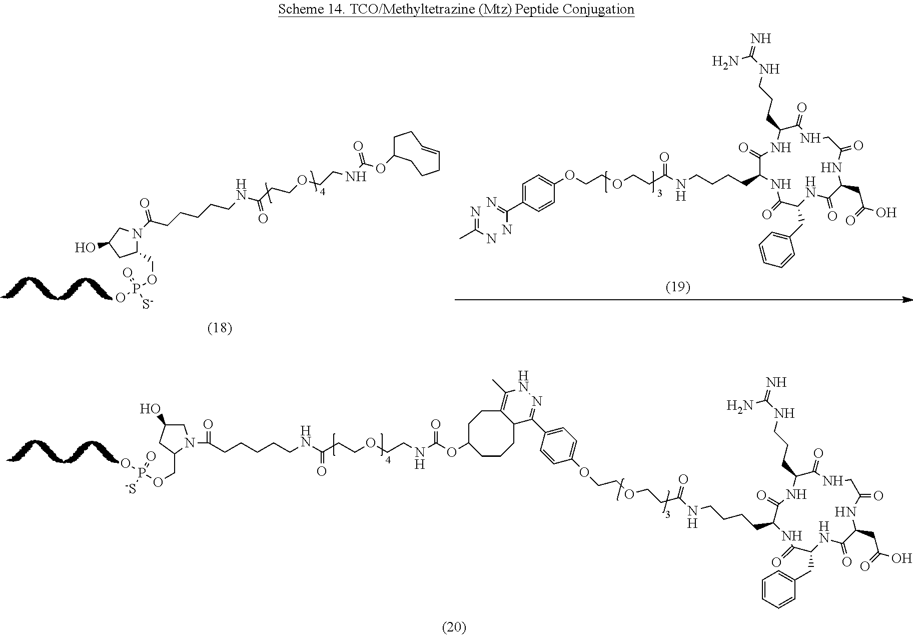

In a typical synthesis, 1 equivalent of sense strand (18) containing 3′-TCO would be diluted to with 1M pH 7.4 HEPES buffer. To this solution, 1.5 equivalents of methyltetrazine (Mtz)-modified cRGD (19) in 1:1 NMP: water would be added. The resulting mixture would be reacted for 2 hrs. Purification would be performed using reverse phase chromatography (C18 column) followed by desalting using SEC. The purified sense strand would then be lyophilized before annealing with the antisense strand.

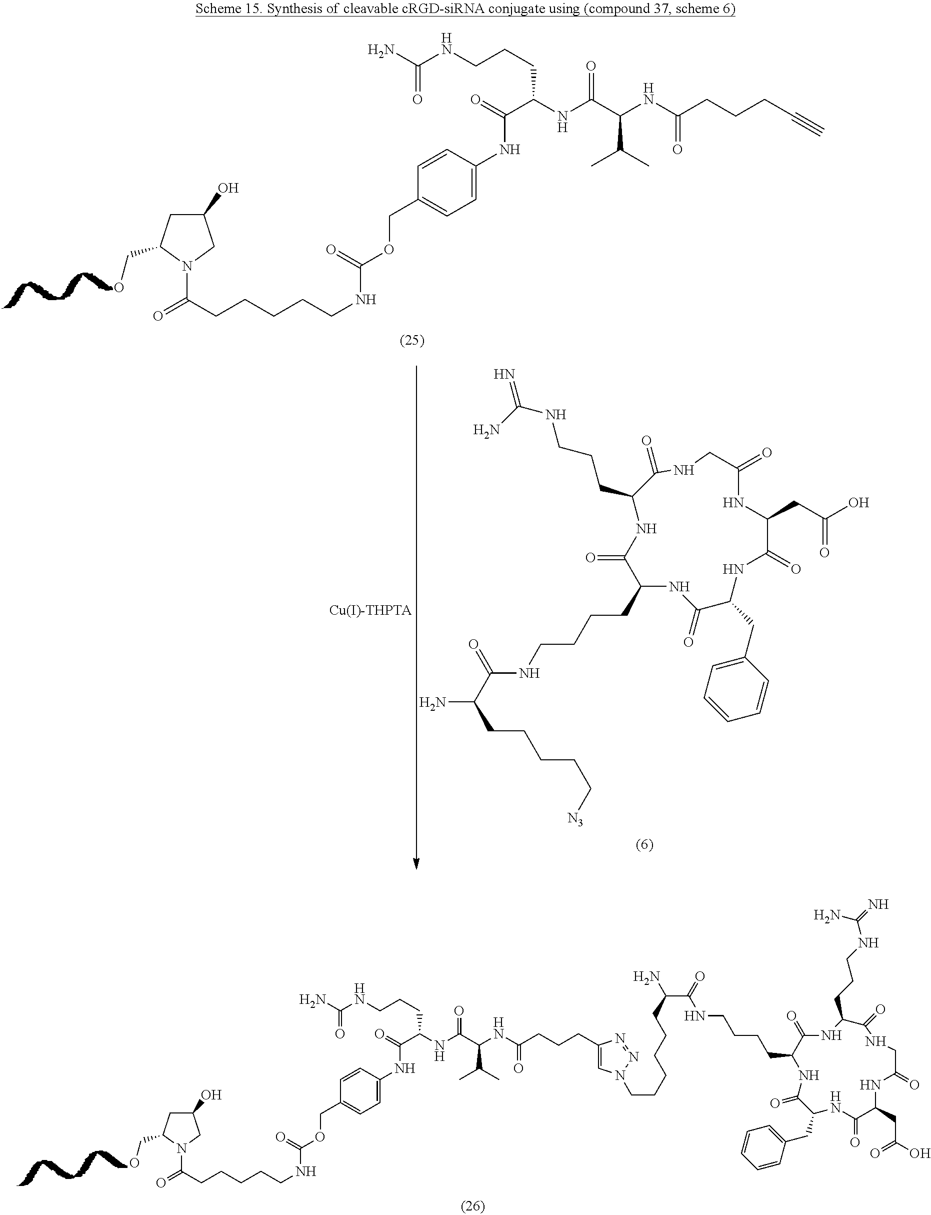 [SMILES: C#CCCCC(=O)N[C@H](C(=O)N[C@@H](CCCNC(N)=O)C(=O)Nc1ccc(COC(=O)NCCCCCC(=O)N2C[C@H](O)C[C@H]2CO)cc1)C(C)C ; [N-]=[N+]=NCCCCC[C@@H](N)C(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O ; CC(C)[C@H](NC(=O)CCCc1cn(CCCCCC[C@@H](N)C(=O)NCCCC[C@@H]2NC(=O)[C@@H](Cc3ccccc3)NC(=O)[C@H](CC(=O)O)NC(=O)CNC(=O)[C@H](CCCNC(=N)N)NC2=O)nn1)C(=O)N[C@@H](CCCNC(N)=O)C(=O)Nc1ccc(COC(=O)NCCCCCC(=O)N2C[C@H](O)C[C@H]2CO)cc1 ; "Cu(I)-THPTA"]

First, (25) was generated by combining 1 equivalent of sense strand (1) and 1.75 equivalents of (21) previously dissolved in 0.5 mL anhydrous NMP, in 1 mL of 0.5 M pH 8.5 NaHCO₃ buffer. 1.75 equivalents of DIPEA were added to solution before incubating at 37° C. for 3 hrs. The crude (25) solution was then desalted using SEC. The desalted (25) solution was resuspended in 1 mL of 0.5 M pH 7.5 HEPES buffer before proceeding to perform Cu(I)-catalyzed click reaction previously described above. [M] calculated for (26) 8550.407 Da, found 8549.86 Da (LC-MS). The purified sense strand conjugate (41 mg, 63% yield, 86% purity via LCMS) was then lyophilized before annealing with the antisense strand.

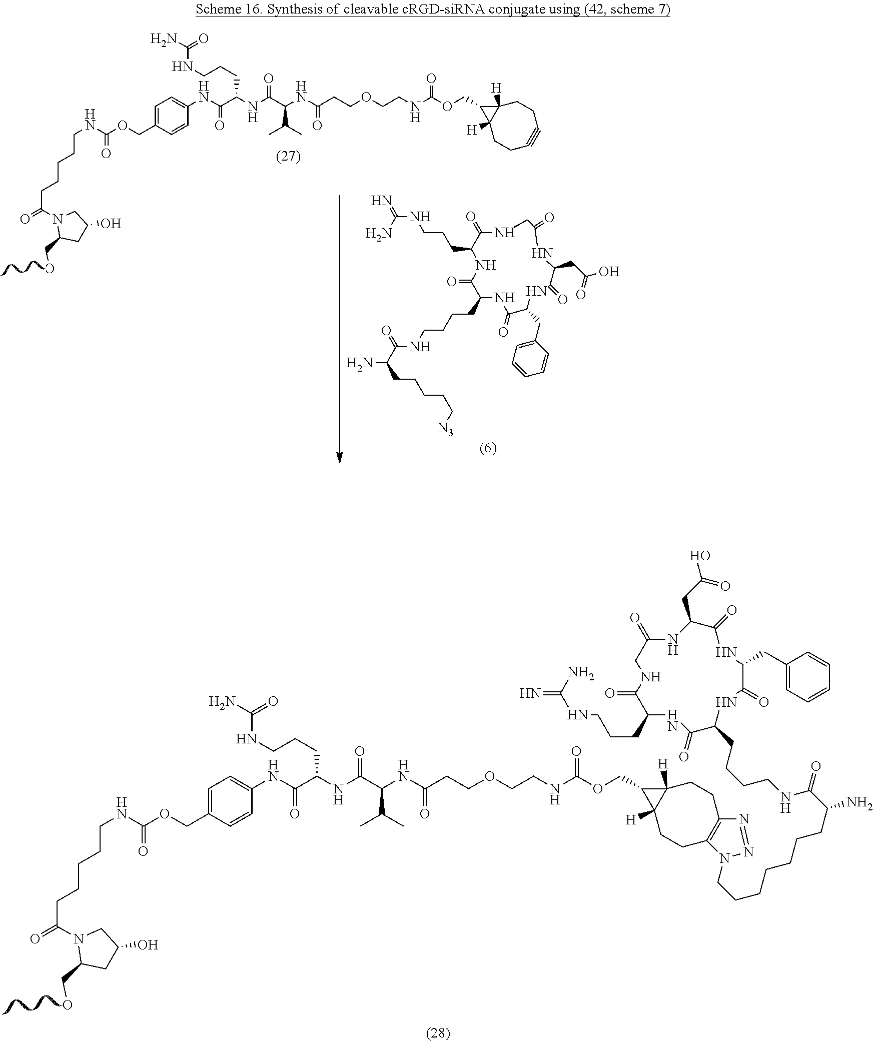

First, (27) was generated by combining 1 equivalent of sense strand (1) and 2.5 equivalents of (22) previously dissolved in 0.5 mL anhydrous NMP, in 1 mL of 0.5 M pH 8.5 NaHCO₃ buffer. 1.75 equivalents of DIPEA were added to solution before incubating at 37° C. overnight. The crude (27) solution was then desalted using SEC. The desalted (27) solution was resuspended in 1 mL of 0.5 M pH 7.5 HEPES buffer before proceeding to perform Cu(I)-free click reaction previously described above. [M] calculated for (28) 8747.641 Da, found 8747.02 Da (LC-MS). The purified sense strand conjugate (22 mg, 34% yield, 96% purity via LCMS) was then lyophilized before annealing with the antisense strand.

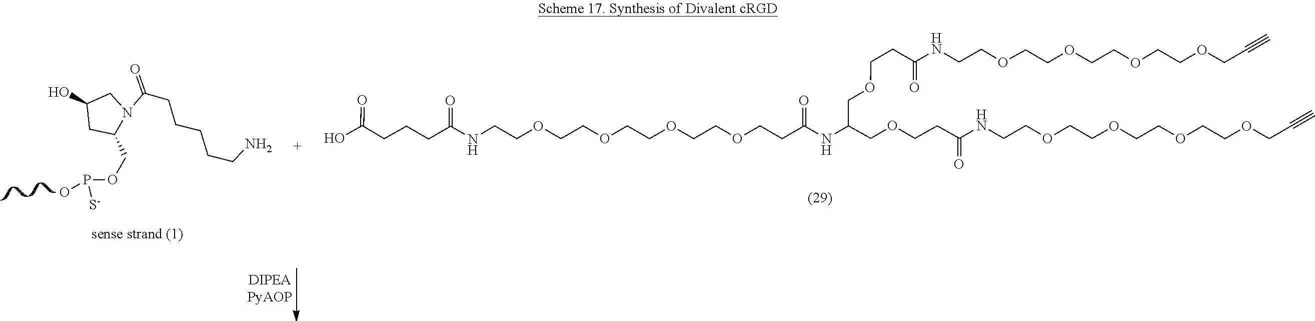 [SMILES: NCCCCCC(=O)N1C[C@H](O)C[C@H]1COP(O)[S-] ; C#CCOCCOCCOCCOCCNC(=O)CCOCC(COCCC(=O)NCCOCCOCCOCCOCC#C)NC(=O)CCOCCOCCOCCOCCNC(=O)CCCC(=O)O]

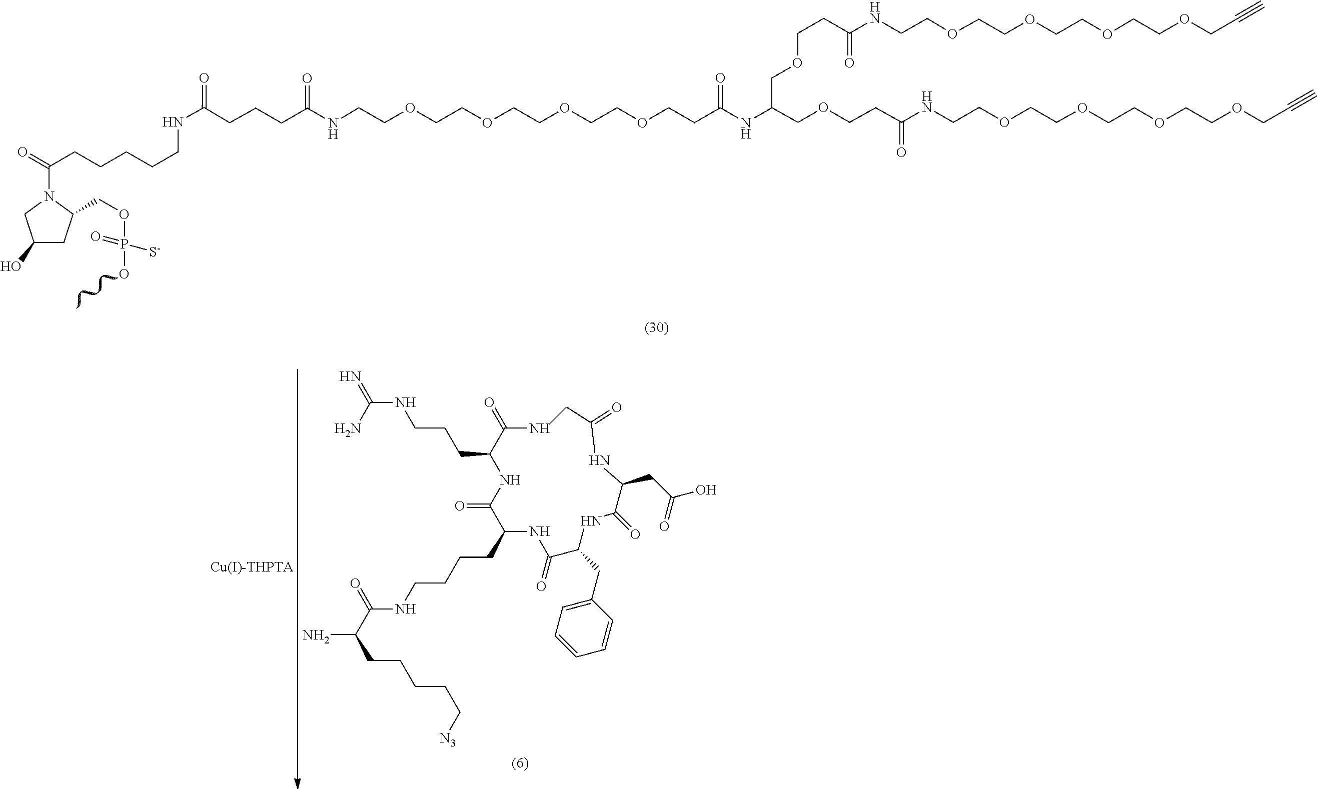 [SMILES: C#CCOCCOCCOCCOCCNC(=O)CCOCC(COCCC(=O)NCCOCCOCCOCCOCC#C)NC(=O)CCOCCOCCOCCOCCNC(=O)CCCC(=O)NCCCCCC(=O)N1C[C@H](O)C[C@H]1COP(=O)(O)[S-] ; [N-]=[N+]=NCCCCC[C@@H](N)C(=O)NCCCC[C@H](NC(=O)[C@@H](Cc1ccccc1)NC(=O)[C@H](CC(=O)O)NC=O)C(=O)N[C@@H](CCCNC(=N)N)C(N)=O ; "Cu(I)-THPTA"]

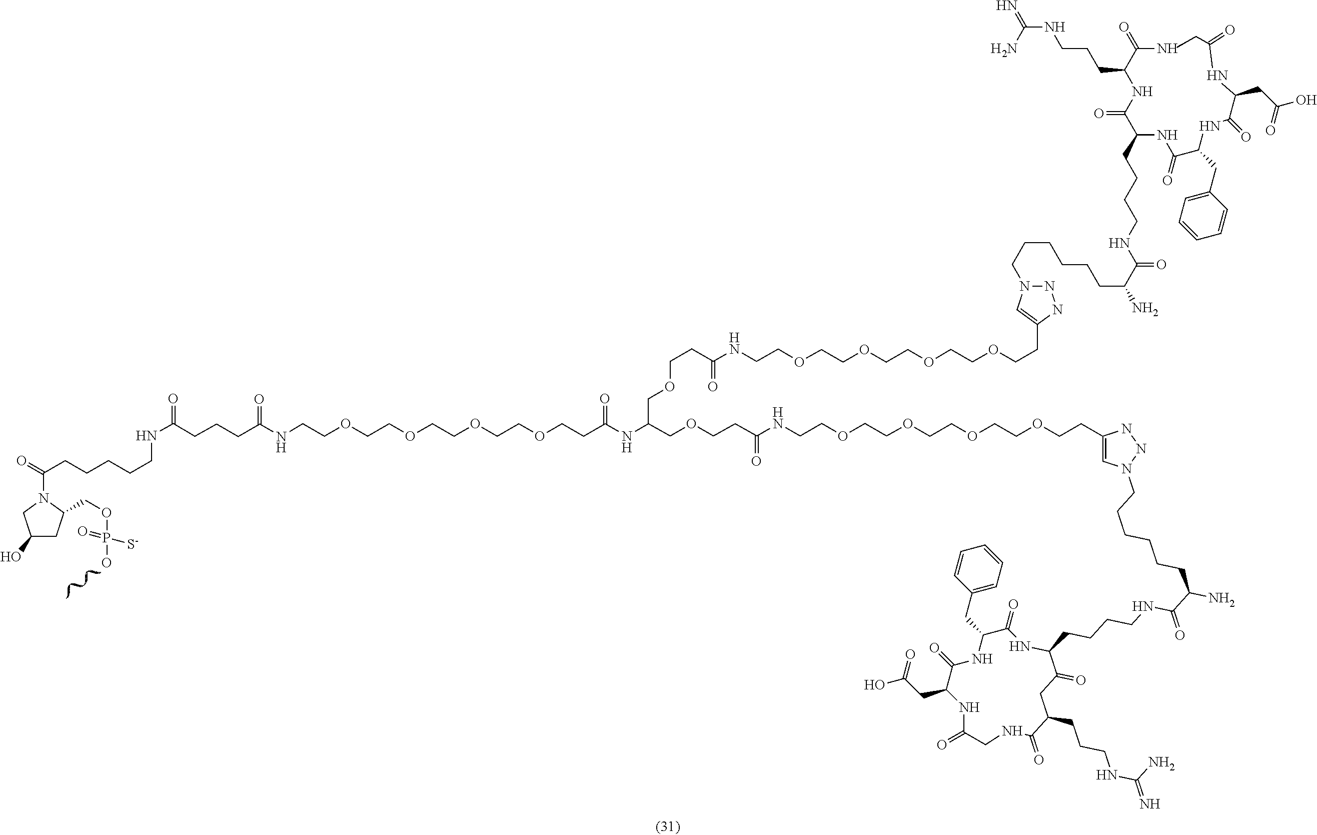 [SMILES: N=C(N)NCCC[C@@H]1CC(=O)[C@H](CCCCNC(=O)[C@H](N)CCCCCCN2C=C(CCOCCOCCOCCOCCNC(=O)CCOCC(COCCC(=O)NCCOCCOCCOCCOCCC3=CN(CCCCCC[C@@H](N)C(=O)NCCCC[C@H](NC(=O)[C@@H](Cc4ccccc4)NC(=O)[C@H](CC(=O)O)NC=O)C(=O)N[C@@H](CCCNC(=N)N)C(N)=O)NN3)NC(=O)CCOCCOCCOCCOCCNC(=O)CCCC(=O)NCCCCCC(=O)N3C[C@H](O)C[C@H]3COP(=O)(O)[S-])NN2)NC(=O)[C@@H](Cc2ccccc2)NC(=O)[C@H](CC(=O)O)NC(=O)CNC1=O]

In a typical synthesis, sense strand (1) is diluted to 1 mL with NMP. In a separate vial, 2.5 equivalents of (29) and 2.5 equivalents of PyAOP were combined and dissolved in anhydrous NMP, followed by the addition of 7.5 equivalents of DIPEA. The activated (29) solution was then mixed with the sense strand (1) solution. The reaction mixture was incubated for 5 minutes at room temperature before desalting via SEC. Afterwards, the desalted (30) was resuspended with 0.5 mL 1M pH 7 HEPES buffer, followed by the addition of 3 equivalents of (6) dissolved 1:1 NMP: HEPES buffer. Next, Cu(I) complex was formulated by combining 9 equivalents of Tetrakis(acetonitrile)copper(I) hexafluorophosphate and 36 equivalents of tris-hydroxypropyltriazolvi-nethylanine (THPTA) in anhydrous NMP. The Cu-TIPTA solution was then added to the oiginal mixture, followed by incubation at 37° C. for 4 hrs. After the reaction wv % as complete, Cu(I) was removed by using copper chelator (QuadraPure IDAX). Purification was performed using reverse phase chromatography (C18 column) followed by desalting using SEC. [M] calculated for (31) 9813.86 Da, found 9811.68 Da (LC-MS). The purified sense strand conjugate (40 mg, 61% yield, 96% purity via LCMS) was then lyophilized before annealing with the antisense strand.

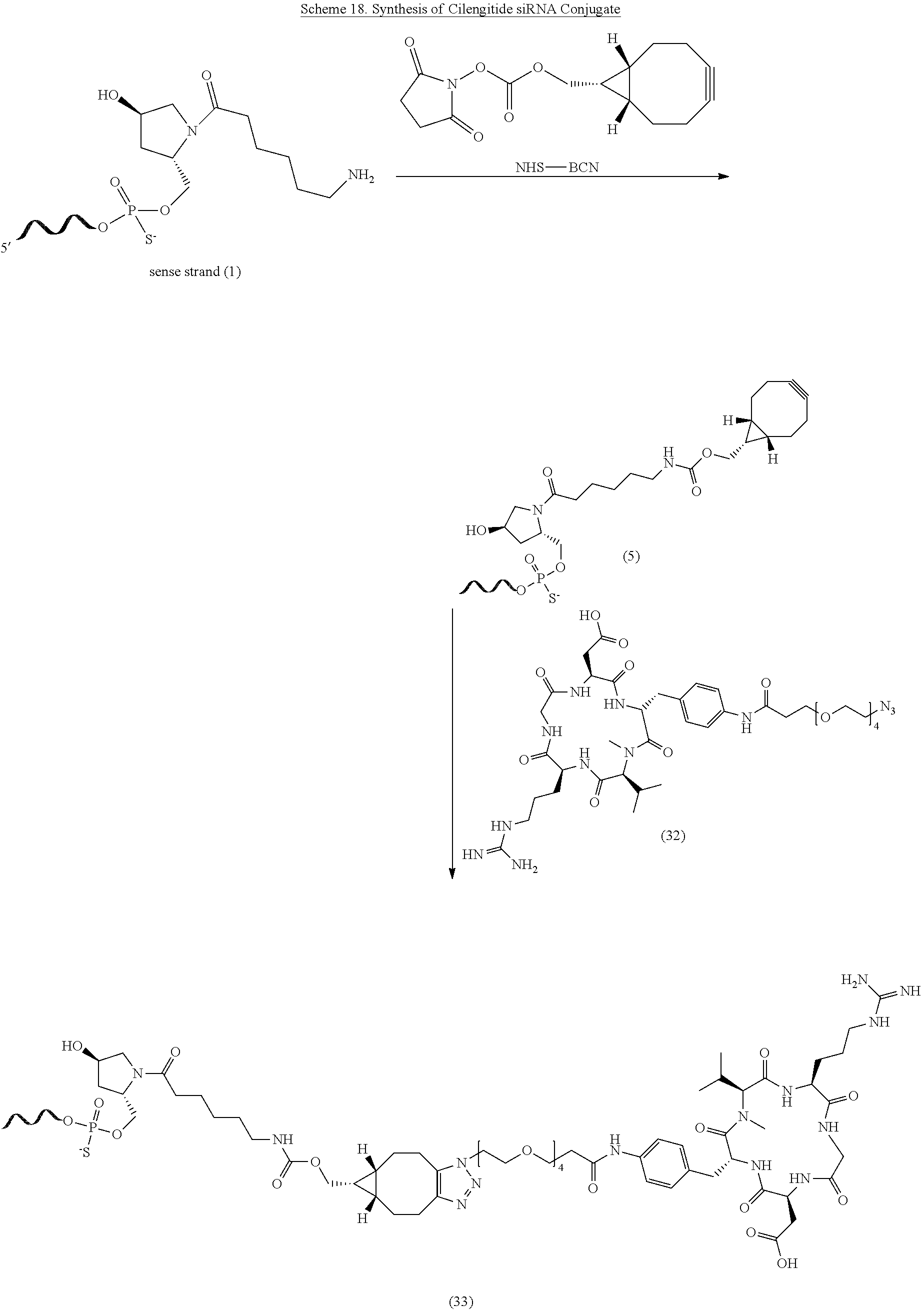

In a typical synthesis, 1 equivalent of sense strand (1) containing 3′-amine was diluted to 1 mL with 1M pH 8.5 NaHCO₃ buffer. To this solution, 2 equivalents of N-[((1R,8S,9s)-Bicyclo [6.1.0]non-4-yn-9-yl)methyloxycarbonyloxy]succinimide (NHS-BCN), dissolved in 0.5 mL of anhydrous acetonitrile (ACN), was added. The reaction mixture was incubated at room temperature for 2 hours prior to desalting via size exclusion chromatography (SEC). Afterwards, the BCN-modified sense strand (5) was resuspended with 1.5 mL of 0.5 M pH 7.5 HEPES buffer, followed by the addition of 1.5 equivalents of azide-modified Cilengitide (31) in 1:1 NMP: 1M pH 7.5 HEPES buffer. The resulting mixture was incubated at 37° C. for 2 hrs. Purification was performed using reverse phase chromatography (C18 column) followed by desalting using SEC and annealing with antisense strand.

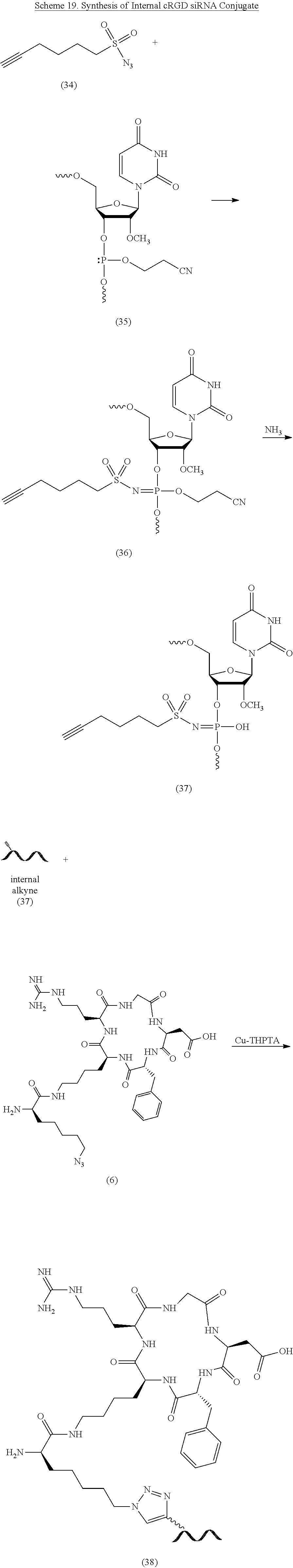

An internal hexynyl group was first introduced in the phosphate backbone of the sense strand during the oligonucleotide synthesis (40 μmol) performed in a K&A synthesizer. In particular, the iodine oxidation step for a specific base addition cycle was replaced by Staudinger reaction with hex-5-yne-1-sulfonyl azide (34) (0.5 M in anhydrous acetonitrile) at 37° C. for 1 hour to afford (36). After ammonia treatment, the sense strand with internal alkyne was purified prior to conjugation with azide-modified cRGD (6) through Cu(I)-catalyzed click reaction previously described. Purification was performed using reverse phase chromatography (C18 column) followed by desalting using SEC. [M] calculated for (31) 7869.723 Da, found 7869.20 Da (LC-MS). The purified sense strand conjugate (51 mg, 85% yield, 95% purity via LCMS) was then lyophilized before annealing with the antisense strand.

Annealing and Preparation of siRNA Duplexes: All oligonucleotides (purified to an HPLC purity of >90% and desalted), were further annealed by mixing a solution of equimolar amounts of complementary sense and antisense strands to form the corresponding siRNAs by heating to 90° C. followed by slow cooling. The siRNA samples were analyzed by mass spectrometry and capillary gel electrophoresis as well as for endotoxin and osmolality.

A detailed list of the modified nucleotide sequences of the sense strand and antisense strand sequences is shown in Table 3.

TABLE 3
Single strand siRNA's synthesized for in vitro and in vivo studies

| | | | | Molecular | Molecular |
|---|---|---|---|---|---|
| Oligo Id | Strand | Target | Oligo Sequence | Weight | Weight Found |
| A-2686466 | sense | MYOC | gsusgaaaUfaAfAfGfuuaucuuacsasL240Z67 | 8219.045 | 8215.615 |
| | | | (SEQ ID NO: 5) | | |
| A-2686467 | sense | MYOC | gsusgaaaUfaAfAfGfuuaucuuacsasL240Z71 | 9764.785 | 9760.411 |
| | | | (SEQ ID NO: 6) | | |
| A-2600332 | sense | MYOC | gsusgaaaUfaAfAfGfuuaucuuacsasL348 | 7842.701 | 7838.527 |
| | | | (SEQ ID NO: 7) | | |
| A-2600333 | sense | MYOC | gsusgaaaUfaAfAfGfuuaucuuacsasL349 | 9528.912 | 9523.595 |
| | | | (SEQ ID NO: 8) | | |
| A-2819653 | sense | SSB | usgsacaaCfaGfAfCfuuuaauguasasL348 | 7841.717 | 7837.543 |
| | | | (SEQ ID NO: 9) | | |
| A-2819654 | sense | SSB | usgsacaaCfaGfAfCfuuuaauguasasL240Z67 | 8218.061 | 8214.631 |
| | | | (SEQ ID NO: 10) | | |
| A-2824431 | sense | SSB | usgsacaaCfaGfAfCfuuuaauguasasL349 | 9527.928 | 9522.611 |
| | | | (SEQ ID NO: 11) | | |
| A-2824432 | sense | SSB | usgsacaaCfaGfAfCfuuuaauguasasL240Z71 | 9763.801 | 9759.427 |
| | | | (SEQ ID NO: 12) | | |
| A-2833825 | sense | RhoA | gsasagugCfuGfUfUfuauuaaucusasL240Z67 | 8188.965 | 8185.555 |
| | | | (SEQ ID NO: 13) | | |
| A-2833826 | sense | RhoA | gsasagugCfuGfUfUfuauuaaucusasL240Z71 | 9734.705 | 9730.351 |
| | | | (SEQ ID NO: 14) | | |
| A-2833827 | sense | RhoA | gsasagugCfuGfUfUfuauuaaucusasL348 | 7812.621 | 7808.467 |
| | | | (SEQ ID NO: 15) | | |
| A-2833828 | sense | RhoA | gsasagugCfuGfUfUfuauuaaucusasL349 | 9498.832 | 9493.536 |
| | | | (SEQ ID NO: 16) | | |
| A-2833835 | sense | MYOC | Z67Q385scsuugaaCfaUfGfGfucacuuaugsasL | 9375.296 | 9371.107 |
| | | | 240Z67 (SEQ ID NO: 17) | | |
| A-2833836 | sense | MYOC | Z67Q385scsuugaaCfaUfGfGfucacuuausgsa | 8156.964 | 8152.569 |
| | | | (SEQ ID NO: 18) | | |
| A-2833837 | sense | MYOC | Z71Q385scsuugaaCfaUfGfGfucacuuausgsa | 9702.704 | 9697.365 |
| | | | (SEQ ID NO: 19) | | |
| A-2833838 | sense | MYOC | csusugaaCfaUfGfGfucacuuaugsasQ157Z67s | 9422.394 | 9418.103 |
| | | | L240Z67 (SEQ ID NO: 20) | | |
| A-2833839 | sense | MYOC | csusuga Y224Z67CfaUfGfGfucacuuausgsa | 7979.875 | 7975.55 |
| | | | (SEQ ID NO: 21) | | |
| A-2833840 | sense | MYOC | csusuga Y224Z71 CfaUfGfGfucacuuausgsa | 9525.615 | 9520.346 |
| | | | (SEQ ID NO: 22) | | |
| A-3177380 | sense | MYOC | gsusgaaaUfaAfAfGfuuaucuuacsasL373Z106 | 8227.054 | 8222.678 |
| | | | (SEQ ID NO: 23) | | |
| A-3464724 | sense | MYOC | gsusgaaaUfaAfAfGfuuaucuuacsasL123Z106 | 8144.946 | 8142.99 |
| | | | (SEQ ID NO: 24) | | |
| A-3507267 | sense | MYOC | gsusgaaaUfaAfAfGfuuaucuuacsasL406Z106 | 8550.407 | 8549.86 |
| | | | (SEQ ID NO: 25) | | |
| A-3507268 | sense | MYOC | gsusgaaaUfaAfAfGfuuaucuuacsasL410Z106 | 8747.641 | 8747.02 |
| | | | (SEQ ID NO: 26) | | |
| A-3507269 | sense | MYOC | gsusgaaaUfaAfAfGfuuaucuuacsasL409Z106 | 9813.86 | 9811.68 |
| | | | (SEQ ID NO: 27) | | |
| A-3196459 | Sense | MYOC | csusuga(apn6)Z106CfaUfGfGfucacuuausgsa | 7869.723 | 7869.20 |
| | | | (SEQ ID NO: 28) | | |
| A-1110699 | sense | MYOC | gsusgaa(Ahd)UfaAfAfGfuuaucuuascsa | 7211.123 | 7207.308 |
| | | | (SEQ ID NO: 29) | | |
| A-1110677 | sense | MYOC | gsasccc(Uhd)GfaCfCfAfucccauucsasa | 7096.059 | 7092.319 |
| | | | (SEQ ID NO: 30) | | |
| A-2036449 | sense | RHOA | gsasagu(Ghd)CfuGfUfUfuauuaaucsusa | 7181.039 | 7177.254 |
| | | | (SEQ ID NO: 31) | | |
| A-1700845 | sense | VEGF | gsasgaa(Ahd)GfuGfUfUfuuauauacsgsa | 7267.146 | 7263.31 |
| | | A | (SEQ ID NO: 32) | | |
| A-3112837 | sense | VEGF | gsasgaaaGfuGfUfUfuuauauacgsasL240Z67 | 8275.068 | 8270.603 |
| | | A | (SEQ ID NO: 33) | | |
| A-3112838 | sense | VEGF | gsasgaaY224Z67GfuGfUfUfuuauauacsgsa | 8067.947 | 8063.579 |
| | | A | (SEQ ID NO: 34) | | |
| A-2898771 | sense | MYOC | Z67Q385sgsugaaaUfaAfAfGfuuaucuuacsasL | 9407.347 | 9402.122 |
| | | | 240Z67 (SEQ ID NO: 35) | | |
| A-2898772 | sense | MYOC | Z67Q385sgsugaaaUfaAfAfGfuuaucuuascsa | 8189.014 | 8184.597 |
| | | | (SEQ ID NO: 36) | | |
| A-2898773 | sense | MYOC | Z71Q385sgsugaaaUfaAfAfGfuuaucuuascsa | 9734.754 | 9729.393 |
| | | | (SEQ ID NO: 37) | | |
| A-2898774 | sense | MYOC | gsusgaaaUfaAfAfGfuuaucuuacsasQ157sZ67 | 9454.445 | 9449.118 |
| | | | L240Z67 (SEQ ID NO: 38) | | |
| A-2898775 | sense | MYOC | gsusgaaY224Z67UfaAfAfGfuuaucuuascsa | 8011.925 | 8007.578 |
| | | | (SEQ ID NO: 39) | | |
| A-2898776 | sense | MYOC | gsusgaaY224Z71UfaAfAfGfuuaucuuascsa | 9557.665 | 9552.373 |
| | | | (SEQ ID NO: 40) | | |
| A-2992766 | sense | LRRK | asgscug(Uhd)agCfAfAfggaguuuugaL96 | 9067.013 | 9062.304 |
| | | 2 | (SEQ ID NO: 41) | | |
| A-3177381 | sense | MYOC | Q420sZ93gsugaaaUfaAfAfGfuuaucuuacsasL | 9661.597 | 9656.361 |
| | | | 373Z93 (SEQ ID NO: 42) | | |
| A-3177382 | sense | MYOC | gsusgaaaUfaAfAfGfuuaucuuacsasQ425sZ93 | 9707.688 | 9702.349 |
| | | | L373Z93 (SEQ ID NO: 43) | | |
| A-1577508 | sense | MYOC | csusuga(Ahd)CfaUfGfGfucacuuausgsa | 7179.072 | 7175.281 |
| | | | (SEQ ID NO: 44) | | |
| A-3019042 | sense | MYOC | csusugaaCfaUfGfGfucacuuaugsasL240Z67 | 8186.994 | 8182.575 |
| | | | (SEQ ID NO: 45) | | |

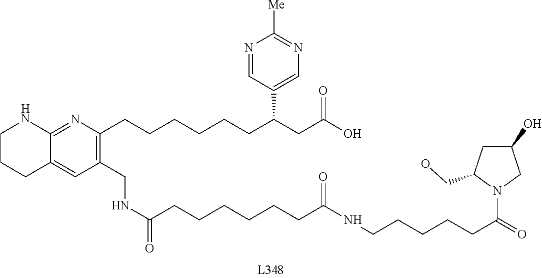 [SMILES: Cc1ncc([C@@H](CCCCCCc2nc3c(cc2CNC(=O)CCCCCCC(=O)NCCCCCC(=O)N2C[C@H](O)C[C@H]2CO)CCCN3)CC(=O)O)cn1]

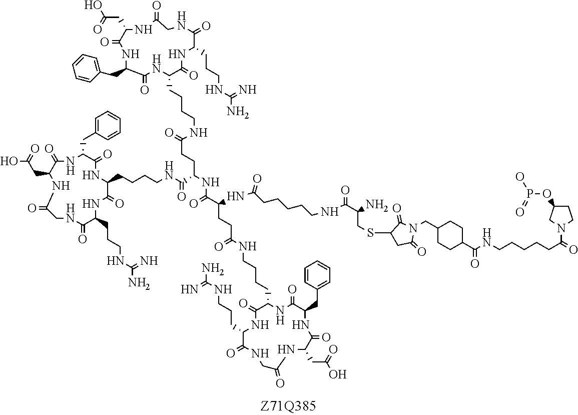

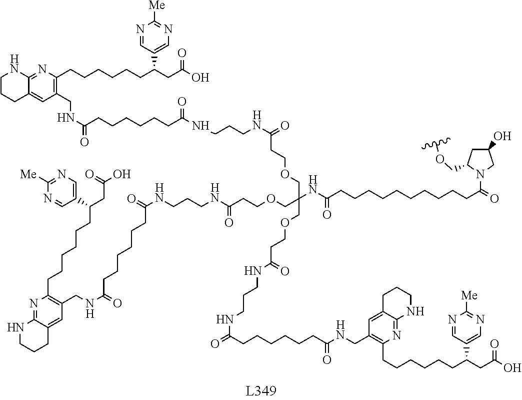 [SMILES: *OC[C@@H]1C[C@@H](O)CN1C(=O)CCCCCCCCCCC(=O)NC(COCCC(=O)NCCCNC(=O)CCCCCCC(=O)NCc1cc2c(nc1CCCCCC[C@@H](CC(=O)O)c1cnc(C)nc1)NCCC2)(COCCC(=O)NCCCNC(=O)CCCCCCC(=O)NCc1cc2c(nc1CCCCCC[C@@H](CC(=O)O)c1cnc(C)nc1)NCCC2)COCCC(=O)NCCCNC(=O)CCCCCCC(=O)NCc1cc2c(nc1CCCCCC[C@@H](CC(=O)O)c1cnc(C)nc1)NCCC2]

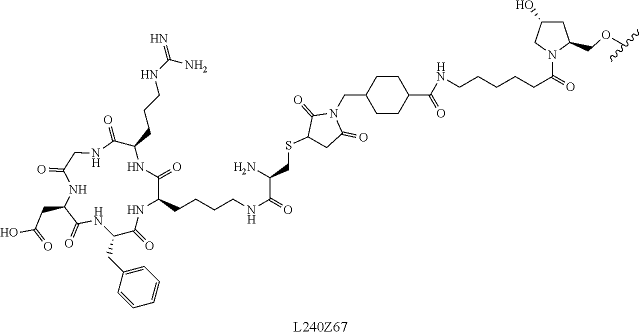 [SMILES: *OC[C@@H]1C[C@@H](O)CN1C(=O)CCCCCNC(=O)C1CCC(CN2C(=O)CC(SC[C@H](N)C(=O)NCCCC[C@H]3NC(=O)[C@H](Cc4ccccc4)NC(=O)[C@@H](CC(=O)O)NC(=O)CNC(=O)[C@@H](CCCNC(=N)N)NC3=O)C2=O)CC1]

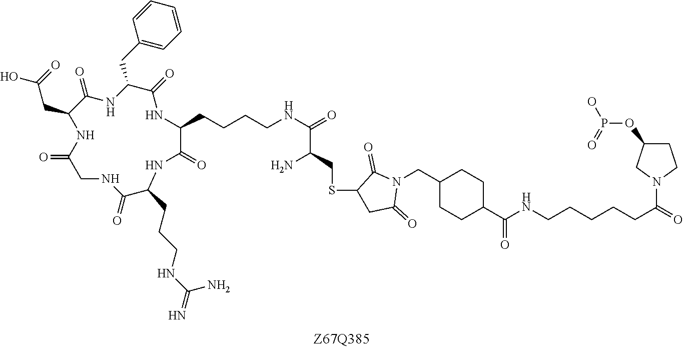

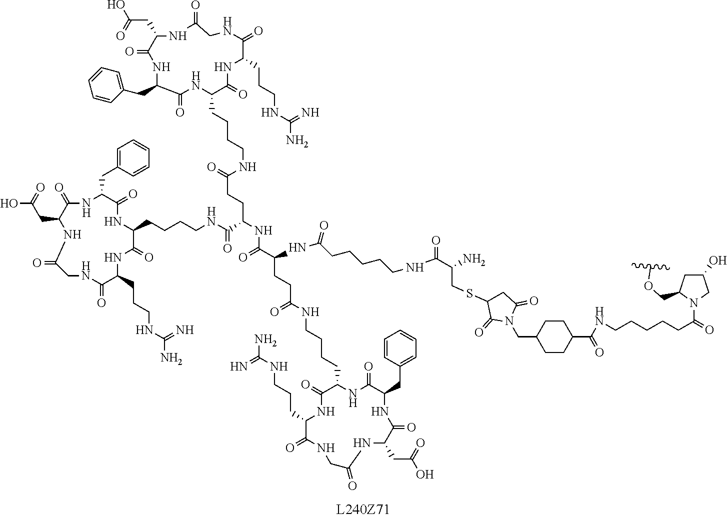 [SMILES: *OC[C@H]1C[C@H](O)CN1C(=O)CCCCCNC(=O)C1CCC(CN2C(=O)CC(SC[C@@H](N)C(=O)NCCCCCC(=O)N[C@@H](CCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)N[C@@H](CCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C2=O)CC1]

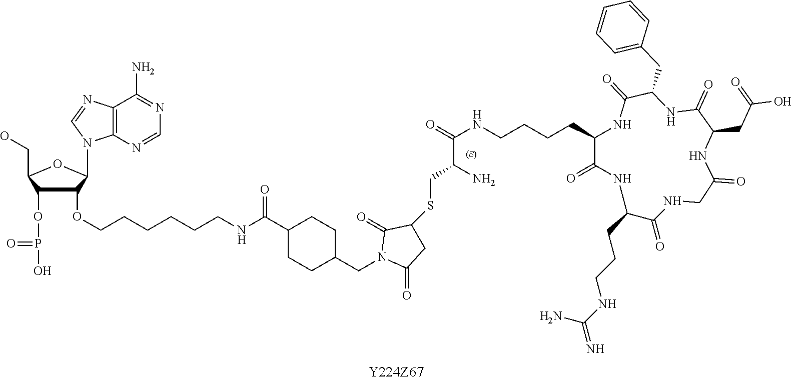

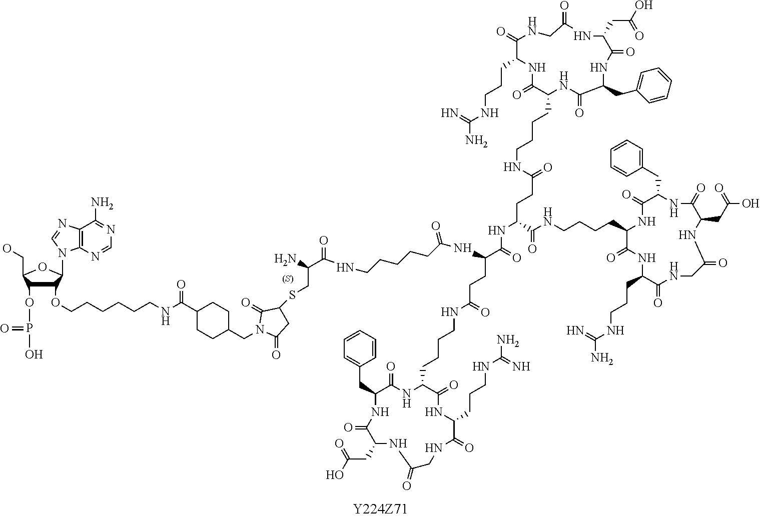

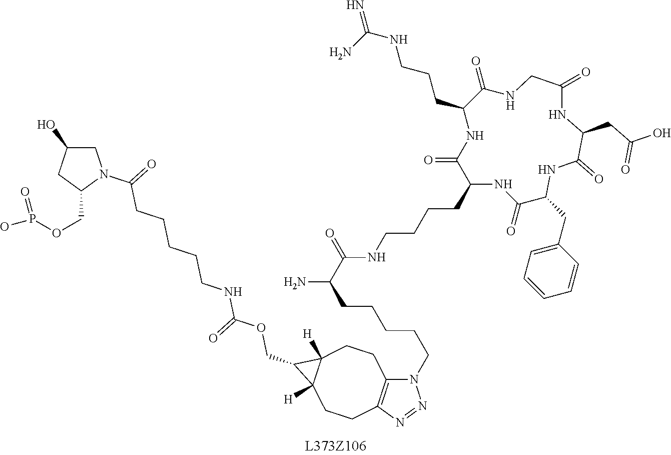

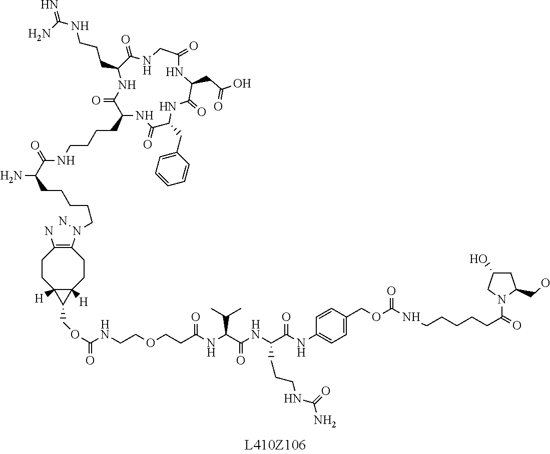 [SMILES: CC(C)[C@H](NC(=O)CCOCCNC(=O)OC[C@@H]1[C@@H]2CCc3nnn(CCCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)c3CC[C@@H]21)C(=O)N[C@@H](CCCNC(N)=O)C(=O)Nc1ccc(COC(=O)NCCCCCC(=O)N2C[C@H](O)C[C@H]2CO)cc1]

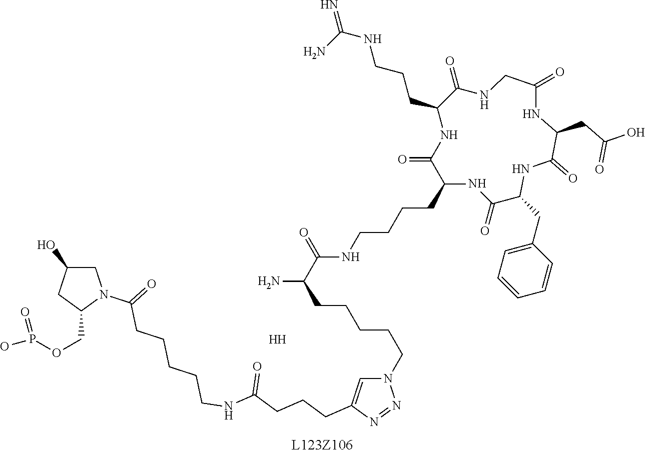

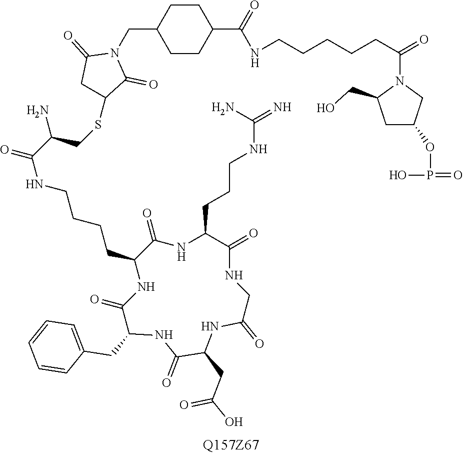

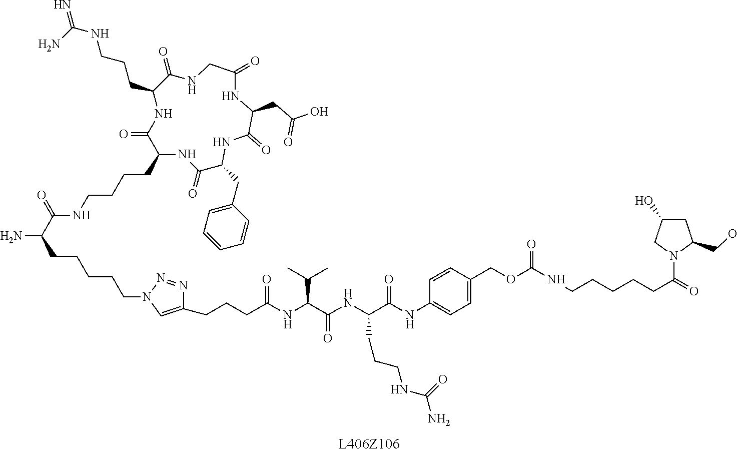 [SMILES: CC(C)[C@H](NC(=O)CCCc1cn(CCCCC[C@@H](N)C(=O)NCCCC[C@@H]2NC(=O)[C@@H](Cc3ccccc3)NC(=O)[C@H](CC(=O)O)NC(=O)CNC(=O)[C@H](CCCNC(=N)N)NC2=O)nn1)C(=O)N[C@@H](CCCNC(N)=O)C(=O)Nc1ccc(COC(=O)NCCCCCC(=O)N2C[C@H](O)C[C@H]2CO)cc1]

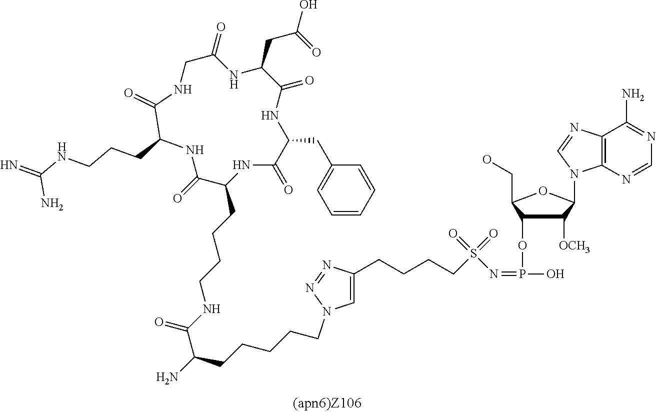

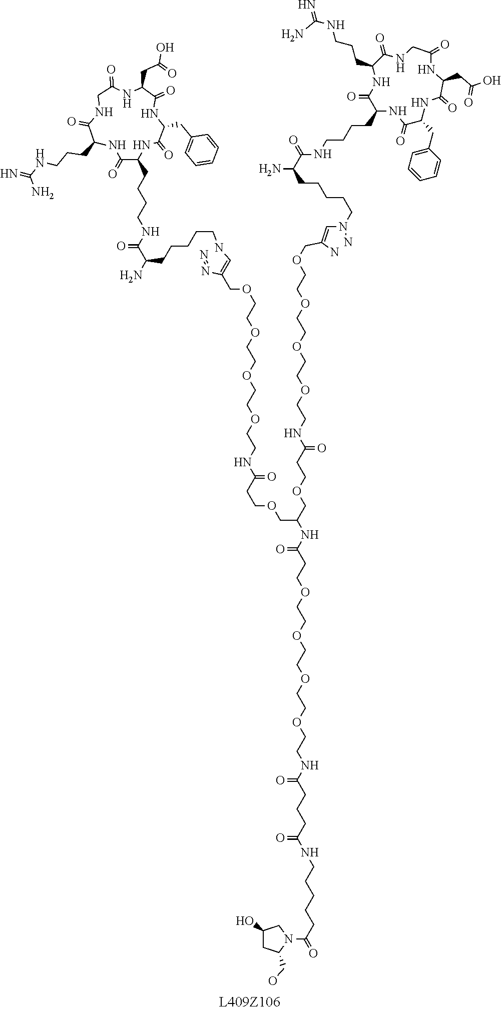 [SMILES: N=C(N)NCCC[C@@H]1NC(=O)[C@H](CCCCNC(=O)[C@H](N)CCCCCn2cc(COCCOCCOCCOCCNC(=O)CCOCC(COCCC(=O)NCCOCCOCCOCCOCc3cn(CCCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)nn3)NC(=O)CCOCCOCCOCCOCCNC(=O)CCCC(=O)NCCCCCC(=O)N3C[C@H](O)C[C@H]3CO)nn2)NC(=O)[C@@H](Cc2ccccc2)NC(=O)[C@H](CC(=O)O)NC(=O)CNC1=O]

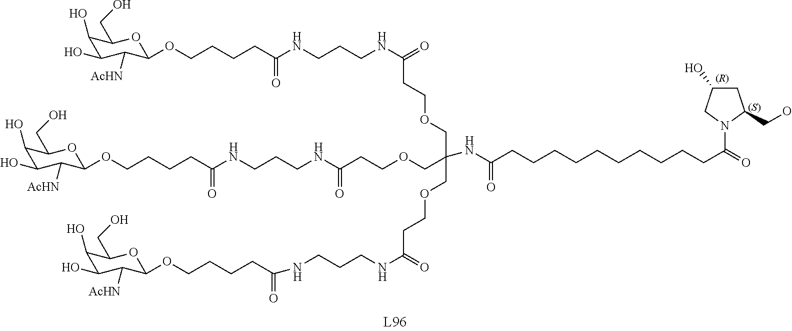

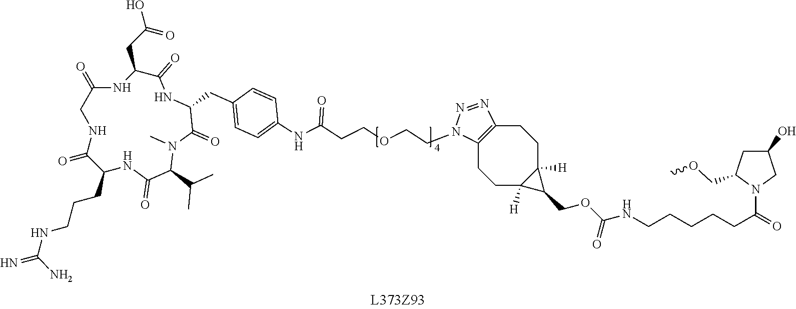

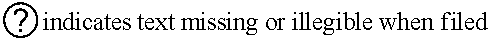

### Example 3. Post-synthetic and on column conjugation of integrin targeting ligands to siRNAs at 3′, 5′ or internal positions utilizing the methods given in example 2

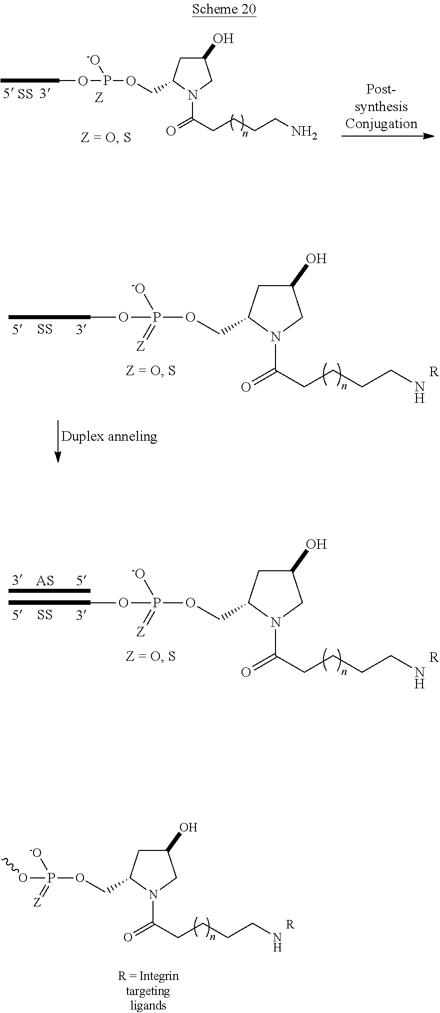

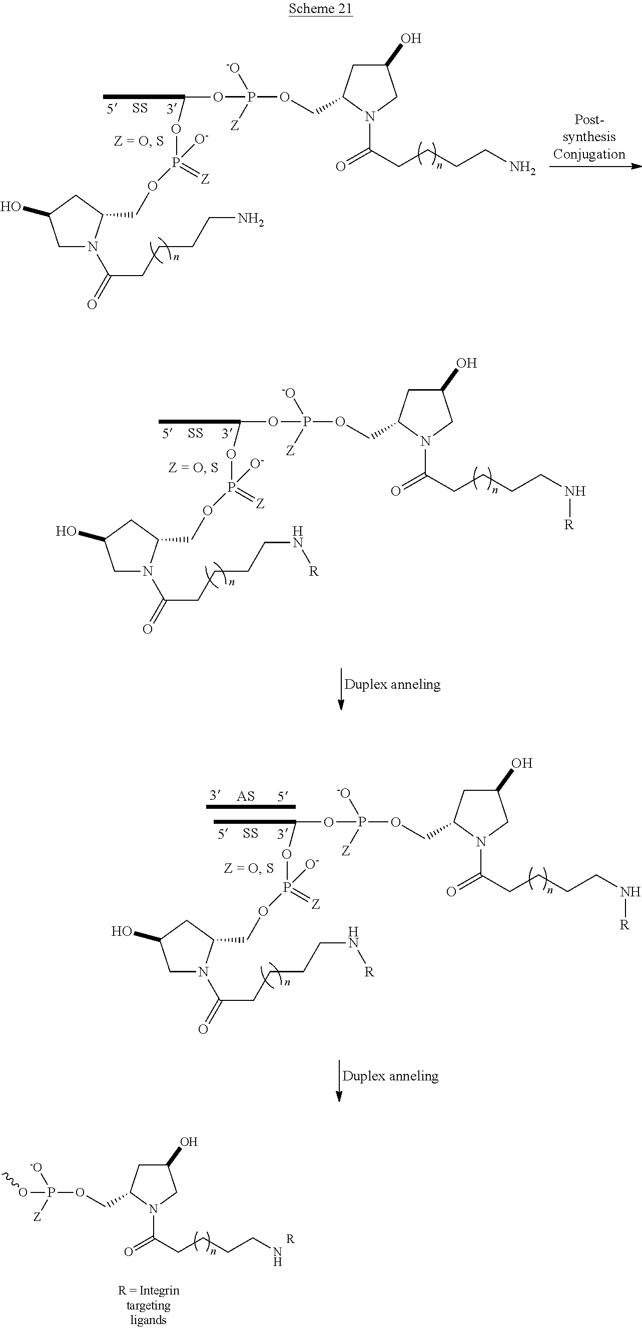

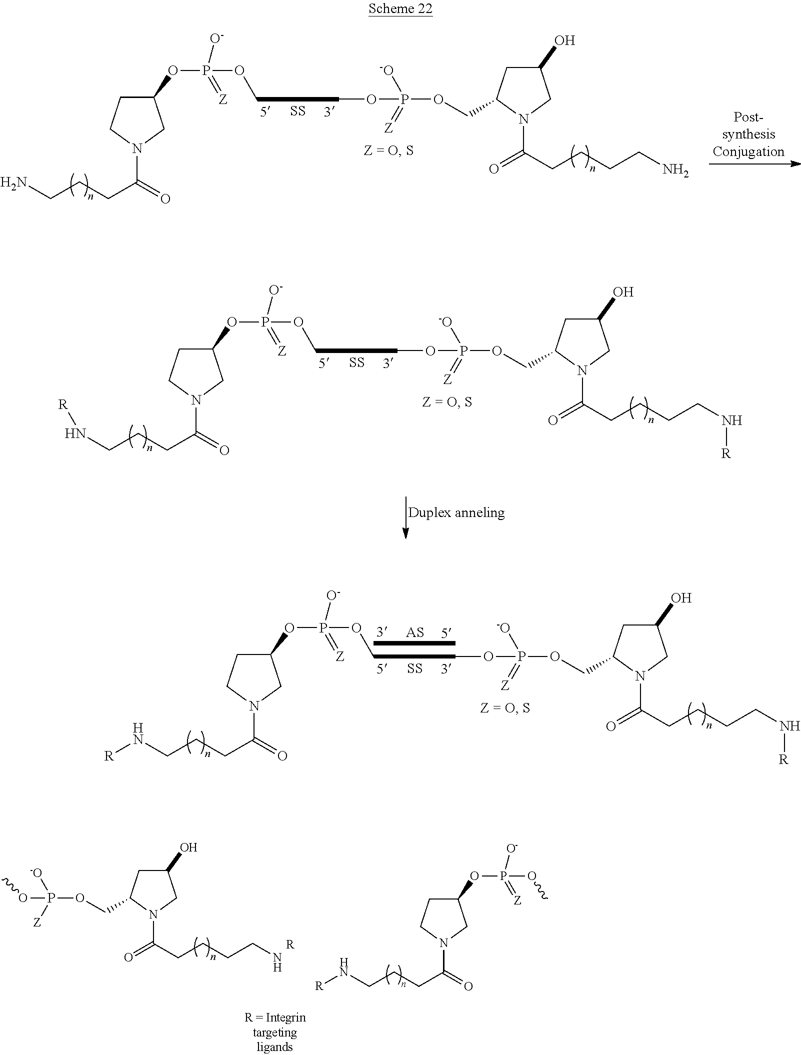

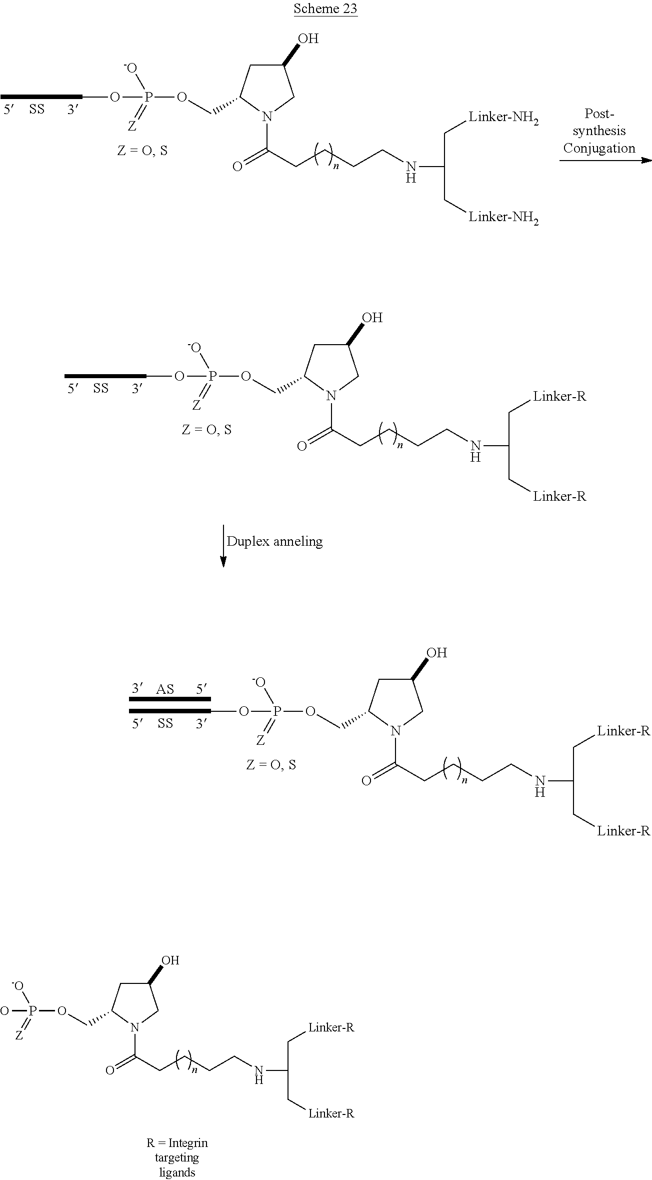

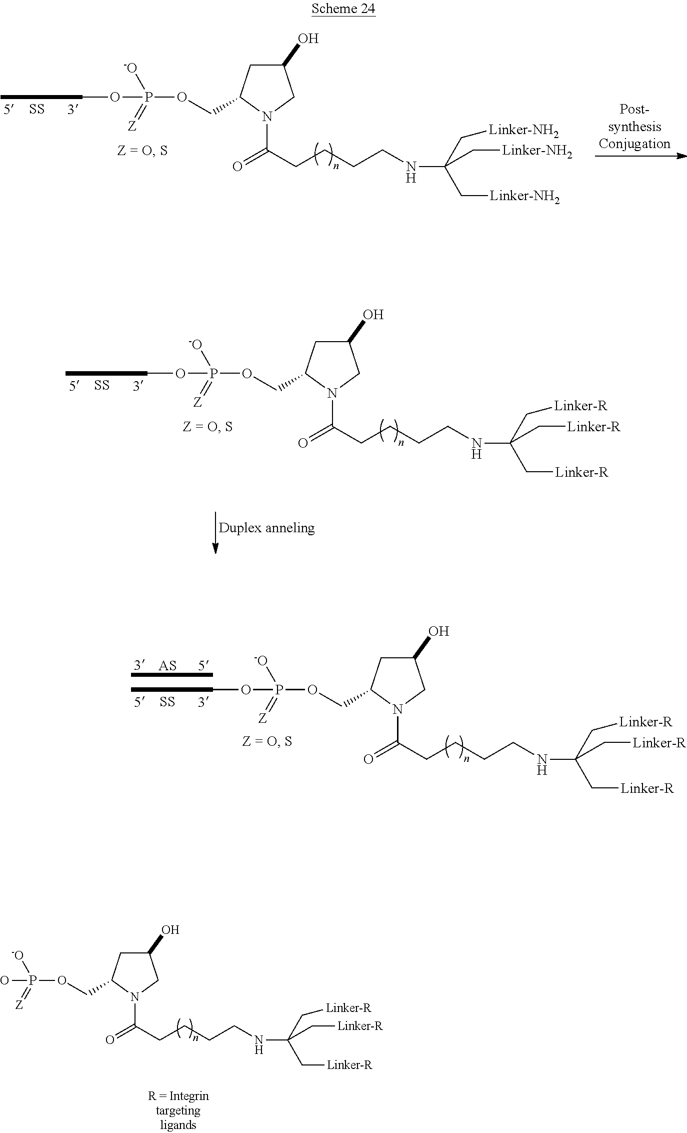

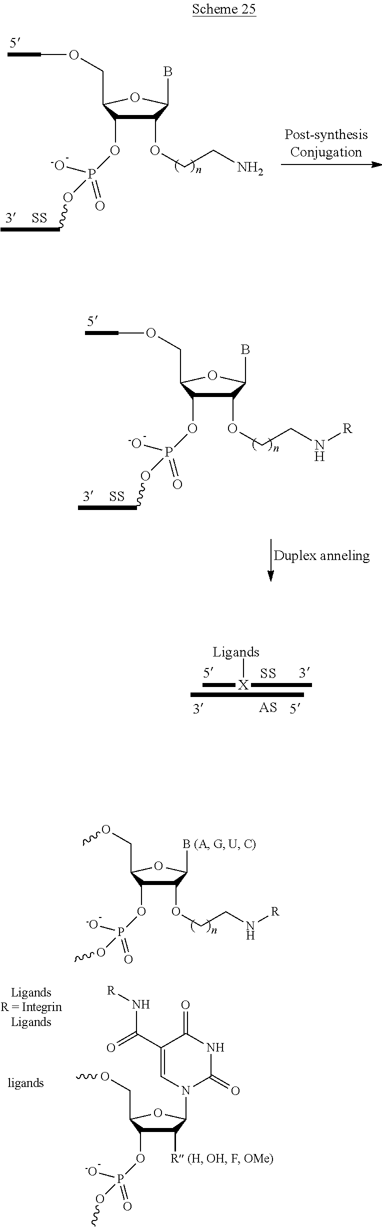

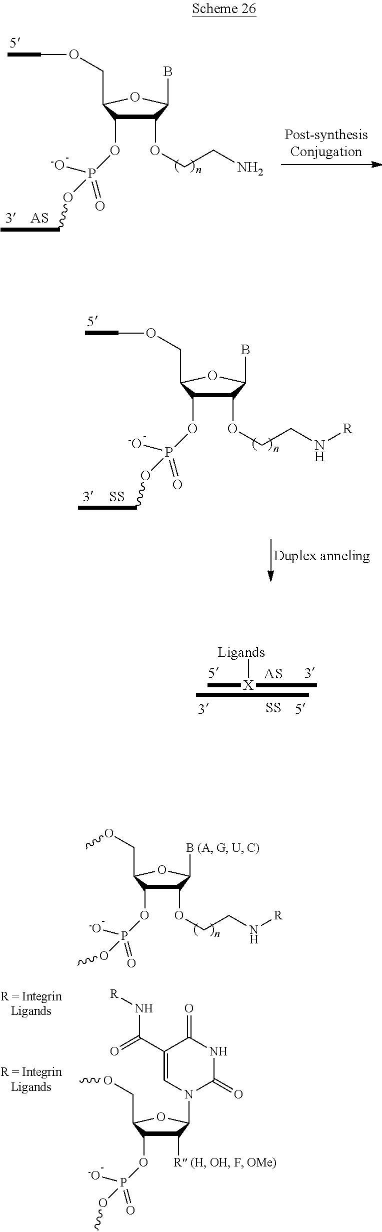

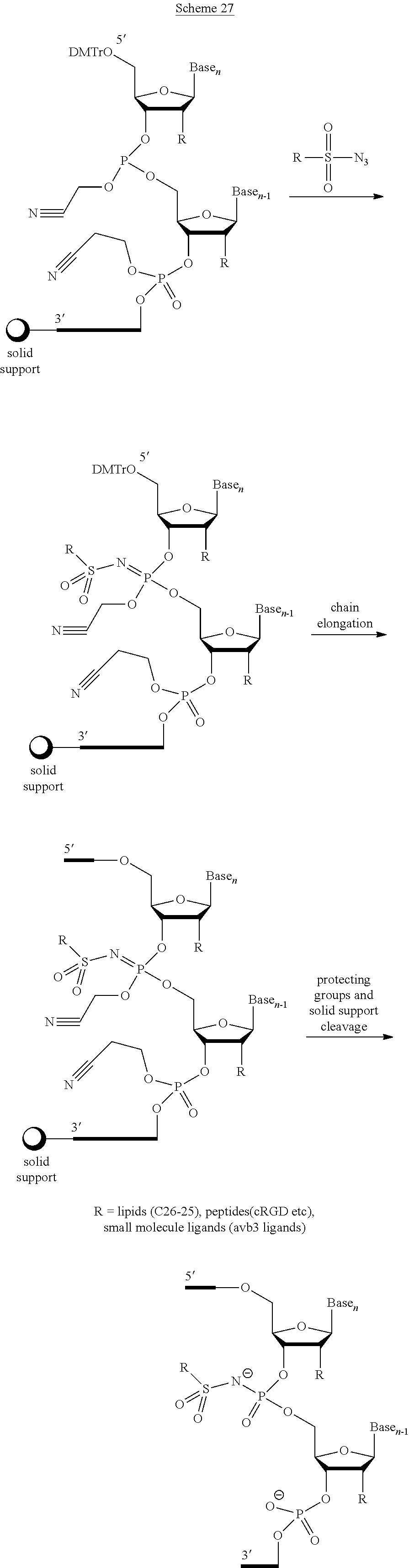

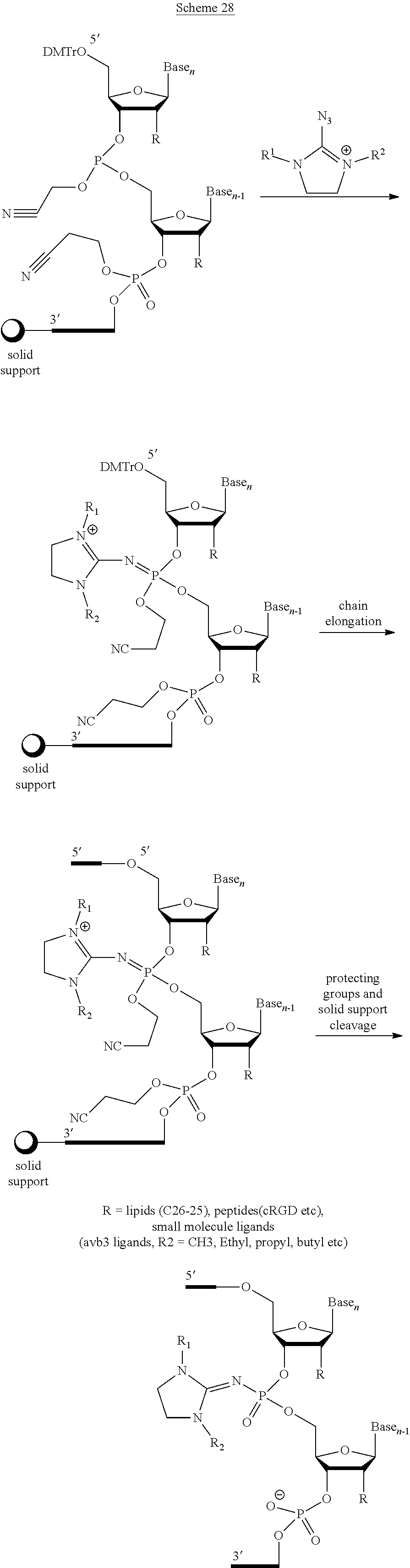

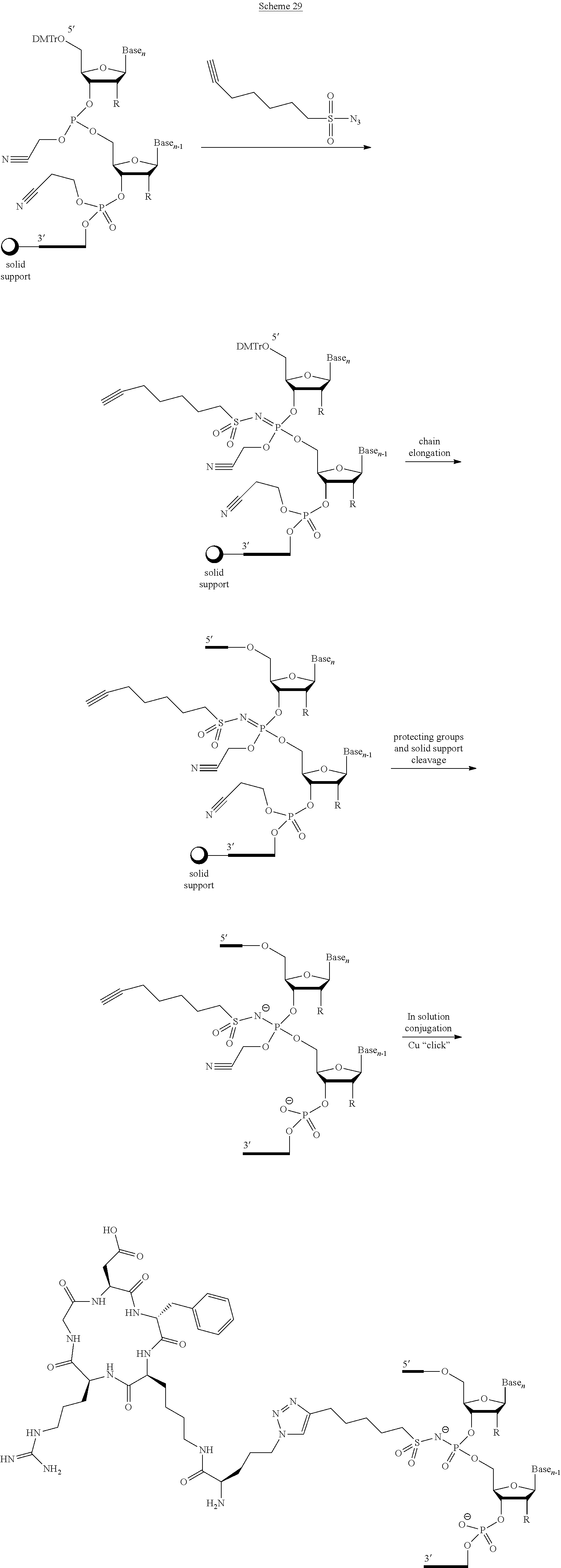

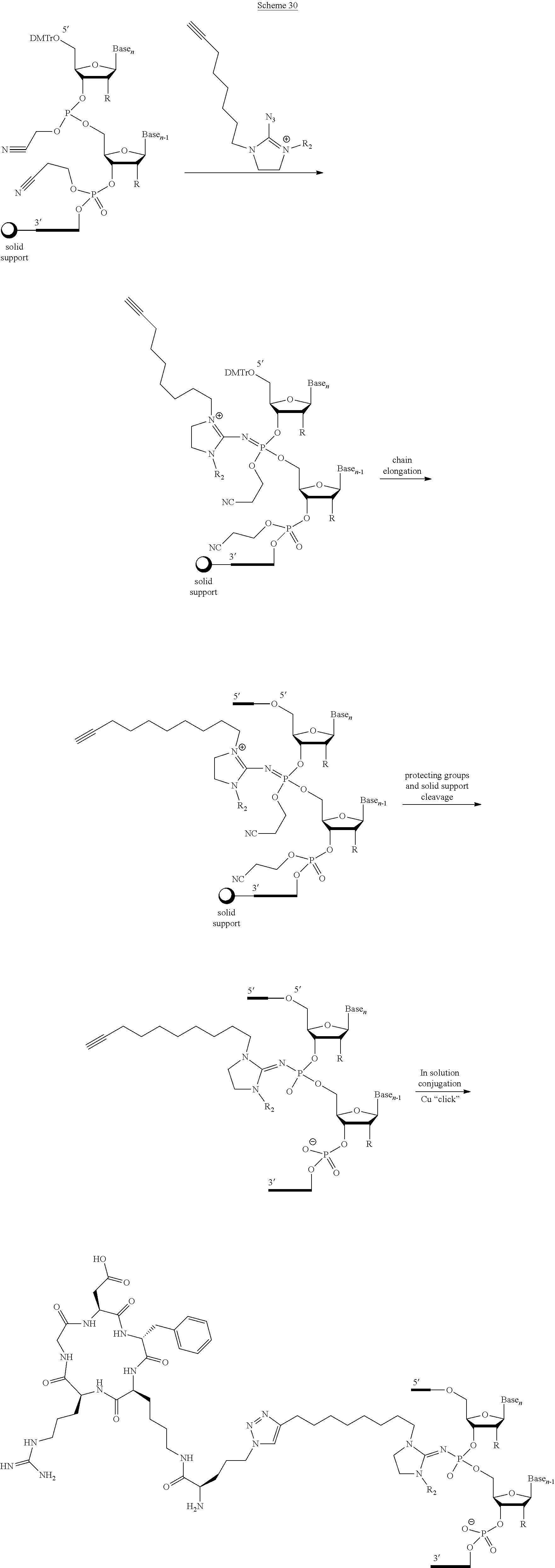

A detailed list of the modified nucleotide sequences of the siRNA duplex sequences is shown in Table 4.

TABLE 4
siRNA duplexes used for in vitro and in vivo studies

| | | | | | Molecular | |
|---|---|---|---|---|---|---|
| Duplex Id | Oligo Id | strand | target | Oligo Seq | Weight | Molecular Weight Found |
| AD-579842 | A- | sense | MYOC | gsusgaa(Ahd)UfaAfAfGfuuauc | 7211.123 | 7207.308 |
| | 1110699 | | | uuascsa (SEQ ID NO: 46) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaaUfaAfAfGfuuaucuuac | 8219.045 | 8215.615 |
| 1488236 | 2686466 | | | sasL240Z67 (SEQ ID NO: 5) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaaUfaAfAfGfuuaucuuac | 9764.785 | 9760.411 |
| 1488237 | 2686467 | | | sasL240Z71 (SEQ ID NO: 6) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaaUfaAfAfGfuuaucuuac | 7842.701 | 7838.527 |
| 1479368 | 2600332 | | | sasL348 (SEQ ID NO: 7) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaaUfaAfAfGfuuaucuuac | 9528.912 | 9523.595 |
| 1479369 | 2600333 | | | sasL349 (SEQ ID NO: 8) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | SSB | usgsacaaCfaGfAfCfuuuaauguas | 7841.717 | 7837.543 |
| 1565878 | 2819653 | | | asL348 (SEQ ID NO: 9) | | |
| | A- | antis | SSB | VPusUfsacaUfuaaagucUfgUfug | 7685.049 | 7681.11 |
| | 2365994 | | | ucasgsc (SEQ ID NO: 48) | | |
| AD- | A- | sense | SSB | usgsacaaCfaGfAfCfuuuaauguas | 8218.061 | 8214.631 |
| 1565879 | 2819654 | | | asL240Z67 (SEQ ID NO: 10) | | |
| | A- | antis | SSB | VPusUfsacaUfuaaaguc Ufg Ufug | 7685.049 | 7681.11 |
| | 2365994 | | | ucasgsc (SEQ ID NO: 48) | | |
| AD- | A- | sense | SSB | usgsacaaCfaGfAfCfuuuaauguas | 9527.928 | 9522.611 |
| 1565880 | 2824431 | | | asL349 (SEQ ID NO: 11) | | |
| | A- | antis | SSB | VPusUfsacaUfuaaaguc Ufg Ufug | 7685.049 | 7681.11 |
| | 2365994 | | | ucasgsc (SEQ ID NO: 48) | | |
| AD- | A- | sense | SSB | usgsacaaCfaGfAfCfuuuaauguas | 9763.801 | 9759.427 |
| 1565881 | 2824432 | | | asL240Z71 (SEQ ID NO: 12) | | |
| | A- | antis | SSB | VPusUfsacaUfuaaaguc Ufg Ufug | 7685.049 | 7681.11 |
| | 2365994 | | | ucasgsc (SEQ ID NO: 48) | | |
| AD- | A- | sense | RhoA | gsasagugCfuGfUfUfuauuaaucu | 8188.965 | 8185.555 |
| 1565882 | 2833825 | | | sasL240Z67 (SEQ ID NO: 13) | | |
| | A- | antis | RHOA | VPusAfsgau UfaAfUfaaacAfgC | 7698.098 | 7694.162 |
| | 2036450 | | | facuucsasa (SEQ ID NO: 49) | | |
| AD | A- | sense | RhoA | gsasagugCfuGfUfUfuauuaaucu | 9734.705 | 9730.351 |
| 1565883 | 2833826 | | | sasL240Z71 (SEQ ID NO: 14) | | |
| | A- | antis | RHOA | VPusAfsgau UfaAfUfaaacAfgC | 7698.098 | 7694.162 |
| | 2036450 | | | facuucsasa (SEQ ID NO: 49) | | |
| AD- | A- | sense | RhoA | gsasagugCfuGfUfUfuauuaaucu | 7812.621 | 7808.467 |
| 1565884 | 2833827 | | | sasL348 (SEQ ID NO: 15) | | |
| | A- | antis | RHOA | VPusAfsgau UfaAfUfaaacAfgC | 7698.098 | 7694.162 |
| | 2036450 | | | facuucsasa (SEQ ID NO: 49) | | |
| AD- | A- | sense | RhoA | gsasagugCfuGfUfUfuauuaaucu | 9498.832 | 9493.536 |
| 1565885 | 2833828 | | | sasL349 (SEQ ID NO: 16) | | |
| | A- | antis | RHOA | VPusAfsgau UfaAfUfaaacAfgC | 7698.098 | 7694.162 |
| | 2036450 | | | facuucsasa (SEQ ID NO: 49) | | |
| AD-579820 | A- | sense | MYOC | gsasccc(Uhd)GfaCfCfAfucccau | 7096.059 | 7092.319 |
| | 1110677 | | | ucsasa (SEQ ID NO: 50) | | |
| | A- | antis | MYOC | VPusUfsgaaUfgGfGfauggUfcA | 7812.064 | 7808.094 |
| | 1099277 | | | fgggucsusu (SEQ ID NO: 51) | | |
| AD- | A- | sense | RHOA | gsasagu(Ghd)CfuGfUfUfuauua | 7181.039 | 7177.254 |
| 1096125 | 2036449 | | | aucsusa (SEQ ID NO: 31) | | |
| | A- | antis | RHOA | VPusAfsgau UfaAfUfaaacAfgC | 7698.098 | 7694,162 |
| | 2036450 | | | facuucsasa (SEQ ID NO: 49) | | |
| AD-901113 | A- | sense | VEGF | gsasgaa(Ahd)GfuGfUfUfuuaua | 7267.146 | 7263.31 |
| | 1700845 | | A | uacsgsa (SEQ ID NO: 32) | | |
| | A- | antis | VEGF | VPusCfsguaUfaUfAfaaacAfcU | 7565.917 | 7562.047 |
| | 1070290 | | A | fuucucsusu (SEQ ID NO: 52) | | |
| AD- | A- | sense | VEGF | gsasgaaaGfuGfUfUfuuauauacg | 8275.068 | 8270.603 |
| 1711739 | 3112837 | | A | sasL240Z67 (SEQ ID NO: 33) | | |
| | A- | antis | VEGF | VPusCfsguaUfaUfAfaaacAfcU | 7565.917 | 7562.047 |
| | 1070290 | | A | fuucucsusu (SEQ ID NO: 52) | | |
| AD- | A- | sense | VEGF | gsasgaa Y224Z67GfuGfUfUfuu | 8067.947 | 8063.579 |
| 1711740 | 3112838 | | A | auauacsgsa (SEQ ID NO: 34) | | |
| | A- | antis | VEGF | VPusCfsguaUfaUfAfaaacAfcU | 7565.917 | 7562.047 |
| | 1070290 | | A | fuucucsusu (SEQ ID NO: 52) | | |
| AD- | A- | sense | MYOC | Z67Q385sgsugaaaUfaAfAfGfu | 9407.347 | 9402.122 |
| 1634439 | 2898771 | | | uaucuuacsasL240Z67 (SEQ ID | | |
| | | | | NO: 35) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | Z67Q385sgsugaaaUfaAfAfGfu | 8189.014 | 8184.597 |
| 1634440 | 2898772 | | | uaucuuascsa (SEQ ID NO: 36) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | Z71Q385sgsugaaaUfaAfAfGfu | 9734.754 | 9729.393 |
| 1634441 | 2898773 | | | uaucuuascsa (SEQ ID NO: 37) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaaUfaAfAfGfuuaucuuac | 9454.445 | 9449.118 |
| 1634442 | 2898774 | | | sasQ157sZ67L240Z67 (SEQ | | |
| | | | | ID NO: 38) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaY224Z67UfaAfAfGfuu | 8011.925 | 8007.578 |
| 1634443 | 2898775 | | | aucuuascsa (SEQ ID NO: 39) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaa Y224Z71UfaAfAfGfuu | 9557.665 | 9552.373 |
| 1634444 | 2898776 | | | aucuuascsa (SEQ ID NO: 40) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | LRRK | asgscug(Uhd)agCfAfAfggaguu | 9067.013 | 9062.304 |
| 1623995 | 2992766 | | 2 | uugaL96 (SEQ ID NO: 41) | | |
| | A- | antis | LRRK | VPusdCsaadAadCuccudTgCfua | 7573.086 | 7569.169 |
| | 2992767 | | 2 | cagcususg (SEQ ID NO: 53) | | |
| AD- | A- | sense | MYOC | gsusgaaaUfaAfAfGfuuaucuuac | 8227.054 | 8222.678 |
| 1751755 | 3177380 | | | sasL373Z106 (SEQ ID NO: 23) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | Q420sZ93gsugaaaUfaAfAfGfu | 9661.597 | 9656.361 |
| 1751756 | 3177381 | | | uaucuuacsasL373Z93 (SEQ ID | | |
| | | | | NO: 42) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | gsusgaaaUfaAfAfGfuuaucuuac | 9707.688 | 9702.349 |
| 1751757 | 3177382 | | | sasQ425sZ93L373Z93 (SEQ | | |
| | | | | ID NO: 43) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD-822899 | A- | sense | MYOC | csusuga(Ahd)CfaUfGfGfucacu | 7179.072 | 7175.281 |
| | 1577508 | | | uausgsa (SEQ ID NO: 44) | | |
| | A- | antis | MYOC | VPusCfsauaAfg UfGfaccaUfgU | 7684.019 | 7680.097 |
| | 1577509 | | | fucaagsusu (SEQ ID NO: 54) | | |
| AD- | A- | sense | MYOC | csusugaaCfaUfGfGfucacuuaugs | 8186.994 | 8182.575 |
| 1646735 | 3019042 | | | asL240Z67 (SEQ ID NO: 45) | | |
| | A- | antis | MYOC | VPusCfsauaAfgUfGfaccaUfgU | 7684.019 | 7680.097 |
| | 1577509 | | | fucaagsusu (SEQ ID NO: 54) | | |
| AD- | A- | sense | MYOC | gsusgaaaUfaAfAfGfuuaucuuac | 8218.037 | 8213.595 |
| 1623955 | 2894709 | | | sasL240Z91 (SEQ ID NO: 55) | | |
| | A- | antis | MYOC | VPusGfsuaaGfaUfAfacuuUfaU | 7669.005 | 7665.087 |
| | 1100335 | | | fuucacsasg (SEQ ID NO: 47) | | |
| AD- | A- | sense | MYOC | csusugaaCfaUfGfGfucacuuaugs | 7669.005 | 7665.087 |
| 1646735 | 3019042 | | | asL240Z67 (SEQ ID NO: 45) | | |
| | A- | antis | MYOC | VPusCfsauaAfgUfGfaccaUfgU | 8186.994 | 8182.575 |
| | 1577509 | | | fucaagsusu (SEQ ID NO: 54) | | |
| AD- | A- | sense | VEGF | gsasgaaaGfuGfUfUfuuauauacg | 7684.019 | 7680.097 |
| 1711739 | 3112837 | | A | sasL240Z67 (SEQ ID NO: 33) | | |
| | A- | antis | VEGF | VPusCfsguaUfaUfAfaaacAfcU | 8275.068 | 8270.603 |
| | 1070290 | | A | fuucucsusu (SEQ ID NO: 52) | | |
| AD- | A- | sense | VEGF | gsasgaa Y224Z67GfuGfUfUfuu | 7565.917 | 7562.047 |
| 1711740 | 3112838 | | A | auauacsgsa (SEQ ID NO: 34) | | |
| | A- | antis | VEGF | VPusCfsguaUfaUfAfaaacAfcU | 8067.947 | 8063.579 |
| | 1070290 | | A | fuucucsusu (SEQ ID NO: 52) | | |
| AD- | A- | sense | MYOC | csusuga(Ahd)CfaUfGfGfucacu | 7179.072 | 7175.281 |
| 1699851 | 1577508 | | | uausgsa (SEQ ID NO: 44) | | |
| | A- | antis | MYOC | VPusCfsauaAfgUfGfaccaUfgU | 8624.185 | 8619.375 |
| | 3041945 | | | fucaY244gsusu (SEQ ID NO: | | |
| | | | | 56) | | |
| AD- | A- | sense | MYOC | csusugaaCfaUfGfGfucacuuaugs | 8186.994 | 8182.575 |
| 1699852 | 3019042 | | | asL240Z67 (SEQ ID NO: 45) | | |
| | A- | antis | MYOC | VPusCfsauaAfgUfGfaccaUfgU | 8624.185 | 8619.375 |
| | 3041945 | | | fucaY244gsusu (SEQ ID NO: | | |
| | | | | 56) | | |
| AD- | A- | sense | MYOC | csusugaaCfaUfGfGfucacuuaugs | 8314.119 | 8309.697 |
| 1720764 | 3006759 | | | asL373Z93 (SEQ ID NO: 57) | | |
| | A- | antis | MYOC | VPusCfsauaAfgUfGfaccaUfgU | 8624.185 | 8619.375 |
| | 3041945 | | | fucaY244gsusu (SEQ ID NO: | | |
| | | | | 56) | | |
| AD- | A- | sense | MYOC | csusugaaCfaUfGfGfucacuuaugs | 7810.65 | 7806.499 |
| 1720765 | 3142179 | | | asL348 (SEQ ID NO: 58) | | |
| | A- | antis | MYOC | VPusCfsauaAfgUfGfaccaUfgU | 8624.185 | 8619.375 |
| | 3041945 | | | fucaY244gsusu (SEQ ID NO: | | |
| | | | | 56) | | |

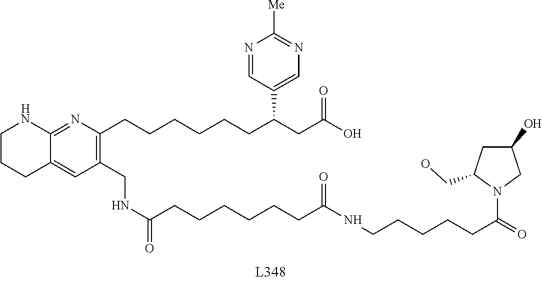 [SMILES: Cc1ncc([C@@H](CCCCCCc2nc3c(cc2CNC(=O)CCCCCCC(=O)NCCCCCC(=O)N2C[C@H](O)C[C@H]2CO)CCCN3)CC(=O)O)cn1]

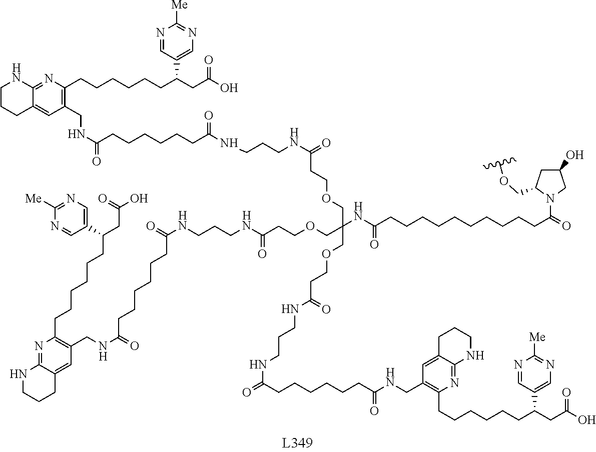 [SMILES: *OC[C@@H]1C[C@@H](O)CN1C(=O)CCCCCCCCCCC(=O)NC(COCCC(=O)NCCCNC(=O)CCCCCCC(=O)NCc1cc2c(nc1CCCCCC[C@@H](CC(=O)O)c1cnc(C)nc1)NCCC2)(COCCC(=O)NCCCNC(=O)CCCCCCC(=O)NCc1cc2c(nc1CCCCCC[C@@H](CC(=O)O)c1cnc(C)nc1)NCCC2)COCCC(=O)NCCCNC(=O)CCCCCCC(=O)NCc1cc2c(nc1CCCCCC[C@@H](CC(=O)O)c1cnc(C)nc1)NCCC2]

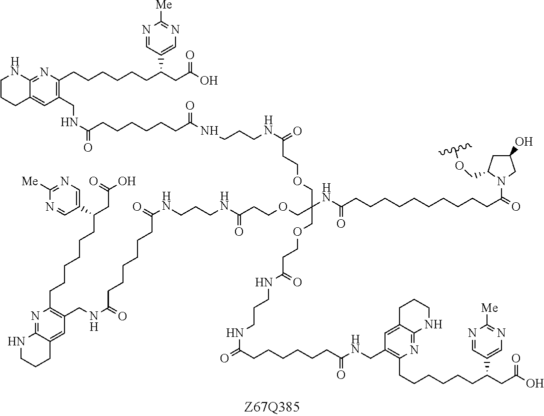 [SMILES: *OC[C@@H]1C[C@@H](O)CN1C(=O)CCCCCCCCCCC(=O)NC(COCCC(=O)NCCCNC(=O)CCCCCCC(=O)NCc1cc2c(nc1CCCCCC[C@@H](CC(=O)O)c1cnc(C)nc1)NCCC2)(COCCC(=O)NCCCNC(=O)CCCCCCC(=O)NCc1cc2c(nc1CCCCCC[C@@H](CC(=O)O)c1cnc(C)nc1)NCCC2)COCCC(=O)NCCCNC(=O)CCCCCCC(=O)NCc1cc2c(nc1CCCCCC[C@@H](CC(=O)O)c1cnc(C)nc1)NCCC2]

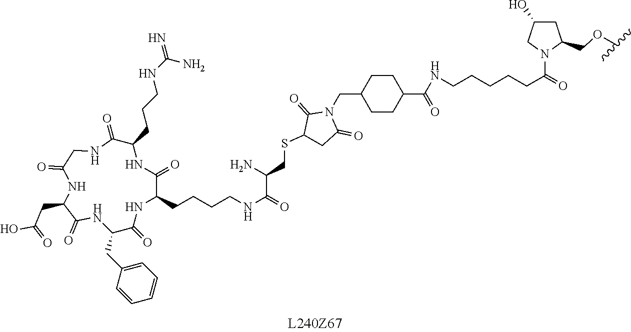 [SMILES: *OC[C@@H]1C[C@@H](O)CN1C(=O)CCCCCNC(=O)C1CCC(CN2C(=O)CC(SC[C@H](N)C(=O)NCCCC[C@H]3NC(=O)[C@H](Cc4ccccc4)NC(=O)[C@@H](CC(=O)O)NC(=O)CNC(=O)[C@@H](CCCNC(=N)N)NC3=O)C2=O)CC1]

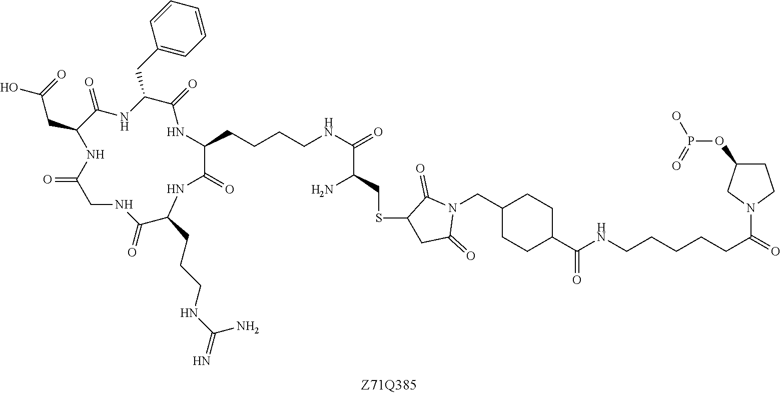

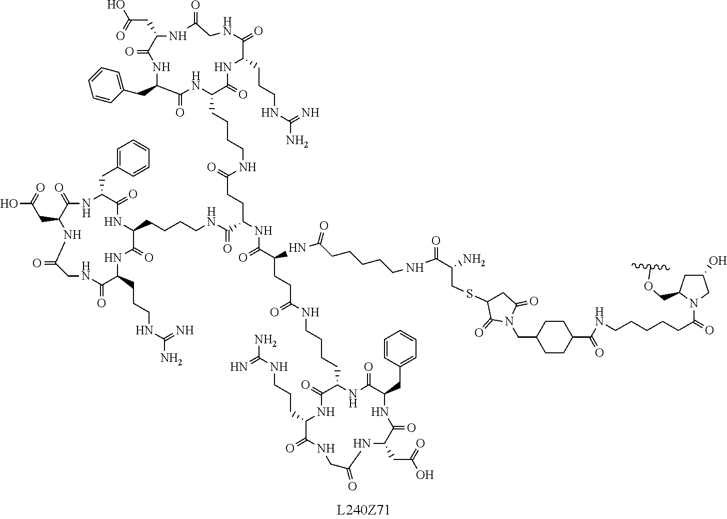 [SMILES: *OC[C@H]1C[C@H](O)CN1C(=O)CCCCCNC(=O)C1CCC(CN2C(=O)CC(SC[C@@H](N)C(=O)NCCCCCC(=O)N[C@@H](CCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)N[C@@H](CCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C2=O)CC1]

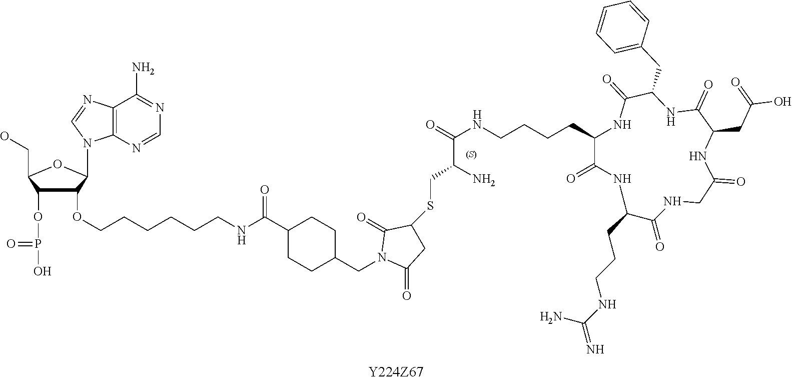

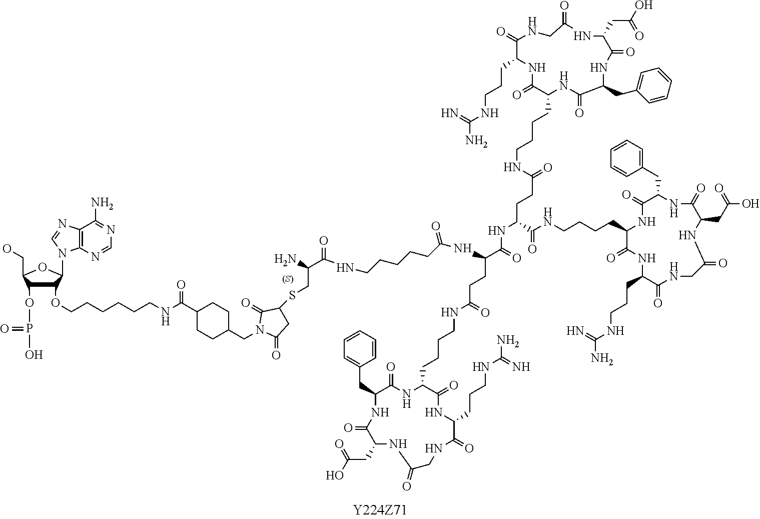

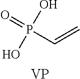

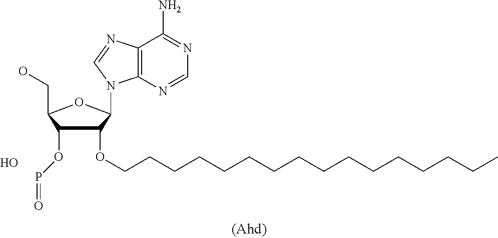

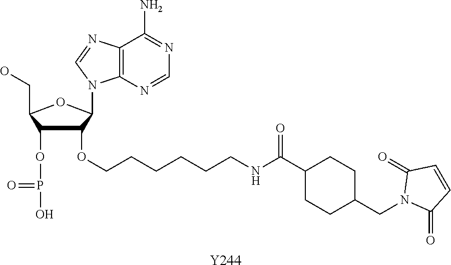

### Example 4: In vivo evaluation of integrin targeting ligands for ocular delivery of siRNA agents

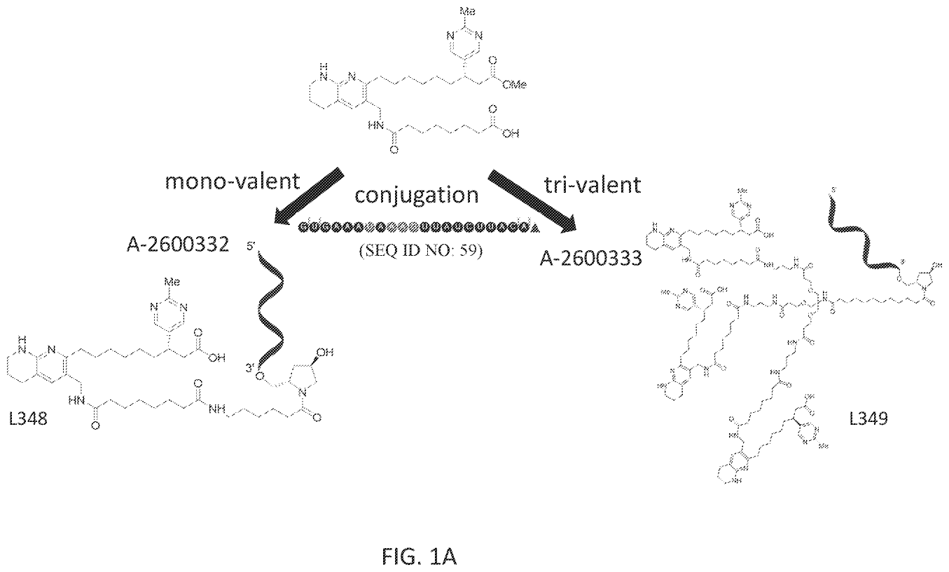

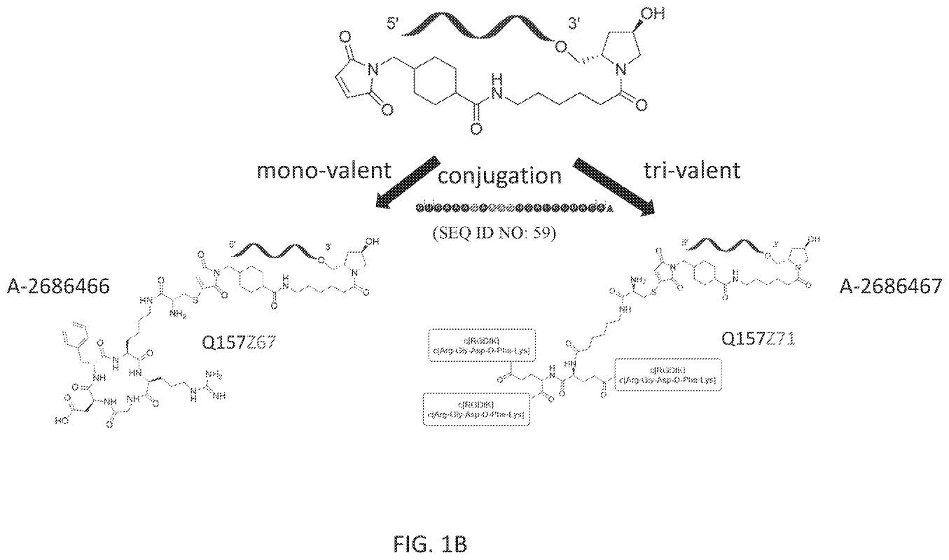

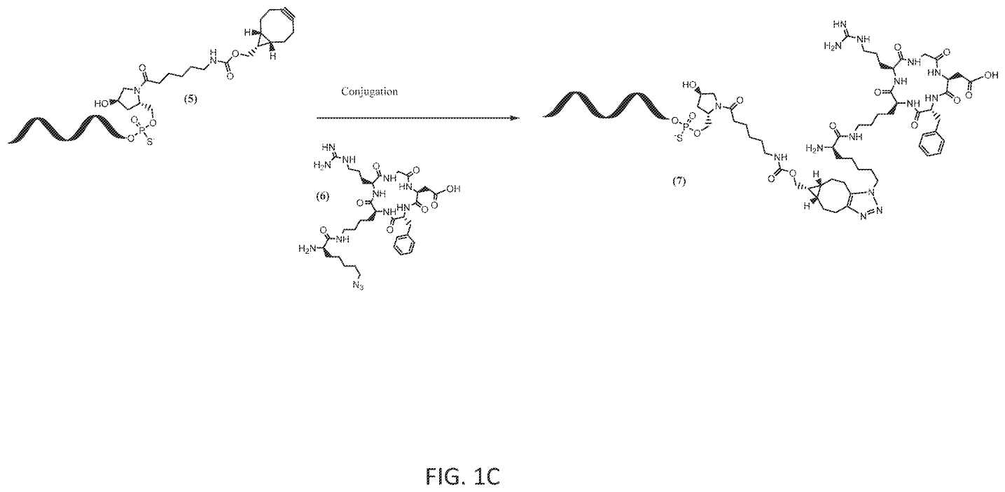

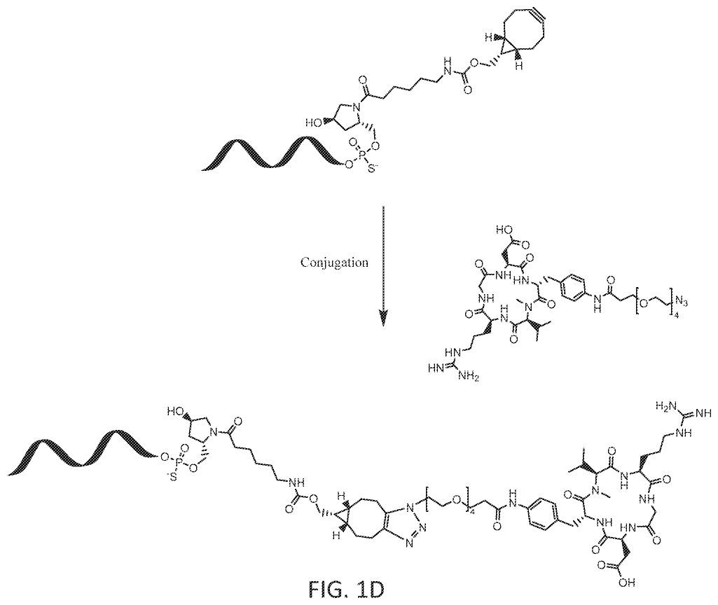

To evaluate the impact of integrin targeting ligands (αvβ₃ ligands or cyclic RGD ligands) on the delivery of siRNA agents to the trabecular meshwork™ of rat eyes in vivo, the MYOC siRNA conjugates described in Examples 2 and 3 and depicted in FIGS. 1A-1D were administered by ocular delivery to rats. MYOC siRNAs including internal C16 ligand chemistry were assessed as a comparator (AD-579842 and AD-579820).

### Experimental Methods

Briefly, Sprague Dawley rats were administered a single dose of 50 μg/eye of an siRNA agent, or PBS, by intravitreal (IVT) injection (5 μL/eye; 4 animals per cohort). The experiment was terminated after 14 days post-dose. Rats were euthanized and the eyes were collected and dissected. Dissections of the eyes included a limbal ring dissection (trabecular meshwork, iris, and cilliary body). The study design is summarized in Table 5.

TABLE 5
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | AD-579842 | | 50 | | 4 | dose, | | |
| | (Internal | | | | | Day 0 | | |
| | C16) | | | | | | | |
| 3 | α_{V}β₃ SM_3′ | | | | 4 | | | |
| | monovalent | | | | | | | |
| | AD-1479368 | | | | | | | |
| 4 | α_{V}β₃ SM_3′ | | | | 4 | | | |
| | trivalent | | | | | | | |
| | AD-1479369 | | | | | | | |
| 5 | Cyclic RGD | | | | 4 | | | |
| | monovalent | | | | | | | |
| | AD-1488236 | | | | | | | |
| 6 | Cyclic RGD | | | | 4 | | | |
| | trivalent | | | | | | | |
| | AD-1488237 | | | | | | | |

Delivery of the siRNA conjugates was evaluated by the measurement of MYOC mRNA in the limbal ring at 14 days post-dose. Eye samples were ground and tissue lysates were prepared. As outlined below, mRNA was isolated from the lysates and MYOC mRNA levels in the lysates were determined by qRT-PCR.

A dose response study was performed as described above using a single IVT administration of AD-1488236 at 10 μg, 50 μg, or 100 μg/eye and the samples were processed at day 14 post injection. MYOC siRNA including internal C16 ligand chemistry was assessed as a comparator (AD-579820). The study design is summarized in Table 6.

TABLE 6
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | AD-579820 | | 50 | | 4 | dose, | | |
| | (Internal | | | | | Day 0 | | |
| | C16) | | | | | | | |
| 3 | α_{V}β₃ | | 10 | | 4 | | | |
| | cRGD_3′ | | | | | | | |
| | monovalent | | | | | | | |
| | AD-1488236 | | | | | | | |
| 4 | α_{V}β₃ | | 50 | | 4 | | | |
| | cRGD_3′ | | | | | | | |
| | monovalent | | | | | | | |
| | AD-1488236 | | | | | | | |
| 5 | α_{V}β₃ | | 100 | | 4 | | | |
| | cRGD_3′ | | | | | | | |
| | monovalent | | | | | | | |
| | AD-1488236 | | | | | | | |

A lower dose response study was performed as described above using a single IVT administration of AD-1488236 at 0.1 μg, 1 μg, or 10 μg/eye and the samples were processed at day 14 post injection. MYOC siRNA including internal C16 ligand chemistry was assessed as a comparator (AD-579820). The study design is summarized in Table 7.

TABLE 7
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | AD-579820 | | 50 | | 4 | dose, | | |
| | (Internal | | | | | Day 0 | | |
| | C16) | | | | | | | |
| 3 | α_{V}β₃ | | 0.1 | | 4 | | | |
| | cRGD_3′ | | | | | | | |
| | monovalent | | | | | | | |
| | AD-1488236 | | | | | | | |
| 4 | α_{V}β₃ | | 1 | | 4 | | | |
| | cRGD_3′ | | | | | | | |
| | monovalent | | | | | | | |
| | AD-1488236 | | | | | | | |
| 5 | α_{V}β₃ | | 10 | | 4 | | | |
| | cRGD_3′ | | | | | | | |
| | monovalent | | | | | | | |
| | AD-1488236 | | | | | | | |

A duration study was performed as described above using a single IVT administration of AD-1488236 at 10 μg/eye versus IVYOC internal C16 ligand chemistry (AD-579820) and the samples were processed at day 14, day 28, day 42 and day 56 post injection. The study design is summarized in Table 8.

TABLE 8
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | AD-579842 | | 50 | | 4 | dose, | | |
| | (Internal | | | | | Day 0 | | |
| | C16) | | | | | | | |
| 3 | α_{V}β₃ | | 10 | | 4 | | | |
| | cRGD_3′ | | | | | | | |
| | monovalent | | | | | | | |
| | AD-1488236 | | | | | | | |
| 4 | PBS | | n/a | | 4 | | D 28 | |
| 5 | AD-579842 | | 50 | | 4 | | | |
| | (Internal | | | | | | | |
| | C16) | | | | | | | |
| 6 | α_{V}β₃ | | 10 | | 4 | | | |
| | cRGD_3′ | | | | | | | |
| | monovalent | | | | | | | |
| | AD-1488236 | | | | | | | |
| 7 | PBS | | n/a | | 4 | | D 42 | |
| 8 | AD-579842 | | 50 | | 4 | | | |
| | (Internal | | | | | | | |
| | C16) | | | | | | | |
| 9 | α_{V}β₃ | | 10 | | 4 | | | |
| | cRGD_3′ | | | | | | | |
| | monovalent | | | | | | | |
| | AD-1488236 | | | | | | | |
| 10 | PBS | | n/a | | 4 | | D 56 | |
| 11 | AD-579842 | | 50 | | 4 | | | |
| | (Internal | | | | | | | |
| | C16) | | | | | | | |
| 12 | α_{V}β₃ | | 10 | | 4 | | | |
| | cRGD_3′ | | | | | | | |
| | monovalent | | | | | | | |
| | AD-1488236 | | | | | | | |

Performance of cRGD 3′ mono-ligand was evaluated with an independent target (RhoA) using a single IVT administration ofAD-1565882 at 50 μg/eye versus RhoA internal C16 ligand chemistry (AD-1096125) and the samples were processed at day 14 post injection. In same study design we tested out the specificity by including animals dosed with MYOC siRNA (cRGD AD-1488236 at 0.1, 1 and 10 μg/eye dose). The study design is summarized in Table 9.

TABLE 9
Study Design

| | Test Article | | | | Number of | Dose | | |
|---|---|---|---|---|---|---|---|---|
| | ID (RhoA | Dose | Doses | Dose | Animals | Regime | End of | |
| Group | or MYOC) | Route | (μg) | Volume | (N = 24) | n | Study | Analysis |
| 1 | PBS | IVT | n/a | 5 μL/eye | 4 | Single | D 14 | qPCR |
| 2 | AD-1096125 | | 50 | | 4 | dose, | | |
| | (Internal | | | | | Day 0 | | |
| | C16-RhoA) | | | | | | | |
| 3 | α_{V}β₃ | | 50 | | 4 | | | |
| | cRGD_3′ | | | | | | | |
| | monovalent | | | | | | | |
| | AD-1565882 | | | | | | | |
| | (RhoA) | | | | | | | |
| 5 | α_{V}β₃ | | 0.1 | | 4 | | | |
| | cRGD_3′ | | | | | | | |
| | monovalent | | | | | | | |
| | AD-1488236 | | | | | | | |
| | (MYOC) | | | | | | | |
| 6 | α_{V}β₃ | | 1 | | 4 | | | |
| | cRGD_3′ | | | | | | | |
| | monovalent | | | | | | | |
| | AD-1488236 | | | | | | | |
| | (MYOC) | | | | | | | |
| 7 | α_{V}β₃ | | 10 | | 4 | | | |
| | cRGD_3′ | | | | | | | |
| | monovalent | | | | | | | |
| | AD-1488236 | | | | | | | |
| | (MYOC) | | | | | | | |

Performance of cRGD 3′ mono-(AD-171 1739) or sense strand position 6 ligand (AD-1711740) was evaluated with posterior cup target (VEGFA) using a single IVT administration at 0.1 or 10 μg/eye versus VEGFA internal C16 ligand chemistry (AD-901113) and the samples were processed at day 21 and 35 post injection. The study design is summarized in Table 10.

TABLE 10
Study Design

| | | | | | Number of | Dose | | |
|---|---|---|---|---|---|---|---|---|
| | Test Article | Dose | Doses | Dose | Animals | Regime | End of | |
| Group | ID (VEGFA) | Route | (μg) | Volume | (N = 24) | n | Study | Analysis |
| 1 | PBS | IVT | n/a | 5 μL/eye | 4 | Single | D 21 | qPCR |
| 2 | AD-901113 | | 1 | | 4 | dose, | | |
| | (Internal | | | | | Day 0 | | |
| | C16) | | | | | | | |
| 3 | AD-901113 | | 10 | | 4 | | | |
| | (Internal | | | | | | | |
| | C16) | | | | | | | |
| 4 | α_{V}β₃ | | 1 | | 4 | | | |
| | cRGD 3′ | | | | | | | |
| | monovalent | | | | | | | |
| | AD-1711739 | | | | | | | |
| 5 | α_{V}β₃ | | 10 | | 4 | | | |
| | cRGD 3′ | | | | | | | |
| | monovalent | | | | | | | |
| | AD-1711739 | | | | | | | |
| 6 | α_{V}β₃ cRGD | | 1 | | 4 | | | |
| | internal SS6 | | | | | | | |
| | monovalent | | | | | | | |
| | AD-1711740 | | | | | | | |
| 7 | α_{V}β₃ cRGD | | 10 | | 4 | | | |
| | internal SS6 | | | | | | | |
| | monovalent | | | | | | | |
| | AD-1711740 | | | | | | | |
| 8 | PBS | | n/a | | 4 | | D 35 | |
| 9 | AD-901113 | | 1 | | 4 | | | |
| | (Internal | | | | | | | |
| | C16) | | | | | | | |
| 10 | AD-901113 | | 10 | | 4 | | | |
| | (Internal | | | | | | | |
| | C16) | | | | | | | |
| 11 | α_{V}β₃ | | 1 | | 4 | | | |
| | cRGD 3′ | | | | | | | |
| | monovalent | | | | | | | |
| | AD-1711739 | | | | | | | |
| 12 | α_{V}β₃ | | 10 | | 4 | | | |
| | cRGD 3′ | | | | | | | |
| | monovalent | | | | | | | |
| | AD-1711739 | | | | | | | |
| 13 | α_{V}β₃ cRGD | | 1 | | 4 | | | |
| | internal SS6 | | | | | | | |
| | monovalent | | | | | | | |
| | AD-1711740 | | | | | | | |
| 14 | α_{V}β₃ cRGD | | 10 | | 4 | | | |
| | internal SS6 | | | | | | | |
| | monovalent | | | | | | | |
| | AD-1711740 | | | | | | | |

cRGD ligand SAR was performed as described above using a single IVT administration of either AD-1488236 at 50 μg/eye, WYOC internal C16 ligand chemistry (AD-579842) or cRGD 3′ mono (AD-1488236) and the samples were processed at day 14 post injection. The study design is summarized in Table 11.

TABLE 11
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | AD-579842 | | 50 | | 4 | dose, | | |
| | (Internal | | | | | Day 0 | | |
| | C16) | | | | | | | |
| 3 | α_{V}β₃ | | 10 | | 4 | | | |
| | cRGD_3′ | | | | | | | |
| | monovalent | | | | | | | |
| | AD-1488236 | | | | | | | |
| 4 | α_{V}β₃ | | 50 | | 4 | | | |
| | cRGD_3′ | | | | | | | |
| | monovalent | | | | | | | |
| | AD-1488236 | | | | | | | |
| 5 | α_{V}β₃ | | 50 | | 4 | | | |
| | cRGD_3′-5′ | | | | | | | |
| | bivalent | | | | | | | |
| | AD-1634439 | | | | | | | |
| 6 | α_{V}β₃ | | 50 | | 4 | | | |
| | cRGD_5′ | | | | | | | |
| | monovalent | | | | | | | |
| | AD-1634440 | | | | | | | |
| 7 | α_{V}β₃ | | 50 | | 4 | | | |
| | cRGD_5′ | | | | | | | |
| | trivalent | | | | | | | |
| | AD-1634441 | | | | | | | |
| 8 | α_{V}β₃ | | 50 | | 4 | | | |
| | cRGD_3′ | | | | | | | |
| | (1 + 1) | | | | | | | |
| | bivalent | | | | | | | |
| | AD-1634442 | | | | | | | |
| 9 | α_{V}β₃ | | 50 | | 4 | | | |
| | cRGD_3′ | | | | | | | |
| | (internal | | | | | | | |
| | SS6) | | | | | | | |
| | monovalent | | | | | | | |
| | AD-1634443 | | | | | | | |
| 10 | α_{V}β₃ | | 50 | | 4 | | | |
| | cRGD_3′ | | | | | | | |
| | (internal | | | | | | | |
| | SS6) trivalent | | | | | | | |
| | AD-1634444 | | | | | | | |
| 11 | α_{V}β₃ | | 10 | | 4 | | | |
| | cRGD_3′ | | | | | | | |
| | mono | | | | | | | |
| | scrambled | | | | | | | |
| | AD-1623955 | | | | | | | |
| 12 | α_{V}β₃ | | 50 | | 4 | | | |
| | cRGD_3′ | | | | | | | |
| | mono | | | | | | | |
| | scrambled | | | | | | | |
| | AD-1623955 | | | | | | | |

cRGD ligand SAR was evaluated at lower doses (10 μg/eye) after a single rat IVT injection and the samples were processed at day 14 post injection. The study design is summarized in Table 12.

TABLE 12
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | α_{V}β₃ | | 10 | | 4 | dose, | | |
| | cRGD_3′ | | | | | Day 0 | | |
| | monovalent | | | | | | | |
| | AD-1488236 | | | | | | | |
| 3 | α_{V}β₃ | | 10 | | 4 | | | |
| | cRGD_3′-5′ | | | | | | | |
| | bivalent | | | | | | | |
| | AD-1634439 | | | | | | | |
| 4 | α_{V}β₃ | | 10 | | 4 | | | |
| | cRGD_5′ | | | | | | | |
| | monovalent | | | | | | | |
| | AD-1634440 | | | | | | | |
| 5 | α_{V}β₃ | | 10 | | 4 | | | |
| | cRGD_5′ | | | | | | | |
| | trivalent | | | | | | | |
| | AD-1634441 | | | | | | | |
| 6 | α_{V}β₃ | | 10 | | 4 | | | |
| | cRGD_3′ | | | | | | | |
| | (1 + 1) | | | | | | | |
| | bivalent | | | | | | | |
| | AD-1634442 | | | | | | | |
| 7 | α_{V}β₃ | | 10 | | 4 | | | |
| | cRGD_3′ | | | | | | | |
| | (internal | | | | | | | |
| | SS6) | | | | | | | |
| | monovalent | | | | | | | |
| | AD-1634443 | | | | | | | |
| 8 | α_{V}β₃ | | 10 | | 4 | | | |
| | cRGD_3′ | | | | | | | |
| | (internal | | | | | | | |
| | SS6) trivalent | | | | | | | |
| | AD-1634444 | | | | | | | |

Dose response cRGD ligand SAR was evaluated at 1 and 10 μg/eye after a single rat IVT injection, and the samples were processed at day 14 post injection. The study design is summarized in Table 13.

TABLE 13
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | α_{V}β₃ | | 10 | | 4 | dose, | | |
| | cRGD_3′ | | | | | Day 0 | | |
| | monovalent | | | | | | | |
| | AD-1488236 | | | | | | | |
| 3 | α_{V}β₃ | | 1 | | 4 | | | |
| | cRGD_3′-5′ | | | | | | | |
| | bivalent | | | | | | | |
| | AD-1634439 | | | | | | | |
| 4 | α_{V}β₃ | | 10 | | 4 | | | |
| | cRGD_3′-5′ | | | | | | | |
| | bivalent | | | | | | | |
| | AD-1634439 | | | | | | | |
| 5 | α_{V}β₃ | | 1 | | 4 | | | |
| | cRGD_5′ | | | | | | | |
| | monovalent | | | | | | | |
| | AD-1634440 | | | | | | | |
| 6 | α_{V}β₃ | | 10 | | 4 | | | |
| | cRGD_5′ | | | | | | | |
| | monovalent | | | | | | | |
| | AD-1634440 | | | | | | | |
| 7 | α_{V}β₃ | | 1 | | 4 | | | |
| | cRGD_5′ | | | | | | | |
| | trivalent | | | | | | | |
| | AD-1634441 | | | | | | | |
| 8 | α_{V}β₃ | | 10 | | 4 | | | |
| | cRGD_5′ | | | | | | | |
| | trivalent | | | | | | | |
| | AD-1634441 | | | | | | | |
| 9 | α_{V}β₃ | | 1 | | 4 | | | |
| | cRGD_3′ | | | | | | | |
| | (1 + 1) | | | | | | | |
| | bivalent | | | | | | | |
| | AD-1634442 | | | | | | | |
| 10 | α_{V}β₃ | | 10 | | 4 | | | |
| | cRGD_3′ | | | | | | | |
| | (1 + 1) | | | | | | | |
| | bivalent | | | | | | | |
| | AD-1634442 | | | | | | | |
| 11 | α_{V}β₃ | | 1 | | 4 | | | |
| | cRGD_3′ | | | | | | | |
| | (internal | | | | | | | |
| | SS6) | | | | | | | |
| | monovalent | | | | | | | |
| | AD-1634443 | | | | | | | |
| 12 | α_{V}β₃ | | 10 | | 4 | | | |
| | cRGD_3′ | | | | | | | |
| | (internal | | | | | | | |
| | SS6) | | | | | | | |
| | monovalent | | | | | | | |
| | AD-1634443 | | | | | | | |
| 13 | α_{V}β₃ | | 1 | | 4 | | | |
| | cRGD_3′ | | | | | | | |
| | (internal | | | | | | | |
| | SS6) trivalent | | | | | | | |
| | AD-1634444 | | | | | | | |
| 14 | α_{V}β₃ | | 10 | | 4 | | | |
| | cRGD_3′ | | | | | | | |
| | (internal | | | | | | | |
| | SS6) trivalent | | | | | | | |
| | AD-1634444 | | | | | | | |

Dose response Cilingetide ligand SAR was evaluated at 10 μg/eye after a single rat IVT injection, and the samples were processed at day 14 post injection. The study design is summarized in Table 14.

TABLE 14
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | α_{V}β₃ | | 10 | | 4 | dose, | | |
| | cRGD_3′ | | | | | Day 0 | | |
| | monovalent | | | | | | | |
| | (BCN) | | | | | | | |
| | AD-1751755 | | | | | | | |
| 3 | α_{V}β₃ | | 10 | | 4 | | | |
| | Cilingetide_5′-3′ | | | | | | | |
| | bivalent | | | | | | | |
| | AD-1751756 | | | | | | | |
| 4 | α_{V}β₃ | | 10 | | 4 | | | |
| | cRGD_3′ | | | | | | | |
| | (1 + 1) | | | | | | | |
| | bivalent | | | | | | | |
| | AD-1751757 | | | | | | | |

cRGD bioconjugation SAR (maleimide vs BCN zide) was evaluated at 1 and 10 μg/eye after a single rat IVT injection, and the samples were processed at day 14 post injection. The study design is summarized in Table 15.

TABLE 15
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 24) | n | Study | Analysis |
| 1 | PBS | IVT | n/a | 5 μL/eye | 4 | Single | D 14 | qPCR |
| 2 | α_{V}β₃ | | 1 | | 4 | dose, | | |
| | cRGD_3′ | | | | | Day 0 | | |
| | monovalent | | | | | | | |
| | (maleimide) | | | | | | | |
| | AD-1488236 | | | | | | | |
| 3 | α_{V}β₃ | | 10 | | 4 | | | |
| | cRGD_3′ | | | | | | | |
| | monovalent | | | | | | | |
| | (maleimide) | | | | | | | |
| | AD-1488236 | | | | | | | |
| 4 | α_{V}β₃ | | 1 | | 4 | | | |
| | cRGD_3′ | | | | | | | |
| | monovalent | | | | | | | |
| | (BCN azide) | | | | | | | |
| | AD-1488236 | | | | | | | |
| 5 | α_{V}β₃ | | 10 | | 4 | | | |
| | cRGD_3′ | | | | | | | |
| | monovalent | | | | | | | |
| | (BCN azide) | | | | | | | |
| | AD-1488236 | | | | | | | |

cRGD 3′ mono was evaluated at 100 μg/eye after a single NHP IVT injection, and the samples were processed at day 64 post injection. The study design is summarized in Table 16. PGP-254JT2

TABLE 16
Study Design

| | | | | | Number of | Dose | | |
|---|---|---|---|---|---|---|---|---|
| | Test Article | Dose | Doses | Dose | Animals | Regime | End of | |
| Group | ID (MYOC) | Route | (μg) | Volume | (N = 8) | n | Study | Analysis |
| 1 | PBS | IVT | n/a | 50 μL/eye | 2 | Single | D 64 | qPCR and |
| 2 | α_{V}β₃ | | 100 | | 2 | dose, | | ELISA |
| | cRGD_3′ | | | | | Day 0 | | |
| | monovalent | | | | | | | |
| | (maleimide) | | | | | | | |
| | AD-822899 | | | | | | | |
| 3 | α_{V}β₃ | | 100 | | 2 | | | |
| | cRGD_3′ | | | | | | | |
| | monovalent | | | | | | | |
| | (maleimide) | | | | | | | |
| | AD-1646735 | | | | | | | |

Total RNA isolation using Qiagen mRNA Isolation Kit:

RNA was isolated from lysates using miRNAeasy 96 kit (Qiagen, catalog number 217084) according to manufacturer's instructions. RNA samples were finally eluted in 45 1d of water, QCd with Nanodrop™ Spectrophotometer (Thermo Scientific, USA) prior to Reverse transcription and qPCR steps.

Synthesis using ThermoFisher Scientific Superscript IV cDNA reverse transcription kit (ThermoFisher Scientific, Cat. No. 11756050):

Twenty μl of a ready to use master mix and 11 μl of H20 per reaction was added to RNA isolated above. Plates were sealed, mixed, and incubated on thermalcycler at 25° C. for 10 min, followed by 42° C. for 10 min and inactivation step at 85° C. for 5 min.

Real time PCR:

Briefly, two μl of cDNA and 10 μl Fast Advanced Mastermix (ThermoFisher Scientific A44359) are added to lx GAPDH probe (ThermoFisher Scientific, Catalog no. 4352338E for rat and Mf04392546_g1 for NHP) and 1x MYOC probe (ThermoFisher Scientific, Catalog no. Rn00578382_ml for rat and Mf04376873_ml for NHP) per well. Real time PCR was done in a QuantStudio5 Real Time PCR system (ThermoFisher Scientific). The mean level of MYOC mRNA was normalized to the mean level of GAPDH mRNA for each sample. Group mean values were then normalized to the mean value for the PBS treated group, to obtain the relative level of MYOC mRNA expression.

### Results

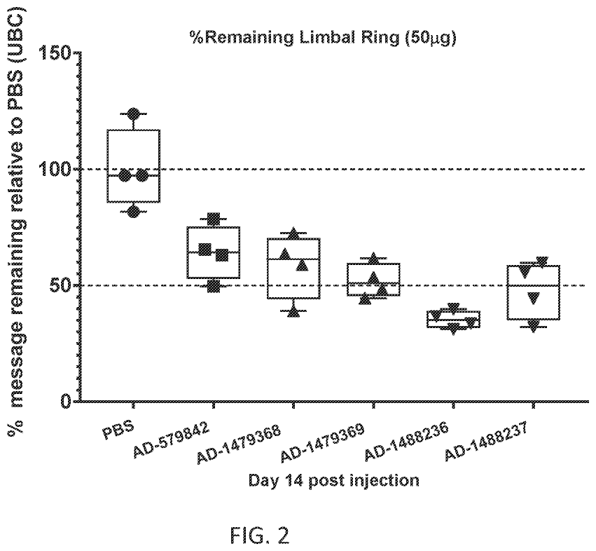

The percentage of MYOC mRNA remaining in the limbal ring (FIG. 2) was assessed fourteen days following administration of each of the indicated siRNA conjugates (50 μg). The results demonstrate a reduction of MYOC mRNA levels in the limbal ring of rats administered the MYOC RNAi conjugates relative to rats treated with PBS. Surprisingly, the siRNA conjugate including 3′ monovalent cyclic RGD (AD-1488236) resulted in the highest percent knockdown of MYOC mRNA in the limbal ring, showing an average knockdown (“KD”) of 68%, whereas siRNA conjugates including 3′ trivalent cyclic RGD (AD-1488237) resulted in an average of 57% KD. Further, siRNA conjugates including 3′ trivalent αvβ3 (AD-147369) resulted in an average of 54% KD, while siRNA conjugates including 3′ monovalent aᵥp3 (AD-1479368) had an average of 44% KD. In contrast, MYOC mRNA including internal C16 resulted in an average of 31% KD.

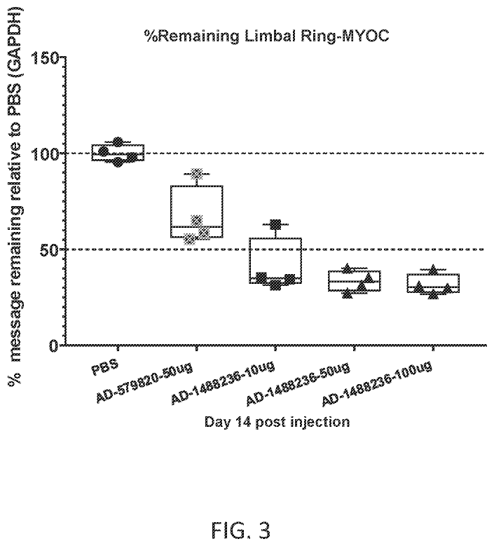

For the dose response experiment, the percentage of MYOC mRNA remaining in the limbal ring was assessed fourteen days following administration. FIG. 3 depicts the inhibition of MYOC expression by qPCR in the limbal ring (trabecular meshwork, iris and ciliary body) in a dose responsive manner.

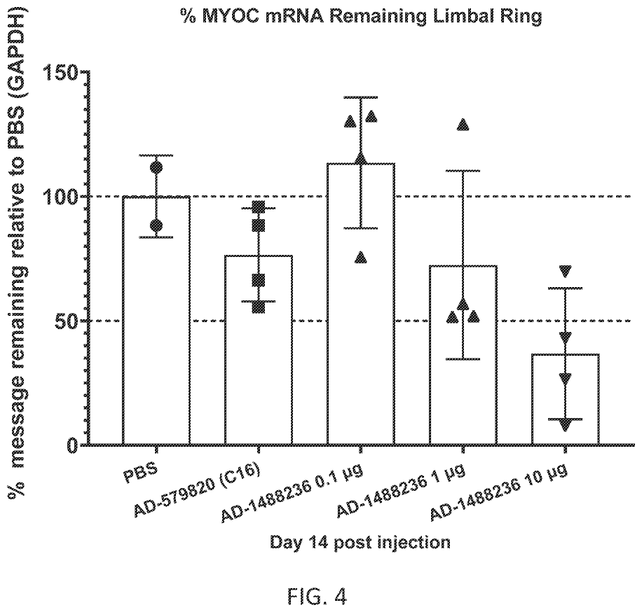

Follow up dose response experiment at lower doses showed a dose-dependent reduction of the percentage of MYOC mRNA transcript levels by qPCR in the limbal ring fourteen days following IVT administration. FIG. 4 depicts the inhibition of MYOC expression by qPCR in the limbal ring (trabecular meshwork, iris and ciliary body) down to 1 μg/eye dose with no MYOC mRNA lowering at the lowest dose tested 0.1 μg/eye.

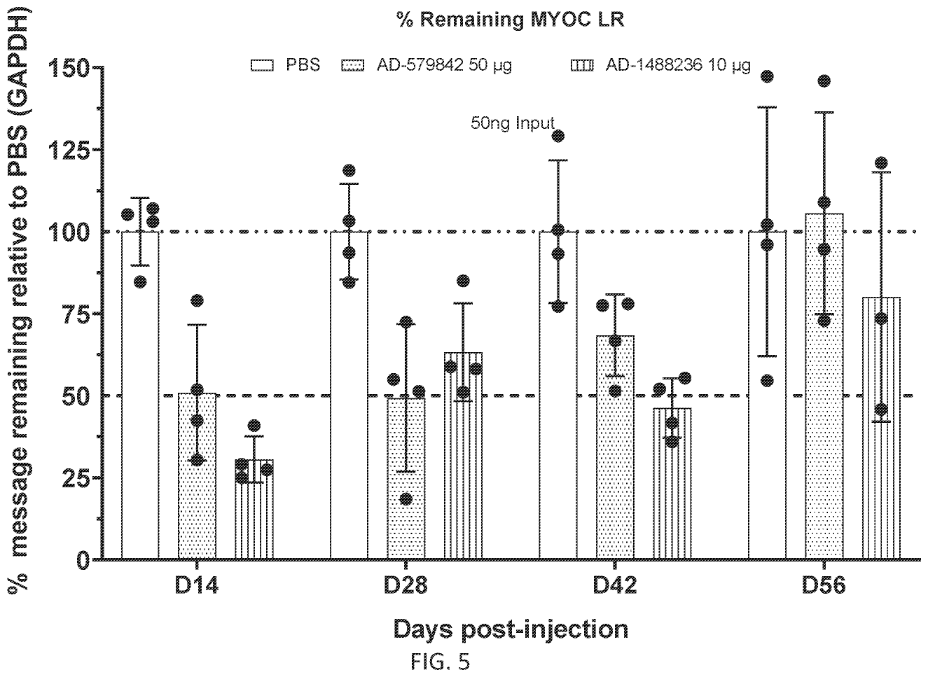

Next, we assessed durability of novel integrin targeting ligand cRGD 3′ monovalent siRNA conjugate compared to its parent duplex with internal C16 ligand at a dose 5x lower (10 versus 50 μg/eye). FIG. 5 demonstrates durable MYOC mRNA reduction with comparable efficacy to parent duplex at 115^{th} of the dose with recovery at day 56 post-single IVT dose administration.

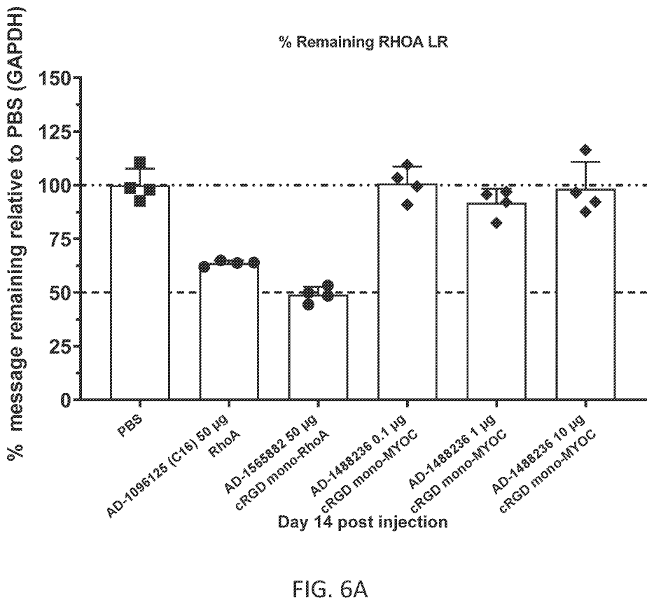

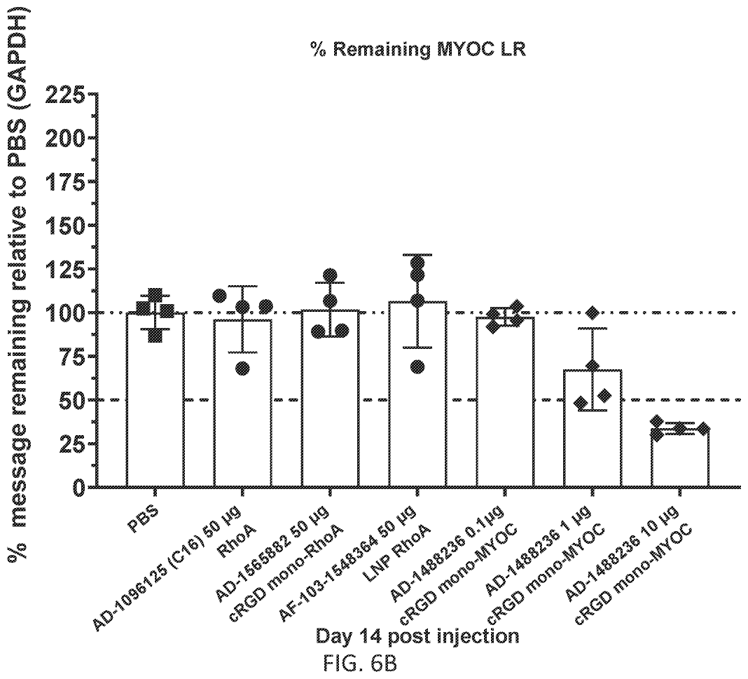

To determine plausibility of utilizing this strategy for other trabecular meshwork enriched targets we evaluated RhoA mRNA reduction by using either cRGD 3′ monovalent siRNA conjugates or parent internal C16 siRNA. FIG. 6A shows improved reduction of RhoA mRNA when siRNA was coupled with integrin targeting ligand. No effect on RhoA mRNA silencing was observed when animals were dosed with MYOC siRNA, desmonstrating target specific silencing. FIG. 6B shows the same observation in MYOC mRNA levels when animals were dosed with RhoA siRNAs (C16 and integrin targeting ligand 3′ monovalent cRGD) and also same dose response MYOC mRNA silencing was observed as in previous experiment with efficacious doses achieved as low as 1 μg/eye.

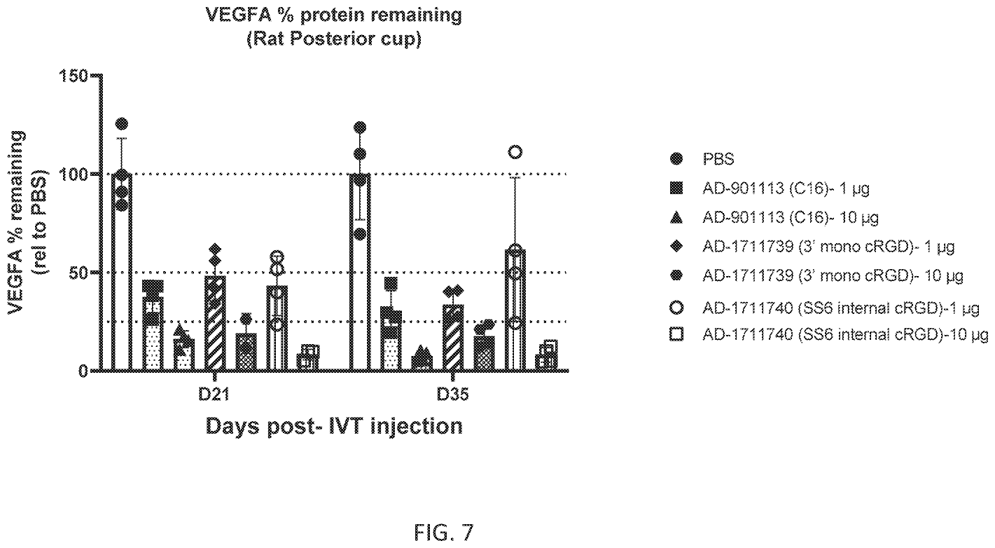

Evaluation of integrin targeting ligands for non-trabecular meshwork enriched targets was assessed next. Silencing of VEGFA, mainly expressed throughout the retina and RPE layers was assessed by measuring VEGFA protein KD via ELISA at days 21 and 35 post-IVT administration in rats. FIG. 7 shows slightly greater reduction of VEGFA protein with cRGD monovalent located in sense strand position 6 at day 21 compared to parent compound and similar activity (with 95% reduction) at day day 35 post-siRNA administration. This demonstrates that integrin targeting ligand conjugate strategies is amenable to other ocular expressed transcripts other than those mainly expressed in the limbal region area.

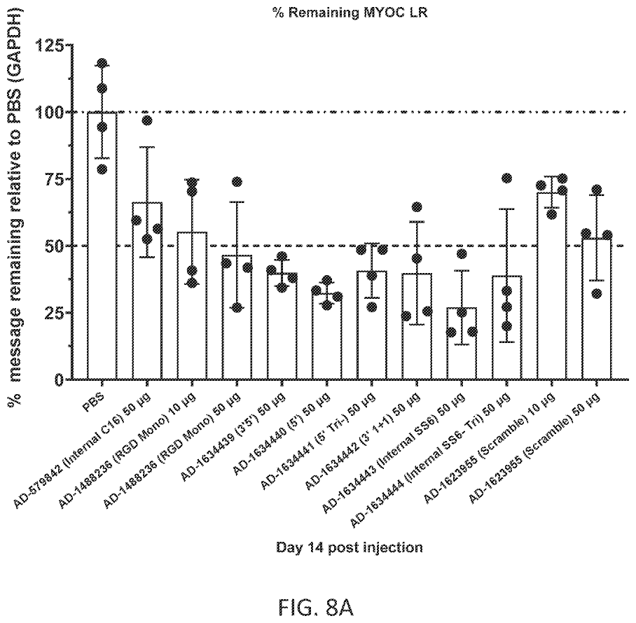

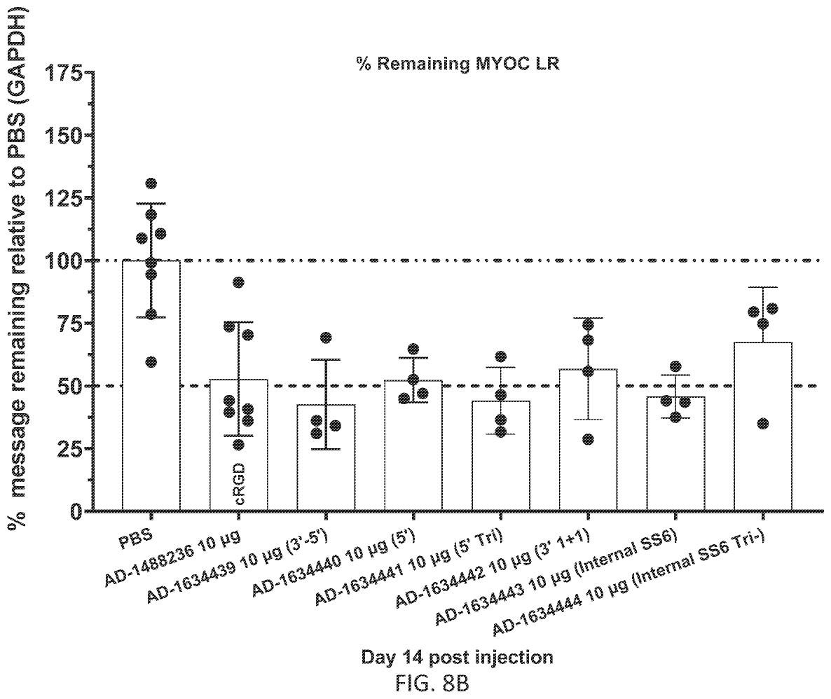

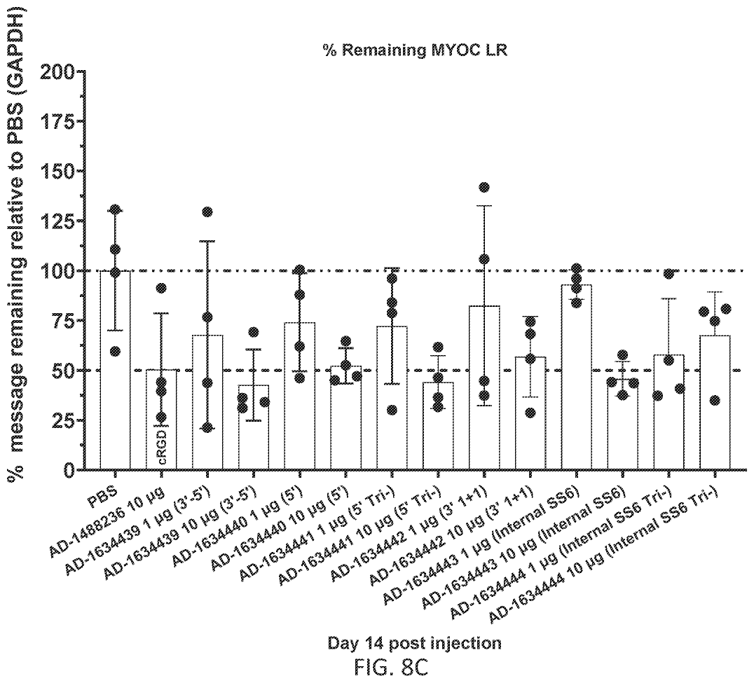

To determine best ligand orientation/configuration we performed a ligand SAR were cRGD was placed on positions 3′-5′ on its bivalent form, 5′ sense strand mono-valent, 5′ sense tri-valent, 3′ 1+1 bivalent and internal position 6 of sense strand as either mono and tri-valent forms. FIG. 8A summarizes the levels of MYOC mRNA silencing with the different ligand configurations. At high doses (50 μg/eye), placement of cRGD in the internal nucleobase seemed to increase the potency compared to other designs. However, when followed up by a lower dose study (10 μg/eye) all new designs seemed comparable to cRGD 3′ monovalent in terms of extend of MYOC mRNA silencing as measured by qPCR (FIG. 8B). Next, in the same experiment we performed a dose response (1 vs 10 μg/eye) and again showed no clear benefit of any of the new SAR designs compared to 3′ monovalent cRGD (FIG. 8C).

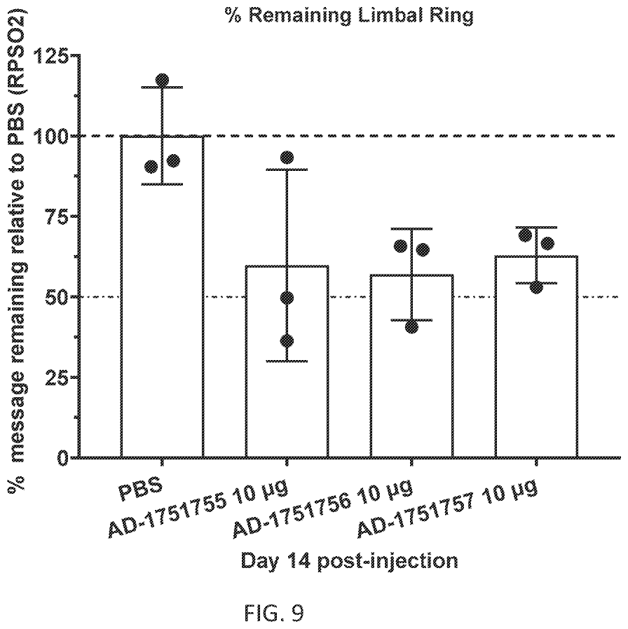

A small SAR evaluation of alternative integrin targeting ligands such as Cilingetide was also evaluated in rats, where animals were dosed with either 3′ monovalent form, 5′-3′ bivalent form or 3′ 1+1 bivalent form. None of the evaluated ligand configurations clearly outperformed Cillingetide 3′ mono valent design as shown in FIG. 9.

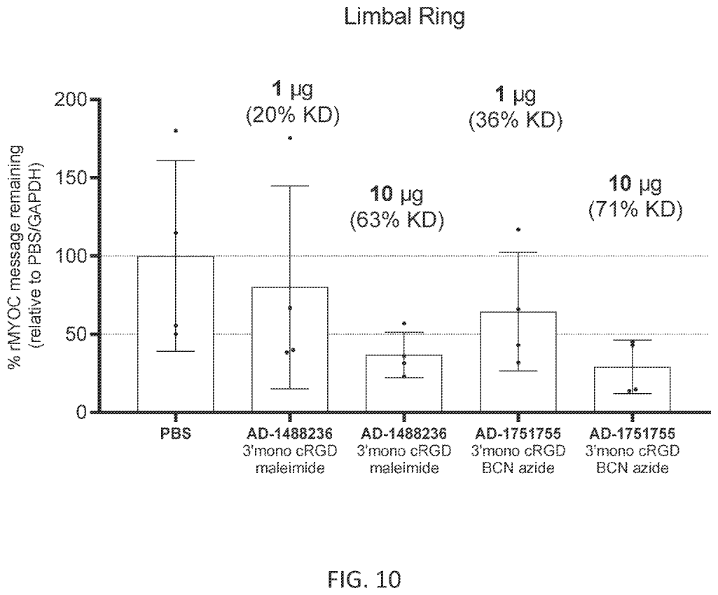

Effect of siRNA biconjugation strategy to cRGD 3′ monovalent ligand on in vivo efficacy was evaluated by dosing at 1 and 10 μg/eye siRNAs conjugated with either maleimide linker or BCN azide. As summarized in FIG. 10 no effect on in vivo potency was observed when a different bioconjugation was used.

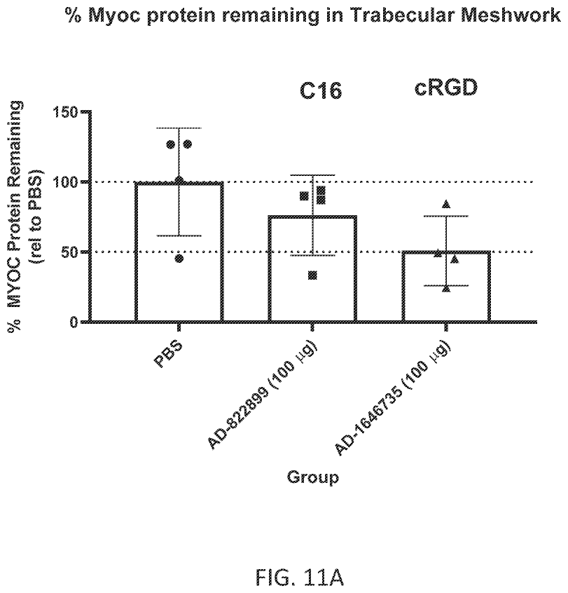

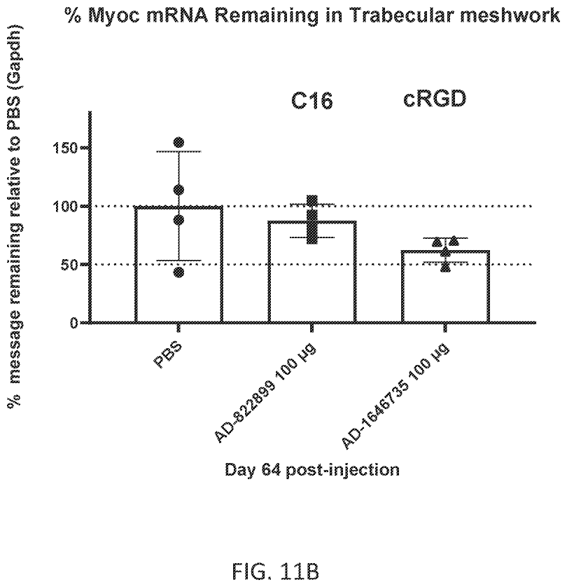

Translatability of rodent findings to non human primates was assessed by measuring MYOC mRNA and protein levels in trabecular meshwork cells dissected out from limbal ring 64 days post IVT administration. As shown in FIGS. 11A and 11B, cRGD 3′ monovalent conjugate outperformed internal C16 ligand at both protein and mRNA levels by an increase in 30% KD.

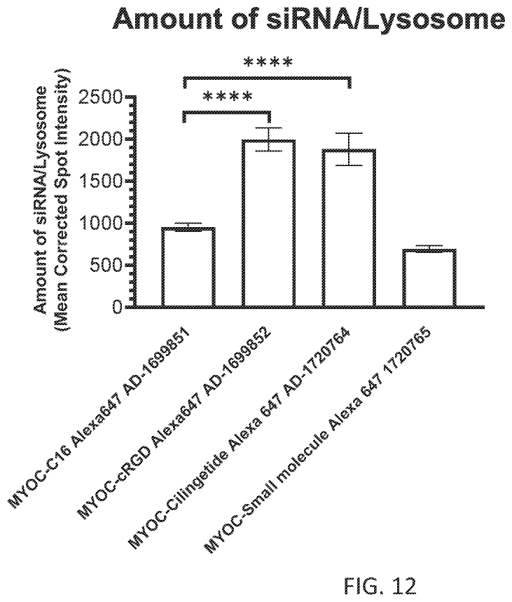

In vitro experiments were also conducted to understand integrin targeting ligand siRNA conjugates and its parent C16 duplex conjugated to Alexa 647 fluorophore. Duplexes were added to iPSC RPE culture and fluorescent intensity was measured at day 3 post siRNA addition to cultured cells inside the lysosome. As despicted in FIG. 12, greater uptake of both cRGD and cilingetide 3′ monovalent conjugates was observed compared to small molecule ligand and parent duplex with internal C16 ligand.

1. A method of inhibiting the expression of a target gene in an ocular cell or tissue comprising providing to the ocular cell or tissue an iRNA agent comprising a sense strand and an antisense strand, wherein at least one of the strands is conjugated to at least one integrin targeting ligand.

2. The method of claim 1, wherein the integrin targeting ligand is selected from the group consisting of RGD peptide ligands, RGD peptide mimetics, fibronectin, collagen, vitronectin, laminin, fibrinogen, thrombospondin, glycoproteins (e.g., tenascin C, osteopontin, and nefronectin), an RGD peptide ligand, a cyclic RGD peptide ligand, a monovalent cyclic RGD peptide ligand, a bivalent cyclic-RGD peptide, a trivalent cyclic-RGD peptide, a tetravalent cyclic-RGD peptide, Cilengitide, and tetrahydronaphthyridinyl nonanoic acid derivatives.

3. The method of claim 2, wherein the integrin targeting ligand is selected from the group consisting of

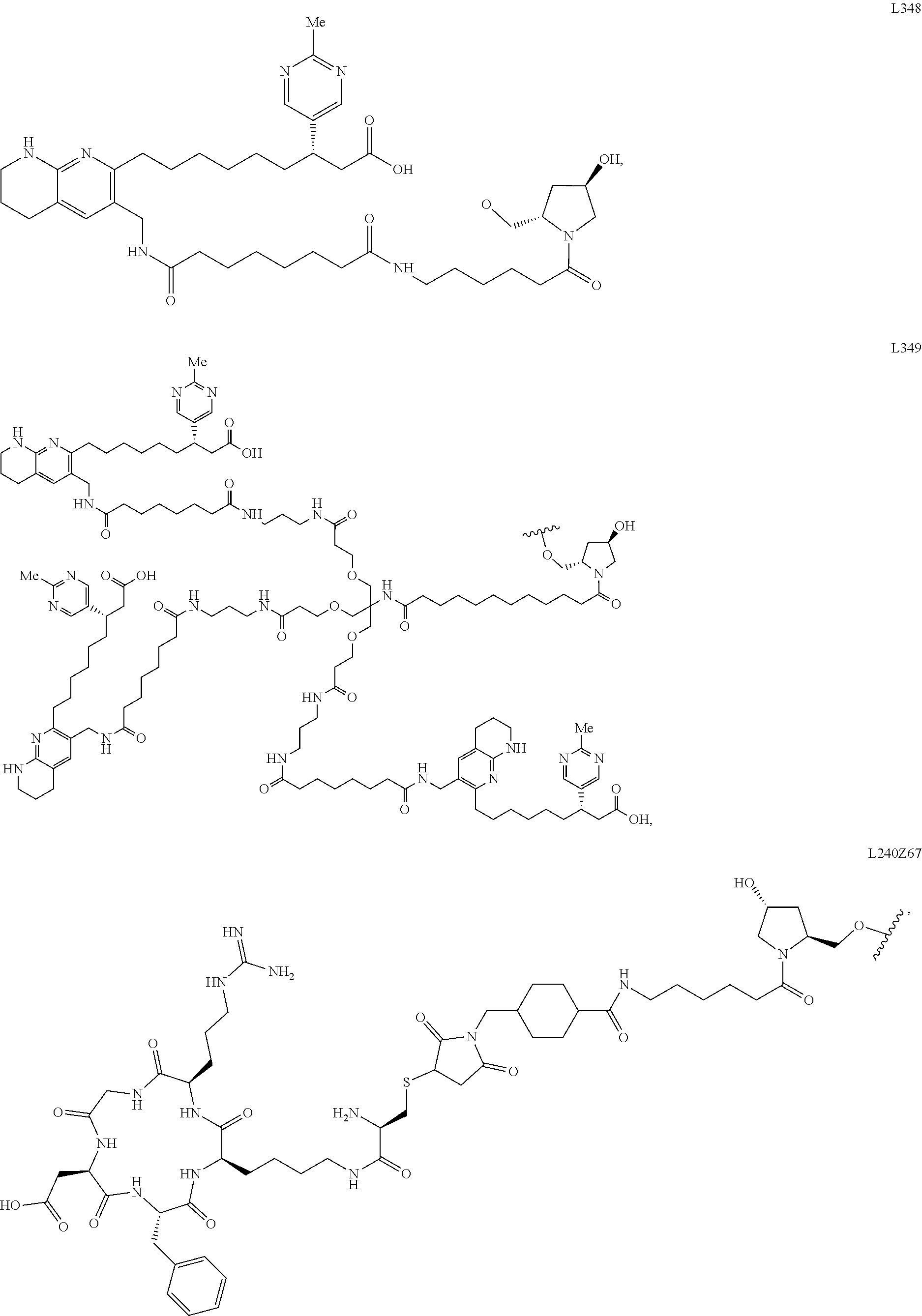

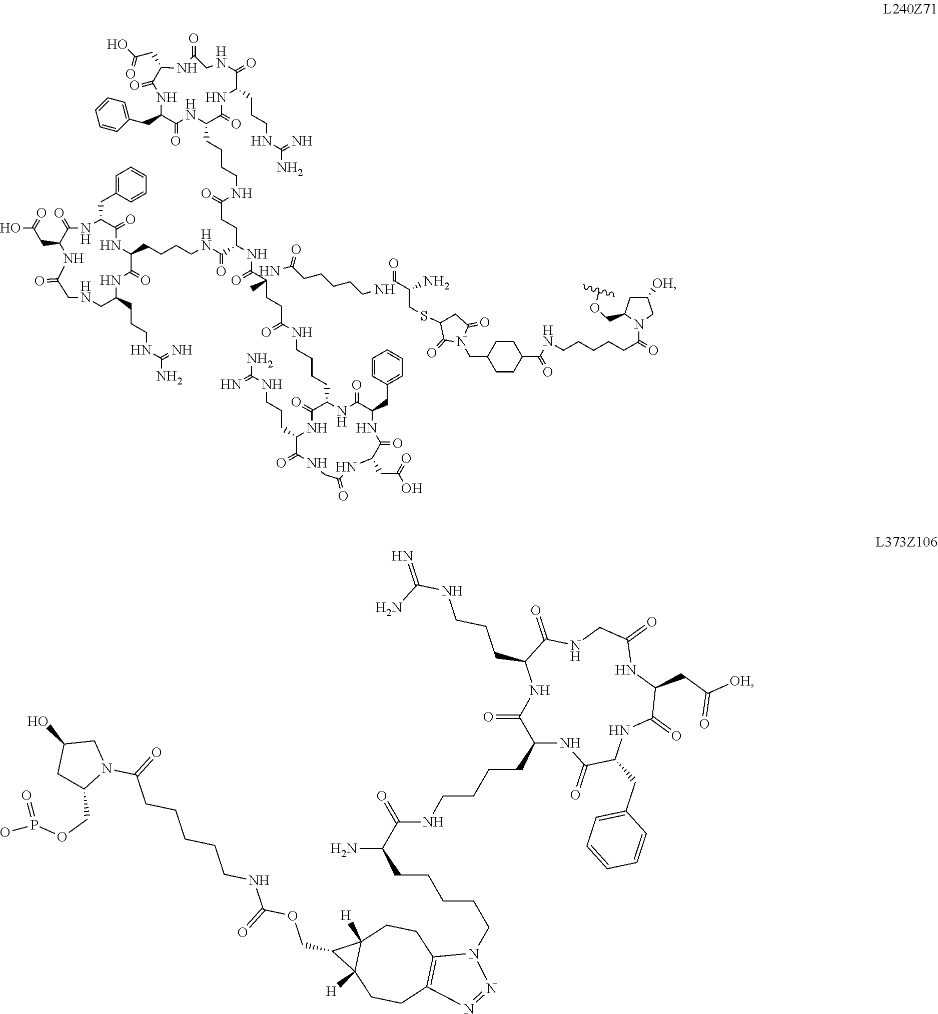

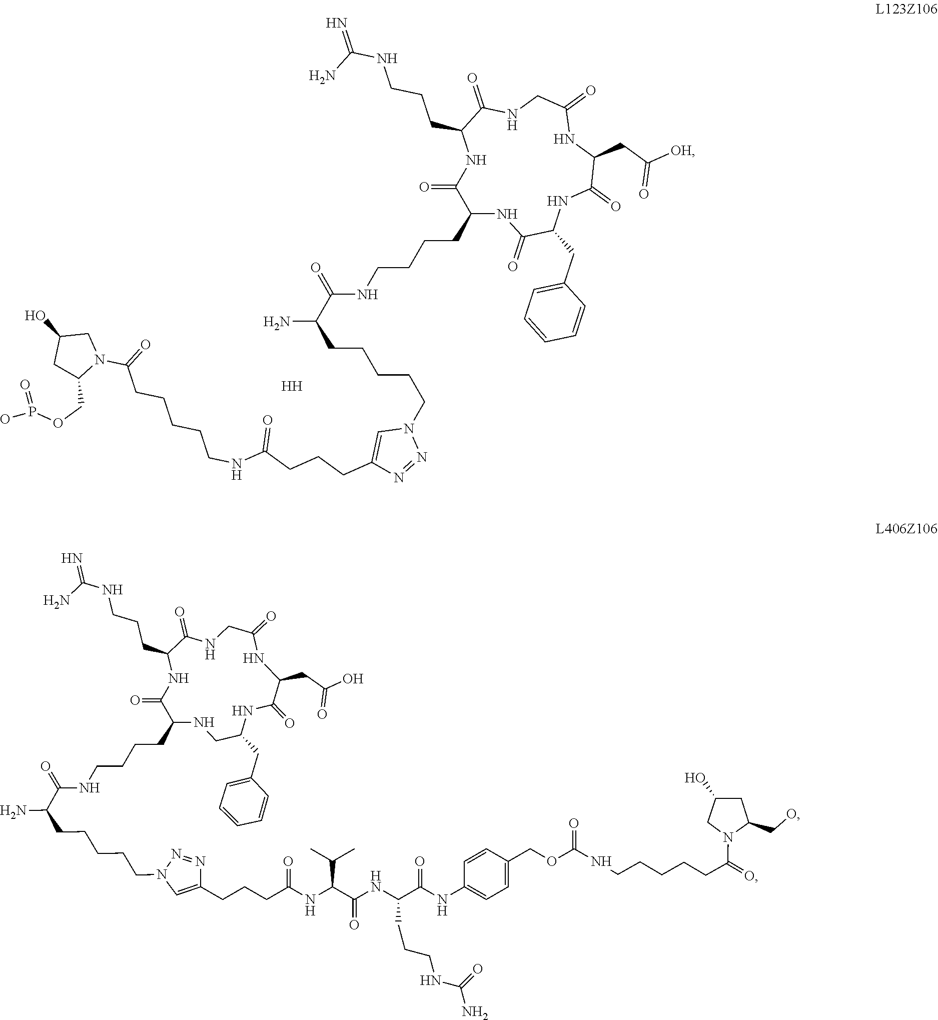

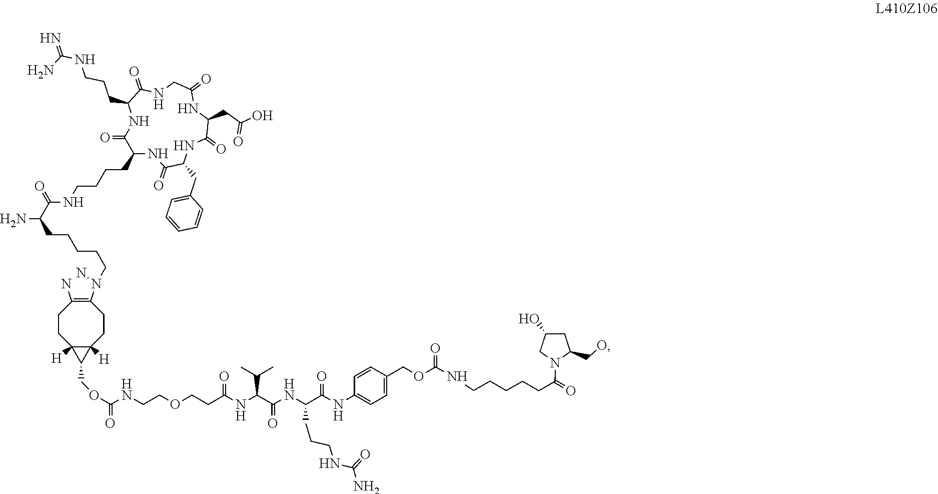

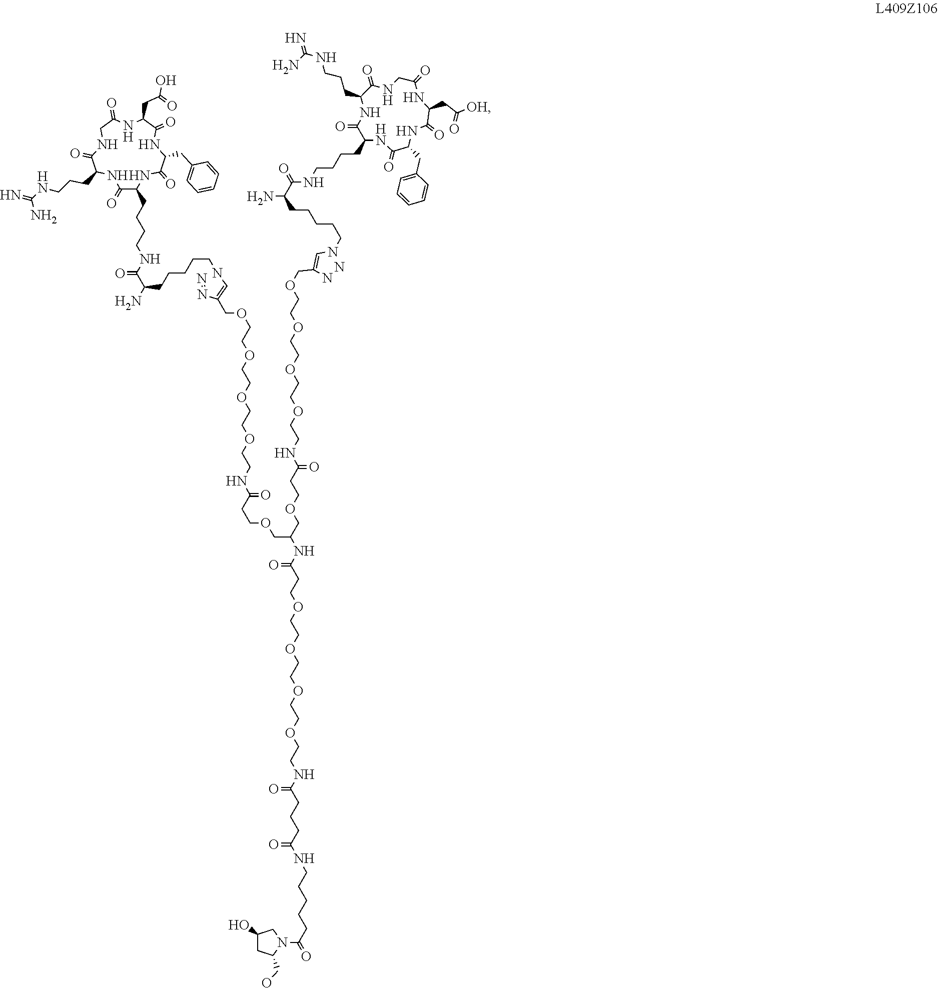 [SMILES: N=C(N)NCCC[C@@H]1NC(=O)[C@H](CCCCNC(=O)[C@H](N)CCCCCn2cc(COCCOCCOCCOCCNC(=O)CCOCC(COCCC(=O)NCCOCCOCCOCCOCc3cn(CCCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)nn3)NC(=O)CCOCCOCCOCCOCCNC(=O)CCCC(=O)NCCCCCC(=O)N3C[C@H](O)C[C@H]3CO)nn2)NC(=O)[C@@H](Cc2ccccc2)NC(=O)[C@H](CC(=O)O)NC(=O)CNC1=O]

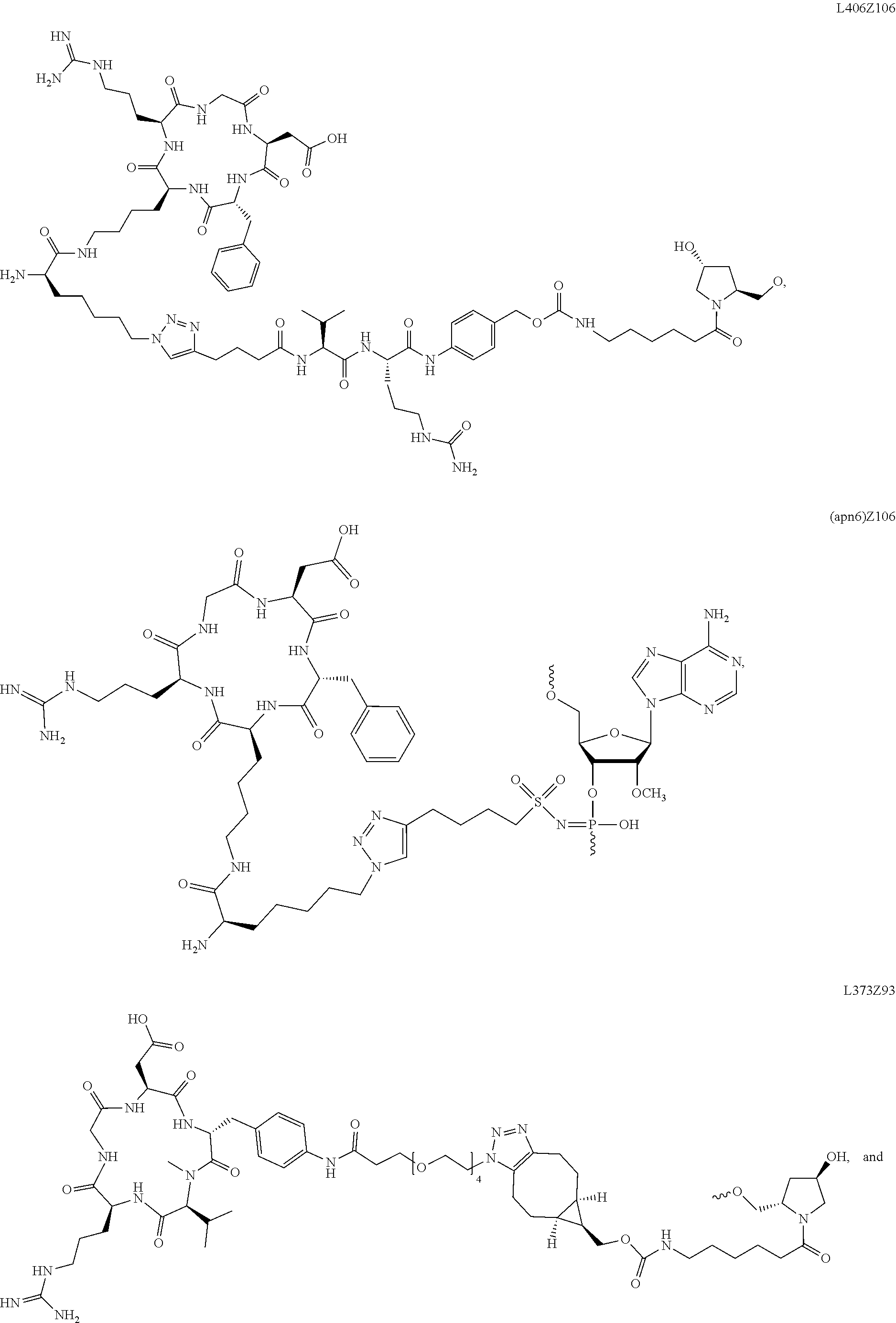

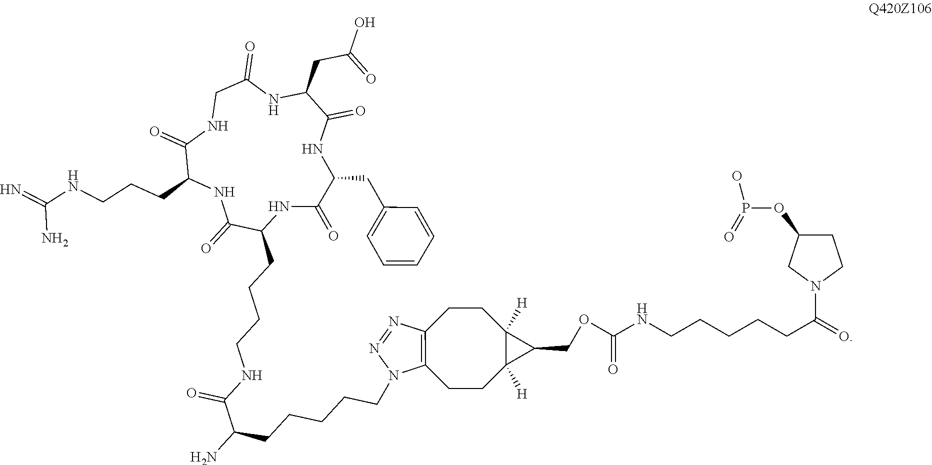

4. The method of any one of claims 1-3, wherein the iRNA agent is conjugated to the integrin targeting ligand by maleimide-thiol conjugation.

5. The method of any one of claims 1-3, wherein the iRNA agent is conjugated to the integrin targeting ligand by Cu(I)-free click conjugation.

6. The method of any one of claims 1-3, wherein the iRNA agent is conjugated to the integrin targeting ligand by Cu(I)-catalyzed click conjugation.

7. The method of any one of claims 1-3, wherein the iRNA agent is conjugated to the integrin targeting ligand by amide coupling conjugation.

8. The method of any one of claims 1-3, wherein the iRNA agent is conjugated to the integrin targeting ligand by thioether conjugation.

9. The method of any one of claims 1-3, wherein the iRNA agent is conjugated to the integrin targeting ligand by oxime conjugation.

10. The method of any one of claims 1-3, wherein the iRNA agent is conjugated to the integrin targeting ligand by TCO/methyltetrazine (Mtz) peptide conjugation.

11. The method of any one of claims 1-3, wherein the iRNA agent and integrin targeting ligand conjugate comprises a cleavable cRGD-iRNA conjugate.

12. The method of any one of claims 1-3, wherein the iRNA agent and integrin targeting ligand conjugate comprises a divalent cRGD-iRNA conjugate.

13. The method of any one of claims 1-3, wherein the iRNA agent and integrin targeting ligand conjugate comprises an internal cRGD-iRNA conjugate.

14. The method of any one of claims 1-13, wherein the integrin targeting ligand is incorporated into the sense strand or anti-sense strand or both strands.

15. The method of claim 14, wherein the integrin ligand is conjugated to an internal position or terminal position of both strands.

16. The method of any one of claims 1-15, wherein the target gene is selected from the group consisting of myocilin (MYOC), Ras homolog family member A (RhoA), vascular endothelial growth factor A (VEGFA), SSB (small RNA binding exonuclease protection factor La), optineurin, Carbonic Anhydrase 2 (CA2), Rho associated coiled-coil containing protein kinase 1 (ROCK1), Rho associated coiled-coil containing protein kinase 2 (ROCK2), Angiopoietin-Like 7 (ANGPTL7), and cytochrome P450 1B1 (CYP1B1).

17. The method of any one of claims 1-16, wherein the ocular cell or tissue is selected from the group consisting of an optic nerve cell, a trabecular meshwork cell, a Schlemm's canal cell, a juxtacanalicular tissue cell, a ciliary muscle cell, a retinal cell, an astrocyte, a pericyte, a Muller cell, a ganglion cell, an endothelial cell, a photoreceptor cell, a retinal blood vessel, episcleral veins or choroid tissue,, cornea, pupil, sclera, conjunctiva, optic nerve, iris, lens, aqueous humor, macula, optic disk, retina, ciliary muscle, vitreous humor, vitreous body, choroid, fovea, ciliary body, blood vessels, muscles (lateral rectus muscle, medial rectus muscle, ciliary muscle), ligaments (suspensory ligaments), anterior chamber, posterior chamber, limbal rings, and fovia.

18. A method of treating a subject having an ocular disorder comprising administering to the subject a therapeutically effective amount of an iRNA agent comprising a sense strand and an antisense strand, wherein at least one of the strands is conjugated to at least one integrin targeting ligand, and wherein the iRNA agent inhibits the expression of a target gene in an ocular cell or tissue.

19. The method of claim 18, wherein the subject is a human.

20. The method of claim 18 or 19, wherein the subject has been diagnosed with an ocular disorder selected from the group consisting of glaucoma, primary open angle glaucoma, secondary glaucoma, pigmentary glaucoma, pseudoexfoliative glaucoma, traumatic glaucoma, neovascular glaucoma, irido corneal endothelial syndrome, uveitic glaucoma, angle closure glaucoma, normal tension glaucoma, juvenile open angle glaucoma, primary open angle glaucoma, macular degeneration, cataracts, diabetic retinopathy, dry eyes, blurred vision, red eyes, blindness, night blindness, lazy eye, strabismus (cross eyes), nystagmus, colorblindness, uveitis, ocular inflammation, presbyopia, floaters in the field of vision, retinal disorders, retinal tear or detachment, conjunctivitis (pink eye), corneal diseases, vision changes, bulging eyes (proptosis), retinitis, diabetic macular edema, keratoconus, lazy eye, ocular hypertension, astigmatism, diabetic eye disease, hyperopia, myopia, macular edema, retinoblastoma, stargardt disease, usher syndrome, vitreous detachment, retinal disease, and cancers of the eye.

21. The method of claim 20, wherein the ocular disorder is glaucoma.

22. The method of any one of claims 19-21, wherein the integrin targeting ligand is selected from the group consisting of RGD peptide ligands, RGD peptide mimetics, fibronectin, collagen, vitronectin, laminin, fibrinogen, thrombospondin, glycoproteins (e.g., tenascin C, osteopontin, and nefronectin), an RGD peptide ligand, a cyclic RGD peptide ligand, a monovalent cyclic RGD peptide ligand, a bivalent cyclic-RGD peptide, a trivalent cyclic-RGD peptide, a tetravalent cyclic-RGD peptide, Cilengitide, and tetrahydronaphthyridinyl nonanoic acid derivatives.

23. The method of claim 22, wherein the integrin targeting ligand is selected from the group consisting of 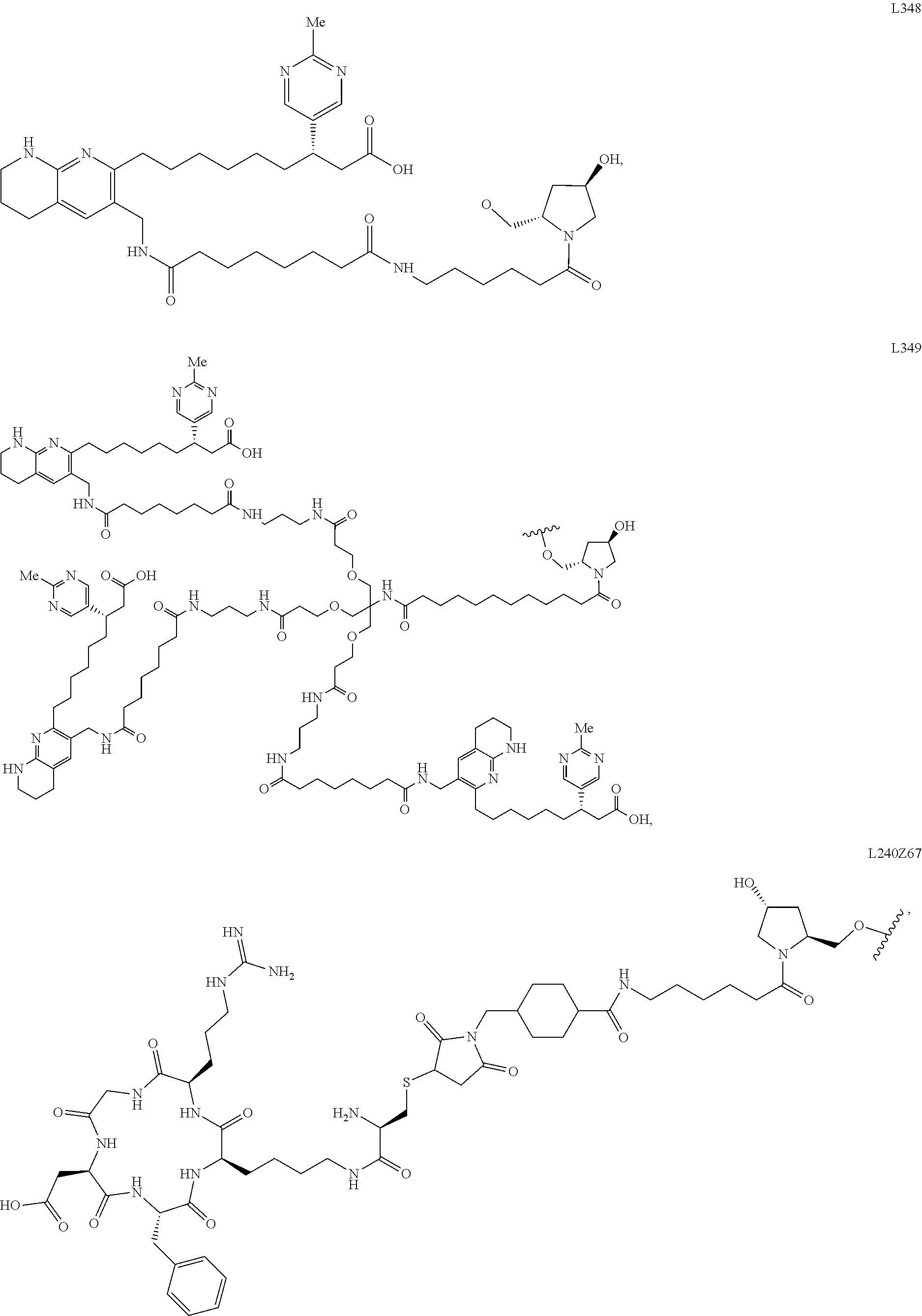 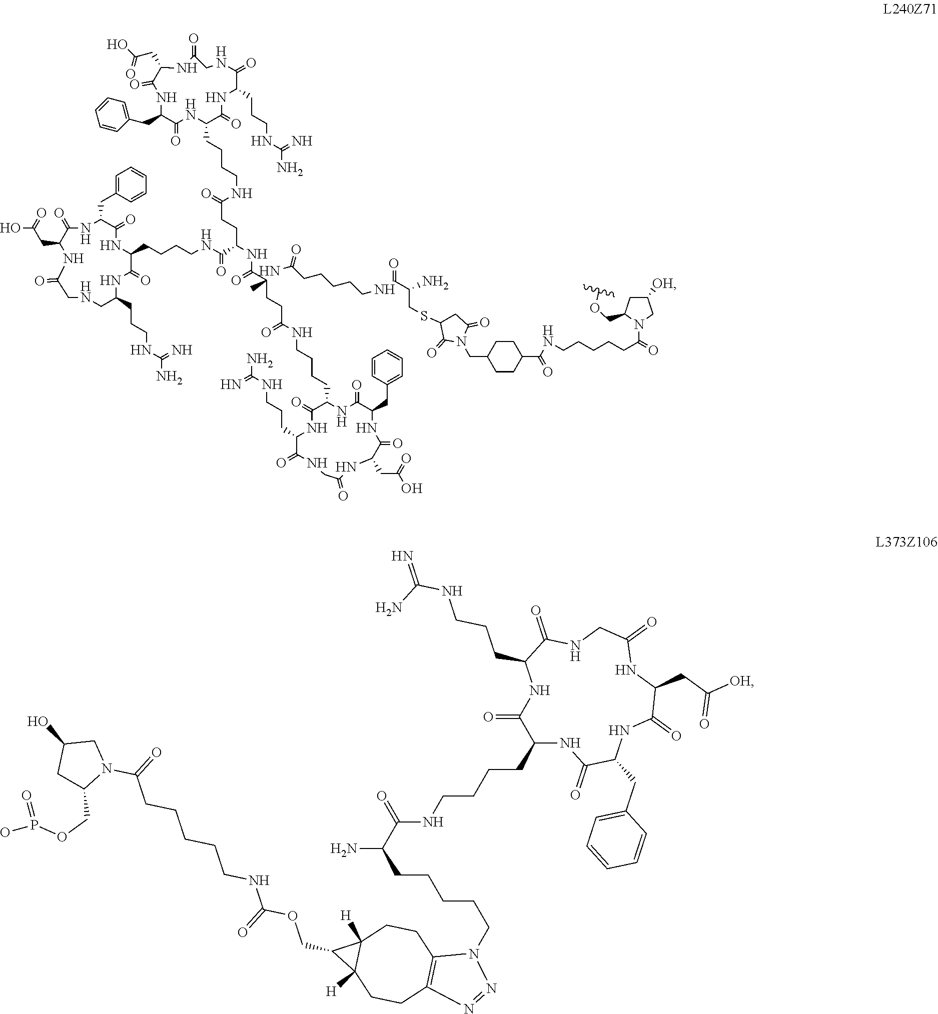 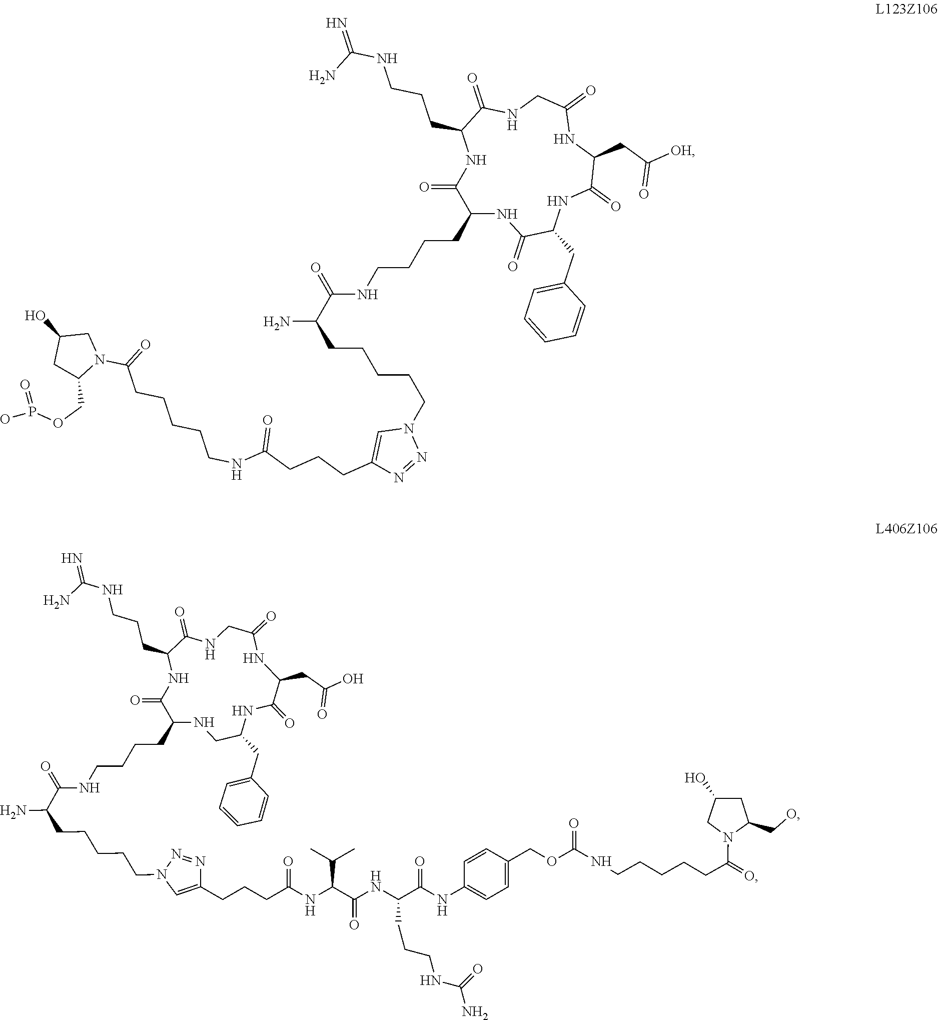 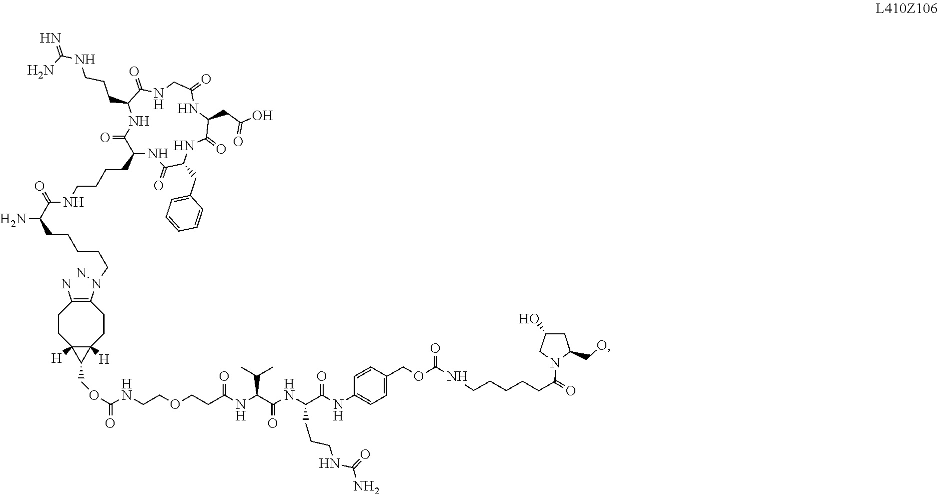 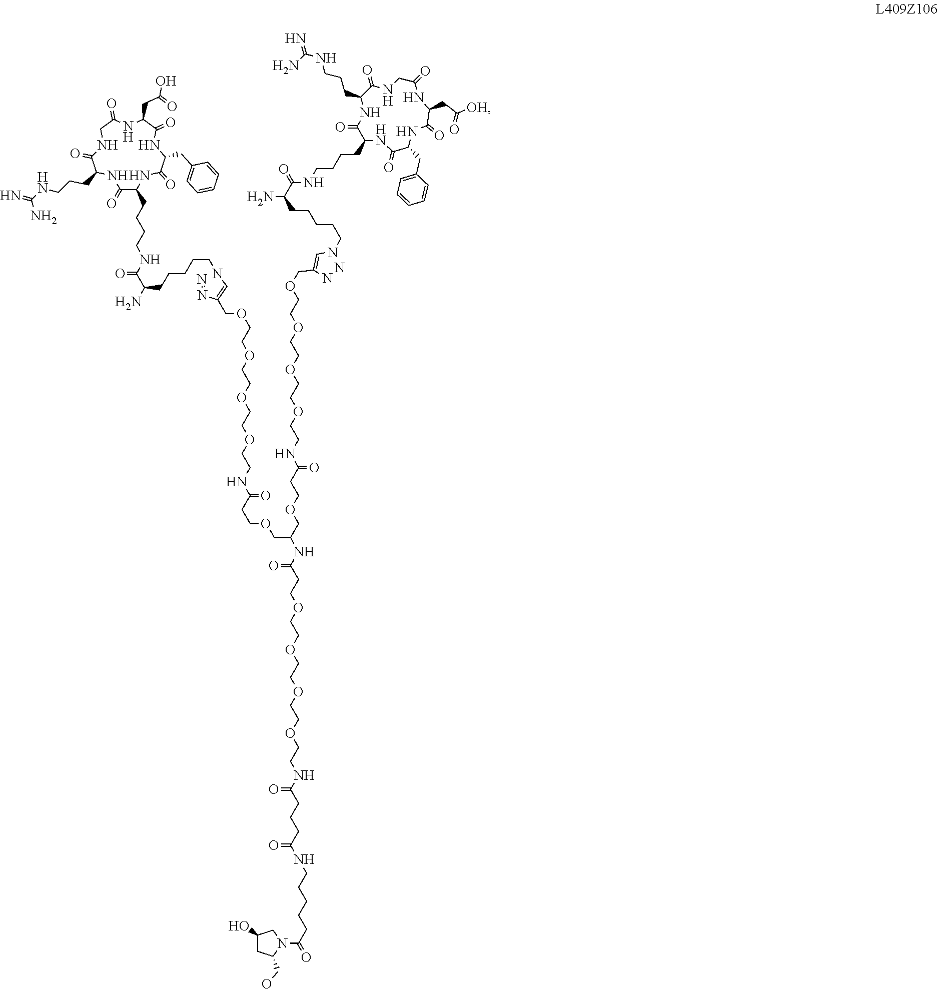 [SMILES: N=C(N)NCCC[C@@H]1NC(=O)[C@H](CCCCNC(=O)[C@H](N)CCCCCn2cc(COCCOCCOCCOCCNC(=O)CCOCC(COCCC(=O)NCCOCCOCCOCCOCc3cn(CCCCC[C@@H](N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)nn3)NC(=O)CCOCCOCCOCCOCCNC(=O)CCCC(=O)NCCCCCC(=O)N3C[C@H](O)C[C@H]3CO)nn2)NC(=O)[C@@H](Cc2ccccc2)NC(=O)[C@H](CC(=O)O)NC(=O)CNC1=O] 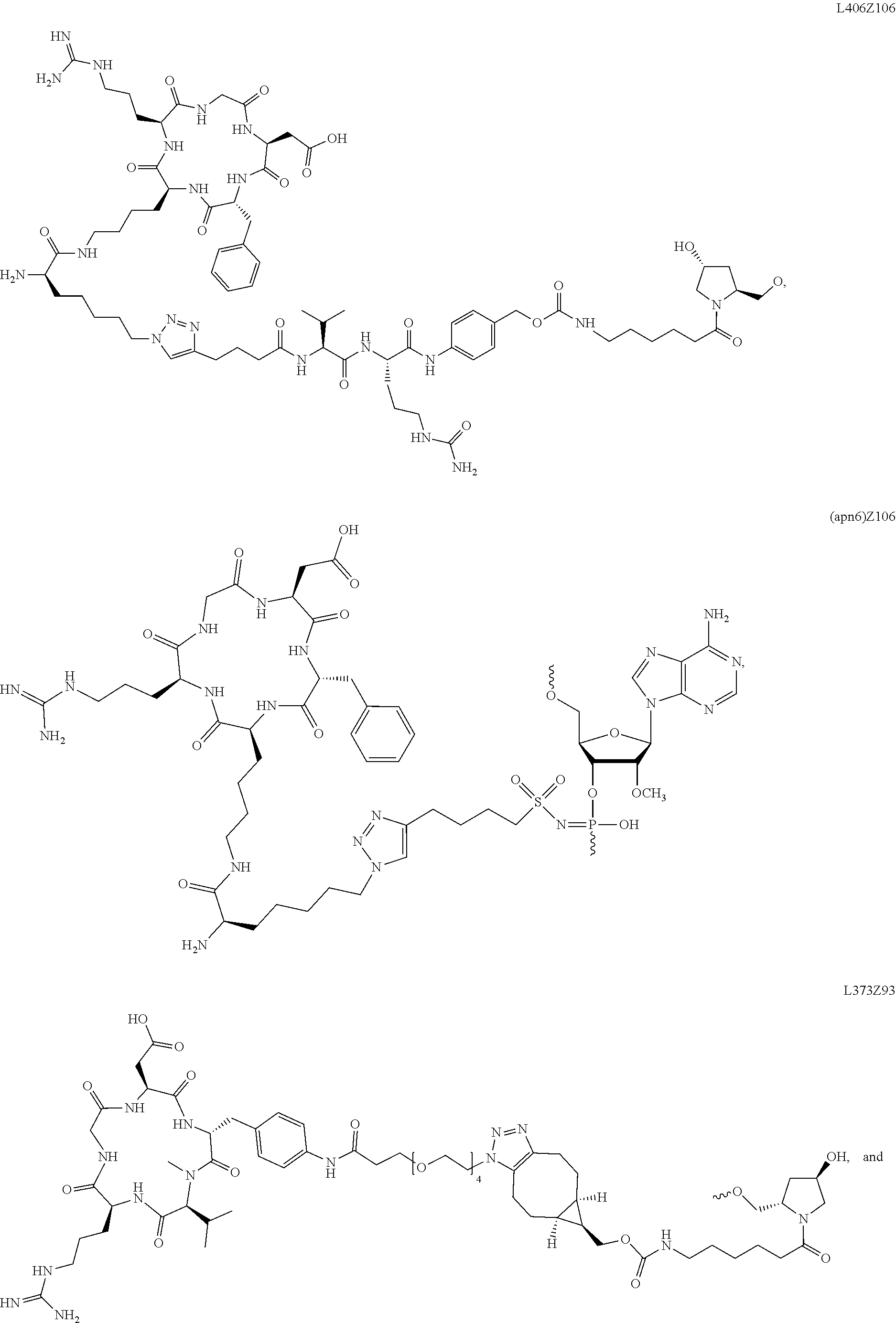 [chemical structure]
